(19) United States
(12) Patent Application Publication
(10) Pub. No.: US 2024/0401036 A1
(43) Pub. Date: Dec. 5, 2024

(54) THERAPEUTICS FOR THE TREATMENT OF NEURODEGENERATIVE DISORDERS

(71) Applicants: UCL BUSINESS LTD, London (GB); THE UNITED STATE OF AMERICA, AS REPRESENTED BY THE SECRETARY, DEPARMENT OF HEALTH AND HUMAN SERVICES, Bethesda, MD (US)

(72) Inventors: Pietro Fratta, London (GB); Anna Leigh Brown, London (GB); Oscar Wilkins, London (GB); Matthew Keuss, London (GB); Michael Ward, Bethesda, MD (US); Sarah Hill, Bethesda, MD (US)

(73) Assignees: UCL BUSINESS LTD, London (GB); THE UNITED STATE OF AMERICA, AS REPRESENTED BY THE SECRETARY, DEPARMENT OF HEALTH AND HUMAN SERVICES, Bethesda, MD (US)

(21) Appl. No.: 18/266,210

(22) PCT Filed: Dec. 9, 2021
(86) PCT No.: PCT/EP2021/084908

§ 371 Date: Jun. 8, 2023

(30) Foreign Application Priority Data
Dec. 9, 2020 (GB) 2019418.9

(51) Int. Cl. C12N 15/113

(52) U.S. Cl. CPC C12N 15/113; C12N 2310/11; C12N 2310/315; C12N 2310/321; C12N 2310/3231; C12N 2320/33

(57) ABSTRACT

Antisense oligonucleotides (ASOs) are provided which are capable of modulating splicing by preventing inclusion of an UNC13A cryptic exon into an UNC13A mature mRNA. Such ASOs may be used as a medicament, for example, to treat neurodegenerative disorders, particularly those associated with TDP-43 pathology.

### CROSS-REFERENCE TO RELATED APPLICATIONS

This application is a national stage application, filed under 35 U.S.C. § 371, of International Application No. PCT/EP2021/084908, filed Dec. 9, 2021, which claims the benefit of priority under 35 U.S.C. § 119 to GB Application No. 2019418.9, filed Dec. 9, 2020, each of which is incorporated herein by reference in its entirety.

### SEQUENCE LISTING

The present application is being filed along with a Sequence Listing in electronic format. The Sequence Listing file, entitled Sequence Listing 54127-505N01US-Corrected-ST25.txt, was created on Nov. 30, 2023, and is 189 kilobytes in size. The information in electronic format of the Sequence Listing is incorporated herein by reference in its entirety.

### FIELD OF THE DISCLOSURE

The present disclosure relates to novel therapeutics for the treatment of neurodegenerative disorders, more particularly, amyotrophic lateral sclerosis (ALS), and frontotemporal dementia (FTD) or those associated with TDP-43 pathology.

### BACKGROUND OF THE DISCLOSURE

Amyotrophic lateral sclerosis (ALS) and frontotemporal dementia (FTD) are two devastating adult-onset neurodegenerative diseases which can co-occur and are considered to be part of one disease spectrum, ALS/FTD. In ALS, neuronal loss affects primarily upper and lower motor neurons (MNs), leading to a rapidly progressive deterioration of muscle function and ultimately death due to respiratory failure. In FTD, frontal and temporal cortical neurons are preferentially lost, inducing cognitive impairment with language and behavioral changes.

The exact etiology of ALS is still largely unknown. Disease-causative genetic mutations are identified in <15% of cases, and a combination of a number of neuronal insults, with potential genetic, viral, autoimmune, and neurotoxic origins, are thought to underlie the remaining cases. Proposed mechanisms for the pathogenesis of ALS includes glutamate excitotoxicity, structural and functional abnormalities with the mitochondria, impaired axonal structure and transport defects, altered protein handling and free-radical mediated oxidative stress.

TDP-43 is an RNA-binding protein that is normally prevalently located in the nucleus of cells but is depleted from the nucleus and accumulated in cytoplasmic inclusions in a number of neurodegenerative disorders, including >95% of amyotrophic lateral sclerosis (ALS) cases, approximately 50% of frontotemporal dementia (FTD) cases, approximately 30% of Alzheimer disease cases, Parkinson Disease and other rare neurodegenerative disorders. TDP-43 participates to numerous RNA processing functions, including repressing the inclusion of unwanted intronic RNA sequences in mature mRNAs.

When TDP-43 is lacking from the nucleus, it cannot perform this repressive action and spurious segments of introns, named “cryptic exons” are included in mRNAs. Cryptic exons may induce a reduction of protein levels either through aberrant RNA degradation or due to the presence of premature stop codons.

There is a need to further understand the mechanisms of TDP-43 depletion and TDP-43 pathology and how this leads to the onset of disease. There is also a need to develop therapeutic strategies to treat neurodegenerative disorders and/or diseases associated with TDP-43 depletion.

### SUMMARY OF THE DISCLOSURE

This summary provides varied embodiments of the subject matter of the present disclosure, whereby the details, examples and preferences provided in relation to one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

The present invention solves the problem of further understanding the mechanisms of TDP-43 depletion and TDP-43 pathology and how this leads to the onset of disease. Provided herein are polynucleotides, i.e., UNC13A antisense compounds, which are useful in the elucidation of TDP-43 pathology and/or UNC13A dysfunction and their role in disease. The present invention also solves the problem of the provision of alternative therapeutic strategies to treat neurodegenerative disorders and/or diseases associated with TDP-43 depletion as the UNC13A ASOs described herein are surprisingly found to prevent the splicing machinery from recognizing the cryptic exon arising from the diseased state and from incorporating the cryptic exon into the resulting UNC13A mRNA. The UNC13A antisense compounds in their effect thereby lead to a rescue effect in the disease state.

According to a first aspect, there is provided an antisense oligonucleotide (ASO) comprising a nucleotide sequence of 13-30 nucleotides, wherein the nucleotide sequence has sequence complementarity with SEQ ID NO 1 (i.e., a portion of SEQ ID NO 1 sequence having the same number of nucleotides). In an embodiment, the nucleotide sequence is 100% complementary with SEQ ID NO 1. In some embodiments, the ASO is complementarity to SEQ ID NO: 1 wherein the base at position 415 is G or C. In some embodiments, the ASO is complementary to SEQ ID NO: 1 wherein the base at position 965 is U or G. In some embodiments, SEQ ID NO: 1 has a G at position 415 and a U at position 965. In some embodiments, SEQ ID NO: 1 has a G at position 415 and a G at position 965. In some embodiments, SEQ ID NO: 1 as a C at position 415 and a U at position 965. In some embodiments, SEQ ID NO: 1 has a C at position 415 and a G at position 965. The ASO is synthetic.

In an embodiment, the ASO is capable of modulating splicing by preventing inclusion of an UNC13A cryptic exon into an UNC13A mature mRNA.

According to a second aspect of the present invention, there is provided an antisense oligonucleotide (ASO) comprising a nucleotide sequence of 13-30 nucleotides, wherein the ASO is capable of modulating splicing by preventing inclusion of an UNC13A cryptic exon into an UNC13A mature mRNA. The ASO may be synthetic.

In an embodiment of the first and second aspect, the ASO comprises 20-24 nucleotides, preferably 21 or 22 nucleotides, preferably 21 nucleotides.

In an embodiment of the first and second aspect, the ASO is capable of binding to a UNC13A cryptic exon or flanking region thereof. In some embodiments, the ASO is complementarity to SEQ ID NO: 1 wherein the base at position 415 is G or C. In some embodiments, the ASO is complementary to SEQ ID NO: 1 wherein the base at position 965 is U or G. In an embodiment, the ASO comprises a sequence corresponding to any one or more of SEQ ID NO 4-546.

In an embodiment, the ASO is capable of binding to a UNC13A cryptic exon, the UNC13A cryptic exon occurring in UNC13A pre-mRNA.

In an embodiment, the UNC13A cryptic exon corresponds to SEQ ID NO 2 or SEQ ID NO 3. In an embodiment, the ASO is complementary to SEQ ID NO 2 or SEQ ID NO 3. In SEQ ID NO: 2 the base at position 112 can be either G or C. In SEQ ID NO: 3 the base at position 162 can be either G or C.

In an embodiment of the first and second aspect, the ASO is capable of binding to a branchpoint of the UNC13A cryptic exon. In an embodiment, the ASO a sequence corresponding to any one or more of SEQ ID 4-104. These ASOs can target the branchpoint such that splicing is less efficient.

In an embodiment of the first and second aspect, the ASO is capable of binding to a splice site of the UNC13A cryptic exon. In an embodiment, the ASO comprises a sequence corresponding to any one or more of SEQ ID 105-189, or SEQ ID NO: 270-352. These ASOs can target the splice sites such that the splice sites are less available for splicing.

In an embodiment of the first and second aspect, the ASO is capable of binding to a splice regulatory element (SRE) associated with the UNC13A cryptic exon in the UNC13A pre-mRNA. In an embodiment, the ASO is capable of binding to i) the cryptic exon, ii) an SNP in the UNC13A intron, iii) a TDP-43 binding site or iv) a splice enhancer. In an embodiment, the ASO comprises a sequence corresponding to i) SEQ ID NO: 190-269 and is capable of binding to the cryptic exon, ii) SEQ ID NO: 353-426 and is capable of binding to a SNP in the UNC13A intron, iii) SEQ ID NO: 427-474 and is capable of binding to a TDP-43 binding site within an UNC13A pre-mRNA, or iv) SEQ ID NO:475-546 and is capable of binding to a splice enhancer associated with UNC13A cryptic exon. The ASOs of the present invention can target these splicing regulatory elements in order to limit the binding of RNA binding proteins that enhance/modulate the inclusion of the cryptic exon.

The ASO is synthetic and is preferably chemically modified (i.e., wherein the ASO comprises one or more modified nucleotides). In an embodiment, the nucleotides may have phosphate (i.e., phosphodiester), phosphorothioate or phosphorodiamidate linkages. In an embodiment of the first and second aspect, the ASO comprises locked, bridged or constrained nucleic acids. In an embodiment of the first and second aspect, the ASO has or comprises a backbone selected from RNA, DNA, LNA (locked nucleic acid), tcDNA (tri-cyclo DNA), HNA (hexitol nucleic acids), TNA (threose nucleic acid), morpholino oligomer (PMO), peptide nucleic acid (PNA), 2′-OMe-RNA, 2′-O,4′-C-Ethylene-bridged nucleic acid (ENA), 2′-O-methoxyethyl (MOE) nucleic acids, or 2-O-(2-methylcarbomoyl (MCE) nucleotides, or any combination thereof. The ASO may further comprise a portion of DNA nucleotides, for example, at least 10%, or at least 20%, or at least 30%, or at least 40%, or at least 50%, or at least 60%, or up to 70% of DNA nucleotides, i.e., in combination with LNA, tcDNA, cET, ENA, HNA, TNA, PMO, PNA, 2′-OMe-RNA, 2′-O-methoxyethyl (MOE) nucleic acids or MCE nucleotides. In some examples, disclosed herein, the ASO has a LNA or a 2-OMe-RNA backbone. In some examples, the ASO comprises from about 20-60% LNA, or preferably from about 30-50% LNA (i.e., wherein the other bases are chemically unmodified, e.g., formed from DNA). In some embodiments, the ASO comprises an LNA base at least every 3 nucleotides, or at least every 2 nucleotides. In some examples, the ASO comprises 100% 2-OMe-RNA.

According to a third aspect of the present invention, there is provided a guide RNA comprising the ASO of the first or second aspect and a scaffold sequence for a Cas nuclease. In an embodiment, the ASO has an RNA backbone. In an embodiment, the scaffold sequence is a scaffold sequence for a Cas13 nuclease.

According to a fourth aspect of the present invention, there is provided a viral vector comprising an ASO of the first or second aspect or a guide RNA of the third aspect. In an embodiment, the viral vector is a retrovirus, lentivirus, adenovirus, or adeno-associated virus.

According to a fifth aspect of the present invention, there is provided a pharmaceutical composition comprising one or more ASOs of the first aspect or second aspect, one or more guide RNAs of the third aspect, or one or more viral vectors of the fourth aspect. In an embodiment, the pharmaceutical composition comprises a pharmaceutical carrier, diluent or excipient. In an embodiment, the pharmaceutical composition comprises a polymer, liposomes, micelles, dendrimers, nanoparticles or a combination thereof.

According to a sixth aspect of the present invention, there is provided the ASO of the first or second aspect, the guide RNA of the third aspect, the viral vector of the fourth aspect or the pharmaceutical composition of the fifth aspect for use as a medicament.

According to a seventh aspect of the present invention, there is provided the ASO of the first or second aspect, the guide RNA of the third aspect, the viral vector of the fourth aspect or the pharmaceutical composition of the fifth aspect for use in a method of treating a neurodegenerative disorder. In an embodiment, the neurodegenerative disorder is associated with TDP-43 pathology. In an embodiment, the neurodegenerative disorder is ALS, frontotemporal dementia (FTD), Alzheimer's disease, Parkinson's disease, FOSMN, Perry Syndrome or any combination thereof. In an embodiment, the neurodegenerative disorder is ALS, wherein the ALS is familial ALS or sporadic ALS.

According to an eighth aspect of the present invention, there is provided a method of delivering to a cell the ASO of the first or second aspect, the guide RNA of the third aspect, the viral vector of the fourth aspect, or the pharmaceutical composition of the fifth aspect, wherein the method comprises contacting the ASO with a cell, wherein the ASO modulates splicing of UNC13A to prevent inclusion of a cryptic exon in UNC13A mature RNA. In an embodiment, this prevents loss of the UNC13A translated protein. In an embodiment, this restores functionality of the UNC13A protein. This may be an in vitro or an in vivo method.

Also disclosed herein is a method of treating a neurodegenerative disorder, the method comprising administering to a subject in need thereof a therapeutically effective amount of the ASO of the first or second aspect, the guide RNA of the third aspect, the viral vector of the fourth aspect or the pharmaceutical composition of the fifth aspect. Also disclosed herein is a method of treating a condition associated with TDP-43 pathology, the method comprising administering to a subject in need thereof a therapeutically effective amount of the ASO of the first or second aspect, the guide RNA of the third aspect, the viral vector of the fourth aspect or the pharmaceutical composition of the fifth aspect. In an example, the ASO, guide RNA, viral vector or pharmaceutical composition can be used to prevent loss of and/or restore functionality of the UNC13A protein. In an example, the ASO, guide RNA, viral vector or pharmaceutical composition can be used to modulate splicing such that the UNC13A cryptic exon is not included in the UNC13A mature RNA.

Also disclosed herein, is the use of the ASO of the first or second aspect, the guide RNA of the third aspect, the viral vector of the fourth aspect or the pharmaceutical composition of the fifth aspect, for the manufacture of a medicament. The medicament may be used to treat a neurodegenerative disorder and/or a disorder associated with TDP-pathology as is otherwise described herein.

Also disclosed herein is an ASO comprising a nucleotide sequence of 13-30 nucleotides, wherein the ASO is used in a method to prevent inclusion of an UNC13A cryptic exon into the UNC13A mature RNA.

Also disclosed herein is an ASO comprising a nucleotide sequence of 13-30 nucleotides, wherein the ASO (or at least a portion thereof) comprises a sequence corresponding to SEQ ID NO 4-546. As described elsewhere herein, U can be interchanged with T in SEQ ID NO 4-546. It is also intended that these sequences may have any nucleotide chemistry.

Also disclosed herein is an ASO according to any one of SEQ ID NO: 555-571 or SEQ ID NO: 579 or 580. In these sequences, U and T are interchangeable. It is also intended that these sequences may have any nucleotide chemistry.

Also disclosed herein is a method of modulating UNC13A splicing in a subject, the method comprising administering to a subject in need thereof an effective amount of the ASO of the first or second aspect, the guide RNA of the third aspect, the viral vector of the fourth aspect or the pharmaceutical composition of the fifth aspect. In an example, the ASO, guide RNA, viral vector or pharmaceutical composition can be used to prevent loss of and/or restore functionality of the UNC13A protein. In an example, the ASO, guide RNA, viral vector or pharmaceutical composition can be used to modulate splicing such that the UNC13A cryptic exon is not included in the UNC13A mRNA.

Also disclosed herein is the isolated cryptic exon sequence of UNC13A and flanking regions thereof, corresponding to SEQ ID NO:1. SEQ ID NO: 1 corresponds to the pre-mRNA sequence to which the ASO is capable of binding. Also disclosed herein is the isolated cryptic exon sequences corresponding to SEQ ID NO: 2 (shorter variant) or SEQ ID NO:3 (longer variant). The cryptic exon sequence of UNC13A and flanking regions thereof may be used to probe TDP-43 pathology and/or UNC13A dysfunction and its role in disease.

The present inventors have discovered a previously unreported cryptic exon in UNC13A, a gene that encodes for a crucial synaptic protein: UNC13A. This novel cryptic exon is found to cause UNC13A downregulation at the transcript and protein level. Notably, the cryptic exon was detected specifically in patient postmortem brain regions affected by TDP-43 proteinopathy, including both ALS and FTD and was found to overlap with the disease-associated variant rs12973192 previously identified in multiple genome-wide association studies linked to ALS/FTLD risk, as well as disease aggressiveness. Whilst at normal levels of TDP-43, the cryptic exon was found to be absent from UNC13A mRNA, the cryptic exon was included when TDP-43 levels were depleted, both in the presence and absence of a risk SNP. The risk SNP was found to induce higher levels of cryptic exon when TDP-43 loss occurs, both in cells and postmortem brain, thus implying that increased levels of this cryptic exon directly contribute to disease, and therefore that inhibiting its inclusion in the mRNA may be of therapeutic benefit.

The UNC13A cryptic exon is associated with TDP pathology, and disease aggressiveness, and therefore represents a novel therapeutic target for such disease. The present inventors have therefore developed novel therapeutics that can target UNC13A and modulate the splicing of UNC13A at the cryptic exon. The ASOs, guide RNAs and viral vectors disclosed herein can be used to prevent inclusion of the UNC13A cryptic exon in the mature mRNA, therefore preventing the loss of UNC13A translated protein such that fully functional UNC13A is produced. The ASOs are used to target motifs within the UNC13A cryptic exon, UNC13A cryptic exon splice sites (e.g., splice 5′ donor or 3′ acceptor sites), the branchpoint, or splice regulatory elements (SREs), including splice enhancers and RNA-binding protein sites, associated with the UNC13A cryptic exon. The ASOs, guide RNAs and viral vectors disclosed herein can be used to mask crucial elements of cryptic exon splicing. The ASOs, guide RNAs and viral vectors disclosed herein can prevent splicing machinery from recognizing the cryptic exon and incorporating the cryptic exon in UNC13A mRNA. The ASOs, guide RNAs and viral vectors disclosed herein are capable of binding to the UNC13A cryptic exon and intronic flanking regions thereof and can therefore be used to probe TDP-43 pathology and/or UNC13A dysfunction and its role in disease.

### DESCRIPTION OF THE FIGURES

The following disclosure will be described with reference to the Figures.

FIG. 1 shows an schematic representation of how an ASO of the present disclosure can modulate splicing of UNC13A: a) an aberrant splicing event, wherein the novel UNC13A cryptic exon is included in the mRNA; the upper portion of FIG. 1(A) showing pre-mRNA and the lower portion showing mature mRNA b) the ASO can target the cryptic exon to modulate splicing, preventing its inclusion in the mature mRNA; the upper portion of FIG. 1(B) showing pre-mRNA and the lower portion showing mature mRNA.

FIG. 2 shows that UNC13A transcripts may comprise a previously unreported novel cryptic cassette exon in TDP-43 depleted cells, as determined by differential splicing analysis between TDP knockdown and control cells.

FIG. 3 shows that the UNC13A cryptic event caused by TDP-43 knockdown is in close proximity to 2 of the polymorphisms (SNPs) which have been which have been previously linked to both ALS and FTD: rs12973192 and rs12608932. One of the polymorphisms, rs12973192, lays 16 bp inside the cryptic event, and the other, rs12608932, is located 534 bp downstream of the 3′ splice site of the cryptic event. Exons 20 and 21 are also shown.

FIG. 4 shows that rs12973192 is the main SNP driving changes in UNC13A cryptic splicing associated to risk of aggressive disease progression. The impact of rs12973192 (exonic SNP) and rs12608932 (intronic SNP) on UNC13A was tested using minigenes carrying the four possible combinations of SNP alleles. Results show that the presence of the risk allele at rs12973192 is able, on its own, to drive an increase in UNC13A CE splicing when TDP-43 is depleted, (see boxes drawn on the gel, and the presence of the higher molecular weight bands for the 2× Risk and REHI combinations).

FIG. 5 shows that expression of the UNC13A cryptic exon was found across multiple datasets from TDP-43 knockdowns in neuronal-like cells.

FIG. 6 shows the strong correlation between the efficiency of the TARDBP depletion, and the amount of UNC13A cryptic exon present, with samples with the greatest reduction in TARDBP RNA relative to control having the greatest inclusion of the UNC13A cryptic exon.

FIG. 7 shows validation by qPCR of the cryptic event in SH-SY5Y TDP-43 KD cells.

FIG. 8 shows how loss of TDP-43 leads to a reduction of UNC13A at the transcript level.

FIG. 9 shows how loss of TDP-43 leads to a reduction of UNC13A at the protein level (A-D).

FIG. 10 shows that the UNC13A cryptic exon is present only in ALS and FTLD brains with TDP-43 pathology.

FIG. 11 shows that the UNC13A cryptic exon is present in FTLD brains with TDP-43 pathology in the cerebellum, frontal cortex and temporal cortex.

FIG. 12 shows that the UNC13A cryptic exon is present in ALS and FTLD brains with TDP-43 pathology in the cerebellum, cervical spinal cord, lumbar spinal cord, motor cortex, temporal cortex and thoracic spinal cord.

FIG. 13 shows the correlation of the UNC13A cryptic exon and the STMN2 cryptic site.

FIGS. 14A-14B shows the results of experiments in which a number of different ASOs were transfected into SK-N-DZ cells and the number or fraction which were correctly spliced when treated with the identified antisense polynucleotides. All cells were treated with doxycycline except for NT (Not treated). All ASOs featured LNA-modified bases, except for OMe sequence(s). Error bars show standard deviation across three replicates. *=p-adjusted<0.05, **=p-adjusted<0.01, ***=p-adjusted<0.001, ****=p-adjusted<0.0001; Tukey Test; significance is calculated relative to Dox.

FIG. 15 shows an example of RT-PCR results showing splicing pattern with and without UNC13A cryptic exon. The bottom band is derived from the correctly spliced product. As expected, it is abundant in the NT (not treated sample) but rare in the Dox-treated sample. 21nt_Don4 (SEQ ID NO. 560) rescues the level of this correctly spliced band, but a scrambled control does not.

FIG. 16 shows qPCR validation of TDP-43 knockdown for the samples in FIG. 14A. Error bars show standard deviation. TDP-43 Ct values were normalized to GAPDH Ct values to account for variation in cDNA quantity. Values are normalized to 1 for the mean TDP-43 abundance in untreated cells.

FIG. 17 shows a schematic diagram showing the binding sites of the example ASOs and the extent of rescue. The shading of the bars indicates the strength of rescue. The number on the bars represents the nucleotide length of the ASO.

FIG. 18 shows that TDP-43 depletion in neurons leads to altered splicing in synaptic genes UNC13A and UNC13B. (A) Differential splicing using MAJIQ and (B) differential expression in control (N=4) and CRISPRi TDP-43 depleted (N=3) iPSC-derived cortical-like i3 Neurons. Each point denotes a splice junction (A) or gene (B). (C) Representative sashimi plots showing cryptic exon (CE) inclusion between exons 20 and 21 of UNC13A upon TDP-43 knockdown (KD). (D, F) Schematics showing intron retention (IR, lower schematic, orange), TDP-43 binding region, and two ALS/FTLD associated SNPs. (E) Representative sashimi plot of UNC13B showing inclusion of the frameshifting exon (fsE) upon TDP-43 KD. (G) LocusZoom plot of the UNC13A locus in the latest ALS GWAS17. Lead SNP rs12973192 plotted as a diamond, other SNPs colored by linkage disequilibrium with rs12973192 in European individuals from 1000 Genomes. (H) BaseScope detection of UNC13A CE in control (top) TDP-43 KD (bottom) i3Neurons co-stained for TDP-43, neuronal processes (TUBB3), and nuclei. (I) Representative image of RT-PCR products using iPSC-derived neurons made from an independent iPSC line, NCRM5, with a non-targeting control sgRNA (sgTARDBP−), an intermediate TDP-43 KD (sgTARDBP+) or stronger TDP-43 KD (sgTARDBP++) (J) Quantification of (I) plotted as means+S.E.M=sgControl (n=6), sgTARDBP+ (n=5), sgTARDBP++ (n=6). One-way ANOVA with multiple comparisons. Significance levels reported as * (p<0.05) ** (p<0.01) *** (p<0.001) **** (p<0.0001). Error bars displaying standard error of the mean. (K) Schematic of nanopore long reads quantified (L) Percentage of targeted UNC13A long reads with TDP-43 regulated splice events that contain either both, CE, or IR in TDP-43 KD SH-SY5Y cells.

FIG. 19 shows UNC13A and UNC13B are downregulated after TDP-43 knockdown due to the production of NMD-sensitive transcripts. (A) Ribosome profiling of TDP-43 knockdown i3 Neurons shows reduction in ribosome occupancy of STMN2, UNC13A and UNC13B transcripts. (B) Mass spectrometry-based proteomic analysis shows dose-dependent reduction in protein abundance of UNC13A and TDP-43 upon TDP-43 knockdown in i3 Neurons. Two-sample t-test. (C) Protein and RNA quantification of TDP-43, UNC13A, and UNC13B in SH-SY5Y with varying levels of DOX-inducible TDP-43 knockdown (D) Transcript expression upon CHX treatment suggests UNC13A and UNC13B, but not STMN2, are sensitive to nonsense-mediated decay. HNRNPL (heterogeneous nuclear ribonucleoprotein L) is a positive control. Shaded bar indicates UNC13B was performed in separate experiment. One-sample t-test. Significance levels reported as * (p<0.05) ** (p<0.01) *** (p<0.001) **** (p<0.0001). Error bars displaying standard error of the mean.

FIG. 20 shows UNC13A CE is highly expressed in ALS/FTLD patient tissue and correlates with known markers of TDP-43 loss of function. (A) UNC13A and STMN2 CE expression from a published dataset of ALS/FTLD patient frontal cortex neuronal nuclei sorted according to the expression of TDP-4327. (B) UNC13A CE expression in bulk RNA-seq from NYGC ALS Consortium normalized by library size across disease and tissue samples. ALS cases stratified by mutation status, FTLD cases stratified by pathological subtype. (C) CE expression throughout ALS/FTLD-TDP cases across tissue, number of tissue samples in brackets (D) BaseScope detection of UNC13A CE (foci) in FTLD-TDP (n=9) but not control (n=5) or FTLD-Tau (n=4) frontal cortex samples and quantification of background corrected foci frequency between groups. Scale bar 10 μm. Error bars displaying standard error of the mean. (E) Correlation in ALS/FTLD-TDP cortex between UNC13A and STMN2 CE PSI in patients with at least 30 spliced reads across the CE locus. (B, C) Wilcoxon test. Significance levels reported as * (p<0.05) ** (p<0.01) *** (p<0.001) **** (p<0.0001).

FIG. 21 shows UNC13A ALS/FTD risk variants enhance UNC13A CE splicing in patients and in vitro by altering TDP-43 pre-mRNA binding. (A) Ratio UNC13A/STMN2 CE PSI, split by genotype for UNC13A risk alleles. (B) Unique cDNAs from targeted RNA-seq in ten CE SNP heterozygous FTLD-TDP patients. p-values from single-tailed binomial tests. FTD1, 5, and 7 are C9orf72 hexanucleotide repeat carriers (C) Diagram of UNC13A minigenes containing exon 20, intron 20, and exon 21 and combinations of UNC13A alleles (D) Representative image of RT-PCR products from UNC13A minigenes in SH-SY5Y±TDP-43 KD. (E) Quantification of (D) plotted as means±S.E. Each variant was compared with the healthy minigene it was co-transfected with and results compared with an unpaired t-test (N=3); (F) TDP-43 iCLIP of SH-SY5Y containing 2R and 2H minigenes: Top—average crosslink density; Middle—average density change 2R-2H (rolling window=20 nt, units=crosslinks per 1,000). Bottom diagram of predicted TDP-43 binding footprints (UGNNUG motif). (G) Average change in E-value (measure of binding enrichment) across proteins for heptamers containing risk/healthy CE SNP allele; TDP-43 is indicated. (H) Binding affinities between TDP-43 and 14-nt RNA containing the CE (n=4) or intronic (n=3) healthy or risk sequences measured by ITC; two-sample t-test. (I) Representative image of RT-PCR products from UNC13A minigenes with mutated UGNNUG TDP-43 binding motifs shown in (F) (J) Quantification of (I) plotted as means±S.E. N=3, Analysis as in D and E.; Significance levels reported as * (p<0.05) ** (p<0.01) *** (p<0.001) **** (p<0.0001)

FIG. 22 shows further evidence that UNC13A is misspliced after TDP-43 knockdown across neuronal lines. (A, B) RNA-seq traces from IGV70 of representative samples from control (top) and TARDBP KD (bottom) in i3Neurons showing intron retention in UNC13A (A) (mean 4.50±1.50 increased IR in KD) and UNC13B (mean 1.86±0.63 increased IR in KD) (B) overlaid with published TDP-43 iCLIP peaks 26; (C) Histogram showing number of basescope cryptic foci per nuclei in control and TDP-43 KD (grey) in WTC11-derived i3Neurons, p<0.0001 unpaired t-test. (D,E) RNA levels of TARDBP and UNC13A with a non-targeting control sgRNA (sgTARDBP−), an intermediate TDP-43 KD (sgTARDBP+) or a higher TDP-43 KD (sgTARDBP++) in WTC11-derived (D) and NCRM-5-derived i3Neurons (D) (F) Representative image of UNC13A CE RT-PCR products (G) Quantification of (F) plotted as means±S.E.M=sgControl (n=6), sgTARDBP+ (n=6), sgTARDBP++ (n=6). One-way ANOVA with multiple comparisons. (H-K) Expression of TDP-43 regulated splicing in UNC13A (H, I) and UNC13B (J, K) across published neuronal datasets in control and TDP-43 KD. Intron retention (IR) (I, K) and CE and fsE PSI (H, J) significantly increase after TDP-43 depletion in most experiments, Wilcoxon test (L) Relative gene expression levels for TARDBP across neuronal datasets 11,25. Normalized RNA counts are shown as relative to control mean. Numbers show log2 fold change calculated by DESeq2. Significance shown as adjusted p-values from DESeq2. Significance levels reported as * (p<0.05) ** (p<0.01) *** (p<0.001) **** (p <0.0001).

FIG. 23 shows the validation of UNC13A and UNC13B misplicing after TDP-43 KD across multiple neuronal cell lines. (A) Sanger sequencing of cryptic bands in both SH-SY5Y and SK-N-DZ cells confirm the CE splice junctions. (B, C) Crosslink density across UNC13A (chr19) (B) and UNC13B (chr9) (C) genomic loci from novel iCLIP on endogenous TDP-43 in SH-SHY5Y cells. Crosslink densities for both genes show peaks at the CE/fsE (left dotted line) and retained introns (right dotted line). Coordinates shown in hg38 percentage of all targeted UNC13A long reads in SH-SY5Y cells containing either neither CE nor IR, both, or either cryptic exon or intronic retention as determined by Sanger sequencing. Most reads in both control and TDP-43 KD contain neither event, and while IR event is present in controls, CE is only detected in TDP-43 KD. (Bottom) Targeted nanopore sequencing reveals UNC13A CE and IR events occur largely independently in-vitro. (A) Percentage of all targeted UNC13A long reads in SH-SY5Y cells containing either neither CE nor IR, both, or either CE or IR. Most reads in both control and TDP-43 KD contain neither event, and while IR event is present in controls, CE is only detected in TDP-43 KD

FIG. 24 shows that the reduction of UNC13A and UNC13B after TDP-43 knockdown correlates with TDP-43 levels and is caused by nonsense-mediated decay. Relative gene expression levels for UNC13A (A) and UNC13B (B) after TDP-43 knockdown across neuronal cell lines 11,25. Normalized RNA counts are shown as relative to control mean. Numbers show log fold change calculated by DESeq2. Significance shown as adjusted p-values from DESeq2. (C, D) RT-qPCR analysis shows TDP-43, UNC13A and UNC13B gene expression is reduced by TARDBP shRNA knockdown in both SH-SY5Y and SK-N-DZ human cell lines. Graphs represent the means±S.E., N=6, one sample t-test. (E) The 5′ ends of 29 nt reads relative to the annotated start codon from a representative ribosome profiling dataset (TDP-43 KD replicate B). As expected, we detected strong three-nucleotide periodicity, and a strong enrichment of reads across the annotated coding sequence relative to the upstream untranslated region. (F) UNC13A, UNC13B, and TDP-43 protein levels, measured by Western Blot, with varying levels of DOX-inducible TDP-43 knockdown in SH-SY5Y cells. Tubulin is used as endogenous control, N=3. (G) Quantification of RT-PCR products from the transcripts containing UNC13A CE, UNC13A intron retention, UNC13B fsE, and UNC13B intron retention, with varying levels of DOX-inducible TDP-43 knockdown in SH-SY5Y cells. Error bars displaying standard error of the mean, N=3. (H) UPF1 siRNA knock-down led to the rescue of hnRNPL (positive control), UNC13A, and UNC13B transcripts, but not STMN2. Error bars displaying standard error of the mean, N=4, one-sample t-test. (I) UN (′13A CE containing-transcript PSI is increased after UPF1 knockdown in i3Neurons. Error bars displaying standard error of the mean, N=6. (J) RT-PCR products from UNC13A in the setting of mild TDP-43 knockdown with the addition of either DMSO (control) or CHX (NMD inhibition). (K) Quantification of (J) plotted as means±S.E, N=4. Significance levels reported as * (p<0.05) ** (p<0.01) *** (p<0.001) **** (p<0.0001).

FIG. 25 shows the differences in sample technical factors where UNC13A CE was detected and undetected vary between cortical and spinal tissues. (A) Detection rate of UNC13A CE across tissues by RNA sequencing platform and read length. UNC13A CE was more likely to be detected in cervical spinal cord and motor cortex when sequenced on machines with 125 bp compared to 100 bp. (B) No significant differences in total RNA-seq library size (log10scaled). (C) RNA integrity score (RIN) was significantly lower in motor and temporal cortices in samples where UNC13A was detected. (D) Cell type decomposition revealed that samples with UNC13A CE detected had a higher proportion of neurons in cervical and lumbar spinal cord, whereas in frontal, temporal, and motor cortex samples with UNC13A CE detected had a lower proportion of neurons, and in motor and temporal cortex samples with UNC13A CE detected had a higher proportion of astrocytes. Astrocy.—Astrocytes, Endothi.—Endothelial, Microgl—Microglia. Neur.—Neurons, Oligiodendr.—Oligodendrycytes. P-values shown are from Fisher's exact test (A) or Wilcoxon test (B-D). N tissue samples show below in brackets.

FIG. 26 shows the targeted long reads in FTLD frontal cortex show that UNC13A CE and IR occur independently in-vivo. (A) Percentage of targeted UNC13A long reads with TDP-43 regulated splice events that contain either both, CE, or IR in four in FTLD frontal cortices. (B) Percentage of all targeted UNC13A long reads in (A) containing either neither CE nor IR, both, or either CE or IR

FIG. 27 shows that TDP-43 regulated UNC13A and UNC13B introns are expressed across human neuronal tissues in NYGC tissue samples. IR ratio in UNC13A exon 31-32 (A) and UNC13B exon 21-22 (B) across NYGC tissue samples. UNC13A IR was lower in ALS-TDP cases than in controls in cervical spinal, frontal and motor cortices, and higher in FTLD-TDP cases than controls in frontal and temporal cortices. This is believed to reflect differences in the effects of cell type composition in disease state. Wilcoxon test, significance levels reported as * (p<0.05) ** (p<0.01) *** (p<0.001) **** (p<0.0001).

FIG. 28 shows STMN2 CE PSI correlates with TDP-43 regulated cryptics across NYGC RNA-seq dataset. (A, B) Previously described CE in RAP1GAP and PFKP regulated by TDP-439-11 correlate with STMN2 CE, suggesting the STMN2 CE PSI could act as a readout of TDP-43 function. Only samples with at least 30 spliced reads across each CE locus are included in correlations. Spearman's correlation.

FIG. 29 shows UNC13A risk alleles increase UNC13A CE expression after TDP-43 depletion by altering TDP-43 binding affinity across the UNC13A CE-containing intron. (A) UNC13A CE PSI by genotype (Wilcoxon test) (B) Effect of CE or intronic SNP on the correlation between STMN2 and UNC13A CE PSI in ALS/FTD cortex in samples with at least 30 junction reads across the CE locus. Spearman's correlation. (C) Raw tape station gel images of UNC13A CE products in 2H and 2R minigenes and quantification of the PCR products (n=3); Two-way ANOVA (D) Raw tape station gel images corresponding to FIG. 4E. Two sets of primers were used to amplify either control (top row) or mutant minigene (bottom row). Left panel: single transfections were performed to ensure primer specificity. Right panel: three replicates of the double transfections; (E) Fractional changes at iCLIP peaks for 2R versus 2H minigene (mean and 75% confidence interval shown). Peaks that are within 50 nt of each SNP are highlighted. (F) Mean crosslink density around the exonic (top) and intronic (bottom) SNPs in the 2H and 2R minigenes, relative to the 5′ end of minigene (error bars=standard deviation; dashed lines show SNP positions). (G, H) Individual TDP-43 E-scores for the CE (G) and intronic (H) heptamers for which there was data 30 (I) Average change in E-value (measure of binding enrichment) across proteins for heptamers containing risk/healthy intronic SNP allele; TDP-43 is indicated. Significance levels reported as * (p<0.05) ** (p<0.01) *** (p<0.001) (p<0.0001).

FIG. 30 shows binding of TDP-43 to SNP-containing intronic RNA. (A-D) ITC measurement of the interaction of TDP-43 with 14-nt RNA containing the CE SNP (A, B) and intronic SNP (C, D) healthy sequence. A representative data set is reported, with raw data (A, C) and integrated heat plot (B, D). Circles indicate the integrated heat, the curve represents the best fit. (E) Raw Tapestation gel images corresponding to FIG. 4J. For each experiment, two RT-PCRs were performed with a different primer set which either amplified a control minigene (top row; minigene 2H) or a mutant minigene (bottom row). Left: single transfections to ensure specificity of primers for either the control or the mutant minigene. Right: Three replicates of double transfections with control minigene 2H and either mutant minigene.

FIG. 31 shows one of the splice junctions for UNC13A CE overlaps with an unannotated exon expressed in control cerebellum (A) Expression of splice junction reads supporting the UNC13A CE across tissues and disease subtypes. Junction counts are normalized by library size in millions (junctions per million). The long novel acceptor junction is expressed across all disease subtypes in the cerebellum. (B) Example RNA-seq traces from IGV showing UNC13A cerebellar exon which shares the long novel acceptor junction as the UNC13A CE.

FIG. 32 shows ASO treatments targeting short and long cryptic acceptors.

FIG. 33 shows SHSY5Y cells treated with 3 concentrations of 21 nt LNA donor ASOs as described herein.

### DETAILED DESCRIPTION OF THE DISCLOSURE

### I. Compositions of the Present Disclosure

The work described herein relates to compositions and methods for suppressing or preventing the inclusion of a cryptic exon in UNC13A mRNA. The inclusion of a cryptic exon in UNC13A mRNA may lead to a truncated transcript and protein.

UNC13A expression may be restored through suppression of a cryptic splicing form of UNC13A that occurs when TDP-43 becomes sequestered or is reduced in functionality, such as by blocking the occurrence or accumulation of the cryptic form and converting it back to or restoring functional UNC13A RNA (e.g., by administration of an ASO or antisense oligonucleotide). In addition, work described herein relates to compositions and methods for increasing protein synthesis of UNC13A, i.e., increasing UNC13A protein expression.

Also provided in the present disclosure are compositions and formulations comprising ASOs or antisense polynucleotides described herein.

In some embodiments, the compositions are formulated for administration through a particular route, such as intravenous injection, intramuscular injection, subcutaneous injection, etc.

In another aspect of the present disclosure, methods for use of the antisense polynucleotides, composition and formulations as described herein, are provided. In some embodiments, the antisense polynucleotides, compositions, and formulations of the present disclosure may be used as tools to regulate gene expression, including but not limited to transcriptional regulation, splicing regulation, translational regulation, and post-translational regulation. In some embodiments, the antisense polynucleotides, compositions, and formulations of the present disclosure may be as therapeutic agents for disease treatment and prevention.

Some aspects of the present disclosure provide naked antisense polynucleotides. Other aspects of the present disclosure provide antisense polynucleotides encapsulated or formulated with a carrier such as a lipid containing carrier.

### Antisense Polynucleotides (ASO Compounds)

In certain embodiments, provided herein are oligomeric compounds comprising oligonucleotide or polynucleotides, which consist of linked nucleosides. As used herein, the terms “oligonucleotide” and “polynucleotide” may be used interchangeably. These may also be described as ASOs throughout this disclosure.

ASOs or polynucleotides may be unmodified (RNA or DNA) or may be modified. Modified ASOs or antisense polynucleotides comprise at least one modification relative to unmodified RNA or DNA. That is, modified polynucleotides comprise at least one modified nucleoside (comprising a modified sugar moiety and/or a modified nucleobase) and/or at least one modified internucleoside linkage.

The antisense polynucleotides (ASO compounds) of the present invention may comprises a nucleotide sequence of 13-30 nucleotides, wherein the nucleotide sequence has sequence complementarity with SEQ ID NO 1 (i.e., the 13-30 nucleotide sequence is complementary to a portion of the SEQ ID NO 1 sequence that has the same length as the nucleotide sequence). SEQ ID NO 1 corresponds to a portion of the pre-mRNA UNC13A sequence and encompasses the UNC13A cryptic exon plus intronic flanking regions.

The ASO can prevent aberrant splicing of the UNC13A pre-mRNA to prevent inclusion of the UNC13A cryptic exon in the mature UNC13A mRNA. In an embodiment the ASO prevents splicing machinery (e.g., protein splicing factors) from recognizing the UNC13A cryptic exon. In an embodiment, the ASO is capable of preventing inclusion of an UNC13A cryptic exon into an UNC13A mature mRNA.

In an embodiment, the ASO is capable of binding to the UNC13A cryptic exon and/or intronic flanking regions thereof (i.e., in the UNC13A pre-mRNA). In an embodiment, the flanking regions correspond to 844 bp upstream or 303 bp downstream of the cryptic exon sequence. In an embodiment, the UNC13A cryptic exon and intronic regions thereof correspond to position chr19: 17,641,557-17642844. In an embodiment, the ASO is capable of binding to the UNC13A cryptic exon. In an embodiment, the UNC13A cryptic exon corresponds to position chr19: 17642414-17,642,541. In an embodiment, the UNC13A cryptic exon corresponds to SEQ ID NO 2. In an embodiment, the ASO comprises a nucleotide sequence that is complementary to SEQ ID NO:2. In an embodiment, the UNC13A cryptic exon is a longer variant of the UN13CA cryptic exon that corresponds to position chr19: 17642414-17642591. In an embodiment, the UNC13A cryptic exon is a longer variant of the cryptic exon that corresponds to SEQ ID NO 3. In an embodiment, the ASO comprises a nucleotide sequence that is complementary to SEQ ID NO: 3.

In an embodiment, the ASO is capable of binding to a splice site of the UNC13A cryptic exon (i.e., and flanking regions thereof). Targeting the splice sites makes them less available for splicing. In an embodiment, the splice sites correspond to positions chr19: 17642414, chr19: 17,642,541 or chr19: 17642591. In an embodiment, the splice site may be a 5′-splice site (i.e., donor splice site) or a 3′ splice site (i.e., acceptor splice site). In some embodiments, the splice site is a 3′ splice site (acceptor site) is the long acceptor site (i.e., corresponding to position chr19: 17642414) or the short acceptor site (i.e., corresponding to position chr19: 17,642,541). In some embodiments, the splice site is a 5′ splice site (i.e., donor site corresponding to position chr19: 17642591).

In an embodiment, the ASO sequence comprises a sequence complementary to a splice site as described herein (e.g., the splice donor site, i.e., corresponding to the phosphodiester bond between chr19: 17,642,413-17,642,414 or a splice acceptor site, i.e., corresponding to the phosphodiester bond between chr19: 17,642,541-17,642,542 or chr19: 17,642,591-17,642,592).

In some embodiments, the ASO comprises a sequence complementary to SEQ ID NO 547-551 (i.e., a sequence complementary to a splice donor site). In some embodiments, the ASO may comprise one or more of SEQ ID NO: 270-352.

In some embodiments, the ASO comprises a sequence complementary to SEQ ID NO: 552 to 554 (i.e., a sequence complementary to a splice acceptor site).

In some embodiments, the ASO may comprise one or more of SEQ ID NO: 105-189.

In some embodiments, the ASO comprises a sequence complementary to the short acceptor site.

In some embodiments, the ASO comprises a sequence comprising one or more of SEQ ID NO: 155-180 or SEQ ID 160-175.

In some embodiments, the ASO comprises a sequence complementary to the long acceptor site.

In some embodiments, the ASO comprises a sequence comprising one or more of SEQ ID NO 105-127 or SEQ ID NO: 110-122.

The ASO capable of binding to a splice site of the UNC13A cryptic exon thereof may have sequence complementarity to a target sequence comprising the splice site (e.g., a donor or acceptor splice site) and flanking regions thereof. In some embodiments, the flanking region may be at least 100 nucleotides (nt) upstream or downstream from the splice site (e.g., donor or acceptor splice site), or at least 90 nucleotides, or at least 80 nucleotides, or at least 70 nucleotides, or at least 60 nucleotides, or at least 50 nucleotides, or at least 40 nucleotides, or at least 30 nucleotides, or at least 25 nucleotides, or at least 20 nucleotides, or at least 15 nucleotides, or at least 10 nucleotides, or at least 5 nucleotides upstream of downstream from the splice site.

In some embodiments, the ASO is capable of binding directly to a splice site.

In an embodiment, the ASO is capable of binding to a branchpoint of the UNC13A cryptic exon. Targeting the branchpoint makes the splicing less efficient. In an embodiment, the branchpoint corresponds to position chr19: 17642800. In an embodiment, the ASO sequence comprises a sequence complementary to the branchpoint as described herein.

In an embodiment, the ASO is capable of binding to a splice regulatory element (SRE) associated with UNC13A to modulate splicing of the UNC13A cryptic exon. Targeting splice regulatory elements limits the binding of RNA binding proteins that enhance the inclusion of the cryptic exon in the UNC13A mature mRNA.

In an embodiment, the SREs may be determined in silico. In an embodiment, the SRE is a splice enhancer, and the ASO is capable of binding to a splice enhancer. In an embodiment, the SRE is a TDP-43 binding site, and the ASO is capable of binding to a TDP-43 binding site to modulate splicing of the UNC13A cryptic exon. In an embodiment, the SRE is the cryptic exon, and the ASO is capable of binding to the cryptic exon. In an embodiment, the SRE is an SNP in the intronic flanking region of the CE. ASOs may be capable of binding to part of the sequence in the UNC13A pre-mRNA transcribed from a sequence comprising the intronic SNP. The intronic SNP is rs12608932. In an embodiment, the SRE is a SNP in the CE (e.g., cryptic exon SNP). ASOs may be capable of binding to part of the sequence in the UNC13A pre-mRNA transcribed from a sequence comprising the CE SNP (e.g., cryptic exon SNP). The CE SNP is rs12973192. In some embodiments, the ASO is capable of binding to a sequence comprising binding to both the CE SNP rs12973192 and the donor splice site, in other words, the ASO is complementary to both the CE SNP and the donor splice site.

In an embodiment, the ASO blocks the interaction of certain proteins with the UNC13A pre-mRNA, for example, splicing factors. In an embodiment, binding of the ASO to UNC13A pre-mRNA prevents loss of the fully translated UNC13A protein (i.e., the ASO corrects the reduced levels of the UNC13A protein).

In an embodiment, the ASO prevents inclusion of the cryptic exon into the UNC13A mRNA. In an embodiment, the ASO prevents loss and/or restores functionality of the UNC13A translated protein.

### UNC13A and the Cryptic Exon

According to the present disclosure, SEQ ID NO: 1 refers to the target sequence of the UNC13A cryptic exon and intronic flanking regions thereof. As described elsewhere herein, the target sequence SEQ ID NO: 1 encompasses the sequence with the minor allele (e.g., risk variant of the SNP) or the major allele at rs12973192, and therefore also encompasses the sequence wherein G at rs12973192 is replaced with C and/or wherein U at rs12608932 is replaced with G. Thus, while SEQ ID NO: 1 described herein is demonstrated with G at rs12973192 (position 415 in SEQ ID NO: 1) and U at rs12608932 (position 965 in SEQ ID NO:1), SEQ ID NO: 1 may comprise either a G or a C at position 415 and/or a U or a G at position 965. The coordinates refer to the hg38 assembly.

SEQ ID NO: 2 and 3 disclosed herein are the short and long sequences of the UNC13A cryptic exon. As described elsewhere herein, the SEQ ID NO: 2 and 3 encompasses the sequence with and without the SNP at rs12973192, and therefore also encompasses the sequence wherein G at rs12973192 is replaced with C. Thus, while SEQ ID NO: 2 and SEQ ID NO: 3 described herein are demonstrated with G at rs12973192 (i.e., corresponding to position 112 and 162 in SEQ ID NO: 2 and SEQ ID NO: 3 respectively), are intended to encompass SEQ ID NO: 2 and SEQ ID NO: 2 may comprise a G or a C at position 112 and 162 in SEQ ID NO: 2 and SEQ ID NO: 3 respectively.

UNC13A cryptic exon as defined herein refers to a cryptic exon sequence that is aberrantly included in UNC13A mRNA, in some examples, due to TDP pathology. The UNC13A cryptic exon may correspond to SEQ ID NO: 2 (shorter UNC13A cryptic variant) corresponding to position chr19: 17642414-17,642,541 or SEQ ID NO: 3 (longer UNC13A cryptic variant) corresponding to position chr19: 17642414-17642591. The UNC13A cryptic exon is positioned between exons 20 and 21 of UNC13A. SEQ ID NO: 1 corresponds to the UNC13A cryptic exon and flanking regions thereof in the UNC13A pre-RNA.

As defined herein, crucial elements involved in cryptic exon splicing encompass the a) branchpoint; b) splicing sites, including i) 3′ splice sites (otherwise referred to as splice acceptor sites), ii) 5′ splice site (otherwise referred to as a splice donor site); and c) splicing regulatory elements (SREs). Splice sites defined herein refer to the sites or sequences where splicing occurs. Branchpoints as defined herein refer to an “A” nucleotide upstream of the splice acceptor, and often its loss can be compensated by another neighboring “A”. Splicing regulator elements (SREs) as referred to herein are sites or sequences where RNA binding proteins bind and promote the splicing event. SREs as defined herein include splice enhancers, TDP-43 binding sites, and RNA-binding protein sites, and/or portions of the transcribed UNC13A pre-mRNA sequence comprising a SNP (e.g., a risk SNP-rs12973192 and/or rs12608932).

### Complementarity

In an embodiment, the antisense polynucleotide (e.g., ASO) has at least 95% sequence complementarity with SEQ ID NO 1, or at least 95.5%, or at least 96%, or at least 96.5%, or at least 97%, or at least 97.5%, or at least 98%, or at least 98.5%, or at least 99%, or at least 99.5%, or at least 100% complementarity with SEQ ID NO 1.

In an embodiment, the antisense polynucleotide (e.g., ASO) has at least 95% sequence complementarity with SEQ ID NO 2, or at least 95.5%, or at least 96%, or at least 96.5%, or at least 97%, or at least 97.5%, or at least 98%, or at least 98.5%, or at least 99%, or at least 99.5%, or at least 100% complementarity with SEQ ID NO 2.

In an embodiment, the antisense polynucleotide (e.g., ASO) has at least 95% sequence complementarity with SEQ ID NO 3, or at least 95.5%, or at least 96%, or at least 96.5%, or at least 97%, or at least 97.5%, or at least 98%, or at least 98.5%, or at least 99%, or at least 99.5%, or at least 100% complementarity with SEQ ID NO 3.

Complementarity with SEQ ID NO 1, SEQ ID NO 2, or SEQ ID NO 3 refers to complementary of the antisense polynucleotide (e.g., ASO) with the portion of the SEQ ID NO 1, SEQ ID NO 2 and SEQ ID NO 3.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo.

In certain embodiments, antisense polynucleotides or antisense oligonucleotides (ASOs) are complementary to the target nucleic acid over the entire length of the oligonucleotide. In certain embodiments, antisense polynucleotides or antisense oligonucleotides (ASOs) are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, or 20 nucleobases in length.

In certain embodiments, antisense polynucleotides or antisense oligonucleotides (ASOs) comprise one or more mismatched nucleobases relative to the target nucleic acid. In certain embodiments, the mismatch is at position 1, 2, 3, 4, 5, 6, 7, 8, 9, 10, 11, 12, 13, 14, 15, 16, 17, 18, 19, or 20 from the 5′-end of the oligonucleotide.

In some embodiments, the nucleic acid-based compositions described herein, including ASOs described herein, comprise an oligo- or polynucleotide that is at least 80% complementary to a region of the target transcript. This region on the target transcript where the nucleic acid-based compositions hybridize or bind to the target transcript is referred to as the “targeted sequence” or “target site.”

The term “complementary to” means being able to hybridize under physiological conditions, or in some cases being able to hybridize under stringent conditions with respect to hybridization temperature and salt concentration. It is to be understood that thymidine (T) of any given DNA sequence is replaced by uridine (U) in its corresponding RNA transcript and that this difference does not alter the understanding of the term “complementarity.”

The nucleic acid-based compositions described (e.g, the antisense polynucleotides or antisense oligonucleotides (ASOs)) (e.g, the antisense polynucleotides or antisense oligonucleotides (ASOs)) can share at least 80% sequence identity, at least 90% sequence identity, at least 95% sequence identity, at least 98% sequence identity, at least 99% sequence identity, or can be 100% identical with, the reverse complement of the targeted sequence. Thus, the reverse complements of the described nucleic acid-based compositions have a high degree of sequence identity with the targeted sequence. The targeted sequence can have the same length, i.e., the same number of nucleotides, as the nucleic acid-based compositions, or the targeted sequence can have a similar length, i.e., within 1 nucleotide, within 2 nucleotides, within 3 nucleotides, within 4 nucleotides, or within 5 nucleotides compared to the length of the nucleic acid-based compositions. The nucleic acid-based compositions may hybridize with all or a portion of the targeted sequence or hybridize intermittently with the targeted sequence. In some embodiments, targeted sequence may hybridize with all or a portion of the nucleic acid-based compositions described herein, or the targeted sequence may hybridize intermittently with the nucleic acid-based compositions.

In some embodiments, the targeted sequence comprises at least 8 nucleotides. Thus, the targeted sequence can be 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. In some cases, the targeted sequence is greater than 30 nucleotides in length. In some embodiments, the targeted sequence is between 6 and 18 nucleotides in length.

In some embodiments, the targeted sequence is between 7 and 18 nucleotides in length. In some embodiments, the targeted sequence is between 8 and 18 nucleotides in length. In some embodiments, the targeted sequence is between 9 and 18 nucleotides in length. In some embodiments, the targeted sequence is between 10 and 18 nucleotides in length. In some embodiments, the targeted sequence is between 11 and 18 nucleotides in length. In some embodiments, the targeted sequence is between 12 and 18 nucleotides in length. In some embodiments, the targeted sequence is between 13 and 18 nucleotides in length. In some embodiments, the targeted sequence is between 14 and 18 nucleotides in length. In some embodiments, the targeted sequence is about 14 nucleotides in length. In some embodiments, the targeted sequence is about 15 nucleotides in length. In some embodiments, the targeted sequence is about 16 nucleotides in length.

For example, in certain embodiments, nucleic acid-based compositions described (e.g, the antisense polynucleotides or antisense oligonucleotides (ASOs)) consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

The ASO described herein has or may comprise a length of 13-30 nucleotides. In an embodiment, the ASO may consist essentially of 13-30 nucleotides that are complementary with SEQ ID NO 1, SEQ ID NO 2 or SEQ ID NO 3.

In an embodiment, the ASO has at least 13-nucleotides, or at least 14 nucleotides, or at least 15 nucleotides, or at least 16 nucleotides, or at least 17 nucleotides, 18 nucleotides, or at least 19 nucleotides, or at least 20 nucleotides, or at least 21 nucleotides, or at least 22 nucleotides, or at least 23 nucleotides, or at least 24 nucleotides. In an embodiment, the ASO has less than 30 nucleotides, or less than 29, or less than 28, or less than 27, or less than 26, or less than 25, or less than 24, or less than 23, or less than 22, or less than 21, or less than 20 nucleotides. In an embodiment, the ASO has from 15 to 30 nucleotides, or from 16 to 30 nucleotides, or from 17 to 30 nucleotides, or from 17 to 28 nucleotides, or from 18 to 30 nucleotides, or from 17 to 28 nucleotides, or from 18 to 28 nucleotides, or from 19 to 26 nucleotides, or from 19 to 25 nucleotides, or from 20 to 25 nucleotides, or from 20 to 24 nucleotides, or from 20 to 23 nucleotides, or from 20 to 22 nucleotides, or from 21 to 24 nucleotides, or from 21 to 23 nucleotides, or from 22 to 24 nucleotides. In an embodiment, the ASO may have 13, 14, 15, 16, 17, 18, 19, 20, 21, 22, 23, 24, 25, 26, 27, 28, 29 or 30 nucleotides.

In an embodiment, the ASO may have 13, 14, 15, 16, 17, 18, 19, 20, 21, 22, 23, 24, 25, 26, 27, 28, 29 or 30 nucleotides that are complementary to SEQ ID NO 1, SEQ ID NO 2 or SEQ ID NO 3. In an embodiment, the ASO has a length of 13-30 nucleotides and the ASO comprises 13-30 nucleotides that are complementary with SEQ ID NO 1, SEQ ID NO 2 or SEQ ID NO 3.

In some embodiments, the ASO may, (i.e., in the context of hybridizing to a target) comprise a nucleotide overhang. A nucleotide overhang is a part of the ASO which is not complementary to the target sequence (e.g., SEQ ID NO 1). In some embodiments, the overhang may comprise 1 to 3 nucleotides. In some embodiments, the overhang is at the 3′ end. In an embodiment, the ASO may comprise no nucleotide overhang, for example, wherein the entire ASO is complementary with SEQ ID NO:1, or SEQ ID NO 2, or SEQ ID NO 3.

### Sequences

The ASO of the present invention comprises a nucleotide sequence of from about 13 to about 30 nucleotides, wherein the nucleotide sequence has sequence complementarity with SEQ ID NO 1. As described elsewhere herein, the target sequence SEQ ID NO: 1 is also intended to encompass the sequence wherein one or more SNP is present in the inside the cryptic exon and/or flanking regions, at rs12973192 and/or rs12608932. The coordinates refer to the hg38 assembly.

In some embodiments, the ASO consists of a nucleotide sequence of 13-30 nucleotides, wherein the nucleotide sequence has sequence complementarity with SEQ ID NO 1.

In an embodiment of the first and second aspect, the ASO is capable of binding to a UNC13A cryptic exon or flanking region thereof. In an embodiment, the ASO (i.e., at least a portion of the ASO) comprises a 13-nucleotide sequence corresponding to any one or one or more of SEQ ID NO 4-546.

In an embodiment, the ASO (i.e., at least a portion of the ASO) comprises at least a 13-nucleotide sequence corresponding to any one or one or more of SEQ ID NO 4-104. These ASOs can target the branchpoint such that splicing is less efficient.

In an embodiment, the ASO (i.e., at least a portion of the ASO) comprises at least a 13-nucleotide sequence corresponding to any one, or one or more of SEQ ID NO: 105-189 or SEQ ID NO: 270-352. These correspond to sequences that target the UNC13A cryptic exon splice sites (i.e., acceptor and donor splice sites respectively).

In an embodiment the ASO (i.e., at least a portion of the ASO) comprises at least a 13-nucleotide sequence corresponding to any one, or one or more of SEQ ID NO: 105-189. These correspond to sequences that target the UNC13A cryptic exon 3′-splice sites (i.e., acceptor sites). In some examples, the ASO comprise at least a 13-nucleotide sequence corresponding to SEQ ID NO: 150 to 185, more preferably SEQ ID NO: 160 to 175. In an embodiment, the ASO (i.e., at least a portion of the ASO) comprises at least a 13-nucleotide sequence corresponding to one or more of SEQ ID NO 270-352. These correspond to sequences that target the UNC13A cryptic exon 5′-splice site. In some examples, the ASO comprises at least a 13-nucleotide sequence corresponding to any one or more of SEQ ID NO: 270 to 345, more preferably SEQ ID NO: 275 to 340, more preferably SEQ ID NO: 280 to 330, more preferably SEQ ID NO: 285 to 325, more preferably SEQ ID NO: 290 to 320, more preferably SEQ ID NO 295 to 324.

In an embodiment, the ASO (i.e., at least a portion of the ASO) comprises at least a 13-nucleotide sequence corresponding to one or more of SEQ ID NO 190-269, SEQ ID NO: 353-426, SEQ ID NO:427-474, or SEQ ID NO:475-546. These correspond to sequences that target the UNC13A splice regulatory elements.

In an embodiment, the SRE is a TDP-43 binding site. The ASO (i.e., at least a portion of the ASO) may comprise a 13-nucleotide sequence corresponding to any one or more of SEQ ID NO 427-474. In an embodiment, the SRE is an enhancer. The ASO may comprise at least a 13-nucleotide sequence corresponding to any one or more of SED ID: 475-546.

In an embodiment, the SRE is within the cryptic exon. In an embodiment, the ASO is capable of binding to a UNC13A cryptic exon. In an embodiment, the UNC13A cryptic exon corresponds to SEQ ID NO 2 or SEQ ID NO 3. As described elsewhere herein, the sequences SEQ ID NO: 2 and 3 are also intended to encompass a potential SNP inside the cryptic exon, rs12973192. In an embodiment, the ASO is complementary to SEQ ID NO 2 or SEQ ID NO 3. In an embodiment, the ASO (i.e., or at least a portion thereof) comprises at least a 13-nucleotide sequence corresponding to any one or more of SEQ ID NO 190-269. These correspond to sequences that target the cryptic exon. Targeting the cryptic exon prevents splicing machinery from recognizing the CE.

In an embodiment, the SRE is an intronic SNP. In an embodiment the ASO (i.e., at least a portion of the ASO) comprises at least a 13-nucleotide sequence corresponding to any one or more of SEQ ID NO 353-426. These correspond to sequences that target the pre-mRNA transcribed from a sequence comprising the intronic SNP. The intronic SNP is linked with disease. The intronic SNP is rs12608932.

In some embodiments, the ASO (i.e., at least a portion of the ASO) may comprise 13-nucleotides and correspond exactly to the sequence of one or more of SEQ ID NO 4-546. In some embodiments, the ASO comprises one or more further nucleotides (1, 2, 3, 4, 5, 6, 7, 8, 9, 10, 11, 12, 13, 14, 15, 16, 17 further nucleotides) that flank the sequence of one of SEQ ID NO 4-546, e.g., wherein the ASO has 14, 15, 16, 17, 18, 19, 20, 21, 22, 23, 24, 25, 26, 27, 28, 29 or 30 nucleotides respectively. The further nucleotides may be present at either the 5′ and/or 3′ end of the 13-nucleotide sequence corresponding to SEQ ID NO 4-546.

In an embodiment, the ASO is longer than 13-nucleotides and comprises more than one sequence that is complementary with SEQ ID NO: 1. In an embodiment, the ASO may comprise one, two, three, four, five, six, seven, eight, nine, ten, eleven, twelve, thirteen, fourteen, fifteen, sixteen, seventeen or eighteen overlapping consecutive sequences, for example, selected from SEQ ID NO: 4-104, SEQ ID NO-105-189, SEQ ID NO: 190-269, SEQ ID NO 270-352, SEQ ID NO 353-426, or SEQ ID NO 427-474, or SEQ ID NO: 475-546, wherein consecutive refers to the number of the SEQ ID. For example, an ASO comprising 20 nucleotides may comprise eight consecutive sequences selected from SEQ ID NO 4-104 (e.g., wherein the ASO comprises SEQ ID NO 4, 5, 6, 7, 8, 9, 10 and 11). In some examples, the ASO comprises SEQ ID NO: n and SEQ ID, n+1, and optionally SEQ ID NO: n+2, and further optionally SEQ ID NO: n+3, and further, optionally SEQ ID NO: n+4, and further optionally SEQ ID NO: n+4, and further optionally SEQ ID NO: n+5; and further optionally SEQ ID NO: n+6; and further optionally SEQ ID NO: n+7, and further optionally SEQ ID NO: n+8, and further optionally SEQ ID NO: n+9, and further optionally SEQ ID NO: n+10, and further optionally SEQ ID NO: n+11, and further optionally SEQ ID NO: n+12, and further optionally SEQ ID NO: n+13, and further optionally SEQ ID NO: n+14, and further optionally SEQ ID NO: n+15, and further optionally SEQ ID NO: n+16, and further optionally SEQ ID NO: n+17, and further optionally SEQ ID NO: n+18, wherein n is the number of the SEQ ID.

In some embodiments, the ASO comprises a nucleotide sequence that has sequence complementarity with one or more of SEQ ID NO: 547 to 551, or one or more of SEQ ID NO: 552 to 554.

### Modifications

Modifications may be introduced into the polynucleotides (e.g., ASO(s)) described herein. The polynucleotides (e.g., ASO(s)) of the present disclosure may include one, two, three, or more modifications. The modifications may be various distinct modifications. In some embodiments, the modifications may locate at various regions and fragments of the polynucleotides of the present disclosure, including but not limited to, the coding region(s), the untranslated region(s), the flanking region(s), and/or the terminal or tailing regions.

The modifications which render the nucleic acid molecules, when introduced to a cell, more resistant to degradation in the cell and/or more stable in the cell as compared to unmodified polynucleotides (e.g., ASO). The modifications may also increase the biological functions of nucleic acid molecules as compared to unmodified polynucleotides, such as binding to an RBP or another polynucleotide (e.g., ASO).

The modifications may be structural and/or chemical modifications. The chemical modification may be a nucleotide and/or nucleoside modification including a nucleobase modification and/or a sugar modification, and a backbone linkage modification (i.e., the internucleoside linkage, e.g., a linking phosphate, a phosphodiester linkage, and a phosphodiester backbone). The structural modification may include a secondary structural modification, and a tertiary structural modification.

Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof.

In some embodiments, one, two, or more (optionally different) nucleoside or nucleotide modifications may be incorporated to the polynucleotides (e.g., ASO(s)) of the present disclosure. As described herein, “nucleoside” is defined as a compound containing a sugar molecule (e.g., a pentose or ribose) or a derivative thereof in combination with an organic base (e.g., a purine or a pyrimidine) or a derivative thereof (also referred to herein as “nucleobase”). Five primary/canonical nucleobases: adenine (A), cytosine (C), guanine (G), thymine (T), and uracil (U) are the fundamental units of nucleic acid molecules, in which Adenine and guanine, referred to purine bases, have a fused-ring skeletal structure derived of purine while uracil, and thymine, derived of pyrimidine, are referred to pyrimidine bases. As described herein, “nucleotide” is defined as a nucleoside including a phosphate group or other backbone linkage (internucleoside linkage).

In some embodiments, the polynucleotide (e.g., ASO or antisense polynucleotide) comprises at least one modification described herein. In other embodiments, the polynucleotides (e.g., ASO) comprise two, three, four, or more (optionally different) chemical modifications described herein. The modifications may be combinations of nucleobase (purine and/or pyrimidine), sugar and backbone (internucleoside) linkage modifications. The modifications may be located on one or more nucleotides of the polynucleotide. In some embodiments, all the nucleotides of the polynucleotide (e.g., ASO) are chemically modified. In some embodiments, all the nucleotides of the nucleic acid sequence with a biological function are chemically modified.

The polynucleotides (e.g., ASO(s)) of the present disclosure may contain from about 1% to about 100% modified nucleotides (either in relation to overall nucleotide content, or in relation to one or more types of nucleotide, i.e. any one or more of A, G, T/U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 85% to 95%, from 85% to 100%, from 90% to 95%, from 90% to 100%, and from 95% to 100%).

In some embodiments, the polynucleotides (e.g., ASO(s) are at least 50% modified, e.g., at least 50% of the nucleotides are modified. In some embodiments, the polynucleotides (e.g., ASO(s) are at least 75% modified, e.g., at least 75% of the nucleotides are modified. It is to be understood that since a nucleotide (sugar, base and phosphate moiety, e.g., linkage) may each be modified, any modification to any portion of a nucleotide, or nucleoside, will constitute a modification.

In some embodiments, the polynucleotides (e.g., ASO(s)) are at least 10% modified in only one component of the nucleotide, with such component being the nucleobase, sugar, or linkage between nucleosides. For example, modifications may be made to at least 10%, 15%, 20%, 25%, 30%, 35%, 40%, 45%, 50%, 55%, 60%, 65%, 70%, 75%, 80%, 85%, 90%, 95% or 100% of the nucleobases, sugars, or linkages of a polynucleotide (e.g., ASO) described herein.

The polynucleotides (e.g., ASO(s)) can be designed with a patterned array of sugar, nucleobase or linkage modifications. In some embodiments, the polynucleotides (e.g., ASO(s)) can comprise modifications to maximize stability.

### Modifications: Sugars

Modifications of the modified nucleosides and nucleotides can be present in the sugar subunit. In some embodiments, the polynucleotide (e.g., ASO) comprises at least one sugar modification. Generally, RNA includes the sugar subunit: ribose, which is a 5-membered ring having an oxygen.

In one example, the 2′ hydroxyl group (OH) can be modified or replaced with a number of different substituents. Exemplary substitutions at the 2′OH-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl) oxy; a sugar (e.g., ribose, pentose, or any described herein); a polyethyleneglycol (PEG)-O (CH₂CH₂O)nCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); and “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, or amino bridges; aminoalkyl; aminoalkoxy; amino; and amino acid.

Other exemplary sugar modifications include replacement of the oxygen (O) in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′->2′)), and peptide nucleic acid (PNA, where 2-amino-ethyl-glycine linkages replace the ribose and phosphodiester backbone).

The sugar subunit can also contain one or more carbons that possess the opposite stereochemical configuration than that of the corresponding carbon in ribose. Thus, antisense polynucleotides (e.g., ASO(s)) as described herein can include nucleotides containing, e.g., arabinose, as the sugar.

In some embodiments, at least one of the 2′ positions of the sugar (OH in RNA or H in DNA) of a nucleotide of the polynucleotides (e.g., ASO) is substituted with —O-Methoxyethyl, referred to as 2′-OMe. In some embodiments, at least one of the 2′ positions of the sugar (OH in RNA or H in DNA) of a nucleotide of the polynucleotides (e.g., ASO) is substituted with —F, referred to as 2′-F. In some embodiments, the sugar modification can be one or more locked nucleic acids (LNAs). In some embodiments, the polynucleotides (e.g., ASO) can be fully 2′-MOE-sugar modified.

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties. In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”), and 2′-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6.

In certain embodiments, 2′-substituent groups are selected from among: halo, allyl, amino, azido, SH, CN, OCN, CF, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rm)-alkyl, O-alkenyl, S-alkenyl, N(Rm)-alkenyl, O-alkynyl, S-alkynyl, N(Rm)-alkynyl, O-alkylenyl-O-alkyl, alkynyl, alkaryl, aralkyl, O-alkaryl, O-aralkyl, O(CH₂)₂SCH₃, O(CH₂)₂₀N(Rm)(Rn) or OCH₂C(═O)—N(Rm)(Rn), where each Rm and Rn is, independently, H, an amino protecting group, or substituted or unsubstituted C₁-C₁₀ alkyl, and the 2′-substituent groups described in Cook et al., U.S. Pat. No. 6,531,584; Cook et ah, U.S. Pat. No. 5,859,221; and Cook et ah, U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃OCH₃, O(CH₂)₃NH₂, CH₂CH═CH₂, OCH₂CH═CH₂, OCH₂CH₂OCH₃, O(CH₂)₂SCH₃, O(CH₂)₂₀N(Rm)(Rn), O(CH₂), ON(CH₃)₂, O(CH₂)₂O(CH₂)₂N(CH₃)₂, and N-substituted acetamide (OCH2C(═O)—N(Rm)(Rn)), where each Rm and Rn is, independently, H, an amino protecting group, or substituted or unsubstituted C₁-C₁₀ alkyl, e.g., for example, OCH2C(═O)—N(H)CH3 (“NMA”).

In certain embodiments, a 2′-substituted non-bicyclic modified nucleoside comprises a sugar moiety comprising a non-bridging 2′-substituent group selected from: F, OCF₃OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”). In certain embodiments, a 2′-substituted non-bicyclic modified nucleoside comprises a sugar moiety comprising a non-bridging 2′-substituent group selected from: F, OCH, OCH₂CH₂OCH₃, and OCH₂C(═O)—N(H)CH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH2-2′, 4′-(CH2)2-2′, 4′-(CH2)3-2′, 4′-CH2-O-2′ (“LNA”), 4′-CH2-S-2′, 4′-(CH2)2-O-2′ (“ENA”), 4′-CH(CH3)-O-2′ (referred to as “constrained ethyl” or “cEt”), 4′-CH2-O—CH2-2′, 4′-CH2-N(R)-2′, 4′-CH(CH2OCH3)-O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH3)(CH3)-O-2′ and analogs thereof (see, e.g., Seth et al., U.S. Pat. No. 8,278,283), 4′-CH2-N(OCH)-2′ and analogs thereof (see, e.g., Prakash et al., U.S. Pat. No. 8,278,425), 4′-CH2-O—N(CH3)-2′ (see, e.g., Allerson et al., U.S. Pat. No. 7,696,345 and Allerson et al., U.S. Pat. No. 8,124,745), 4′-CH2-C(H)(CH3)-2′ (see, e.g., Zhou, et al., J. Org. Chem., 2009, 74, 118-134), 4′-CH2-C(═CH2)-2′ and analogs thereof (see e.g., Seth et al., U.S. Pat. No. 8,278,426), 4, —C(RaRb)—N(R)—O-2′, 4′-C(RaRb)—O—N(R)-2′, 4′-CH2-O—N(R)-2′, and 4′-CH2-N(R)—O-2′, wherein each R, Ra, and Rb is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Ra)(R)]n-, —[C(Ra)(Rb)]n-O—, —C(Ra)═C(Rb)—, —C(Ra)═N—, —C(═NRa)—, —C(═O)—, —C(═S)—, —O—, —Si(Ra)2-, —S(═O)x-, and —N(Ra)—; wherein: x is 0, 1, or 2; n is 1, 2, 3, or 4; each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJi, NJIJ2, SJi, N3, COOJi, acyl (C(═O)—H), substituted acyl, CN, sulfonyl (S(═O)2-Ji), or sulfoxyl (S(═O)-Ji); and each Ji and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al, U.S. Pat. No. 8,268,980; Seth et al, U.S. Pat. No. 8,546,556; Seth et al, U.S. Pat. No. 8,530,640; Migawa et al, U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the b-D configuration. LNA (β-D-configuration) α-L-LNA (α-L-configuration) bridge=4′-CH2-O-2′ bridge=4′-CH2-O-2′ α-L-methyleneoxy (4′-CH2-O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the b-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see. e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA: (“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et ah, U.S. Pat. No. 5,698,685; Summerton et ah, U.S. Pat. No. 5,166,315; Summerton et ah, U.S. Pat. No. 5,185,444; and Summerton et ah, U.S. Pat. No. 5,034,506). As used here, the term “morpholino” means a sugar surrogate having the following structure:

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et ah, Org. Biomol. Chem., 2013, 11, 5853-5865), and nucleosides and oligonucleotides described in Manoharan et ah, WO2011/133876.

Many other bicyclic and tricyclic sugar and sugar surrogate ring systems are known in the art that can be used in modified nucleosides.

### Modifications: Nucleosides

In some embodiments, the ASOs comprise nucleosides that comprise, or consist of, non-modified nucleosides, for example, adenine, guanine, uracil, thymine, or cytosine.

In one embodiment, the ASOs may comprise modified variants of nucleosides (i.e., provided that Watson-Crick base-pairing of the base nucleoside is not affected). In an embodiment, the ASOs may comprise modified A, modified C, modified G or modified U. In one embodiment, the modified ASOs may comprise, but are not limited to, modified C such as 5-methylcytosine, or 5-hydroxymethylcytosine, modified U such as 5-methyluridine or replacement with thymine, or modified A such as N₆-methyladenine. In one embodiment, the ASO may comprise a mixture of non-modified and modified nucleosides. For SEQ ID NOS; 4-546 set out herein, whilst shown with non-modified nucleosides, the present invention also embraces modified variants of the nucleosides.

In certain embodiments, oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside.

The polynucleotide (e.g., ASO) of the present disclosure may comprise a nucleoside modification. One or more atoms of a pyrimidine nucleobase may be replaced or substituted, for example, with optionally substituted amino, optionally substituted thiol, optionally substituted alkyl (e.g., methyl or ethyl), optionally substituted or halo (e.g., chloro or fluoro) atoms or groups.

As non-limiting examples, the uracil nucleosides of the polynucleotide (e.g. ASO) of the present disclosure are all modified. The modifications may be the same or different. In some embodiments, the guanine nucleosides of the polynucleotide (e.g., ASO) of the present disclosure are all modified. The modifications may be the same or different. In some embodiments, the guanine nucleosides of the polynucleotide (e.g., ASO) of the present disclosure are all modified. The modifications may be the same or different. In some embodiments, the cytosine nucleosides of the polynucleotide (e.g., ASO) of the present disclosure are all modified. The modifications may be the same or different. In some embodiments, the adenine nucleosides of the polynucleotide (e.g., ASO) of the present disclosure are all modified. The modifications may be the same or different.

### Modifications: Nucleobases

The modified nucleosides and nucleotides can include a modified nucleobase. Examples of nucleobases in RNA include, but are not limited to, adenine (A), guanine (G), cytosine (C), and uracil (U). Examples of nucleobases in DNA include, but are not limited to, adenine (A), guanine (G), cytosine (C), and thymine (T).

In some embodiments, the modified nucleobase is a modified uracil (U).

Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine (I⁵U) or 5-bromo-uridine (br⁵U)), 3-methyl-uridine (m³U), 5-methoxy-uridine (mo⁵U), uridine 5-oxyacetic acid (cmoSU), uridine 5-oxyacetic acid methyl ester (mcmoSU), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nms²U), 5-methylaminomethyl-uridine (mnm⁵U), 5-methylaminomethyl-2-thio-uridine (mnmns²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine, 5-methyl-uridine (m⁵U, i.e., having the nucleobase deoxythymine), 1-methylpseudouridine (m¹W), 5-methyl-2-thio-uridine (m⁵s²U), pseudouracil (ψ), 1-methyl-4-thio-pseudouridine (mis⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ)), 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine.

In some embodiments, the modified nucleobase is a modified cytosine (C).

Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine (A).

Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine (G).

Exemplary nucleobases and nucleosides having a modified guanine include inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2,N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (mGm), N2,7-dimethyl-2′-O-methyl-guanosine (m^{2,7}Gm), 2′-O-methyl-inosine (Im), 1,2′-O-dimethyl-inosine (m¹Im), and 2′-O-ribosylguanosine (phosphate) (Gr(p)).

In some embodiments, the nucleobase of the nucleotide can be independently selected from a purine, a pyrimidine, a purine or pyrimidine analog. The nucleobase and/or analog may be each be independently selected from adenine, cytosine, guanine, thymine, uracil, naturally-occurring and synthetic derivatives of a base, including but not limited to pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine.

In certain embodiments, modified nucleobases are selected alone or in combination from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyl adenine, 2-thiouracil, 2-thiothymine and 2-thiocytosine, 5-propynyl (—C═C—CH) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et ah, Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et ah, U.S. Pat. No. 4,845,205; Spielvogel et al., U.S. Pat. No. 5,130,302; Rogers et ah, U.S. Pat. No. 5,134,066; Bischofberger et al., U.S. Pat. No. 5,175,273; Urdea et ah, U.S. Pat. No. 5,367,066; Benner et ah, U.S. Pat. No. 5,432,272; Matteucci et ah, U.S. Pat. No. 5,434,257; Gmeiner et ah, U.S. Pat. No. 5,457,187; Cook et al., U.S. Pat. No. 5,459,255; Froehler et ah, U.S. Pat. No. 5,484,908; Matteucci et ah, U.S. Pat. No. 5,502,177; Hawkins et ah, U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et ah, U.S. Pat. No. 5,614,617; Froehler et ah, U.S. Pat. No. 5,645,985; Cook et ah, U.S. Pat. No. 5,681,941; Cook et ah, U.S. Pat. No. 5,811,534; Cook et al., U.S. Pat. No. 5,750,692; Cook et ah, U.S. Pat. No. 5,948,903; Cook et ah, U.S. Pat. No. 5,587,470; Cook et ah, U.S. Pat. No. 5,457,191; Matteucci et al., U.S. Pat. No. 5,763,588; Froehler et ah, U.S. Pat. No. 5,830,653; Cook et ah, U.S. Pat. No. 5,808,027; Cook et al., 6,166,199; and Matteucci et al., U.S. Pat. No. 6,005,096.

### Modifications: Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond, P(O₂)═O, (also referred to as unmodified or naturally occurring linkages); phosphotriesters; methylphosphonates; methoxypropylphosphonates (“MOP”); phosphoramidates; mesyl phosphoramidates; phosphorothioates (P(O₂)═S); and phosphorodithioates (HS—P═S). Representative non-phosphorus containing internucleoside linking groups include but are not limited to methylenemethylimino (—CH2-N(CH)—O—CH2-); thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereo configuration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al, JACS, 2003, 125, 8307, Wan et al. Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH2-N(CH3)-O-5′), amide-3 (3′-CH2-C(═O)—N(H)-5′), amide-4 (3′-CH2-N(H)—C(═O)-5′), formacetal (3′-O—CH2-O-5′), methoxypropyl, and thioformacetal (3′-S-CH2-O-5′). Further neutral internucleoside linkages include nonionic linkages comprising siloxane (dialkylsiloxane), carboxylate ester, carboxamide, sulfide, sulfonate ester and amides (see e.g., Carbohydrate Modifications in Antisense Research, Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, a modified internucleoside linkage is any of those described in WO 2021/030778, incorporated by reference herein.

### Modifications: Backbone Phosphate Groups

Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, methylphosphonates phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polynucleotides through the unnatural phosphorothioate backbone linkages. Phosphorothioate DNA and RNA have increased nuclease resistance and subsequently a longer half-life in a cellular environment. Phosphorothioate linked polynucleotide molecules are expected to also reduce the innate immune response through weaker binding/activation of cellular innate immune molecules.

In some embodiments, the polynucleotides (e.g., ASO(s)) of the present disclosure comprise at least one phosphorothioate linkage, methylphosphonate linkage between nucleotides, 5′-(E)-vinylphosphonate (5′-E-VP), a phosphate mimic, as a modification.

The ASO defined herein may have any suitable backbone (i.e., any suitable nucleoside linkage and/or any suitable nucleoside, wherein the nucleoside may have any suitable sugar and/or any suitable nucleobase). In an embodiment, the ASO defined herein is resistant to RNase H cleavage. In an embodiment, the ASO may be a steric block ASO. In an embodiment, the ASO may be resistant to cleavage and/or a poor substrate for RNase H when bound to a crucial element or target sequence. In an embodiment, the ASO does not cause degradation of the UNC13A pre-mRNA or mRNA.

In one embodiment, the backbone is formed from RNA (alternating phosphate and ribose), LNA (locked nucleic acid), tcDNA (tri-cyclo DNA), cEt (constrained ethyl bridged nucleic acid); ENA (ethylene-bridged nucleic acid), HNA (hexitol nucleic acids), TNA (threose nucleic acid), PMO (phosphorodiamidate morpholino oligomer) PMO, PNA (peptide nucleic acid), 2-OMe-RNA, 2′-O-methoxyethyl (MOE) nucleic acids, or 2-O-(2-methylcarbomoyl (MCE) nucleotides, or any combination thereof. The ASO may further comprise a portion of DNA nucleotides, for example, at least 10%, or at least 20%, or at least 30%, or at least 40%, or at least 50%, or at least 60%, or up to 70% of DNA nucleotides, i.e., in combination with LNA, tcDNA, cET, ENA, HNA, TNA, PMO, PNA, 2′-OMe-RNA, 2′-O-methoxyethyl (MOE) nucleic acids or MCE nucleotides. The backbone may consist entirely of one nucleotide, or a mixture of one or more nucleotides. In an embodiment, the ASO has a bridged nucleic acid (i.e., locked or constrained) backbone, for example, LNA, cET or ENA backbone. These backbones may have good stability, high binding constants with RNA and/or resistance to RNase H cleavage. In some embodiments, the ASO comprises a phosphate (i.e., phosphodiester) nucleotide linkage. In some embodiments, the ASO comprises a phosphorodiamidate nucleotide linkage. other embodiments, the ASO comprises a phosphorothioate nucleotide linkage (i.e., otherwise known as a PS-ASO).

In some embodiments, the ASO comprises LNA, i.e., comprising LNA and DNA and more particularly phosphorothioate LNA and DNA. In some examples, disclosed herein, the ASO comprises LNA or 2-OMe-RNA.

In some examples, the ASO comprises only a portion of a bridged nucleic acid, such as LNA. In some examples, the ASO comprises from about 20-60% bridged nucleic acid (e.g., LNA), or preferably from about 30-50% bridged nucleic acid (e.g., LNA). In some embodiments, the ASO having an LNA backbone comprises bridge nucleic acids (e.g., LNA bases) in combination with DNA bases. In some examples, the ASO comprises at least 20% bridged nucleic acids (e.g., LNA) or at least 30%, or at least 40% bridged nucleic acids (e.g., LNA). In some examples, the ASO comprises less than 100% bridged nucleic acids (e.g., LNA), or less than 90%, or less than 80%, or less than 70%, or less than 60%, or less than 50% bridged nucleic acids (e.g., LNA). In some embodiments, the ASO comprises a bridged nucleic acid (e.g., LNA) at least or equal to every 3 nucleotides, or at least or equal to every 2 nucleotides.

In some examples, the ASO comprises 2-OMe-RNA. In some examples, the ASO comprises 100% 2-OMe-RNA, more particularly 100% phosphorothioated 2-OMe-RNA. In some embodiments, the ASO comprises only a portion of 2-OMe-RNA.

In an embodiment, the ASO has an RNA backbone. While the ASO sequences/portions of ASO sequences provided with the sequence listing herein have an RNA backbone, any other suitable backbone may be used provided the nucleoside sequence is the same (as is elsewhere described herein, in the ASOs disclosed herein “U” and “T” nucleosides, e.g. uracil or thymine, may be used interchangeably. Therefore “U” in any of the ASO sequences described herein (e.g., including SEQ ID NOS: 4-546) may be replaced by “T”).

### Modifications: Motifs

Different sugar modifications, nucleobase modifications, and/or internucleoside linkages (e.g., backbone structures) may be introduced at various positions in a polynucleotide (e.g., ASO) described herein. One of ordinary skill in the art will appreciate that the nucleotide analogs or other modification(s) may be located at any position(s) of a polynucleotide (e.g., ASO) such that the function of the polynucleotide (e.g., ASO) is not substantially decreased.

### Modifications: Sugar Motifs

In certain embodiments, oligonucleotides comprise one or more type of modified sugar and/or unmodified sugar moiety arranged along the oligonucleotide, or portion thereof, in a defined pattern or sugar motif. In certain instances, such sugar motifs include but are not limited to any of the sugar modifications discussed herein.

Certain modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer). In certain embodiments, modified oligonucleotides of the present invention are not gapmers.

In certain embodiments, the wings of a gapmer comprise 1-6 nucleosides. In certain embodiments, each nucleoside of each wing of a gapmer comprises a modified sugar moiety. In certain embodiments, at least one, at least two, at least three, at least four, at least five, or at least six nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, at least one nucleoside of the gap of a gapmer comprises a modified sugar moiety and each remaining nucleoside comprises a 2′-deoxyribosyl sugar moiety.

Herein, the lengths (number of nucleosides) of the three regions of a gapmer may be provided using the notation [# of nucleosides in the 5′-wing]-[# of nucleosides in the gap]-[# of nucleosides in the 3′-wing] Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise a 2′-deoxyribosyl sugar moiety. Thus, a 5-10-5 MOE gapmer consists of 5 linked 2′-MOE nucleosides in the 5′-wing, 10 linked 2′-deoxyribonucleosides in the gap, and 5 linked 2′-MOE nucleosides in the 3′-wing.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, abicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, abicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, abicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, modified oligonucleotides have a sugar motif comprising at least 1, at least 2, at least 3, or at least 42′-deoxyribonucleosides, but are otherwise fully modified. In certain embodiments, modified oligonucleotides having at least one fully modified sugar motif may also comprise not more than 1, not more than 2, not more than 3, or not more than 42′-deoxyribonucleosides. In certain embodiments, modified oligonucleotides having at least one fully modified sugar motif may also comprise exactly 1, exactly 2, exactly 3, or exactly 42′-deoxyribonucleosides. In certain embodiments, modified oligonucleotides comprise more than 42′-deoxyribonucleosides, provided they do not include a region comprising 4 or more contiguous 2′-deoxyribonucleosides

### Modifications: Nucleobase Motifs

In certain embodiments, the ASOs or oligonucleotides comprise modified and/or unmodified nucleobases arranged along the ASO or oligonucleotide, or portion thereof, in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified ASOs or oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide. In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-deoxyribosyl sugar moiety. In certain embodiments, the modified nucleobase is selected from: a 2-thiopyrimidine and a 5-propynepy rimidine.

### Modifications: Internucleoside Linkage Motifs

In certain embodiments, ASOs or oligonucleotides comprise modified and/or unmodified internucleoside linkages arranged along the oligonucleotide, or portion thereof, in a defined pattern or motif. In certain embodiments, each internucleoside linking group is a phosphodiester internucleoside linkage. In certain embodiments, each internucleoside linking group of a modified oligonucleotide is a phosphorothioate internucleoside linkage. In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain such embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphodiester internucleoside linkages. In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphorothioate internucleoside linkages. In certain embodiments, modified oligonucleotides comprise at least 1, at least 2, at least 3, at least 4, or at least 5 phosphodiester internucleoside linkages and the remainder of the internucleoside linkages are phosphorothioate internucleoside linkages.

### Conjugates

In certain embodiments, ASOs or oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol orundecyl residues (Saison-Behmoaras et al., EMBOJ., 1991, 10, 1111-11 18; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-0-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster {e.g., WO2014/179620).

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (‘S’)-(+)-pranoprofen. carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

### Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are subunits making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxy late (SMCC) and 6-aminohexanoic acid (AHEX or AHA). Other conjugate linkers include but are not limited to substituted or unsubstituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxyribonucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate internucleoside linkage.

In certain such embodiments, the cleavable moiety is 2′-deoxyadenosine.

### Preferred ASOs

Also disclosed herein is an ASO comprising 17-24 nucleotides which is capable of binding to a UNC13A splice site or flanking regions thereof (i.e., to modulate UNC13A cryptic exon splicing). In an embodiment, the ASO is capable of binding to a UNC13A splice donor site or flanking regions thereof (e.g., capable of binding and/or substantially complementary to one or more of SEQ ID NO: 547, SEQ ID NO: 548, SEQ ID NO: 549, or SEQ ID NO: 551) to modulate UNC13A cryptic splicing. In some embodiments, the ASO comprises a) at least SEQ ID NO: 295, SEQ ID NO: 297, SEQ ID NO: 299, SEQ ID NO: 300 and SEQ ID NO: 301, or b) at least SEQ ID NO: 296, SEQ ID NO: 298, SEQ ID NO: 299, SEQ ID NO: 300 and SEQ ID NO: 301, or c) at least SEQ ID NO: 297, SEQ ID NO: 299 and SEQ ID NO: 300, and SEQ ID NO: 301 and SEQ ID NO: 302, or d) at least SEQ ID NO: 298, SEQ ID NO: 299 and SEQ ID NO: 300, and SEQ ID NO: 301 and SEQ ID NO: 302, or e) at least SEQ ID NO: 299, SEQ ID NO: 300, SEQ ID NO: 301 and SEQ ID NO: 302 and SEQ ID NO: 303 or f) at least SEQ ID NO: 300, SEQ ID NO: 301 and SEQ ID NO: 302, and SEQ ID NO: 303 and SEQ ID NO: 304, or g) at least SEQ ID NO: 301, SEQ ID NO: 302 and SEQ ID NO: 303, and SEQ ID NO: 304 and SEQ ID NO: 305, or h) at least SEQ ID NO: 302, SEQ ID NO: 303 and SEQ ID NO: 304, and SEQ ID NO: 305 and SEQ ID NO: 306 or i) at least SEQ ID NO: 303, SEQ ID NO: 304 and SEQ ID NO: 305, and SEQ ID NO: 306 and SEQ ID NO: 307, or j) at least SEQ ID NO: 304, SEQ ID NO: 305 and SEQ ID NO: 306, and SEQ ID NO: 307 and SEQ ID NO: 308, k) at least SEQ ID NO: 305, SEQ ID NO: 306 and SEQ ID NO: 307, and SEQ ID NO: 308 and SEQ ID NO: 309 or l) at least SEQ ID NO: 306, SEQ ID NO: 307 and SEQ ID NO: 308, and SEQ ID NO: 309 and SEQ ID NO: 310, or m) at least SEQ ID NO: 307, SEQ ID NO: 308 and SEQ ID NO: 309, and SEQ ID NO: 310 and SEQ ID NO: 311, or n) at least SEQ ID NO: 308, SEQ ID NO: 309 and SEQ ID NO: 310, and SEQ ID NO: 311 and SEQ ID NO: 312, or o) at least SEQ ID NO: 312, SEQ ID NO: 313 and SEQ ID NO: 314, and SEQ ID NO: 315 and SEQ ID NO: 316, or p) at least SEQ ID NO: 313, SEQ ID NO: 314 and SEQ ID NO: 315, and SEQ ID NO: 316 and SEQ ID NO: 317, or q) at least SEQ ID NO: 314, SEQ ID NO: 315 and SEQ ID NO: 316, and SEQ ID NO: 317 and SEQ ID NO: 318, or r) at least SEQ ID NO: 315, SEQ ID NO: 316 and SEQ ID NO: 317, and SEQ ID NO: 318 and SEQ ID NO: 319, or s) at least SEQ ID NO: 316, SEQ ID NO: 317 and SEQ ID NO: 318, and SEQ ID NO: 319 and SEQ ID NO: 320, or t) at least SEQ ID NO: 317, SEQ ID NO: 318 and SEQ ID NO: 319, and SEQ ID NO: 320 and SEQ ID NO: 321, or u) at least SEQ ID NO: 318, SEQ ID NO: 319 and SEQ ID NO: 320, and SEQ ID NO: 321 and SEQ ID NO: 322, or v) at least SEQ ID NO: 319, SEQ ID NO: 320 and SEQ ID NO: 321, and SEQ ID NO: 322 and SEQ ID NO: 323, or w) at least SEQ ID NO: 320, SEQ ID NO: 321 and SEQ ID NO: 322, and SEQ ID NO: 323 and SEQ ID NO: 324 or a combination thereof. In an embodiment, the ASO is capable of binding to a splice acceptor site or flanking regions thereof, more specifically a short acceptor site, (e.g., capable of binding to or substantially complementary to SEQ ID NO: 552 or SEQ ID NO: 554), and wherein the ASO comprises at least aa) SEQ ID NO: 159, SEQ ID NO: 160, SEQ ID NO: 161, SEQ ID NO: 162 and SEQ ID NO: 163, or bb) SEQ ID NO: 160, SEQ ID NO 161, SEQ ID NO 162, SEQ ID NO 163 and SEQ ID NO: 164, or cc) SEQ ID NO: 161, SEQ ID NO: 162. SEQ ID NO: 163, SEQ ID NO: 164, and SEQ ID NO: 165, or dd) SEQ ID NO: 162, SEQ ID NO: 163. SEQ ID NO: 164, SEQ ID NO: 165, and SEQ ID NO: 166, or ee) SEQ ID NO: 163, SEQ ID NO: 164. SEQ ID NO: 165, SEQ ID NO: 166, and SEQ ID NO: 167, or ff) SEQ ID NO: 164, SEQ ID NO: 165. SEQ ID NO: 166, SEQ ID NO: 167, and SEQ ID NO: 168, or gg) SEQ ID NO: 165, SEQ ID NO: 166, SEQ ID NO: 167, SEQ ID NO: 168, and SEQ ID NO: 169, or hh) SEQ ID NO: 166, SEQ ID NO: 167. SEQ ID NO: 168, SEQ ID NO: 169, and SEQ ID NO: 170, or ii) SEQ ID NO: 167, SEQ ID NO: 168. SEQ ID NO: 169, SEQ ID NO: 170, and SEQ ID NO: 171, or jj) SEQ ID NO: 168, SEQ ID NO: 169, SEQ ID NO: 170, SEQ ID NO: 171, and SEQ ID NO: 172, or kk) SEQ ID NO: 169, SEQ ID NO: 170, SEQ ID NO: 171, SEQ ID NO: 172, and SEQ ID NO: 173, or ll) SEQ ID NO: 169, SEQ ID NO: 170, SEQ ID NO: 171, SEQ ID NO: 172, and SEQ ID NO: 173, or mm) SEQ ID NO: 170, SEQ ID NO: 171, SEQ ID NO: 172, SEQ ID NO: 173, and SEQ ID NO: 174, or nn) SEQ ID NO: 171, SEQ ID NO: 172, SEQ ID NO: 173, SEQ ID NO: 174, and SEQ ID NO: 175, or oo) SEQ ID NO: 172, SEQ ID NO: 173, SEQ ID NO: 174, SEQ ID NO: 175, and SEQ ID NO: 176, or pp) SEQ ID NO: 173, SEQ ID NO: 174, SEQ ID NO: 175, SEQ ID NO: 176, and SEQ ID NO: 177, or a combination thereof. In an embodiment, the ASO is capable of binding to a splice acceptor site, more specifically a long acceptor site, (e.g., capable of binding to or substantially complementary to SEQ ID NO: 552 or SEQ ID NO: 553), and wherein the ASO comprises at least aaa) SEQ ID NO: 105, SEQ ID NO: 106, SEQ ID NO: 107 and SEQ ID NO: 108, and SEQ ID NO: 109 or bbb) SEQ ID NO: 106, SEQ ID NO: 107, SEQ ID NO: 108, SEQ ID NO: 109 and SEQ ID NO: 110, or ccc) SEQ ID NO: 107, SEQ ID NO: 108, SEQ ID NO: 109, SEQ ID NO: 110 and SEQ ID NO: 111, or ddd) SEQ ID NO: 108, SEQ ID NO: 109, SEQ ID NO: 110, SEQ ID NO: 111 and SEQ ID NO: 112, or eee) SEQ ID NO: 109, SEQ ID NO: 110, SEQ ID NO: 111 and SEQ ID NO: 112 and SEQ ID NO: 113 or fff) SEQ ID NO: 110, SEQ ID NO: 111, SEQ ID NO: 112, SEQ ID NO 113 and SEQ ID NO: 114, or ggg) SEQ ID NO: 111, SEQ ID NO: 112, SEQ ID NO 113, SEQ ID NO: 114 and SEQ ID NO: 115, or hhh) SEQ ID NO: 112, SEQ ID NO 113, SEQ ID NO: 114, SEQ ID NO: 115 and SEQ ID NO: 116, or iii) SEQ ID NO: 113, SEQ ID NO: 114, SEQ ID NO: 115, SEQ ID NO: 116 and SEQ ID NO: 117, or jjj) SEQ ID NO: 114, SEQ ID NO: 115, SEQ ID NO: 116, SEQ ID NO: 117, and SEQ ID NO: 118, or kkk) SEQ ID NO: 115, SEQ ID NO: 116, SEQ ID NO: 117, SEQ ID NO: 118 and SEQ ID NO: 119, or lll) SEQ ID NO: 116, SEQ ID NO: 117, SEQ ID NO: 118, SEQ ID NO: 119 and SEQ ID NO: 120, or mmm) SEQ ID NO: 117, SEQ ID NO: 118, SEQ ID NO: 119, SEQ ID NO: 120 and SEQ ID NO: 121, or nnn) SEQ ID NO: 118, SEQ ID NO: 119, SEQ ID NO: 120, SEQ ID NO: 121 and SEQ ID NO: 122, or ooo) SEQ ID NO: 119, SEQ ID NO: 120, SEQ ID NO: 121, SEQ ID NO: 122 and SEQ ID NO: 123, or ppp) SEQ ID NO: 120, SEQ ID NO: 121, SEQ ID NO: 122, SEQ ID NO: 123 and SEQ ID NO: 124, or qqq) SEQ ID NO: 120, SEQ ID NO: 121, SEQ ID NO: 122, SEQ ID NO: 123 and SEQ ID NO: 124, or rrr) SEQ ID NO: 121, SEQ ID NO: 122, SEQ ID NO: 123, SEQ ID NO: 124 and SEQ ID NO: 125 or a combination thereof.

In a preferred embodiment, the ASO comprises a bridged nucleic acid, preferably from 30-50% bridged nucleic acid, and preferably wherein the bridged nucleic acid is LNA. In a preferred embodiment, the ASO comprises phosphothioate linkages.

Also disclosed herein, is an ASO comprising 20-24 nucleotides which is capable of binding to a UNC13A splice site or flanking regions thereof. In an embodiment, the ASO is capable of binding to a donor splice site or flanking regions thereof (e.g., capable of binding to or substantially complementary to one or more of SEQ ID NO: 547, 548, 549, 550 or 551). In an embodiment, the ASO comprises SEQ ID NO: 295, SEQ ID NO: 297, SEQ ID NO: 299, SEQ ID NO: 300, SEQ ID NO: 301, SEQ ID NO: 302, SEQ ID NO: 303, SEQ ID NO: 304, and optionally SEQ ID NO: 305 (i.e., for a 21 nucleotide ASO), and further optionally SEQ ID NO 306 (i.e., for a 22 nucleotide ASO), and further optionally SEQ ID NO 307 (i.e., for a 23 nucleotide ASO), and further optionally SEQ ID NO: 308 (i.e., for a 24 nucleotide ASO). In an embodiment, the ASO comprises SEQ ID NO: 296, SEQ ID NO: 298, SEQ ID NO: 299, SEQ ID NO: 300, SEQ ID NO: 301, SEQ ID NO: 302, SEQ ID NO: 303, SEQ ID NO: 304, and optionally SEQ ID NO: 305 (i.e., for a 21 nucleotide ASO), and further optionally SEQ ID NO 306 (i.e., for a 22 nucleotide ASO), and further optionally SEQ ID NO 307 (i.e., for a 23 nucleotide ASO), and further optionally SEQ ID NO: 308 (i.e., for a 24 nucleotide ASO). In an embodiment, the ASO comprises SEQ ID NO: 297, SEQ ID NO: 299, SEQ ID NO: 300, SEQ ID NO: 301, SEQ ID NO: 302, SEQ ID NO: 303, SEQ ID NO: 304, SEQ ID NO: 305, and optionally SEQ ID NO: 306 (i.e., for a 21 nucleotide ASO), and further optionally SEQ ID NO 307 (i.e., for a 22 nucleotide ASO), and further optionally SEQ ID NO 308 (i.e., for a 23 nucleotide ASO), and further optionally SEQ ID NO: 309 (i.e., for a 24 nucleotide ASO). In an embodiment, the ASO comprises SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, and optionally SEQ ID NO: 306 (i.e., for a 21 nucleotide ASO), and further optionally SEQ ID NO 307 (i.e., for a 22 nucleotide ASO), and further optionally SEQ ID NO 308 (i.e., for a 23 nucleotide ASO), and further optionally SEQ ID NO: 309 (i.e., for a 24 nucleotide ASO). In an embodiment, the ASO comprises SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, and optionally SEQ ID NO: 307 (i.e., for a 21 nucleotide ASO), and further optionally SEQ ID NO 308 (i.e., for a 22 nucleotide ASO), and further optionally SEQ ID NO 309 (i.e., for a 23 nucleotide ASO), and further optionally SEQ ID NO: 310 (i.e., for a 24 nucleotide ASO). In an embodiment, the ASO comprises SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307 and optionally SEQ ID NO: 308 (i.e., for a 21 nucleotide ASO), and further optionally SEQ ID NO 309 (i.e., for a 22 nucleotide ASO), and further optionally SEQ ID NO 310 (i.e., for a 23 nucleotide ASO), and further optionally SEQ ID NO: 311 (i.e., for a 24 nucleotide ASO). In an embodiment, the ASO comprises SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308 and optionally SEQ ID NO: 309 (i.e., for a 21 nucleotide ASO), and further optionally SEQ ID NO 310 (i.e., for a 22 nucleotide ASO), and further optionally SEQ ID NO 311 (i.e., for a 23 nucleotide ASO), and further optionally SEQ ID NO: 312 (i.e., for a 24 nucleotide ASO). In an embodiment, the ASO comprises SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309 and optionally SEQ ID NO: 310 (i.e., for a 21 nucleotide ASO), and further optionally SEQ ID NO 311 (i.e., for a 22 nucleotide ASO), and further optionally SEQ ID NO 312 (i.e., for a 23 nucleotide ASO), and further optionally SEQ ID NO: 313 (i.e., for a 24 nucleotide ASO). In an embodiment, the ASO comprises SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310 and optionally SEQ ID NO: 311 (i.e., for a 21 nucleotide ASO), and further optionally SEQ ID NO 312 (i.e., for a 22 nucleotide ASO), and further optionally SEQ ID NO 313 (i.e., for a 23 nucleotide ASO), and further optionally SEQ ID NO: 314 (i.e., for a 24 nucleotide ASO). In an embodiment, the ASO comprises SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311 and optionally SEQ ID NO: 312 (i.e., for a 21 nucleotide ASO), and further optionally SEQ ID NO 313 (i.e., for a 22 nucleotide ASO), and further optionally SEQ ID NO 314 (i.e., for a 23 nucleotide ASO), and further optionally SEQ ID NO: 315 (i.e., for a 24 nucleotide ASO). In an embodiment, the ASO comprises SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312 and optionally SEQ ID NO: 313 (i.e., for a 21 nucleotide ASO), and further optionally SEQ ID NO 314 (i.e., for a 22 nucleotide ASO), and further optionally SEQ ID NO 315 (i.e., for a 23 nucleotide ASO), and further optionally SEQ ID NO: 316 (i.e., for a 24 nucleotide ASO). In an embodiment, the ASO comprises SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313 and optionally SEQ ID NO: 314 (i.e., for a 21 nucleotide ASO), and further optionally SEQ ID NO 315 (i.e., for a 22 nucleotide ASO), and further optionally SEQ ID NO 316 (i.e., for a 23 nucleotide ASO), and further optionally SEQ ID NO: 317 (i.e., for a 24 nucleotide ASO). In an embodiment, the ASO comprises SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314 and optionally SEQ ID NO: 315 (i.e., for a 21 nucleotide ASO), and further optionally SEQ ID NO 316 (i.e., for a 22 nucleotide ASO), and further optionally SEQ ID NO 317 (i.e., for a 23 nucleotide ASO), and further optionally SEQ ID NO: 318 (i.e., for a 24 nucleotide ASO). In an embodiment, the ASO comprises SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315 and optionally SEQ ID NO: 316 (i.e., for a 21 nucleotide ASO), and further optionally SEQ ID NO 317 (i.e., for a 22 nucleotide ASO), and further optionally SEQ ID NO 318 (i.e., for a 23 nucleotide ASO), and further optionally SEQ ID NO: 319 (i.e., for a 24 nucleotide ASO). In an embodiment, the ASO comprises SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316 and optionally SEQ ID NO: 317 (i.e., for a 21 nucleotide ASO), and further optionally SEQ ID NO 318 (i.e., for a 22 nucleotide ASO), and further optionally SEQ ID NO 319 (i.e., for a 23 nucleotide ASO), and further optionally SEQ ID NO: 320 (i.e., for a 24 nucleotide ASO). In an embodiment, the ASO comprises SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317 and optionally SEQ ID NO 318 (i.e., for a 21 nucleotide ASO), and further optionally SEQ ID NO 319 (i.e., for a 22 nucleotide ASO), and further optionally SEQ ID NO 320 (i.e., for a 23 nucleotide ASO), and further optionally SEQ ID NO: 321 (i.e., for a 24 nucleotide ASO). In an embodiment, the ASO comprises SEQ ID NO: 311, SEQ ID NO: 312, SEQ ID NO: 313, SEQ ID NO: 314, SEQ ID NO: 315, SEQ ID NO: 316, SEQ ID NO: 317 and SEQ ID NO: 318 and optionally SEQ ID NO 319 (i.e., for a 21 nucleotide ASO), and further optionally SEQ ID NO 320 (i.e., for a 22 nucleotide ASO), and further optionally SEQ ID NO 321 (i.e., for a 23 nucleotide ASO), and further optionally SEQ ID NO: 322 (i.e., for a 24 nucleotide ASO). In an embodiment, the ASO comprises SEQ ID NO: 312, SEQ ID NO: 313, SEQ ID NO: 314, SEQ ID NO: 315, SEQ ID NO: 316, SEQ ID NO: 317 and SEQ ID NO: 318 and SEQ ID NO: 319 and optionally SEQ ID NO 320 (i.e., for a 21 nucleotide ASO), and further optionally SEQ ID NO 321 (i.e., for a 22 nucleotide ASO), and further optionally SEQ ID NO 322 (i.e., for a 23 nucleotide ASO), and further optionally SEQ ID NO: 323 (i.e., for a 24 nucleotide ASO). In an embodiment, the ASO comprises SEQ ID NO: 313, SEQ ID NO: 314, SEQ ID NO: 315, SEQ ID NO: 316, SEQ ID NO: 317 and SEQ ID NO: 318 SEQ ID NO: 319 and SEQ ID NO: 320 and optionally SEQ ID NO 321 (i.e., for a 21 nucleotide ASO), and further optionally SEQ ID NO 322 (i.e., for a 22 nucleotide ASO), and further optionally SEQ ID NO 323 (i.e., for a 23 nucleotide ASO), and further optionally SEQ ID NO: 324 (i.e., for a 24 nucleotide ASO). In an embodiment, the ASO comprises SEQ ID NO: 314, SEQ ID NO: 315, SEQ ID NO: 316, SEQ ID NO: 317 and SEQ ID NO: 318 SEQ ID NO: 319, SEQ ID NO: 320 and SEQ ID NO: 321 and optionally SEQ ID NO 322 (i.e., for a 21 nucleotide ASO), and further optionally SEQ ID NO 323 (i.e., for a 22 nucleotide ASO), and further optionally SEQ ID NO 324 (i.e., for a 23 nucleotide ASO), and further optionally SEQ ID NO: 325 (i.e., for a 24 nucleotide ASO). In an embodiment, the ASO comprises SEQ ID NO: 315, SEQ ID NO: 316, SEQ ID NO: 317 and SEQ ID NO: 318 SEQ ID NO: 319, SEQ ID NO: 320, SEQ ID NO: 321 and SEQ ID NO: 322 and optionally SEQ ID NO 323 (i.e., for a 21 nucleotide ASO), and further optionally SEQ ID NO 324 (i.e., for a 22 nucleotide ASO), and further optionally SEQ ID NO 325 (i.e., for a 23 nucleotide ASO), and further optionally SEQ ID NO: 326 (i.e., for a 24 nucleotide ASO). In an embodiment, the ASO comprises SEQ ID NO: 316, SEQ ID NO: 317 and SEQ ID NO: 318 SEQ ID NO: 319, SEQ ID NO: 320, SEQ ID NO: 321, SEQ ID NO: 322 and SEQ ID NO: 323 and optionally SEQ ID NO 324 (i.e., for a 21 nucleotide ASO), and further optionally SEQ ID NO 325 (i.e., for a 22 nucleotide ASO), and further optionally SEQ ID NO 326 (i.e., for a 23 nucleotide ASO), and further optionally SEQ ID NO: 327 (i.e., for a 24 nucleotide ASO). In a preferred embodiment, the ASO comprises a bridged nucleic acid, preferably from 30-50% bridged nucleic acid, and preferably wherein the bridged nucleic acid is LNA. In a preferred embodiment, the ASO comprises phosphothioate linkages.

Also disclosed herein, is an ASO comprising 20-24 nucleotides which is capable of binding to a UNC13A splice site, wherein the ASO is an acceptor splice site (e.g., capable of binding to SEQ ID NO: 552). In an embodiment, the ASO comprises SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, and optionally SEQ ID NO: 167 (i.e., for a 21 nucleotide ASO), and further optionally SEQ ID NO 168 (i.e., for a 22 nucleotide ASO), and further optionally SEQ ID NO 169 (i.e., for a 23 nucleotide ASO), and further optionally SEQ ID NO: 170 (i.e., for a 24 nucleotide ASO). In an embodiment, the ASO comprises SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167 and optionally SEQ ID NO: 168 (i.e., for a 21 nucleotide ASO), and further optionally SEQ ID NO 169 (i.e., for a 22 nucleotide ASO), and further optionally SEQ ID NO 170 (i.e., for a 23 nucleotide ASO), and further optionally SEQ ID NO: 171 (i.e., for a 24 nucleotide ASO). In an embodiment, the ASO comprises SEQ ID NO: 161, SEQ ID NO: 162, SEQ ID NO: 163, SEQ ID NO: 164, SEQ ID NO: 165, SEQ ID NO: 166, SEQ ID NO: 167 and SEQ ID NO: 168 and optionally SEQ ID NO: 169 (i.e., for a 21 nucleotide ASO), and further optionally SEQ ID NO 170 (i.e., for a 22 nucleotide ASO), and further optionally SEQ ID NO 171 (i.e., for a 23 nucleotide ASO), and further optionally SEQ ID NO: 172 (i.e., for a 24 nucleotide ASO). In an embodiment, the ASO comprises SEQ ID NO: 161, SEQ ID NO: 162, SEQ ID NO: 163, SEQ ID NO: 164, SEQ ID NO: 165, SEQ ID NO: 166, SEQ ID NO: 167 and SEQ ID NO: 168 and optionally SEQ ID NO: 169 (i.e., for a 21 nucleotide ASO), and further optionally SEQ ID NO 170 (i.e., for a 22 nucleotide ASO), and further optionally SEQ ID NO 171 (i.e., for a 23 nucleotide ASO), and further optionally SEQ ID NO: 172 (i.e., for a 24 nucleotide ASO). In an embodiment, the ASO comprises SEQ ID NO: 162, SEQ ID NO: 163, SEQ ID NO: 164, SEQ ID NO: 165, SEQ ID NO: 166, SEQ ID NO: 167 and SEQ ID NO: 168, SEQ ID NO: 169 and optionally SEQ ID NO: 170 (i.e., for a 21 nucleotide ASO), and further optionally SEQ ID NO 171 (i.e., for a 22 nucleotide ASO), and further optionally SEQ ID NO 172 (i.e., for a 23 nucleotide ASO), and further optionally SEQ ID NO: 173 (i.e., for a 24 nucleotide ASO). In an embodiment, the ASO comprises SEQ ID NO: 163, SEQ ID NO: 164, SEQ ID NO: 165, SEQ ID NO: 166, SEQ ID NO: 167 and SEQ ID NO: 168, SEQ ID NO: 169 and SEQ ID NO: 170 optionally SEQ ID NO: 171 (i.e., for a 21 nucleotide ASO), and further optionally SEQ ID NO 172 (i.e., for a 22 nucleotide ASO), and further optionally SEQ ID NO 173 (i.e., for a 23 nucleotide ASO), and further optionally SEQ ID NO: 174 (i.e., for a 24 nucleotide ASO). In an embodiment, the ASO comprises SEQ ID NO: 164, SEQ ID NO: 165, SEQ ID NO: 166, SEQ ID NO: 167 and SEQ ID NO: 168, SEQ ID NO: 169 SEQ ID NO: 170 and SEQ ID NO: 171 optionally SEQ ID NO: 172 (i.e., for a 21 nucleotide ASO), and further optionally SEQ ID NO 173 (i.e., for a 22 nucleotide ASO), and further optionally SEQ ID NO 174 (i.e., for a 23 nucleotide ASO), and further optionally SEQ ID NO: 175 (i.e., for a 24 nucleotide ASO). In an embodiment, the ASO comprises SEQ ID NO: 165, SEQ ID NO: 166, SEQ ID NO: 167 and SEQ ID NO: 168, SEQ ID NO: 169 SEQ ID NO: 170 SEQ ID NO: 171 and SEQ ID NO: 172 and optionally SEQ ID NO: 173 (i.e., for a 21 nucleotide ASO), and further optionally SEQ ID NO 174 (i.e., for a 22 nucleotide ASO), and further optionally SEQ ID NO 175 (i.e., for a 23 nucleotide ASO), and further optionally SEQ ID NO: 176 (i.e., for a 24 nucleotide ASO). In an embodiment, the ASO comprises SEQ ID NO: 166, SEQ ID NO: 167 and SEQ ID NO: 168, SEQ ID NO: 169 SEQ ID NO: 170, SEQ ID NO: 171, SEQ ID NO: 172 and SEQ ID NO: 173 optionally SEQ ID NO: 174 (i.e., for a 21 nucleotide ASO), and further optionally SEQ ID NO 175 (i.e., for a 22 nucleotide ASO), and further optionally SEQ ID NO 176 (i.e., for a 23 nucleotide ASO), and further optionally SEQ ID NO: 177 (i.e., for a 24 nucleotide ASO). In an embodiment, the ASO comprises SEQ ID NO: 167 SEQ ID NO: 168, SEQ ID NO: 169 SEQ ID NO: 170, SEQ ID NO: 171, SEQ ID NO: 172, SEQ ID NO: 173 and SEQ ID NO: 174 optionally SEQ ID NO: 175 (i.e., for a 21 nucleotide ASO), and further optionally SEQ ID NO 176 (i.e., for a 22 nucleotide ASO), and further optionally SEQ ID NO 177 (i.e., for a 23 nucleotide ASO), and further optionally SEQ ID NO: 178 (i.e., for a 24 nucleotide ASO). In an embodiment, the ASO comprises SEQ ID NO: 168, SEQ ID NO: 169 SEQ ID NO: 170, SEQ ID NO: 171, SEQ ID NO: 172, SEQ ID NO: 173 and SEQ ID NO: 174 and SEQ ID NO: 175 optionally SEQ ID NO: 176 (i.e., for a 21 nucleotide ASO), and further optionally SEQ ID NO 177 (i.e., for a 22 nucleotide ASO), and further optionally SEQ ID NO 178 (i.e., for a 23 nucleotide ASO), and further optionally SEQ ID NO: 179 (i.e., for a 24 nucleotide ASO). In an embodiment, the ASO comprises SEQ ID NO: 169 SEQ ID NO: 170, SEQ ID NO: 171, SEQ ID NO: 172, SEQ ID NO: 173 and SEQ ID NO: 174, SEQ ID NO: 175 and SEQ ID NO: 176 optionally SEQ ID NO: 177 (i.e., for a 21 nucleotide ASO), and further optionally SEQ ID NO 178 (i.e., for a 22 nucleotide ASO), and further optionally SEQ ID NO 179 (i.e., for a 23 nucleotide ASO), and further optionally SEQ ID NO: 180 (i.e., for a 24 nucleotide ASO). In a preferred embodiment, the ASO comprises a bridged nucleic acid, preferably from 30-50% bridged nucleic acid, and preferably wherein the bridged nucleic acid is LNA. In a preferred embodiment, the ASO comprises phosphothioate linkages.

In a preferred embodiment, the ASO is capable of binding to a UNC13A donor splice site and the ASO comprises 21 nucleotides. In an embodiment, the ASO comprises SEQ ID NO: 295, SEQ ID NO: 297, SEQ ID NO: 299, SEQ ID NO: 300, SEQ ID NO: 301, SEQ ID NO: 302, SEQ ID NO: 303, SEQ ID NO: 304, and SEQ ID NO: 305. In an embodiment, the ASO comprises SEQ ID NO: 296, SEQ ID NO: 298, SEQ ID NO: 299, SEQ ID NO: 300, SEQ ID NO: 301, SEQ ID NO: 302, SEQ ID NO: 303, SEQ ID NO: 304, and SEQ ID NO: 305. In an embodiment, the ASO comprises SEQ ID NO: 297, SEQ ID NO: 299, SEQ ID NO: 300, SEQ ID NO: 301, SEQ ID NO: 302, SEQ ID NO: 303, SEQ ID NO: 304, SEQ ID NO: 305, and SEQ ID NO: 306. In an embodiment, the ASO comprises SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, and SEQ ID NO: 306. In an embodiment, the ASO comprises SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, and SEQ ID NO: 307. In an embodiment, the ASO comprises SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307 and SEQ ID NO: 308. In an embodiment, the ASO comprises SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308 and SEQ ID NO: 309. In an embodiment, the ASO comprises SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309 and SEQ ID NO: 310. In an embodiment, the ASO comprises SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310 and SEQ ID NO: 311. In an embodiment, the ASO comprises SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311 and SEQ ID NO: 312. In an embodiment, the ASO comprises SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312 and SEQ ID NO: 313. In an embodiment, the ASO comprises SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313 and SEQ ID NO: 314. In an embodiment, the ASO comprises SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314 and SEQ ID NO: 315. In an embodiment, the ASO comprises SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315 and SEQ ID NO: 316. In an embodiment, the ASO comprises SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316 and SEQ ID NO: 317. In an embodiment, the ASO comprises SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317 and SEQ ID NO 318. In an embodiment, the ASO comprises SEQ ID NO: 311, SEQ ID NO: 312, SEQ ID NO: 313, SEQ ID NO: 314, SEQ ID NO: 315, SEQ ID NO: 316, SEQ ID NO: 317 and SEQ ID NO: 318 and SEQ ID NO 319. In an embodiment, the ASO comprises SEQ ID NO: 312, SEQ ID NO: 313, SEQ ID NO: 314, SEQ ID NO: 315, SEQ ID NO: 316, SEQ ID NO: 317 and SEQ ID NO: 318 and SEQ ID NO: 319 and SEQ ID NO 320. In an embodiment, the ASO comprises SEQ ID NO: 313, SEQ ID NO: 314, SEQ ID NO: 315, SEQ ID NO: 316, SEQ ID NO: 317 and SEQ ID NO: 318 SEQ ID NO: 319 and SEQ ID NO: 320 and SEQ ID NO 321. In an embodiment, the ASO comprises SEQ ID NO: 314, SEQ ID NO: 315, SEQ ID NO: 316, SEQ ID NO: 317 and SEQ ID NO: 318 SEQ ID NO: 319, SEQ ID NO: 320 and SEQ ID NO: 321 and SEQ ID NO 322. In an embodiment, the ASO comprises SEQ ID NO: 315, SEQ ID NO: 316, SEQ ID NO: 317 SEQ ID NO: 318 SEQ ID NO: 319, SEQ ID NO: 320, SEQ ID NO: 321 and SEQ ID NO: 322 and SEQ ID NO 323. In an embodiment, the ASO comprises SEQ ID NO: 316, SEQ ID NO: 317 and SEQ ID NO: 318 SEQ ID NO: 319, SEQ ID NO: 320, SEQ ID NO: 321, SEQ ID NO: 322 and SEQ ID NO: 323 and SEQ ID NO 324. In a preferred embodiment, the ASO comprises a bridged nucleic acid, preferably from 30-50% bridged nucleic acid, and preferably wherein the bridged nucleic acid is LNA. In a preferred embodiment, the ASO comprises phosphothioate linkages.

Also disclosed herein is a pharmaceutical composition comprising a first ASO selected from the above, and a second ASO different to the first ASO selected from the above. In some embodiments, the pharmaceutical composition comprises a first ASO capable of binding to an UNC13A donor splice site or flanking regions thereof, and a second ASO capable of binding to an UNC13A acceptor splice site or flanking regions thereof. In some embodiments, the pharmaceutical composition comprises a first ASO capable of binding to an UNC13A long acceptor splice site or flanking regions thereof, and a second ASO capable of binding to a short acceptor splice site or flanking regions thereof. In some embodiments, the pharmaceutical composition comprises a first ASO is capable of binding the risk (i.e., minor) allele of the cryptic exon SNP and a second ASO capable of binding to the major allele of the cryptic exon SNP

As disclosed above, the flanking regions may be less than 100 nucleotides upstream or downstream of the splice site, more preferably less than 75, more preferably less than 50, more preferably less than 25, more preferably less than 20, and more preferably less than 10. As disclose above “capable of binding” means “complementary to” or “substantially complementary to”.

Manufacture and/or Synthesis of Antisense Polynucleotides

The polynucleotides (e.g., ASO(s)) described herein may be synthesized using any methods known in the art or those described herein.

### Evaluation

Purification of the antisense polynucleotides (e.g., ASO(s)) described herein may include, but is not limited to, polynucleotide clean-up, quality assurance and quality control. Clean-up may be performed by methods known in the arts such as, but not limited to, AGEN-COURT® beads (Beckman Coulter Genomics, Danvers, Mass.), poly-T beads, LNA™ oligo-T capture probes (EX-IQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method

In some embodiments, the antisense polynucleotides (e.g., ASOs) may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified polynucleotide may be analyzed in order to determine if the polynucleotide may be of proper size, check that no degradation of the polynucleotide has occurred.

Degradation of the antisense polynucleotide (e.g., ASO) may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### II. Pharmaceutical Compositions

Provided by the present disclosure include compositions such as pharmaceutical compositions comprising at least one antisense polynucleotide (e.g., ASO) as described herein. Compositions comprising the antisense polynucleotides (e.g., ASO(s)) described herein may be formulated for administration to a particular target cell, a target tissue, or a target organ and/or a subject.

The pharmaceutical composition of the present invention may comprise one or more polynucleotides (e.g., ASO, guide RNA, vectorized construct) as described herein, or two or more, or three or more or four or more polynucleotides (e.g., ASO, guide RNA, vectorized construct) as described herein.

In some embodiments, the pharmaceutical composition comprises two or more, three or more, or four or more ASOs as described herein. In some embodiments, the pharmaceutical composition comprises two or more ASOs which are capable of binding to different parts of the SEQ ID NO: 1 (i.e., UNC13A cryptic exon or intronic flanking regions thereof).

In some embodiments, the pharmaceutical composition comprises one or more ASOs capable of binding to the UNC13A donor splice site, and one or more ASOs capable of binding to an UNC13A acceptor splice site. In some embodiments, the pharmaceutical composition comprises two or more ASOs capable of binding to the UNC13A donor site.

In some embodiments pharmaceutical composition comprises two or more ASOs capable of binding to an UNC13A acceptor site, for example, where one or more ASOs is capable of binding to the short acceptor site, and one or more ASOs is capable of binding to the long acceptor site. In some embodiments, the pharmaceutical composition comprises a first ASO capable of binding (e.g., substantially complementary to) the risk (i.e., minor) allele of the CE SNP and a second ASO capable of binding (e.g., substantially complementary to) the major allele of the CE SNP. This ensures enhanced binding is obtained against both alleles.

In some embodiments, the pharmaceutical composition comprises two or more, three or more, or four or more guide RNAs as described herein. In some embodiments, the pharmaceutical composition comprises two or more, three or more, or four or more viral vectors as described herein. In some embodiments, the pharmaceutical composition may comprise a combination of one or more ASOs, one or more guide RNAs and/or one or more viral vectors as described herein.

The antisense polynucleotide (e.g., ASO) compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

### Formulations

Pharmaceutical formulations may additionally comprise a pharmaceutically acceptable excipient, which, as used herein, includes, but is not limited to, any and all solvents including water, dispersion media, diluents, or other liquid vehicles, dispersion or suspension aids, surface active agents, isotonic agents, a solubilizing agent, a tonicity agent, a pH adjuster, a buffering agent, thickening or emulsifying agents, preservatives, and the like, as suited to the particular dosage form desired.

Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Formulations of the pharmaceutical compositions described herein may be prepared by any method known or hereafter developed in the art of pharmacology. In general, such preparatory methods include the step of bringing the active ingredient (e.g., antisense polynucleotide) into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1 and 30%, between 5 and 80%, between 10 and 50%, between 20 and 90%, at least 70% (w/w), or at least 80% (w/w) active ingredient.

In some embodiments, the formulations described herein may contain at least one antisense polynucleotide (e.g., ASO). In some embodiments, the formulations may contain one, two, three, four or five antisense polynucleotides (e.g., ASO(s)) with different sequences. In one embodiment, the formulation contains at least two antisense polynucleotides (e.g., ASO(s)). In one embodiment, the formulation contains at least three antisense polynucleotides (e.g., ASO(s)). In another embodiment, the formulation contains at least four antisense polynucleotides (e.g., ASO(s)). In yet another embodiment, the formulation contains at least five antisense polynucleotides (e.g., ASO(s)).

The pharmaceutical compositions and formulations of the present disclosure can be formulated with one or more excipients to increase the stability of the antisense polynucleotide (e.g., ASO); increase cell penetration; permit the sustained, controlled or delayed release; alter the biodistribution (e.g., target the nucleic acid vaccine composition to specific tissues or cell types); increase the translation of encoded protein in vivo; and/or alter the release of encoded protein in vivo.

In addition to traditional excipients, excipients of the present disclosure can include, without limitation, lipids, lipidoids, liposomes, lipid nanoparticles, polymers, lipoplexes, core-shell nanoparticles, peptides, proteins, nucleic acid molecules, cells, organelles, explants, nanoparticle mimics and combinations thereof.

In vivo delivery of nucleic acids may be affected by many parameters, including, but not limited to, the formulation composition, nature of particle, degree of loading, polynucleotide (e.g., ASO) to lipid/lipidoid ratio, nature of polynucleotides (e.g., ASO) such as sequence contents, single-stranded or double-stranded, linear or circular, length and modifications, particle sizes and charges, and administration routes, etc.

The present disclosure contemplates the formulation and use in delivering at least one antisense polynucleotide (e.g., ASO) compositions and at least one pharmaceutically acceptable carrier. Complexes, micelles, liposomes or particles can be prepared containing any suitable lipids and lipidoids and therefore, can result in an effective delivery of the antisense polynucleotide compositions following the injection of a formulation via localized and/or systemic routes of administration, e.g., by various means including, but not limited to those described herein.

### Lipids and Lipidoids

In some embodiments, the antisense polynucleotides (ASO compounds) and compositions of the present disclosure may be formulated using one or more lipids and/or lipidoids. As used herein, the term “lipidoid” refers to any material having characteristics of a lipid. Lipidoids can be lipid-like structures containing multiple secondary and tertiary amine functionalities, which confer highly efficient interaction with nucleic acid molecules.

The synthesis of lipids and lipidoids has been extensively discussed and formulations containing the lipids and lipidoids are particularly suitable for delivery of nucleic acids. Use of the lipids and lipidoids to formulate and effectively deliver double stranded small RNAs (siRNAs), singled stranded mRNAs and gene therapy has been described in mice and non-human primates (e.g., Lvins et al., 2010); Akinc et al., Nat Biotechnol. 2008 26:561-569; Love et al., Proc Natl Acad Sci USA. 2010, 107:1864-1869; Siegwart et al., Proc Natl Acad Sci USA. 2011, 108:12996-3001; Leuschner et al., Nat Biotechnol. 2011, 29:1005-1010; Roberts et al., Methods Mol. Biol., 2016, 1364:2991-310; Ball et al. Nato. Lett. 2018, 18(6):3814-3822; Lokras et al., Methods Mol. Biol., 2021, 2282:137-157; Schrom et al., Mol. Ther. Nucleic Acids, 2017, 7:350-365; the contents of all of which are incorporated herein by references in their entirety).

The lipids and lipidoids can be cationic lipids and lipidoids. Cationic lipids typically features a positively charged head group followed by hydrophobic tails of varying compositions, wherein the head and tail are connected by a linker, such as an ether, ester or amide. Without wishing to be bound by any theory, their cationic head groups neutralize the anionic charges of the nucleic acids that they transport.

In some embodiments, ionizable cationic lipids can be used for formulations.

In some embodiments, the lipids can be anionic lipids.

In some embodiments, the lipids and lipidoids can be neutral lipids.

In some embodiments, ionizable lipids such as Dlin-MC3-DMA (MC3), Dlin-KC2-DMA (KC2), and cKK-E12 may be used for package circular nucleic acid molecules.

As non-limiting examples, lipidoids for formulation may include: “98N12-5” that is disclosed by Akinc et al., Mol Ther. 2009, 17:872-879; “C12-200” that is disclosed by Love et al., Proc Natl Acad Sci USA. 2010, 107:1864-1869 and Liu and Huang, Molecular Therapy. 2010, 669-670.

### Polymers and Polymeric Nanoparticles (NPs)

In some embodiments, the polynucleotide (e.g., ASO) compositions of the present disclosure may be formulated using one or more polymers, or polymer containing nanoparticles (NPs). In some embodiments, the polymer may be biocompatible and biodegradable.

The physicochemical properties of polymers (e.g., composition, molecular weight, and polydispersity) can be modified to achieve specialized formulations for nucleic acid delivery. Polymers may be naturally derived or synthetic. In some embodiments, the polymers used in the present disclosure have undergone processing to reduce and/or inhibit the attachment of unwanted substances such as, but not limited to, bacteria, to the surface of the polymer. The polymer may be processed by methods known and/or described in the art and/or described in PCT Patent Application Publication No. WO2012150467; the contents of which are herein incorporated by reference in their entirety.

Many polymer approaches have demonstrated efficacy in delivering nucleic acids in vivo into the cell cytoplasm (reviewed in de Fougerolles Hum Gene Ther. 2008, 19:125-132). An approach using dynamic polyconjugates has been shown in vivo in mice to effectively deliver siRNA and silence endogenous target mRNA in hepatocytes. In this approach, a multicomponent polymer system includes a membrane-active polymer to which nucleic acid, is covalently coupled via a disulfide bond and where both PEG (for charge masking) and N-acetylgalactosamine (for hepatocyte targeting) groups are linked via pH-sensitive bonds. On binding to the hepatocyte and entry into the endosome, the polymer complex disassembles in the low-pH environment, with the polymer exposing its positive charge, leading to endosomal escape and cytoplasmic release of the siRNA from the polymer. Replacing the N-acetylgalactosamine group with a mannose group can alter targeting sinusoidal endothelium and Kupffer cells (Rozema et al., Proc Natl Acad Sci USA. 2007, 104:12982-12887). Another approach using cyclodextrin-containing polycation nanoparticles to formulate siRNAs demonstrates targeted silencing of the EWS-FLI1 gene product in Ewing's sarcoma tumor cells (Hu-Lieskovan et al., Cancer Res. 2005, 65: 8984-8982); the contents of each of which are incorporated by reference in their entirety. Both of these delivery strategies incorporate rational approaches using polymers for both targeted delivery and endosomal escape mechanisms.

In some embodiments, the antisense polynucleotides (e.g., ASO(s)) of the present disclosure may be formulated using naturally derived polymers, structural proteins and polysaccharides, such as cationic collagen derivatives and chitosan. Cationic collagenous proteins have been used for nucleic acid delivery to articular cartilage and bone for regenerative medicine and metastatic tumor treatment (Capito et al., Gene Ther., 2007, 14:721-732; Curtin et al., Adv. Healthc. Mater., 2015, 4:223-227). Chitosan, a linear cationic polysaccharide, is produced by the deactylation of chitin (poly-d-glucosamine), which is non-toxic even at a high concentration and can be formulated into polyplexes. A non-limiting example of chitosan-based formulation includes a core of positively charged chitosan and an outer portion of negatively charged substrate (U.S. Pub. No. US20120258176; the contents of which are herein incorporated by reference in their entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

Cyclodextrins (CDs) are another family of naturally derived carbohydrate-based polymers with favorable physiochemical properties. α-, β-, or γ-CD forms can be used in combination with other cationic polymers for delivering nucleic acids, e.g., to the liver, and metastatic tumors.

The antisense polynucleotides (e.g., ASO(s)) of the present disclosure may be formulated using synthetic polymers which may incorporate versatile chemistries in a controlled manner providing flexibility and more options for polynucleotide formulations. Various synthetic strategies exist in the art to control polymerization reactions and, therefore, the properties of the resulting polymer. Examples of methods include controlled free-radical polymerizations such as reversible addition-fragmentation chain transfer (RAFT) polymerization and atom transfer radical polymerization (ATRP) (Boyer et al., Chem. Rev., 2009, 109:5402-5436). The polymers formulated with the polynucleotide (e.g., ASO) compositions of the present disclosure may be synthesized by the methods described in PCT Patent Application Publication Nos. WO2012082574 or WO2012068187; the contents of each of which are herein incorporated by reference in their entirety.

In some embodiments, cationic groups may be incorporated to polymers for formulating nucleic acid molecules. Without wishing to be bound by any theory, cationic groups can aid with the loading of negatively charged nucleic acid cargo and facilitate the interaction with negatively charged glycoproteins on the cell membrane when delivering the loaded polynucleotides to a cell.

In some embodiments, the antisense polynucleotide (e.g., ASO) compositions of the present disclosure may comprise at least one polymeric compound such as but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

In some embodiments, the synthetic polymers are biodegradable. Synthetic biodegradable polymers may be generated by assembling low molecular weight monomers into polymers via bioreversible linkages such as sulfide or ester bonds. Examples of synthetic biodegradable polymers include, but are not limited to, poly(lactic acid) (PLA), poly(glycolic acid) (PGA), poly(lactic-co-glycolic acid) (PLGA), poly(beta amino) esters (PBAEs), Poly(amine-co-esters) (PACEs).

Biodegradable polymers have been previously used to protect nucleic acids from degradation and been shown to result in sustained release of payloads in vivo (Rozema et al., Proc Natl Acad Sci USA. 2007, 104:12982-12887; Sullivan et al., Expert Opin Drug Deliv. 2010, 7:1433-1446; Convertine et al., Biomacromolecules. 2010 Oct. 1; Chu et al., Acc Chem Res. 2012 Jan. 13; Manganiello et al., Biomaterials. 2012, 33:2301-2309; Benoit et al., Biomacromolecules. 2011, 12:2708-2714; Singha et al., Nucleic Acid Ther. 2011, 2:133-147; de Fougerolles Hum Gene Ther. 2008, 19:125-132; Schaffert and Wagner, Gene Ther. 2008, 16:1131-1138; Chaturvedi et al., Expert Opin Drug Deliv. 2011, 8:1455-1468; Davis, Mol Pharm. 2009, 6:659-668; Davis, Nature, 2010, 464:1067-1070; the contents of each of which are herein incorporated by reference in their entirety).

The biodegradable polymers may be polymers comprising a polyethylenimine group as described in U.S. Pat. No. 7,700,542. The polymers may be the biodegradable cationic lipopolymer made by methods described in U.S. Pat. No. 6,696,038, and U.S. Pub. Nos. US20030073619 and US20040142474; the contents of each of which are incorporated herein by reference in their entirety.

The antisense polynucleotides (e.g., ASO(s)) of the present disclosure may be formulated in polymeric carriers using polymers-containing different nanoparticles. For example, the therapeutic polymer nanoparticles may be identified by the methods described in US Pub No. US20120140790, the contents of which are herein incorporated by reference in their entirety. In some embodiments, the therapeutic nanoparticles may comprise a polymeric matrix. As a non-limiting example, the nanoparticle may comprise two or more polymers and diblock copolymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. Polymers may also include those described in PCT Patent Application Publication Nos. WO2011115862, WO2012082574 and WO2012068187 and U.S. Pub. No. US20120283427; a polymer of formula Z as described in WO2011115862; a polymer of formula Z, Z′ or Z″ as described in PCT Patent Application Publication Nos. WO2012082574 and WO2012068187 and U.S. Pub. No. US2012028342; the contents of each of which are herein incorporated by reference in their entirety.

As a non-limiting example, the antisense polynucleotide (e.g., ASO) compositions of the present disclosure are formulated in a therapeutic nanoparticle comprising a PLGA-PEG block copolymer (see US Pub. No. US20120004293 and U.S. Pat. No. 8,236,330), or PEG-PLA diblock copolymer or PEG-PLGA copolymer (see U.S. Pat. No. 8,246,968 and PCT Patent Application Publication No. WO2012166923), or a multiblock copolymer described in U.S. Pat. Nos. 8,263,665 and 8,287,910; the contents of each of which are herein incorporated by reference in their entirety.

In some embodiments, the block copolymers may include those of formula I, formula II, formula III, formula IV, formula V, formula VI and formula VII of PCT Patent Application Publication No. WO2015017519, the contents of which are herein incorporated by reference in their entirety. The block copolymers may be included in a polyion complex comprising a non-polymeric micelle and the block copolymer. (See e.g., U.S. Pub. No. US20120076836; the contents of which are herein incorporated by reference in their entirety).

In some embodiments, the antisense polynucleotide (e.g., ASO) compositions of the present disclosure may be formulated using acrylic polymers including but not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof. In other embodiments, the antisense polynucleotide (e.g., ASO) compositions of the present disclosure may be formulated using amine-containing polymers such as, but not limited to polylysine, polyethylene imine, poly(amidoamine) dendrimers, poly(beta-amino esters) (See e.g., U.S. Pat. No. 8,287,849; the contents of which are herein incorporated by reference in their entirety).

In some embodiments, the nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. The degradable polyesters may include a PEG conjugation to form a PEGylated polymer. In other embodiments, the polynucleotide (e.g., ASO) compositions of the present disclosure may be formulated with at least one cross-linkable polyester. Crosslinkable polyesters include those known in the art and described in US Pub. No. US20120269761; the contents of which herein are incorporated by reference in their entirety.

In some embodiments, the polymer formulations comprising the polynucleotides (e.g., ASO(s)) of the present disclosure may include branched PEG molecules as described in or made by the methods described in PCT Patent Application Publication No. WO20180126084. The branched PEG which may be used in the formulations described in WO20180126084 may have the formula I, formula II, formula III, formula IV, formula V, and formula VI; the contents of which are incorporated herein by reference in their entirety.

In some embodiments, the antisense polynucleotide (e.g., ASO) is covalently attached to a carrier molecule. In an example, the ASO is covalently attached to a carbohydrate, a protein, a small molecule (e.g., α-tocopherol), a peptide (e.g., a cell-penetrating peptide), an antibody, a lipid (e.g., cholesterol) or a polymer (e.g., PEG). In an embodiment, the ASO is not covalently attached to a carrier molecule.

In some embodiments, the polymer nanoparticles for formulating the antisense polynucleotides (e.g., ASO(s)) of the present disclosure may include a conjugation of at least one targeting ligand. The targeting ligand may be any ligand known in the art such as, but not limited to, a monoclonal antibody, a peptide and a nucleic acid (e.g., aptamer). In other embodiments, the polymer nanoparticles can be selectively targeted to cells, tissues and/or organs through expression of different ligands (e.g., folate, transferrin, and N-acetylgalactosamine (GalNAc)).

In some embodiments, the polymer nanoparticles (NPs) for formulating the antisense polynucleotide (e.g., ASO) compositions of the present disclosure may permit the sustained or delayed release of the polynucleotide compositions. The altered release profile for the antisense polynucleotide compositions can result in regulation over an extended period of time. In some embodiments, the polymeric formulations for sustained release may include, but are not limited to, PLGA microspheres, ethylene vinyl acetate (EVAc), poloxamer, and fibrinogen polymers. For example, the antisense polynucleotide compositions may be formulated in PLGA microspheres by preparing the PLGA microspheres with tunable release rates (e.g., days and weeks) and encapsulating the polynucleotide (e.g., ASO(s)) compositions in the PLGA microspheres while maintaining the integrity of the polynucleotides during the encapsulation process.

In some embodiments, the antisense polynucleotide (e.g., ASO) compositions of the present disclosure may be formulated for controlled release in polymeric formulations comprising copoly(lactic/glycolic) acid (see, e.g., U.S. Pat. No. 4,675,189 to Kent et al.), or block copolymers of lactic acid and PEG, which is injected subcutaneously or intramuscularly to achieve a depot formulation for controlled release.

As non-limiting examples, the antisense polynucleotides (e.g., ASO(s)) of the present disclosure may be formulated in a polymeric formulation comprising polymeric compound of PEG-PLL as described in U.S. Pat. No. 6,177,274, or in a formulation comprising PLGA-PEG-PLGA block copolymers as described in U.S. Pat. No. 6,004,573, or in a dry formulation or in a solution that is capable of being dried as described in U.S. Pub. Nos. US20090042829 and US20090042825; the contents of each of which are herein incorporated by reference in their entirety.

### Liposomes

The polynucleotides and polynucleotide (e.g., ASOs and ASO) compositions of the disclosure can be formulated using one or more liposomes. As used herein, the term “liposome” refers to an artificially prepared vesicle which may primarily be composed of one or several lipid bilayers and may be used as a delivery vehicle. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

Liposomes may be lipid-based liposomes, polymer-based liposomes, or hybrids. Liposomes can be cationic liposomes, neutral liposomes. Cationic liposomes have been used to deliver siRNA to various cell types (e.g., US Patent Application Publication No.: US2004/0204377).

In some embodiments, the liposome may contain a sugar-modified lipid disclosed in US Pat. No.; U.S. Pat. No. 5,595,756 to Bally et al.; the contents of which are incorporated herein by reference in their entirety. The lipid may be a ganglioside and cerebroside in an amount of about 10 mol percent.

In some embodiments, liposomes are formed by the self-assembly of dissolved lipid molecules and/or polymers. The polynucleotides (e.g., ASO(s)) of the present disclosure may be entrapped passively into the lipid bilayers through the preparation of liposomes, e.g., encapsulated in the aqueous core of the liposome or the aqueous phase between bilayers (in the case of multilamellar vesicles) using passive loading methods, such as reverse phase evaporation, dehydration-rehydration method, or active loading involving pH-gradient across the liposome membrane (Szoka and Papahadjopoulos, PNAS, 1978; 9:4194-4198; Shew and Deamer, Biochim. Biophy Acta., 1985; 1:1-8; and Mayer et al., Biochim. Biophy Acta., 1986; 1:123-126).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010, 28:172-176; the contents of which are herein incorporated by reference in their entirety), the liposome formulation was composed of 57.1% cationic lipid, 7.1% dipalmitoylphosphatidylcholine, 34.3% cholesterol, and 1.4% PEG-c-DMA.

In some embodiments, liposomes may be targeted liposomes with surface-attached ligands, capable of recognizing and binding to cells of interest. The targeted liposomes may increase delivery and accumulation of liposomes and entrapped polynucleotides (e.g., ASO(s)) in the desired tissues and organs. The surface targeting ligands may include immunoglobolins (Ig) and their fragments, peptides and aptamers.

In some embodiments, the surface of liposomes may be coated with inert, biocompatible polymers such as PEG. The polymer coating forms a protective layer over the liposomal surface and slows down the liposome recognition by opsonins; thereby increasing circulation of liposomes in vivo.

In some embodiments, the polynucleotides (e.g., ASO(s)) and pharmaceutical compositions comprising the polynucleotides (e.g., ASO(s)) described herein may include, without limitation, liposomes such as those formed from 1,2-dioleyloxy-N,N-dimethylaminopropane (DODMA) liposomes, DiLa2 liposomes from Marina Biotech (Bothell, WA), SMARTICLES®/NOV340 (Marina Biotech, Bothell), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US Patent Application Publication US20100324120; the contents of which are herein incorporated by reference in their entirety), neutral DOPC (1,2-dioleoyl-sn-glycero-3-phosphocholine) based liposomes (e.g., siRNA delivery for ovarian cancer (Landen et al. Cancer Biology & Therapy 2006, 5(12): 1708-1713); the contents of which is herein incorporated by reference in its entirety), hyaluronan-coated liposomes (Quiet Therapeutics, Israel), and liposomes which may deliver small molecule drugs such as, but not limited to, DOXIL® from Janssen Biotech, Inc. (Horsham, PA).

In some embodiments, the polynucleotides (e.g., ASO(s)) and pharmaceutical compositions comprising the polynucleotides (e.g., ASO(s) described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999, 6:271-281; Zhang et al. Gene Therapy. 1999, 6:1438-1447; Jeffs et al. Pharm Res. 2005, 22:362-372; Morrissey et al., Nat Biotechnol. 2005, 2:1002-1007; Zimmermann et al., Nature. 2006, 441:111-114; Heyes et al. J Contr Rel. 2005, 107:276-287; Semple et al. Nature Biotech. 2010, 28:172-176; Judge et al. J Clin Invest. 2009, 119:661-673; deFougerolles Hum Gene Ther. 2008, 19:125-132; the contents of each of which are incorporated herein in their entireties). The original manufacturing method by Wheeler et al. was a detergent dialysis method, which was later improved by Jeffs et al. and is referred to as the spontaneous vesicle formation method. The liposome formulations may be composed of 3 to 4 lipid components in addition to the nucleic acid vaccine compositions. As a non-limiting example, a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. In another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al. In another example, the nucleic acid-lipid particle may comprise a cationic lipid comprising from about 50 mol % to about 85 mol % of the total lipid present in the particle; a non-cationic lipid comprising from about 13 mol % to about 49.5 mol % of the total lipid present in the particle; and a conjugated lipid that inhibits aggregation of particles comprising from about 0.5 mol % to about 2 mol % of the total lipid present in the particle as described in WO2009127060 to Maclachlan et al; the contents of which are incorporated herein by reference in their entirety. In another example, the nucleic acid-lipid particle may be any nucleic acid-lipid particle disclosed in US2006008910 to Maclachlan et al.; the contents of which are incorporated herein by reference in their entirety. As a non-limiting example, the nucleic acid-lipid particle may comprise a cationic lipid of Formula I, a non-cationic lipid, and a conjugated lipid that inhibits aggregation of particles.

In some embodiments, the polynucleotide (e.g. ASO) compositions of the present disclosure may be formulated in a liposome comprising a cationic lipid. The liposome may have a molar ratio of nitrogen atoms in the cationic lipid to the phosphates in the nucleic acid vaccine compositions (N:P ratio) of between 1:1 and 20:1 as described in PCT Patent Application Publication No. WO2013006825, the contents of which are herein incorporated by reference in their entirety. In some embodiments, the liposome may have a N:P ratio of greater than 20:1 or less than 1:1.

In some embodiments, the polynucleotide (e.g., ASO) compositions may be formulated with any amphoteric liposome disclosed in PCT Patent Application Publication No.: WO 2008043575 to Panzner and U.S. Pat. No. 8,580,297 to Essler et al. (Marina Biotech), the contents of which are incorporated herein by reference in their entirety. The amphoteric liposome may comprise a mixture of lipids including a cationic amphiphile, an anionic amphiphile and optional one or more neutral amphiphiles. The amphoteric liposome may comprise amphoteric compounds based on amphiphilic molecules, the head groups of which being substituted with one or more amphoteric groups. In some embodiments, the pharmaceutical compositions may be formulated with an amphoteric lipid comprising one or more amphoteric groups having an isoelectric point between 4 and 9, as disclosed in US Patent Application Publication No.: US20140227345 to Essler et al. (Marina Biotech), the contents of which are incorporated herein by reference in their entirety. In some embodiments, the pharmaceutical composition may be formulated with amphoteric liposomes comprising at least one amphipathic cationic lipid, at least one amphipathic anionic lipid, and at least one neutral lipid, or liposomes comprise at least one amphipathic lipid with both a positive and a negative charge, and at least one neutral lipid, wherein the liposomes are stable at pH 4.2 and pH 7.5, as disclosed in U.S. Pat. No. 7,780,983 to Panzner et al. (Novosom), the contents of which are incorporated herein by reference in their entirety.

In some embodiments, the polynucleotide (e.g., ASO) composition may be formulated with liposomes comprising a sterol derivative as disclosed in U.S. Pat. No. 7,312,206 to Panzner et al. (Novosom), the contents of which are incorporated herein by reference in their entirety.

In some embodiments, the pharmaceutical composition may be formulated with liposomes comprising a serum-stable mixture of lipids taught in US Patent Application Publication No.: US 20110076322 to Panzner et al, the contents of which are incorporated herein by reference in their entirety, capable of encapsulating the nucleic acid vaccine compositions of the present disclosure. The lipid mixture comprises phosphatidylcholine and phosphatidylethanolamine in a ratio in the range of about 0.5 to about 8. The lipid mixture may also include pH sensitive anionic and cationic amphiphiles, such that the mixture is amphoteric, being negatively charged or neutral at pH 7.4 and positively charged at pH 4. The drug/lipid ratio may be adjusted to target the liposomes to particular organs or other sites in the body. In some embodiments, liposomes loaded with the nucleic acid vaccine compositions of the present disclosure as cargo, are prepared by the method disclosed in US Patent Application Publication No.: US 20120021042 to Panzner et al., the contents of which are incorporated herein by reference in their entirety. The method comprises steps of admixing an aqueous solution of a polyanionic active agent (e.g., polynucleotides) and an alcoholic solution of one or more amphiphiles and buffering said admixture to an acidic pH, wherein the one or more amphiphiles are susceptible of forming amphoteric liposomes at the acidic pH, thereby to form amphoteric liposomes in suspension encapsulating the active agent.

### Lipoplexes

The compositions of the present disclosure can be formulated using one or more lipoplexes. In some embodiments, the antisense polynucleotide (e.g., ASO) compositions may be formulated as a lipoplex, such as, without limitation, the ATUPLEX™ system, the DACC system, the DBTC system and other RNA-lipoplex technology from Silence Therapeutics (London, United Kingdom), STEMFECT™ from STEMGENT® (Cambridge, MA), and polyethylenimine (PEI).

In some embodiments, the lipoplex-formulated RNA (RNA-LPX) may be generated by complexing RNA with liposomes containing consisting of the cationic lipid DOTMA and the helper lipid DOPE (Kranz et al., Nature, 2016; 534(7607):396-401). The RNA-lipoplexes may be formed for intravenous injection, with pharmaceutical and physiological characteristics that allow selective targeting of antisense polynucleotides (e.g., ASO(s)) to target cells, tissues and/or organs. The RNA-lipoplex product may be formed from RNA and dedicated cationic (positively charged) liposomes in a self-assembly process, comprising a topological transition from liposomes into compact RNA-lipoplex nanoparticles with a distinct internal molecular organization. The effect of parameters such as particle charge, size molecular organization, lipid composition and phase state on the biological activity is individually investigated to evaluate efficacy of the lipoplex formulation in vitro and in vivo. The ratio between the cationic lipids and the RNA, expressed as the charge ratio, will be determined for the particle characteristics and the targeting selectivity. RNA-lipoplex formulations may be formed either with an excess of positive (cationic liposomes) or negative (RNA) charge. Positive charged or negative charged lipoplexes may affect delivery targets. Therefore, variation of the characteristics of the liposomes used for lipoplex assembly, the biological activity could be further controlled. In some embodiments, lipoplex formation may be achieved in the presence of various monovalent and divalent ions, peptides and buffers.

### Lipid Nanoparticles (LNPs)

In some embodiments, the antisense polynucleotides (e.g., ASO(s)) and compositions of the present disclosure may be formulated in a lipid nanoparticle (LNP).

In general, LNPs can be characterized as small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers.

The LNPs for formulating the antisense polynucleotides (e.g., ASO(s)) of the present disclosure may have a diameter from 10-1000 nm. The nanoparticle may be 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 nm, or greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the lipid nanoparticles formulating the antisense polynucleotides (e.g., ASO(s)) of the present disclosure may have a diameter from about 1 to about 100 nm, such as but not limited to, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, and/or from about 5 nm to about 100 nm.

In some embodiments, the lipid nanoparticles formulating the antisense polynucleotides (e.g., ASO(s)) of the present disclosure may have a diameter from about 10 to about 100 nm, such as, but not limited to, about 10 nm to about 20 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 50 nm to about 60 nm, about 50 nm to about 70 nm about 50 nm to about 80 nm, about 50 nm to about 90 nm, about 50 nm to about 100 nm, about 60 nm to about 70 nm, about 60 nm to about 80 nm, about 60 nm to about 90 nm, about 60 nm to about 100 nm, about 70 nm to about 80 nm, about 70 nm to about 90 nm, about 70 nm to about 100 nm, about 80 nm to about 90 nm, about 80 nm to about 100 nm and/or about 90 nm to about 100 nm.

In some embodiments, the lipid nanoparticles formulating the antisense polynucleotides (e.g., ASO(s)) of the present disclosure may have a diameter from about 10 to about 500 nm.

In some embodiments, the LNPs formulating the antisense polynucleotides (e.g., ASO(s)) of the present disclosure are smaller LNPs, having a diameter from below 0.1 pm up to 100 nm such as, but not limited to, less than 0.1 pm, less than 1.0 pm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 pm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 pm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μM, less than 950 μm, less than 975 μm.

LNPs useful herein are known in the art and generally comprise cholesterol (aids in stability and promotes membrane fusion), a helper lipid (e.g., a phospholipid which provides structure to the LNP bilayer and also may aid in endosomal escape), a polyethylene glycol (PEG) derivative (which reduces LNP aggregation and “shields” the LNP from non-specific endocytosis by immune cells and reduce opsonization by serum proteins and reticuloendothelial clearance), and an ionizable lipid (complexes negatively charged RNA and enhances endosomal escape), which form the LNP-forming composition. The components of the LNP may be selected based on the desired target, tropism, cargo (e.g., a antisense polynucleotide), size, or other desired feature or property. The relative amounts (ratio) of ionizable lipid, helper lipid, cholesterol and PEG substantially affect the efficacy of lipid nanoparticles and may be optimized for a given application and administration route.

In general, the antisense polynucleotide (e.g., ASO) compositions of the present disclosure may be formulated using LNPs into their interior space, into the inter membrane space, onto their exterior surface, or any combination thereof.

The LNP for formulating the antisense polynucleotides (e.g., ASO(s)) of the present disclosure comprises at least one cationic lipid. The cationic lipid may be selected from any lipid known in the art such as, but not limited to, DLin-MC3-DMA, DLin-DMA, C12-200 and DLin-KC2-DMA.

In some embodiments, the cationic lipid which may be used in formulations of the present disclosure may be selected from, but not limited to, a cationic lipid described in PCT Patent Application Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; the contents of each of which are herein incorporated by reference in their entirety. The cationic lipid may be also selected from, but not limited to, formula A described in PCT Patent Application Publication Nos. WO2012040184, WO2011153120, WO2011149733, WO2011090965, WO2011043913, WO2011022460, WO2012061259, WO2012054365 and WO2012044638; the contents of each of which are herein incorporated by reference in their entirety. Alternatively, the cationic lipid may be selected from, but not limited to, formula CLI-CLXXIX of PCT Patent Application No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; the contents of each of which are herein incorporated by reference in their entirety. The cationic lipid may be a multivalent cationic lipid disclosed in U.S. Pat. No. 7,223,887 (the contents of which are incorporated herein by reference in their entirety), which has a positively charged head group including two quaternary amine groups and a hydrophobic portion including four hydrocarbon chains. The cationic lipid may be biodegradable as the biodegradable lipids disclosed in US Patent Application Publication No.: US20130195920 to Maier et al. (the contents of which are incorporated herein by reference in their entirety), which have one or more biodegradable groups located in a lipidic moiety of the cationic lipid. In some embodiments, the cationic lipid may also be the cationic lipids disclosed in US20130156845 and US 20130129785 to Manoharan et al., WO 2012047656 to Wasan et al., WO 2010144740 to Chen et al., WO 2013086322 to Ansell et al., and WO 2012016184 to Manoharan et al., the contents of each of which are incorporated herein by reference in their entirety.

As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimethylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl] pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl} dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In some embodiments, the LNP for formulating the antisense polynucleotide (e.g., ASO) compositions of the present disclosure may comprise a plurality of cationic lipids, such as a first and a second cationic lipid as described in US Patent Application Publication No.: US20130017223 to Hope et al. (the contents of which are incorporated herein by reference in their entirety). The first cationic lipid can be selected on the basis of a first property and the second cationic lipid can be selected on the basis of a second property. The first and second properties may be complementary. In some embodiments, the LNP may comprise one or more first cationic lipids and one or more second lipids, wherein the lipid particle comprises a solid core, as described in US Patent Publication No. US20120276209 to Cullis et al. (the contents of which are incorporated herein by reference in their entirety).

In some embodiments, the LNPs may contain one or more ionizable lipids such as C 12-200, DLin-KC2-DMA, and/or HGT5001, helper lipids, structural lipids, PEG-modified lipids, MC3, DLinDMA, DLinkC2DMA, cKK-E12, ICE, HGT5000, DODAC, DDAB, DMRIE DOSPA, DOGS, DODAP, DODMA, DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, HGT4003, and combinations thereof.

In some embodiments, the cationic lipid may be synthesized by methods known in the art and/or as described in PCT Patent Application Publication Nos. WO2012040184, WO2011153120, WO2011149733, WO2011090965, WO2011043913, WO2011022460, WO2012061259, WO2012054365, WO2012044638, WO2010080724 and WO201021865; the contents of each of which are herein incorporated by reference in their entirety.

In some embodiments, the nanoparticles described herein may comprise at least one cationic polymer described herein and/or known in the art.

The LNP for formulating the antisense polynucleotides (e.g., ASO(s)) of the present disclosure may comprise at least one helper lipid. The helper lipids in LNPs may contribute to their stability and delivery efficiency, and/or mitigate the toxicity owing to the cationic lipids. In some embodiments, the helper lipid is a lipid having cone-shape geometry, e.g., dioleoylphosphatidylethanolamine (DOPE). In some embodiments, the helper lipid is a cylindrical-shaped lipid such as phosphatidylcholine.

In some embodiments, the LNP for formulating the antisense polynucleotides (e.g., ASO(s)) of the present disclosure may comprise a phospholipid such as a synthetic phospholipid, including but not limited to, DDPC, DLPC, DMPC, DPPC, DSPC, DOPC, POPC and DEPC; DMPG, DPPG, DSPG and POPG; DMPA, DPPA and DSPA; DMPE, DPPE, DSPE and DOPE; DOPS; and polyglycerin attached phospholipids (PG phospholipid). The phospholipid may be selected based on administration routes, e.g., DPPC, POPC and POPG used in LNPs for injection and DOPC, POPC and DDPC used in LNPs for pulmonary delivery. In some embodiments, the phospholipid may be a purified lipid from a natural source.

In some embodiments, the LNP for formulating the antisense polynucleotides (e.g., ASO(s)) of the present disclosure may comprise one or more neutral helper lipids such as dioleoyl phosphoethanolamine (DOPE), prostaglandins, eicosanoids, glycerides, glycosylated diacyl glycerols, oxygenated fatty acids, NAGly and PAHSA.

The LNP for formulating the antisense polynucleotides (e.g., ASO(s)) of the present disclosure comprises a cholesterol, a naturally occurring cholesterol analogue, or a synthetic cholesterol like compound and the cholesterol derivatives. In some embodiments, a naturally occurring cholesterol analog may be selected from those by Patel et al., (Nature Communications, 2020; 983: doi.org/10.1038/s41467-020-14527-2); the contents of which are incorporated herein by reference in their entirety. In some embodiments, the LNPs comprise one or more cholesterol derivatives, e.g., PtdChol.

The LNP for formulating the antisense polynucleotides (e.g., ASO(s)) of the present disclosure comprises at least a PEGylated compound, such as a PEG polymer and a PEGylated lipid.

In some embodiments, the antisense polynucleotide (e.g., ASO) compositions of the preset disclosure may include at least one of the PEGylated lipids described in PCT Patent Application Publication No. WO2012099755, the contents of which are herein incorporated by reference in their entirety.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations.

In some embodiments, The LNP for formulating the antisense polynucleotides (e.g., ASO(s)) of the present disclosure comprise PEG-c-DOMG. In some embodiments, the PEG-c-DOMG may be replaced with a PEG lipid such as, but not limited to, PEG-DSG (1,2-Distearoyl-sn-glycerol, methoxypolyethylene glycol), PEG-DPG (1,2-Dipalmitoyl-sn-glycerol, methoxypolyethylene glycol), or PEG-DMG 2000 (1,2-dimyristoyl-sn-glycero-3-phophoethanolamine-N-[methoxy(polyethylene glycol)-2000). As a non-limiting example, the LNP formulation may contain PEG-DMG 2000, DLin-DMA, DSPC and cholesterol. As another non-limiting example, the LNP formulation may contain PEG-DMG 2000, DLin-DMA, DSPC and cholesterol in a molar ratio of 2:40:10:48 (see e.g., Geall et al., PNAS, 2012, 109(36): 14604-14609; herein incorporated by reference in its entirety).

In some embodiments, the LNP formulations described herein may comprise a polycationic composition. As a non-limiting example, the polycationic composition may be selected from formula 1-60 of US Patent Publication No. US20050222064; the contents of which are herein incorporated by reference in their entirety. The LNP formulations comprising a polycationic composition may be used for the delivery of the nucleic acid vaccine compositions described herein in vivo and/or in vitro.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the LNP for formulating the antisense polynucleotides (e.g., ASO(s)) of the present disclosure may comprise a cleavable lipid such as those described in PCT Patent Application Publication No. WO2012170889, the contents of which are herein incorporated by reference in their entirety.

In some embodiments, the LNP for formulating the antisense polynucleotide (e.g., ASO) compositions of the present disclosure may comprises a conjugated lipid. In a non-limiting example, the conjugated lipid may have a formula such as described in US Pub. No. US 20120264810 to Lin et al., the contents of which are incorporated herein by reference in their entirety. The conjugate lipid may form a lipid particle which further comprises a cationic lipid, a neutral lipid, and a lipid capable of reducing aggregation.

In some embodiments, the LNP for formulating the antisense polynucleotides (e.g., ASO(s)) of the present disclosure may comprise a mixture of cationic compounds and neutral lipids. As a non-limiting example, the cationic compounds may be formula (I) disclosed in PCT Patent Application Publication No.: WO 1999010390 to Ansell et al., the contents of which are described herein by reference in their entirety, and the neutral lipid may be selected from the group consisting of diacylphosphatidylcholine, diacylphosphatidylethanolamine, ceramide and sphingomyelin.

In some embodiments, the LNP formulations described herein may additionally comprise a permeability enhancer molecule. Non-limiting permeability enhancer molecules are described in US Patent Publication No. US20050222064; the contents of which are herein incorporated by reference in their entirety.

In some embodiments, the LNP for formulating the antisense polynucleotide (e.g., ASO) compositions of the present disclosure may be encapsulated into any polymer known in the art which may form a gel when injected into a subject. As another non-limiting example, the lipid nanoparticle may be encapsulated into a polymer matrix which may be biodegradable.

In some embodiments, the LNP for formulating the antisense polynucleotide (e.g., ASO) compositions of the present disclosure may be encapsulated in the lipid formulation to form a stable nucleic acid-lipid particle (SNALP) such as described in U.S. Pat. No. 8,546,554 to de Fougerolles et al., the contents of which are incorporated here by reference in their entirety. In one non-limiting example, the SNALP includes 40% 2,2-Dilinoleyl-4-dimethylaminoethyl-[1,3]-dioxolane (Lipid A), 10% dioleoylphosphatidylcholine (DSPC), 40% cholesterol, 10% polyethylene glycol (PEG)-C-DOMG (mole percent) with a particle size of 63.0±20 nm and a 0.027 nucleic acid/lipid ratio.

In some embodiments, the LNPs for formulating the antisense polynucleotide (e.g., ASO) compositions of the present disclosure may comprise an endosomal membrane destabilizer as disclosed in U.S. Pat. No. 7,189,705 to Lam et al., the contents of which are incorporated herein by reference in their entirety. As a non-limiting example, the endosomal membrane destabilizer may be a Ca²⁺ ion.

In some embodiments, the LNPs for formulating the antisense polynucleotide (e.g., ASO) compositions of the present disclosure may comprise a charged lipid or an amino lipid. As used herein, the term “charged lipid” is meant to include those lipids having one or two fatty acyl or fatty alkyl chains and a quaternary amino head group. The quaternary amine carries a permanent positive charge. The head group can optionally include an ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine). In a non-limiting example, the charged lipid used in any of the formulations described herein may be any charged lipid described in EP2509636 to Manoharan et al., the contents of which are incorporated herein by reference in their entirety. In some embodiments, a charged lipid is referred to as an “amino lipid.” In a non-limiting example, the amino lipid may be any amino lipid described in US Pub. No. US20110256175 to Hope et al., the contents of which are incorporated herein by reference in their entirety. For example, the amino lipids may have the structure disclosed in Tables 3-7 of Hope, such as structure (II), DLin-K-C2-DMA, DLin-K2-DMA, DLin-K6-DMA, etc. In another non-limiting example, the amino lipids may be any amino lipid described in US 20110117125 to Hope et al., the contents of which are incorporated herein by reference in their entirety, such as a lipid of structure (I), DLin-K-DMA, DLin-C-DAP, DLin-DAC, DLin-MA, DLin-S-DMA, etc. In another non-limiting example, the amino lipid may have the structure (I), (II), (III), or (IV), or 4-(R)-DLin-K-DMA (VI), 4-(S)-DLin-K-DMA (V) as described in PCT Patent Application Publication No. WO2009132131 to Manoharan et al., the contents of which are incorporated herein by reference in their entirety.

In some embodiments, the LNPs for formulating the antisense polynucleotide (e.g., ASO) compositions of the present disclosure may comprise reverse head group lipids, e.g., formulated with a zwitterionic lipid comprising a headgroup wherein the positive charge is located near the acyl chain region and the negative charge is located at the distal end of the head group, such as a lipid having structure (A) or structure (I) described in PCT Patent Application Publication No. WO2011056682 to Leung et al., the contents of which are incorporated herein by reference in their entirety.

In some embodiments, the lipid components of the LNP to nucleic acid ratio (mass/mass ratio) (e.g., lipids to polynucleotide (e.g., ASO) compositions ratio) may be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or 1:1, 2:1, 3:1, 4:1, 5:1, 6:1, 7:1, 8:1. 9:1, 10:1, 11:1, 12:1, 13:1. 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 38:1, 39:1, 40:1, 41:1, 42:1, 43:1, 44:1, 45:1, 46:1, 47:1, 48:1, 49:1, or 50:1.

In some embodiments, the LNP formulation may be formulated by the methods described in International Publication Nos. WO2011127255 or WO2008103276. As a non-limiting example, the antisense polynucleotide (e.g., ASO) compositions of the present disclosure may be encapsulated in any of the lipid nanoparticle (LNP) formulations described in WO2011127255 and/or WO2008103276; the contents of each of which are herein incorporated by reference in their entirety.

As a non-limiting example, the LNP may be the lipid nanoparticles described in PCT Patent Application Publication No. WO2012170930, the contents of which are herein incorporated by reference in their entirety. As another non-limiting example, the lipid formulation may comprise a cationic lipid of formula A, a neutral lipid, a sterol and a PEG or PEG-modified lipid disclosed in US Patent Publication No.: US 20120101148 to Akinc et al., the contents of which are incorporated herein by reference in their entirety.

As another non-limiting example, the LNP may be a nanoparticle to be delivered by a parenteral route as described in U.S. Patent Application Publication No. US20120207845; the contents of which are herein incorporated by reference in their entirety.

In some embodiments, the antisense polynucleotide (e.g., ASO) compositions may be encapsulated into a rapidly eliminated lipid nanoparticle and the rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc., Deerfield, IL), PEG-based sealants, and COSEAL® (Baxter International, Inc., Deerfield, IL).

In some embodiments, the antisense polynucleotide (e.g., ASO) compositions may be formulated in a solid lipid nanoparticle (SLN). A solid lipid nanoparticle (SLN) may be spherical with an average diameter between 10 to 1000 nm, or between 100 to 1000 nm. SLN possess a solid lipid core matrix that can solubilize lipophilic molecules and may be stabilized with surfactants and/or emulsifiers. The lipid nanoparticle may be a self-assembly lipid-polymer nanoparticle (see Zhang et al., ACS Nano, 2008, 2 (8):1696-1702; the contents of which are herein incorporated by reference in their entirety.

In some embodiments, the LNPs for formulating the antisense polynucleotide (e.g., ASO) composition of the present disclosure may comprise a targeting lipid with a targeting moiety such as the targeting moieties disclosed in US Patent Application Publication No.: US20130202652 to Manoharan et al., the contents of which are incorporated herein by reference in their entirety. As a non-limiting example, the targeting moiety of formula I of US 20130202652 to Manoharan et al. may be selected in order to favor the lipid being localized with a desired organ, tissue, cell, cell type or subtype, or organelle. Non-limiting targeting moieties that are contemplated in the present disclosure include transferrin, anisamide, an RGD peptide, prostate specific membrane antigen (PSMA), fucose, an antibody, or an aptamer.

### Micelles

In some embodiments, the antisense polynucleotide (e.g., ASO) compositions may be formulated in micelles. The term “micelle”, as used herein, refers to any water-soluble aggregate which is spontaneously and reversibly formed from amphiphilic compounds or ions. The size of micelles may be small, less than 10 nm, or less than 9 nm, or less than 8 nm, or less than 7 nm, or less than 6 nm.

In some embodiments, the antisense polynucleotide (e.g., ASO) compositions may be formulated in reverse micelles. The reverse micelle is a microemulsion comprising a dispersion of water-nanodroplets in oil. The reverse micelles can be defined as a system wherein water forms the internal phase and the hydrophobic tails of the lipids form the continuous phase. The reverse micelle may comprise a phospholipid or a sphingolipid. Reverse micelles containing oil(s), surfactant(s), co-surfactant(s), and an aqueous phase are also characterized as water-in-oil microemulsions. As a non-limiting example, the reverse micelle may be prepared using methods described in U.S. Pat. No. 8,877,237; the contents of which are incorporated herein by reference in their entirety. The reverse micelle may be formulated for absorption of the antisense polynucleotides to be delivered across mucosa, such as mouth, nasal and/or rectal mucosa.

In some embodiments, the antisense polynucleotide compositions may be formulated in a micelle using a temperature sensitive polymer. The micelle may be covered with glucose which allows significant delivery of the antisense polynucleotide compositions into the brain. The temperature-sensitive copolymer comprises a cationic block (e.g., a cationic amino acid polymer block) and a temperature-sensitive block (e.g., polyethylene glycol). As a non-limiting example, the temperature sensitive polymer may be made by methods described in the PCT Publication No.: WO2016186204; the contents of which are incorporated herein by reference in their entirety.

### Exosomes and ECVs

Exosomes and extracellular vesicles (ECVs) are small membrane vesicles of endocytic origin that are released into the extracellular environment following fusion of multivesicular bodies with the plasma membrane. Exosomes and ECVs can transport large molecules which are appropriated as nucleic acid delivery vehicles (e.g., Raposo, 2013, J Cell Biol, 200:373; and Validi, 2007, Nat Cell Biol, 9:654). Exosomes may be small in size from 10 to 200 nm, for from 10-150 nm, or from 20-180 nm, or from 40-120 nm, or 10 nm, 20 nm, 30 nm, 40 nm, 50 nm, 60 nm, 70 nm, 80 nm, 100 nm, 120 nm, 140 nm, 160 nm, 180 nm, 200 nm. In some embodiments, exosomes and ECVs may be large in size from 100-1000 nm, or from 100-600 nm.

In some embodiments, the antisense polynucleotide (e.g., ASO) compositions may be formulated using extracellular vesicles (ECVs) and/or exosomes. Exosomes may be made using exosome producing cells. As a non-limiting example, the antisense polynucleotide-exosome formulations may be made using methods known in the art and/or as described in the PCT Publication No.: WO2017054085; the contents of which are incorporated herein by reference in their entirety.

In some embodiments, the antisense polynucleotide (e.g., ASO) compositions formulated into exosomes by transferring the antisense polynucleotides (e.g., ASO constructs) into exosome-producing cells using any methods known in the art such as but not limited to electroporation, transfection using a transfection agent such as lipofection, transformation using heat shock and viral infection. As a non-limiting example, the polynucleotide-exosome formulations may be made using methods described in U.S. Pat. No. 10,695,443; the contents of which are incorporated herein by reference in their entirety.

In some embodiments, the antisense polynucleotide (e.g., ASO) compositions of the present disclosure may be formulated using a targeted and modular exosome loading system. The targeted exosome may comprise a fusion protein that includes an RNA-binding domain and an exosome targeting domain. The RNA biding domain of the fusion protein can bind to the antisense polynucleotide (e.g., ASO) of the present disclosure such that the antisense polynucleotides are packaged inside of the exosome. The exosome targeting domain may include exosome targeting domains of lysosome-associated proteins (e.g., LAMPs and LIMPs).

### Other Nanoparticles and Delivery Agents

In some embodiments, the antisense polynucleotide (e.g., ASO) compositions of the present disclosure may be complexed with a cationic amphiphile in an oil-in-water (o/w) emulsion such as described in European Publication No.: EP2298358 to Satishchandran et al., the contents of which are incorporated herein by reference in their entirety. The cationic amphiphile may be a cationic lipid, modified or unmodified spermine, bupivacaine, or benzalkonium chloride and the oil may be a vegetable or an animal oil. As a non-limiting example, at least 10% of the nucleic acid-cationic amphiphile complex is in the oil phase of the oil-in-water emulsion (see e.g., the complex described in. EP2298358 to Satishchandran et al.; the contents of which are incorporated herein by reference in its entirety.

The antisense polynucleotide (e.g., ASO) compositions of the present disclosure may be formulated in a carbohydrate nanoparticle comprising a carbohydrate carrier. As a non-limiting example, the carbohydrate carrier may include, but is not limited to, an anhydride-modified phytoglycogen or glycogen-type material, phytoglycogen octenyl succinate, phytoglycogen beta-dextrin, anhydride-modified phytoglycogen beta-dextrin. (See e.g., PCT Patent Application Publication No. WO2012109121; the contents of which are herein incorporated by reference in their entirety).

The antisense polynucleotide (e.g., ASO) compositions of the present disclosure may be encapsulated in, linked to and/or associated with synthetic nanocarriers. Synthetic nanocarriers include, but are not limited to, those described in PCT Patent Application Publication Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222. The synthetic nanocarriers may be formulated using methods known in the art and/or described herein, for example, by the methods described in PCT Patent Application Publication Nos. WO2010005740, WO2010030763 and WO201213501 and US Pub. Nos. US20110262491, US20100104645, US20100087337 and US2012024422. The synthetic nanocarrier formulations may be lyophilized by methods described in PCT Patent Application Publication Pub. No. WO2011072218 and U.S. Pat. No. 8,211,473; the contents of each of which are herein incorporated by reference in their entirety.

In some embodiments, the synthetic nanocarriers may contain reactive groups to release the antisense polynucleotide (e.g., ASO) compositions of the present disclosure (see PCT Patent Application Publication No. WO20120952552 and US Pub No. US20120171229, the contents of each of which are herein incorporated by reference in their entirety). The synthetic nanocarrier may be formulated to release the antisense polynucleotide (e.g., ASO) compositions at a specified pH and/or after a desired time interval, for example, after 24 hours and/or at a pH of 4.5 (see PCT Patent Application Publication Nos. WO2010138193 and WO2010138194 and US Pub Nos. US20110020388 and US20110027217, the contents of each of which are herein incorporated by reference in their entirety).

In some embodiments, the synthetic nanocarriers may be formulated for targeted release, controlled and/or sustained release of the antisense polynucleotide (e.g., ASO) compositions of the present disclosure. As a non-limiting example, the synthetic nanocarriers for sustained release may be formulated by methods known in the art, described herein and/or as described in PCT Patent Application Publication No. WO2010138192 and US Pub No. US20100303850, the contents each of which are herein incorporated by reference in their entirety.

In some embodiments, the antisense polynucleotide (e.g., ASO) compositions of the present disclosure may be formulated in a modular composition such as described in U.S. Pat. No. 8,575,123 to Manoharan et al., the contents of which are herein incorporated by reference in their entirety. The modular composition may comprise at least one endosomolytic component, and at least one targeting ligand.

In some embodiments, the antisense polynucleotide (e.g., ASO) composition of the present disclosure may be formulated with formulated lipid particles (FLiPs) disclosed in U.S. Pat. No. 8,148,344 to Akinc et al., the contents of which are herein incorporated by reference in their entirety.

In some embodiments, the antisense polynucleotide (e.g., ASO) composition of the present disclosure may be fully encapsulated in a lipid particle disclosed in US Pub. No. US 20120276207 to Maurer et al., the contents of which are incorporated herein by reference in their entirety. The lipid particles may include a cationic lipid having the formula A, or a lipid composition comprising preformed lipid vesicles and a destabilizing agent which forms a mixture with an active agent.

In some embodiments, the antisense polynucleotide (e.g., ASO) composition of the present disclosure may be formulated in a neutral liposomal formulation such as disclosed in US Pub. No. US 20120244207 to Fitzgerald et al., the contents of which are incorporated herein by reference in their entirety. The phrase “neutral liposomal formulation” refers to a liposomal formulation with a near neutral or neutral surface charge at a physiological pH (e.g., about 7.0).

In some embodiments, the antisense polynucleotide (e.g., ASO) compositions of the present disclosure may be formulated with a lipid bilayer carrier, wherein the antisense polynucleotide (e.g., ASO) compositions may be combined with a lipid-detergent mixture comprising a lipid mixture of an aggregation-preventing agent, a cationic lipid and a fusogenic lipid and a detergent. The nucleic acid-lipid-detergent mixture is dialyzed against a buffered salt solution to remove the detergent and to encapsulate the nucleic acid in a lipid bilayer carrier and provide a lipid bilayer-nucleic acid composition (see, e.g., PCT Patent Application Publication No. WO1999018933 to Cullis et al., the contents of which are incorporated herein by reference in their entirety).

In some embodiments, formulations comprising the antisense polynucleotide (e.g., ASO) compositions described herein may also be constructed or altered such that they passively or actively are directed to different cell types in vivo, including but not limited to immune cells, endothelial cells, antigen presenting cells, and leukocytes (Akinc et al. Mol Ther. 2010, 18:1357-1364; Song et al., Nat Biotechnol. 2005, 23:709-717; Judge et al., J Clin Invest. 2009, 119:661-673; Kaufmann et al., Microvasc Res, 2010, 80:286-293; Santel et al., Gene Ther 2006, 13:1222-1234; Santel et al., Gene Ther, 2006, 13:1360-1370; Gutbier et al., Pulm Pharmacol. Ther. 2010, 23:334-344; Basha et al., Mol. Ther. 2011, 19:2186-2200; Fenske and Cullis, Expert Opin Drug Deliv. 2008, 5:25-44; Peer et al., Science. 2008, 319:627-630; Peer and Lieberman, Gene Ther. 2011, 18:1127-1133; the contents of each of which are incorporated herein by reference in their entirety).

One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010, 18:1357-1364). The LNPs may also be coated on their surface with cell-specific ligands to selective targeting. Exemplary ligands may include folate, transferrin, N-acetylgalactosamine (GalNAc), and antibody and/or its fragment targeted approaches (Kolhatkar et al., Curr Drug Discov Technol. 2011, 8:197-206; Musacchio and Torchilin, Front Biosci. 2011, 16:1388-1412; Yu et al., Mol Membr Biol. 2010, 27:286-298; Patil et al., Crit Rev Ther Drug Carrier Syst. 2008, 25:1-61; Benoit et al., Biomacromolecules. 2011, 12:2708-2714; Zhao et al., Expert Opin Drug Deliv. 2008, 5:309-319; Akinc et al., Mol Ther. 2010, 18:1357-1364; Srinivasan et al., Methods Mol Biol. 2012, 820:105-116; Ben-Arie et al., Methods Mol Biol. 2012, 757:497-507; Peer J Control Release. 2010, 20:63-68; Peer et al., Proc Natl Acad Sci USA. 2007, 104:4095-4100; Kim et al., Methods Mol Biol. 2011, 721:339-353; Subramanya et al., Mol Ther. 2010, 18:2028-2037; Song et al., Nat Biotechnol. 2005, 23:709-717; Peer et al., Science. 2008, 319:627-630; Peer and Lieberman, Gene Ther. 2011, 18:1127-1133); the contents of each of which are incorporated herein by reference in their entirety).

In some embodiments, formulations comprising the antisense polynucleotide (e.g., ASO) compositions described herein are disease target specific.

In some embodiments, formulations comprising the polynucleotide (e.g., ASO) compositions described herein may also be constructed or altered such that their properties are suitable for different administration routes, such as parenteral (intravenously, intramuscularly or subcutaneously), oral, rectal, ophthalmic and/or topical administration. As non-limiting examples, the formulations described herein may be optimized for oral administration by including at least one cationic biopolymer such as, but not limited to, chitosan or a derivative thereof (see, U.S. Pub. No. US20120282343; the contents of which are herein incorporated by reference in their entirety).

In some embodiments, the antisense polynucleotide (e.g., ASO) compositions of the present disclosure can be formulated for controlled release and/or targeted delivery. As used herein, “controlled release” refers to a pharmaceutical composition or compound release profile that conforms to a particular pattern of release to affect a therapeutic outcome. As used herein, “sustained release” refers to a pharmaceutical composition or compound that conforms to a release rate over a specific period of time. The period of time may include, but is not limited to, hours, days, weeks, months and years. In some embodiments, the antisense polynucleotide (e.g., ASO) compositions may be encapsulated into a delivery agent described herein and/or known in the art for controlled release and/or targeted delivery. As used herein, the term “encapsulate” means to enclose, surround, or encase. As it relates to the formulation of the compositions of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition of the disclosure may be enclosed, surrounded or encased within the delivery agent. “Partially encapsulated” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition of the disclosure are encapsulated in the delivery agent.

In some embodiments, the formulations comprising the antisense polynucleotide (e.g., ASO) compositions for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®). In some embodiments, the controlled release and/or targeted delivery formulation may comprise at least one degradable polyester which may contain polycationic side chains.

In some embodiments, the formulations comprising the antisense polynucleotide (e.g., ASO) compositions may be formulated with a sustained release nanoparticle comprising a polymer as described in PCT Patent Application Publication No. WO2010075072 and US Pub Nos. US20100216804, US20110217377 and US20120201859; the contents of each of which are herein incorporated by reference in their entirety.

### Excipients and Adjuvants

In some embodiments, pharmaceutical formulations may additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, anti-oxidants, osmolality adjusting agents, pH adjusting agents and the like, as suited to the particular dosage form desired. Various excipients for formulating pharmaceutical compositions and techniques for preparing the composition are known in the art (see Remington: The Science and Practice of Pharmacy, 21” Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, crosslinked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g., acacia, agar, alginic acid, sodium alginate, tragacanth, chon-drux, cholesterol, xanthan, pectin, gelatin, egg yolk, casein, wool fat, cholesterol, wax, and lecithin), colloidal clays (e.g., bentonite (aluminum silicate) and VEEGUM® (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (TWEEN® 20), polyoxyethylene sorbitan (TWEEN® 60), polyoxyethylene sorbitan monooleate (TWEEN® 80), sorbitan monopalmitate (SPAN® 40), sorbitan monostearate (SPAN® 60), sorbitan tristearate (SPAN®65), glyceryl monooleate, sorbitan monooleate (SPAN® 80)), polyoxyethylene esters (e.g., polyoxyethylene monostearate (MYRJ®45), polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether (BRIJ® 30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER® 188, cetrimonium bromide, cetylpyridinium chloride, benzalkonium chloride, docusate sodium, etc. and/or combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenyl ethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®!!, NEOLONE™, KATHON™, and/or EUXYL®.

Exemplary binding agents include, but are not limited to, starch (e.g., cornstarch and starch paste); gelatin; sugars (e.g., sucrose, glucose, dextrose, dextrin, molasses, lactose, lactitol, mannitol); amino acids (e.g., glycine); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and combinations thereof.

In some embodiments, the pH of the pharmaceutical solutions are maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions; however, these are not preferred due to complex formation and/or mRNA degradation.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

In some embodiments, the polynucleotide (e.g., antisense polynucleotide) of the present disclosure that encodes an antigen may be formulated with adjuvants as nucleic acid vaccines. Adjuvants may be used to enhance the immunogenicity of the nucleic acid vaccine, modify the immune response, reduce the amount of nucleic acid vaccine needed for immunization, reduced the frequency of additional or “booster” immunizations needed or to create an improved immune response in those with weakened or immunocompromised immune systems or the elderly. Co-administration of the adjuvant may be any method known in the art or described herein such as, but not limited to, intravenous (IV), intramuscular (IM), subcutaneous (SC) or intradermal (ID).

Adjuvants may be selected for use with the nucleic acid vaccines by one of ordinary skill in the art. The adjuvants may be natural or synthetic. The adjuvants may also be organic or inorganic. In some embodiments, the adjuvant used with the polynucleotide vaccine is from a class of adjuvants such as, but not limited to carbohydrates, microorganisms, mineral salts (e.g., aluminum hydroxide, aluminum phosphate gel, or calcium phosphate gel), emulsions (e.g., oil emulsion, surfactant based emulsion, purified saponin, and oil-in water emulsion), inert vehicles, particulate adjuvants (e.g., unilamellar liposomal vehicles such as virosomes or a structured complex of saponions and lipids such as polylactide co-glycolide (PLG)), microbial derivatives, endogenous human immunomodulators, and tensoactive compounds. Listings of adjuvants which may be used with the nucleic acid vaccines described herein may be found on the web-based vaccine adjuvant database Vaxjo (see e.g., Sayers et al., Journal ofBiomedicine and Biotechnology; 2012; 2012:831486; the contents of which are herein incorporated by reference in their entirety).

Other exemplary adjuvants may include but are not limited to, interferons, TNF-alpha, TNF-beta, chemokines (e.g., CCL21, eotaxin, HMGB1, SA100-8alpha, GCSF, GMCSF, granulysin, lactoferrin, ovalbumin, CD40L, CD28 agonists, PD1, soluble PD1, PDL1, PDL2) or interleukins (e.g., IL1, IL2, IL4, IL6, IL7, IL10, IL12, IL13, IL15, IL17, IL18, IL21, and IL23), Abisco-100 vaccine adjuvant, Adamantylamide Dipeptide Vaccine Adjuvant, Adjumer™, AF03, Albumin-heparin microparticles vaccine adjuvant, Algal Glucan, Algammulin, alhydrogel, aluminum hydroxide vaccine adjuvant, aluminum phosphate vaccine adjuvant, aluminum potassium sulfate adjuvant, Aluminum vaccine adjuvant, amorphous aluminum hydroxyphosphate sulfate adjuvant, Arlacel A, AS0, AS04, AS03, AS-2 vaccine adjuvant, Avridine®, B7-2 vaccine adjuvant, Bay R1005, Bordetella pertussis component Vaccine Adjuvant, Bupivacaine vaccine adjuvant, Calcium Phosphate Gel, Calcium phosphate vaccine adjuvant, Cationic Liposomal Vaccine Adjuvant, cationic liposome-DNA complex JVRS-100, Cholera toxin, Cholera toxin B subunit, Corynebacterium-derived P40 Vaccine Adjuvant, CpG DNA Vaccine Adjuvant, CRL1005, CTA1-DD gene fusion protein, DDA Adjuvant, DHEA vaccine adjuvant, DL-PGL (Polyester poly (DL-lactide-co-glycolide)) vaccine adjuvant, DOC/Alum Complex, E. coli heat-labile toxin, Etx B subunit Adjuvant, Flagellin, Freund's Complete Adjuvant, Freund's Incomplete Adjuvant, Gamma Inulin, Gerbu Adjuvant, GM-CSF, GMDP, Imiquimod, Immunoliposomes Containing Antibodies to Costimulatory Molecules, ISCOM(s)™, ISCOMA-TRIX®, Killed Corynebacterium parvum Vaccine Adjuvant, Lipopolysaccharide, Liposomes, Loxoribine, LTK63 Vaccine Mutant Adjuvant, LTK72 vaccine adjuvant, LTR192G Vaccine Adjuvant, Matrix-S, MF59, Montanide Incomplete Seppic Adjuvant, Montanide ISA 51, Montanide ISA 720 Adjuvant, MPL-SE vaccine adjuvant, MPL™ Adjuvant, MTP-PE Liposomes, Murametide, Muramyl Dipeptide Adjuvant, Murapalmitine, D-Murapalmitine, NAGO, nanoemulsion vaccine adjuvant, Non-Ionic Surfactant Vesicles, non-toxic mutant El 12K of Cholera Toxin mCT-E112K, PMMA, Poly(LC), Polygen Vaccine Adjuvant, Protein Cochleates, QS-21, Quil-A vaccine adjuvant, RC529 vaccine adjuvant, Recombinant hlFN-gamma/Interferon-g, Rehydragel EV, Rehydragel HPA, Resiquimod, Ribi Vaccine Adjuvant, SAF-1, Saponin Vaccine Adjuvant, Sclavo peptide, Sendai Proteoliposomes, Sendai-containing Lipid Matrices, Specol, SPT (Antigen Formulation), Squalene-based Adjuvants, Stearyl Tyrosine, Theramide®, Threonyl muramyl dipeptide (TMDP), Titer-Max Gold Adjuvant, Ty Particles vaccine adjuvant, and VSA-3 Adjuvant.

### Viral Vectors

The present invention also provides for a viral vector which encodes or delivers the antisense polynucleotides (e.g., ASO) of the invention. The viral vector may comprise or encode an ASO and/or guide RNA as described herein. In an embodiment, the ASO has an RNA backbone. The viral vector can be used to deliver the ASO and/or guide RNA to a target cell. The viral vector may improve the delivery of the ASO and/or guide RNA to a target cell (e.g., a neuronal cell).

In an embodiment, the viral vector comprises a sequence that expresses a nucleic acid comprising an ASO or a guide RNA as described herein.

Viral vectors can be designed to deliver and/or encode the ASO or guide RNA sequences using standards practices known in the art.

The viral vector may comprise an expression cassette wherein the expression cassette encodes a transcript comprising the ASO or guide RNA. In some embodiments, the viral vector comprises a sequence encoding the ASO or guide RNA described herein and one or more regions comprising inverted terminal repeat (ITR) sequences flanking the ASO or guide RNA sequence. In some embodiments, the sequence is operably linked to a promoter. In some embodiments, the promoter is a tissue-specific (e.g., CNS-specific) promoter.

In an embodiment, the viral vector is a retrovirus, lentivirus, adenovirus (AV), or adeno-associated virus (AAV), or a herpes simplex virus. The viral vectors may be derived from any suitable serotype or subgroup. The viral vector may be a human viral vector or a non-human viral vector.

### III. Routes of Administration, Dosage and Delivery

The present disclosure encompasses the delivery of one or more antisense polynucleotide (e.g., ASO) compositions for any therapeutic, prophylactic, pharmaceutical, diagnostic or research use by any appropriate route taking into consideration likely advances in the sciences of drug delivery.

### Delivery

The present disclosure provides for a method of delivering an antisense polynucleotide (ASO) to a cell, wherein the ASO modulates splicing of UNC13A to prevent inclusion of a cryptic exon in UNC13A RNA. This may prevent loss of the UNC13A translated protein and/or prevent loss of functional UNC13A protein. In an embodiment, this is an in vitro method. In an embodiment, the cell is a cell of the central nervous system. In an embodiment, the cell is a neuronal cell. In an embodiment, the ASO is delivered to the cell by a vector, for example, a viral vector, as may be described herein. The method may be an in vitro or an in vivo method. The in vitro method can be used to probe or modulate UNC13A function. The in vivo method may be used to treat a neurodegenerative disorder, as is otherwise described herein.

The antisense polynucleotides (e.g., ASO) and compositions comprising the antisense polynucleotides (e.g., ASO) of the present disclosure may be loaded to vehicles such as those formulation components discussed herein in order to be administered to target cells, tissues and/or organs. The formulations may contain antisense polynucleotide (e.g., ASO) compositions which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated antisense polynucleotide (e.g., ASO) compositions may be delivered to the cell using routes of administration known in the art and described herein

Delivery may also be naked. The antisense polynucleotides (e.g., ASO) and compositions may be delivered to a cell naked. As used herein in, “naked” refers to delivering antisense polynucleotide (e.g., ASO) compositions free from agents which promote transfection. For example, the antisense polynucleotide (e.g., ASO) compositions delivered to the cell may contain no modifications. The naked antisense polynucleotide (e.g., ASO) compositions may be delivered to the cell using routes of administration known in the art and described herein.

### Route of Administration

The antisense polynucleotides (e.g., ASO) or pharmaceutical composition when used as a medicament or used in a method of treatment as described herein may be administered to a subject by any suitable administration method, for example, by injection.

The antisense polynucleotide (e.g., ASO) compositions of the present disclosure may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them to cross the blood-brain barrier, vascular barrier, or other epithelial barrier.

### Dosage

A pharmaceutical composition described herein can be formulated into a dosage form and for a route of administration as described herein.

The polynucleotides and/or pharmaceutical composition may be administered to a subject in a single dose, or a multiple dose. In an embodiment, the multiple dose comprises two, three, or four or more doses. In an embodiment, the ASO, guide RNA, viral vector or pharmaceutical composition is administered to the subject at regular intervals, for example, weekly, biweekly, monthly, every 2 months, every 3 months, every 4 months, every 5 months, every 6 months or yearly. In some embodiments, the first set of doses (e.g., two, three, four, five, six, seven, eight or ten doses) are administered monthly, with further doses administered less frequently (e.g., every 3 months, every 6 months or yearly).

### IV. Methods of Use

The present disclosure provides methods for use of the antisense polynucleotides (e.g., ASOs), compositions and formulations comprising the antisense polynucleotides of the present disclosure.

The polynucleotides, compositions and formulations comprising the polynucleotides of the present disclosure may be used for regulating gene expression at multiple levels. Some aspects of the present disclosure provide methods for regulation of gene expression in a cell comprising administering to the cell the antisense polynucleotides (ASO compounds), compositions and formulations comprising the polynucleotide described herein.

In some embodiments, the gene expression is regulated at the transcription level, or post-transcription level, or translational level, or post-translational level.

### Therapeutic Applications

The antisense polynucleotides (e.g., ASO(s), compositions and formulations comprising the polynucleotides of the present disclosure may be used as therapeutic agents for disease treatment. The therapeutic use of the antisense polynucleotide(s) (e.g., ASO), compositions and formulations comprising the antisense polynucleotide(s) (ASO) of the present disclosure may involve in modulation of endogenously existing RNAs to provide protection from the disease.

In some embodiments, the therapeutic use of the antisense polynucleotides, compositions and formulations comprising the antisense polynucleotides (e.g., ASO) of the present disclosure may relate to administration of in vitro engineered and produced polynucleotides. Synthetic antisense polynucleotides (e.g., ASO) described herein may be delivered into target cells for therapeutic functions.

The polynucleotides (e.g., ASO) or pharmaceutical composition described herein may be for use, or used, as a medicament, for example, in therapy.

Also disclosed herein is a method of treating a neurodegenerative disorder, the method comprising administering to a subject in need thereof a therapeutically effective amount of the ASO of the first or second aspect, the guide RNA of the third aspect, the viral vector of the fourth aspect or the pharmaceutical composition of the fifth aspect.

Also disclosed herein is a method of treating a condition associated with TDP-43 pathology, the method comprising administering to a subject in need thereof a therapeutically effective amount of the ASO of the first or second aspect, the guide RNA of the third aspect, the viral vector of the fourth aspect or the pharmaceutical composition of the fifth aspect

The ASO, guide RNA, viral vector or pharmaceutical composition described in the methods of treatment herein can be used to prevent loss of and/or restore functionality of the UNC13A protein. In an example, the ASO, guide RNA, viral vector or pharmaceutical composition can be used to modulate splicing such that the UNC13A cryptic exon is not included in the UNC13A mRNA.

In an embodiment, the medicament is for use or used to modulate splicing of a UNC13 A pre-mRNA. In an embodiment, the medicament is for use or used to prevent inclusion of the UNC13A cryptic exon in the UNC13A mature RNA, such that the functionality of the UNC13A protein is restored. In an embodiment, the medicament is for use or used in a method of treatment of a disease associated with TDP-43 pathology.

The ASO, guide RNA, viral vector or pharmaceutical composition described herein may be for use or used in a method of treating a neurodegenerative disorder. In an embodiment, the neurodegenerative disorder is associated with reduced nuclear TDP-43. In an embodiment, the neurodegenerative disorder is caused by nucleus-cytoplasmic mislocalization of TDP-43 In an embodiment, the neurodegenerative disorder is associated with TDP-43 pathology (e.g., pathological TDP-43).

In an embodiment, the method of treating comprises first diagnosing a subject with a neurodegenerative disorder associated with TDP-43 pathology ahead of the method of treating. In an embodiment, this is determined using a biomarker of TDP-43 pathology. In an embodiment, this may be determined by genetics, for example, a genetic mutation. In an embodiment, TDP-43 pathology associated with ALS may be determined if FUS and SOD1 mutations are not found in the subject. In an embodiment, TDP-pathology associated with FTD may be determined if C9orf72 or PGRN mutations are not found in the subject. In an embodiment, the biomarker of TDP-43 pathology may include mutant TDP-43. In some embodiments, TDP-43 pathology may be determined with TDP-43 phosphorylation. In some embodiments, TDP-43 pathology may be determined by expression of the STMN2 cryptic exon.

In an embodiment, the method of treating comprises first identifying in a subject whether they possess a SNP variant associated with rs12973192 and/or rs12608932 ahead of the method of treating. This may be determined by genomics.

In an embodiment, the neurodegenerative disorder may be selected from ALS, frontotemporal dementia, Alzheimer's disease, Parkinson's disease, FOSMNN (Facial onset sensory and motor neuronopathy), Perry Syndrome, or a combination thereof.

In an embodiment, the neurodegenerative disorder is ALS (amyotrophic lateral sclerosis). ALS is a chronic and fatal form of motor neuron disease (MND) and may otherwise be referred to as MND, Charcot disease or Lou Gehrig's disease. In some embodiments, the ALS may be ALS is familial ALS or sporadic (idiopathic) ALS. Familial ALS (FALS) is ALS that runs in the family, and accounts for about 10% of ALS cases. Sporadic ALS is non-familial ALS. In an embodiment, the ALS may not be a ALS-FUS and ALS-SOD1 which are genetically-defined forms of ALS. The ASOs, guide RNAs, viral vectors, or pharmaceutical compositions for use, or the method of treatment described herein, may ameliorate one or more symptoms associated with ALS. Symptoms of ALS may include fasciculation (muscle twitches); muscle cramps; tight and stiff muscles (spasticity), muscle weakness, slurred and nasal speech and a difficulty chewing or swallowing. ALS leads to progressive deterioration of muscle function and ultimately often leads to death due to respiratory failure.

In an embodiment, the neurodegenerative disorder is frontotemporal dementia (FTD). Frontotemporal dementia is a type of dementia that affects the frontal and temporal lobes of the brain. The ASOs, guide RNAs viral vectors, or pharmaceutical compositions for use, or the method of treatment described herein, may ameliorate one or more symptoms associated with FTD. Symptoms of FTD may include personality and behavior changes, language problems, problems with mental abilities, memory problems and physical problems (e.g., difficulties with movement). The FTD may be characterized by frontotemporal lobar degeneration (FTLD). The FTLD may be FTLD-TDP, which is an FTLD associated with TDP-43 pathology. This may be characterized by ubiquitin and TDP-43 positive, tau negative, FUS negative inclusion bodies. The FTLD-TDP may be of Type A, Type B, Type C or Type D. Type A is a type of FTLD-TDP that presents with small neurites and neuronal cytoplasmic inclusion bodies in the upper (superficial) cortical layers. Bar-like neuronal intranuclear inclusions may also be seen, although comparatively fewer in number. Type B is a type of FTLD-TDP that presents with neuronal and glial cytoplasmic inclusions in both the upper (superficial) and lower (deep) cortical layers, and lower motor neurons. Neuronal intranuclear inclusions may be absent or are in comparatively small number. Type B may be associated with ALS and C9ORF92 mutations. Type C is a type of FTLD-TDP that presents long neuritic profiles found in the superficial cortical laminae. There may be comparatively few or no neuronal cytoplasmic inclusions, neuronal intranuclear inclusions or glial cytoplasmic inclusions. FTLD-TDP is often associated with semantic dementia. Type D is a type of FTLD-TDP that presents with neuronal intranuclear inclusion and dystrophic neurites. There may be no inclusions in the granule cell layer of the hippocampus. Type D may be associated with VCP mutations. In an embodiment, the FTLD may not be of type FTLD-FUS or FTLD-tau.

In an embodiment, the neurodegenerative disorder is Alzheimer's disease. Alzheimer's disease is a chronic neurodegenerative disease that starts slowly and gradually worsens over time and is the main cause of dementia. The ASOs, guide RNAs, viral vectors, or pharmaceutical compositions for use, or the method of treatment described herein, may ameliorate one or more symptoms associated with Alzheimer's disease. Symptoms of Alzheimer's may include memory problems, confusion and disorientation, problems with speech and language, problems with movement, personality changes and a combination thereof. The Alzheimer's disease may be associated with TDP pathology.

In an embodiment, the neurodegenerative disorder is Parkinson's disease. Parkinson's disease is progressive nervous system disorder that affects movement Parkinson's symptoms usually begin gradually and worsen over time. The ASOs, guide RNAs, viral vectors, or pharmaceutical compositions for use, or the method of treatment as described herein, may ameliorate one or more symptoms associated with Parkinson's disease. Symptoms may include a tremor, slowness of movement (bradykinesia) and muscle stiffness. The Parkinson's disease may be associated with TDP pathology.

In an embodiment, the neurodegenerative disorder is FOSMN (Facial onset sensory and motor neuronopathy). FOSMN is a rare and slowly progressive motor neuron disorder. The ASOs, guide RNAs, viral vectors, or pharmaceutical compositions for use, or the method of treatment described herein, may ameliorate one or more symptoms associated with FOSMN. Symptoms include sensory and motor loss in the face (e.g., facial tingling or numbness), which may further extend to sensory and motor loss in the scalp, neck, upper trunk and arms. The FOSMN may be associated with TDP pathology.

In an embodiment, the neurodegenerative disorder is Perry Syndrome. Perry syndrome is a progressive brain disease. The ASOs, guide RNAs, viral vectors, or pharmaceutical compositions for use, or the method of treatment as described herein, may ameliorate one or more symptoms associated with Perry Syndrome. Symptoms include parkinsonism (a pattern of movement abnormalities), psychiatric changes, weight loss, and hypoventilation. The Perry syndrome may be associated with TDP pathology.

In an embodiment, the neurodegenerative disorder is a hereditary motor neuropathy. The hereditary motor neuropathy may be associated with TDP-43. In an embodiment, the hereditary motor neuropathy may be hereditary motor and sensory neuropathy (HMSN), which may otherwise be known as Charcot-Marie-Tooth (CMT) disease or peroneal muscular atrophy (PMA). The ASOs, guide RNAs, viral vectors, or pharmaceutical compositions for use, or the method of treatment described herein, may ameliorate one or more symptoms associated with HMSN. Symptoms include muscle weakness in the feet, ankles, legs or hands; awkward gait and numbness in the feet arms and hands. pathology.

The ASO, guide RNA, viral vector or pharmaceutical composition when used as a medicament or used in a method of treatment as described herein may be administered to any suitable subject. In a preferred embodiment, the subject is human. In an embodiment, the subject possesses a SNP variant associated with rs12973192 and/or rs12608932. The human subject is any suitable age, for example, an infant (less than 1 year of age) a child (younger than 18 years of age) including adolescents (10 to 18 years of age inclusive), or adults (older than 18 years of age) including elderly subjects (older than 65 years of age).

### Modulation of Splicing

Disclosed herein is a method of modulating UNC13A splicing, the method comprising administering to a cell or subject in need thereof an effective amount of the ASO of the first or second aspect, the guide RNA of the third aspect, the viral vector of the fourth aspect or the pharmaceutical composition of the fifth aspect. In an example, the ASO, guide RNA, viral vector or pharmaceutical composition can be used to prevent loss of and/or restore functionality of the UNC13A protein. In an example, the ASO, guide RNA, viral vector or pharmaceutical composition can be used to modulate splicing such that the UNC13A cryptic exon is not included in the UNC13A mRNA.

### CRISPR Systems: Gene Therapy

The present invention also provides for a guide RNA comprising an ASO of the invention and a scaffold sequence for a Cas nuclease. In an embodiment the ASO has an RNA backbone. The guide RNA can be used in a CRISPR/Cas system to modulate UNC13A splicing. In a preferred embodiment, the guide RNA is a single guide RNA (sgRNA) comprising both the ASO and the scaffold sequence. In an embodiment, the sgRNA comprises the scaffold sequence upstream of the ASO.

Guide RNAs can be designed to include these sequences using standard practices known in the art. In this regard, the ASO serves the function of the crRNA, i.e., the part of the guide RNA that is complementary to the nucleic acid target, and the scaffold sequence is the tracr RNA, i.e., the part of the guide RNA that serves as a binding scaffold for the Cas nuclease.

The scaffold sequence will comprise a binding sequence for a Cas nuclease for use in a CRISPR/Cas system. Any suitable scaffold sequence specific to a Cas nuclease can be selected. The Cas nuclease may be any suitable Cas nuclease that can bind to RNA and is dead or inactivated (e.g., a dCas nuclease). In an embodiment, the Cas nuclease is a Cas 13 nuclease. The Cas 13 nuclease may be of any suitable sub-type. In an embodiment, the Cas13 nuclease is of 13a or 13d subtype.

The ASOs and/or guide RNAs (i.e., as part of a CRISPR/dCas system) are further used to mask crucial elements of the CE splicing (splice donors and acceptors sites), and/or the splice regulatory elements (SRE)s. These therapeutics prevent the splicing machinery from recognizing the CE and thereby preventing incorporation of the cryptic exon in the mature UNC13A RNA.

The disclosure also provides for a Cas system, comprising the guide RNA as described herein and a dCas nuclease. The Cas nuclease may be as described above.

The guide RNA and associated Cas systems described herein can be used to mask the crucial elements of the cryptic exon splicing, including splice sites (splice donors and acceptors), and/or splice regulatory elements.

### VI. Definitions

To facilitate an understanding of the present invention, a number of terms and phrases are defined below.

About: As used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. It is understood that, the term “about” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

Analog: As used herein, the term “analog” is meant to include polypeptide variants which differ by one or more amino acid alterations, e.g., substitutions, additions or deletions of amino acid residues that still maintain one or more of the properties of the parent or starting polypeptide.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

Antisense polynucleotide: As used herein, an “antisense polynucleotide” or “antisense oligonucleotide” (ASO) has its normal meaning in the art and refers to a synthetic single stranded string of nucleosides joined by internucleoside linkages. The nucleosides may be linked with phosphate-based linkages, phosphodiester, phosphoramidite or phosphorothioate linkages or a combination thereof and the like as described herein. ASOs are used in the art as therapeutics, e.g., for targeting mRNA. Canonically, they bind complementarily (‘antisense’) through Watson-Crick base pairing to a region of a nucleotide sequence of the pre-messenger ribonucleic acid (pre-mRNA) or mature mRNA to modulate mRNA function or splicing. “Capable of binding” referred to herein refers to any sequence that is sufficiently complementary to the target sequence or crucial element to form an association with that target sequence or element thereof. In most cases, ASOs are synthetic in origin and therefore the term, in that instance, is intended to exclude any naturally occurring or transcribed RNA products.

The ASOs defined herein bind UNC13A pre-mRNA. The ASO sequences described herein are synthetic oligonucleotides and isolated from and distinguished from any genomic or transcriptome sequence. The ASOs may nevertheless form part of another longer synthetic sequence (e.g., a guide RNA as disclosed herein).

Associated: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Co-Administered: As used herein, the term “co-administered” or “co-administering” means administering a first construct or system with one or more additional constructs or systems or other therapeutic agents or moieties sufficiently close in time such that the effect of the first construct or system or other therapeutic agents or moieties is enhanced.

Complementarity: As used herein, the term “complementarity” refers to Watson-Crick base pairing in RNA, e.g., wherein A binds with U (or modified variants thereof), and wherein C binds with G (or modified variants thereof). Strands of complementary sequence are referred to as sense and antisense, with the sense strand being the pre-mRNA that was generated after transcription, with the antisense sequence (e.g., ASO or therapeutic) being complementary to the sense sequence. In the ASOs disclosed herein “U” and “T” nucleosides, e.g., uracil or thymine, may be used interchangeably. Therefore “U” in any of SEQ ID NOS: 4-546 may be replaced by “T”. Complementarity need not be 100% or “perfect” in order for there to be binding between two nucleic acid-based compounds. Complementarity may be 30%, 40%, 50%, 60%, 70%, 80%, 90%, 91%, 92%, 93%, 94%, 95%, 96%, 97%, 98%, 99%, or 100%, or any whole integer between 30-100%. Complementarity may be relative to the entire length of the polynucleotide or antisense polynucleotide or relative to the target sequence. As such complementarity may be any degree of 1-100% over the length of one nucleotide to the full-length of the antisense polynucleotide or target, or any length in between, e.g., 30%, 40%, 50%, 60%, 70%, 80%, 90%, 91%, 92%, 93%, 94%, 95%, 96%, 97%, 98%, 99%, or 100% of the whole.

Compound: As used herein, the term “compound” or “structure,” is meant to include all stereoisomers, geometric isomers, tautomers, and isotopes of the structures depicted.

The compounds or structures described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. Compounds or structures of the present disclosure also include tautomeric forms. Tautomeric forms result from the swapping of a single bond with an adjacent double bond and the concomitant migration of a proton. Tautomeric forms include prototrophic tautomers which are isomeric protonation states having the same empirical formula and total charge. Examples prototrophic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. Compounds or structures of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium. The compounds or structures and salts of the present disclosure can be prepared in combination with solvent or water molecules to form solvates and hydrates by routine methods.

Cryptic Exon: As used herein a “cryptic exon” refers to a splicing variant that are incorporated into a mature mRNA, introducing frameshifts or stop codons, among other changes in the resulting mRNA. Cryptic exons are absent in the normal form of mRNA, and are usually skipped by the spliceosome, but arise in an aberrant form. A cryptic exon may otherwise be referred to as “CE”, “cryptic” or “cryptic event” herein or elsewhere in the art.

Delivery: As used herein, “delivery” refers to the act or manner of delivering a compound, substance, entity, moiety, cargo or payload.

DNA and RNA: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Effective amount or therapeutically effective amount: As used herein, the terms “effective amount or therapeutically effective amount” refers to the amount of the polynucleotides or pharmaceutical composition needed to bring about an acceptable outcome of the therapy as determined by reducing the likelihood of disease as measurable by clinical, biochemical or other indicators that are familiar to those trained in the art. The therapeutically effective amount may vary depending upon the condition, the severity of the condition, the subject, e.g., the weight and age of the subject and the mode of administration and the like, which can readily be determined by one of ordinary skill in the art.

Encapsulate: As used herein, the term “encapsulate” means to enclose, surround or encase.

Encode: As used herein the term “encode” refers broadly to any process whereby the information in a polymeric macromolecule is used to direct the production of a second molecule that is different from the first. The second molecule may have a chemical structure that is different from the chemical nature of the first molecule.

Enhance: As used herein, the terms “enhance” and “enhancement” refers to an increase of at least about 5%, 10%, 20%, 25%, 50%, 75%, 100%, 150%, 200%, 300%, 400%, 500% or more of a reference; the reference may be a biological function of a nucleic acid or protein and a gene expression level, etc.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element. when referring to polypeptides are defined as distinct amino acid sequence-based components of a molecule. Features of the polypeptides encoded by the present polynucleotide, such as surface manifestations, local conformational shape, folds, loops, half-loops, domains, half-domains, sites, termini or any combination thereof.

Formulation: As used herein, a “formulation” includes at least one compound, substance, entity, moiety, cargo or payload and a delivery agent.

Fragment: A “fragment,” as used herein, refers to a portion. For example, fragments of proteins may comprise polypeptides obtained by digesting full-length protein isolated from cultured cells.

Guide RNA: As used herein, the term “guide RNA” refers to one part of a CRISPR/Cas genome editing system, the other part being a CRISPR associated endonuclease (Cas protein). The guide RNA comprises a scaffold sequence for Cas-binding (e.g., known as tracr RNA) and a nucleotide sequence that is complementary to and recognises the target (crRNA).

Homology: As used herein, the term “homology” refers to the overall relatedness between polymeric molecules, e.g., between polynucleotide molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Inactive Ingredient: As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Ionizable Lipid: As used herein “ionizable lipid” refers to any of a number of lipid species that carry a net positive charge at a selected pH.

Lipid Nanoparticle: As used herein “lipid nanoparticle” or “LNP” refers to a delivery vehicle comprising one or more lipids (e.g., cationic lipids, non-cationic lipids, PEG-modified lipids).

Liposome: As used herein “liposome” generally refers to a vesicle composed of lipids (e.g., amphiphilic lipids) arranged in one or more spherical bilayers or bilayers.

Modified: As used herein “modified” or, as appropriate “modification” refers to a changed state or structure of a molecule. Molecules may be modified in many ways including chemically, structurally, and functionally. With respect to nucleic acid molecules (e.g., DNA and RNA), the modifications are A, G, C, U or T nucleotides. Generally, herein, these terms are not intended to refer to the ribonucleotide modifications in naturally occurring 5′-terminal mRNA cap moieties. With respect to polypeptides, the term “modification” refers to a modification as compared to the canonical set of 20 amino acids. [0448] mRNA: As used herein, the term “messenger RNA” (mRNA) means a polynucleotide which encodes a polypeptide of interest and which is capable of being translated to produce the encoded polypeptide of interest in vitro, in vivo, in situ or ex vivo. [0449] miRNA: As used herein, the term “miRNA” or “microRNA” refers to a class of small noncoding RNAs approximately 22 nucleotides long. They act as negative regulators of gene expression at the post-transcriptional level, by means of binding their target mRNAs through imperfect base pairing with the respective 3′-untranslated region (3′-UTR).

Non-Cationic Lipid: As used herein “non-cationic lipid” refers to any neutral, zwitterionic or anionic lipid.

Pharmaceutical Composition: As used herein the term “pharmaceutical composition” refers to compositions comprising at least one active ingredient and optionally one or more pharmaceutically acceptable excipients.

PEG: As used herein “PEG” means any polyethylene glycol or other polyalkylene ether polymer.

Prophylactic benefit: As used herein, “prophylactic benefit” refers to delaying or eliminating the appearance of a disease or condition, delaying or eliminating the onset of symptoms of a disease or condition, slowing, halting, or reversing the progression of a disease or condition, or any combination thereof. In the context of the present invention, the prophylactic benefit or effect may involve the prevention of the condition or disease. The polynucleotides or pharmaceutical composition may be administered to a subject at risk of developing a particular disease, or to a subject reporting one or more of the physiological symptoms of a disease, even though a diagnosis of this disease may not have been made.

Reduce: As used herein, the terms “reduce” and “reduction” refers to a decrease of at least about 25%, 35%, 50%, 75%, 80%, 85%, 90%, 95%, 97% or more of a reference; the reference may be a biological activity of a nucleic acid or protein, and a gene expression level, etc.

RNA-seq: As used herein, the term “RNA-seq” otherwise known as “RNA sequencing”, refers to a next-generation sequencing technology which reveals the presence and quantity of RNA in a sample which can be used to analyze the cellular transcriptome.

Splicing: As used herein, the term “splicing” refers to the process wherein pre-mRNAs are transformed into mature mRNAs, wherein introns are removed and exons are joined together. Aberrant splicing with respect to UNC13A as referred to herein refers to a splicing event resulting in inclusion of the novel UNC13A cryptic exon in the mature mRNA. Modulated splicing or modulating splicing as referred to herein refers to preventing the aberrant splicing of UNC13A such that the novel UNC13A cryptic exon is not included in the mature UNC13A mRNA.

Sterol: As used herein “sterol” is a subgroup of steroids consisting of steroid alcohols.

Structural Lipid: As used herein “structural lipid” refers to sterols and lipids containing sterol moieties.

Subject: As used herein, the term “subject” refers to any suitable subject, including any animal, such as a mammal. In preferred embodiments described herein, the subject is a human.

TDP-43: As used herein, the term “TDP-43” refers to TAR DNA Binding protein 43 (Transactive response DNA binding protein 43 kDa), which in humans is a protein encoded by the TARDBP gene. TDP-43 has been shown to bind both DNA and RNA and have multiple functions in transcriptional repression, pre-mRNA splicing and translational regulation, among other functions. Pathological TDP-43 may refer to a TDP-43 protein that is associated with a disease state. Pathological TDP-43 may be a hyper-phosphorylated, ubiquitinated or cleaved form of TDP-43, a TDP-43 form with decreased solubility, or a misfolded forn of TDP-43, a mutant form of TDP-43, or a TDP-43 with altered cellular location.

Transcription: As used herein the term “transcription” refers to the formation or synthesis of an RNA molecule by an RNA polymerase using a DNA molecule as a template.

Translation: As used herein the term “translation” refers to the formation of a polypeptide molecule by a ribosome based upon an RNA template.

Treat and Prevent: As used herein the terms “treat” or “prevent” as well as words stemming therefrom do not necessarily imply 100% or complete treatment or prevention. Rather there are varying degrees of treatment or prevention of which one of ordinary skill in the art recognizes as having a potential benefit or therapeutic effect. Also, “prevention” can encompass delaying the onset of the disease, symptom or condition thereof. The terms refer to an approach for obtaining beneficial or desired results in a subject, which includes a prophylactic benefit and a therapeutic benefit.

Therapeutic benefit: As used herein, “therapeutic benefit” refers to eradication, amelioration or slowing the progression of the underlying disorder being treated. Also, a therapeutic benefit is achieved with the eradication or amelioration of one or more of the physiological symptoms associated with the underlying disorder such that an improvement is observed in the subject, notwithstanding that the patient may still be afflicted with the underlying disorder.

UNC13A: As used herein, the term “UNC13A” refers to a gene that encodes for the UNC13A protein. UNC13 proteins play an important role in neurotransmitter release at synapses.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise viral parent or reference sequence.

Viral vector: As used herein, a “viral vector” refers to any virus vector that can be used to deliver the nucleic acid material of interest (e.g., ASO or guide RNA) into cells.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

For any sequence described herein, the complementary sequence or reverse complement is also considered part of the disclosure.

### Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” (and related terms such as “comprise” or “comprises” or “having” or “including”) is intended to be open and permits but does not require the inclusion of additional elements or steps. When the term “comprising” is used herein, the term “consisting of” “consist of” or “consist essentially of” is thus also encompassed and disclosed. The term “comprises” or “comprising” can be used interchangeably with “includes”.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

Any genomic or chromosomal position described herein refers to the position on the human genome and associated transcriptome (hg38, e.g., hg38 assembly). And for any sequence described herein, the complementary sequence or reverse complement is also considered part of the disclosure.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure. The present disclosure is further illustrated by the following non-limiting examples.

### EXAMPLES

### Example 1: Knockdown of TDP-43 Leads to Inclusion of a Cryptic Exon in UNC13A

To discover novel splicing events caused by TDP-43 depletion on neurons, RNA-seq was performed on human induced pluripotent stem cell-derived (iPSC) i3N neurons stably expressing CRISPRi machinery. Expression of a sgRNA targeting TARDBP reduced TARDBP RNA and protein and was confirmed by RNA sequencing and tandem mass spectroscopy. Differential gene expression analysis revealed widespread transcriptome changes, with 4,844 genes significantly differentially expressed after TDP-43 knockdown (2,497 up regulated, 2,347 downregulated). To find genes affected by cryptic events, differential splicing analysis was performed, and focused on genes which were both differentially spliced and downregulated. 126 genes were both differentially spliced and downregulated, including genes which have previously described to contain cryptic events, such as PFKP, SETD5, and STMN2.

Of the genes which were changed, we noted that 2 members of the UNC13 family, UNC13A and UNC13B, were both affected by TDP-43 knockdown. These genes are of particular interest not only because they encode critical synaptic proteins, but also because previous studies have genetically linked UNC13A polymorphisms as both a risk factor and survival modifier in ALS and FTD.

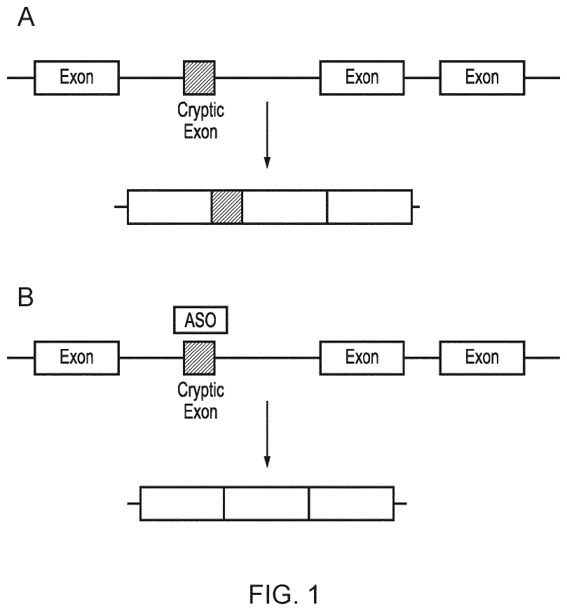

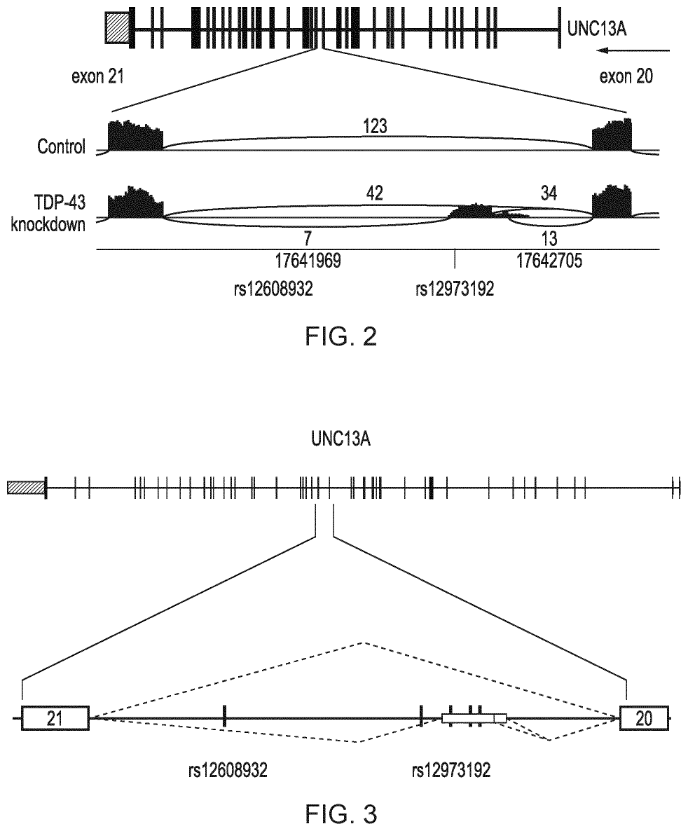

Closer inspection of the significant splicing events in the UNC13A gene, revealed that UNC13A contained a previously unreported novel cryptic cassette exon (FIG. 1 and FIG. 2). The CE after TDP-43 knockdown had both a shorter (SEQ ID NO 2), and longer (SEQ ID NO 3) form, between exons 20 and 21, and increased intronic retention between exons 31 and 32. This cryptic exon has a single novel donor splice site and two novel acceptor splice sites, and iPSC neurons expressed both the shorter and longer form of the cryptic exon upon TDP-43 KD. To assess if TDP-43 binding could be directly linked to these splice changes, we downloaded publicly available TDP-43 individual nucleotide resolution Cross-Linking and ImmunoPrecipitation (iCLIP) data performed in neuronal-like SH-SY5Y and NSC human cell lines (Tollervey et al. 2011). As has been found with other cryptic exons regulated by TDP-43, multiple TDP-43 binding peaks were found both downstream and within the body of the cryptic cassette exon in UNC13A. It was also observed that the cryptic event in UNC13A caused by TDP-43 knockdown was in close proximity to 2 of the polymorphisms which have been previously linked to both ALS and FTD: rs12973192 and rs12608932.

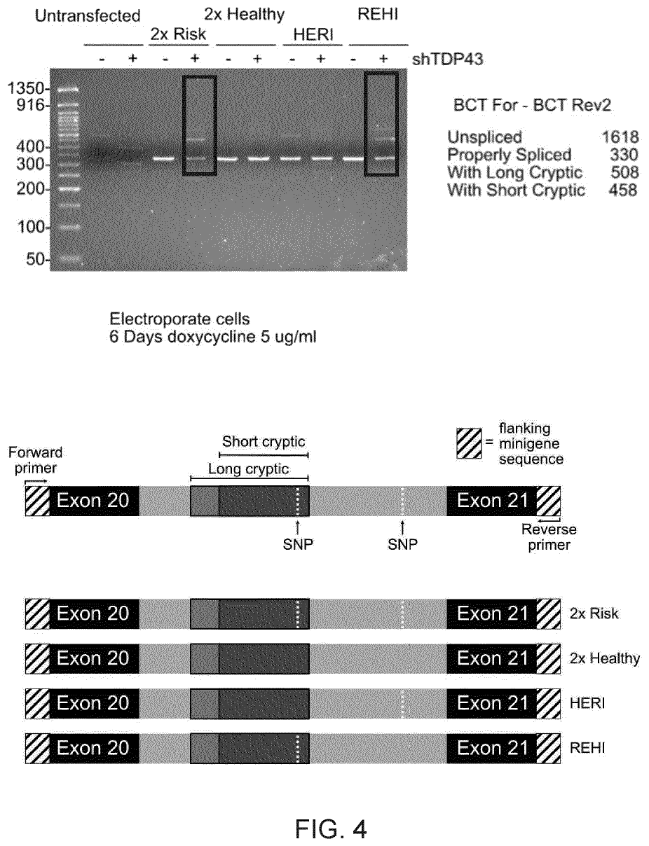

One of the polymorphisms, rs12973192, lays 16 bp inside the cryptic event, and the other, rs12608932, is located 534 bp downstream of the 3′ splice site of the cryptic event (FIG. 3). While there are multiple polymorphisms along UNC13A that have been clinically linked to ALS, it was noted that all of the clinically relevant ones are in high linkage disequilibrium (LD) with the SNP inside the cryptic exon, rs12973192 (FIG. 4, showing that rs12973192 is the main SNP driving changes in UNC13A cryptic splicing associated to risk of aggressive disease progression).

### Example 2: Neuronal Cell Expression of Cryptic Exon in UNC13A

It was next assessed whether this cryptic event could be reliably found across TDP-43 knockdowns in neuronal-like cells. Publicly available RNA-seq datasets were downloaded from induced human motor neurons (Klim, GEO series accession number GSE121569), as well as another high quality TDP-43 siRNA knockdown in a neuroblastoma (NB) cell line (Appocher, GEO series accession number GSE97262).

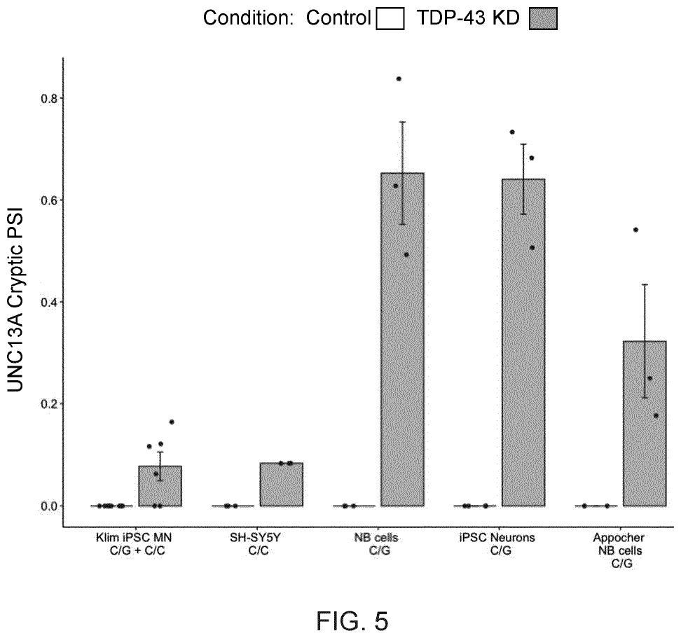

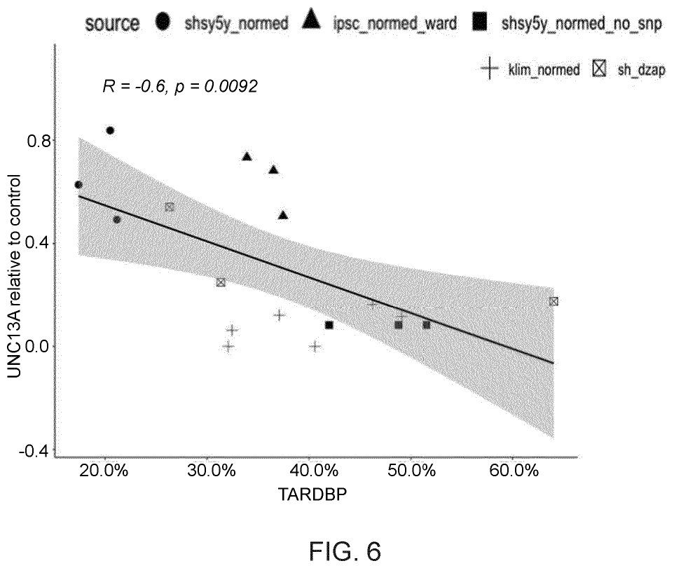

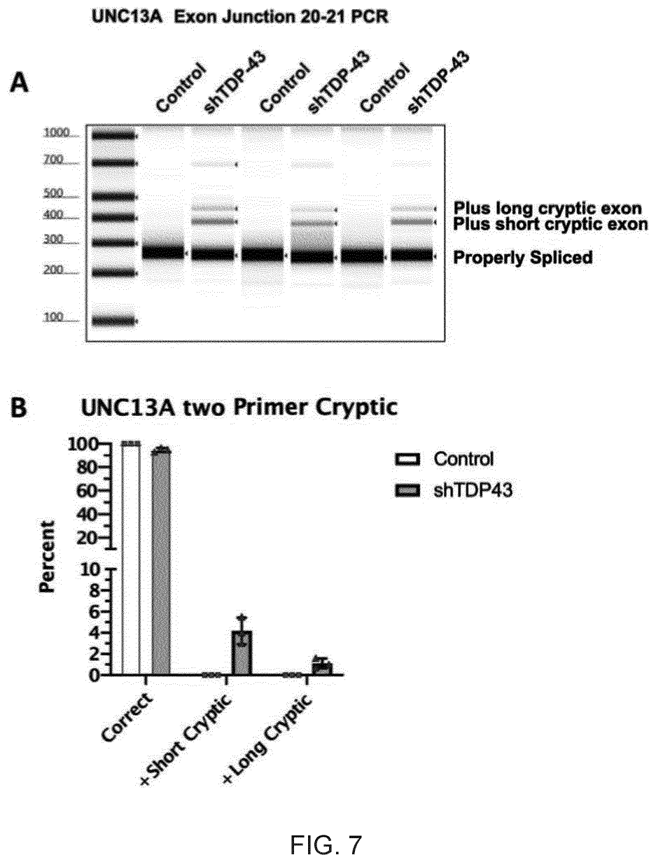

In addition to these public datasets, TDP-43 siRNA knockdown and RNA-sequencing was performed on the NB cell line, as well as an siRNA knockdown of TDP-43 in SH-SY5Y cells. Expression of the UNC13A cryptic exon was found across all datasets, with varying levels of inclusion (FIG. 5). Variation on the allelic expression of rs12973192 was observed within these datasets. For rs12973192 the major allele is C and the minor G allele is the risk allele which has been associated with FTLD and ALS. SH-SY5Y cells were homozygous for the major C allele, while the NB cells line and our iPSC neurons expressed both C and G allele in RNA. The hMN from Klim were variable, with 2 knockdowns expressing both C and G allele, while the other four either expressed the major C allele or had no coverage on rs12973192. It was noted that expression of the cryptic exon was lowest in the SH-SY5Y cell lines, which were homozygous on the C allele, although this could also be due to variable levels of knockdown efficiency. Across the five datasets, there was a strong correlation (R=−0.6, p=0.0092) between the efficiency of the TARDBP depletion, and the amount of UNC13A cryptic present, with those samples with the greatest reduction in TARDBP RNA relative to control having the greatest inclusion of the UNC13A cryptic (FIG. 6). The presence of the cryptic event was also validated by qPCR (FIG. 7) in SH-SY5Y TDP-43 KD.

### Example 3: UNC13A is Downregulated in TDP-43 KD on Both RNA and Protein Level

UNC13A is critical for synaptic transmission, with previous reports that UNC13A knockout loses 90% of synaptic transmission, with the remaining 10% coming from UNC13B, and with complete loss of both UNC13A and UNC13B there is no synaptic transmission.

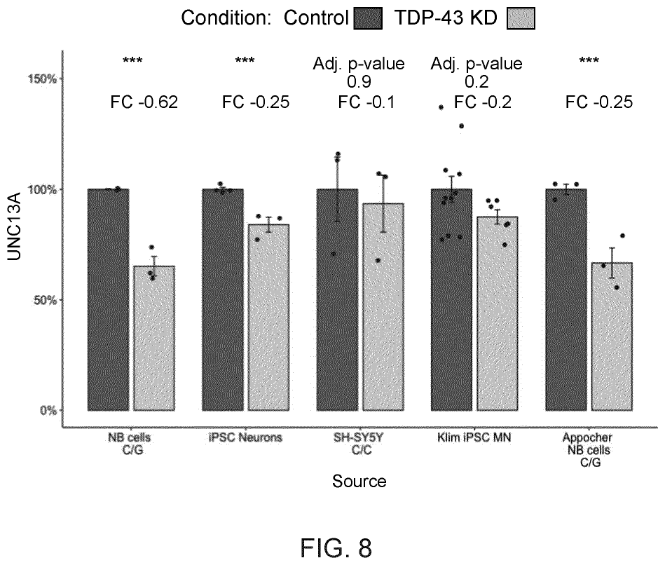

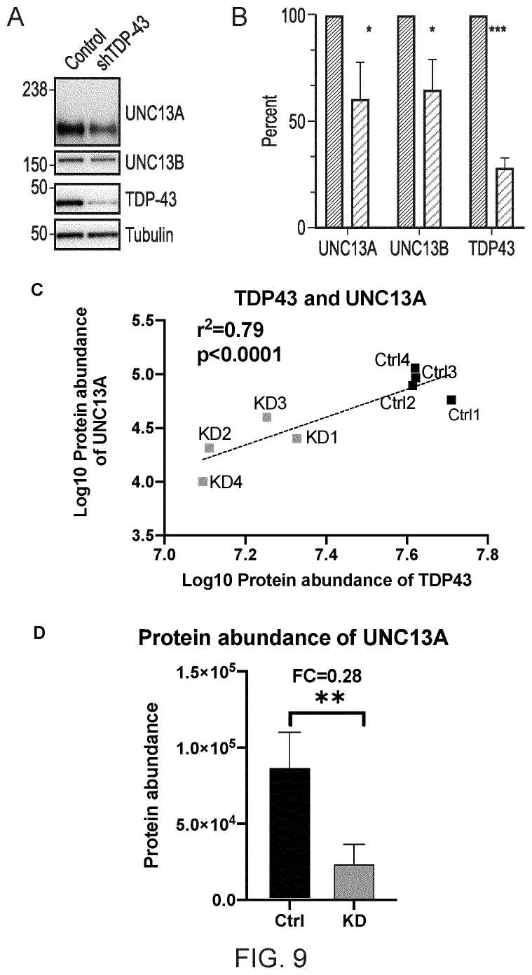

Given that the cryptic event in UNC13A creates a frameshift in UNC13A transcript and contains multiple premature stop codons, it was assessed if TDP-43 KD would affect the levels of UNC13A. Differential gene expression analysis was performed on the five different TDP-43 knockdown experiments and confirmed that TARDBP RNA levels were reduced in all experiments. UNC13A RNA was significantly reduced in the iPSC neurons (−0.25-fold), the Appocher NB cells (−0.25-fold), and the additional TDP-43 siRNA on the same NB lines (−0.62 fold). In SH-SY5Y and Klim hMN, there was a very slight but nonsignificant decrease in UNC13A RNA (−0.1 and −0.2-fold respectively) (FIG. 8). The reduction RNA level of UNC13A after TDP-43 knockdown was confirmed by qPCR in SH SY5Y cells. FIG. 9 shows how loss of TDP-43 leads to a reduction of UNC13A at the protein level.

### Example 4: UNC13A Cryptic Event Occurs in Patient Tissue Affect by TDP-43 Proteinopathy

TDP-43 nuclear depletion and aggregation is a hallmark of ALS, as well certain subtypes of FTLD. It was therefore explored if the UNC13A cryptic event could be found in patient tissues affected by TDP-43 pathology. To assess the presence of the UNC13A cryptic, we quantified the number of spliced reads supporting its inclusion in the NYGC ALS Consortium RNA-Seq dataset. The NYCG ALS dataset contains 377 patients with 1349 neurological tissue samples, including non-neurological disease controls, FTLD, ALS, FTD with ALS (ALS-FTLD), or ALS with suspected Alzheimer's disease (ALS-AD). FTLD cases were further categorized based on neuropathological diagnosis, those with TDP-43 aggregates, FTLD-TDP-A, B, C or those with FUS or TAU aggregates. As the presence of TDP-43 proteinopathy has not been systematically assessed in the ALS cases, patients were separated into those with SOD1 or FUS mutations, which are presumed not to have TDP-43 proteinopathy, and all others.

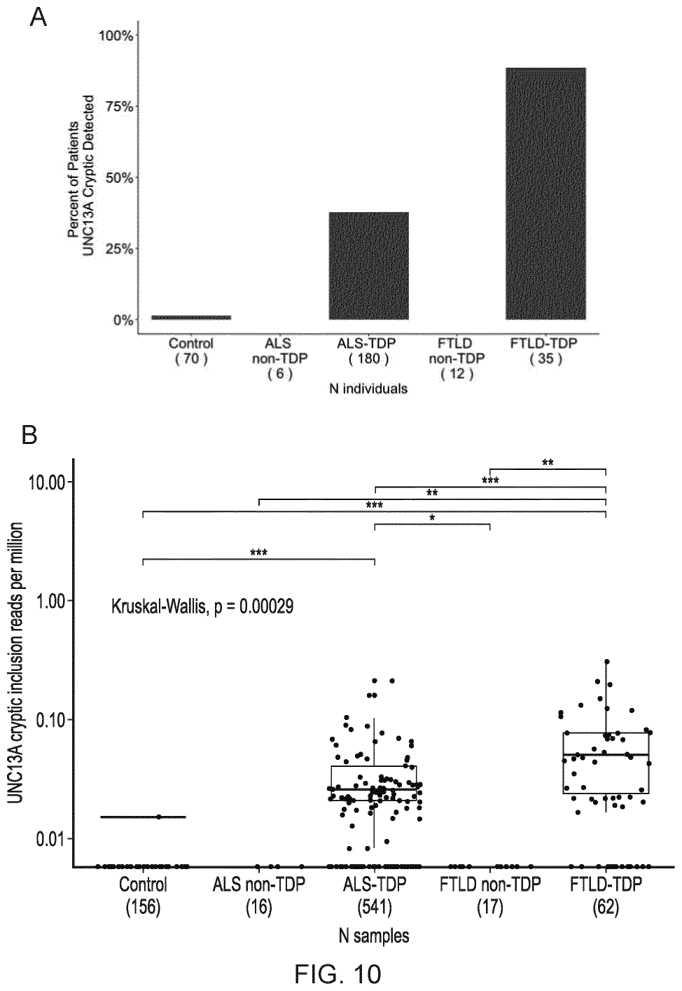

It was first explored the presence of the UNC13A cryptic in disease relevant tissues, specifically frontal and temporal cortices for FTLD, and lumbar, cervical, and thoracic spinal cord samples in ALS. We also looked for the presence of UNC13A cryptic in all six tissues in the non-neurological controls. Strikingly, the UNC13A cryptic was detected in 88% of the FTLD-TDP patients and 38% of ALS-TDP patients (FIG. 10, Panel A), and in none of the non-TDP FTLD or ALS patients, although there was a single non-neurological control with detection of the UNC13A cryptic. The level of the UNC13A cryptic was assessed with the number of inclusion reads supporting the cryptic. UNC13A cryptic was found at the highest rate in the FTLD-TDP samples, followed by the ALS-TDP group (FIG. 10, Panel B).

### Example 5. Identification of UNC13A Cryptic Exon in Tissue

Using the NYCG dataset, patients with pathological diagnosis of TDP-43 proteinopathy were investigated. This included FTLD cases which had been assessed by pathologists with any of the FTD TDP-A-B-C subtypes (i.e., those known to be affected by TDP-43 protein aggregates in frontal and temporal areas), as well as FUS and TAU FTLD subtypes, which are not affected by TDP43 aggregates.

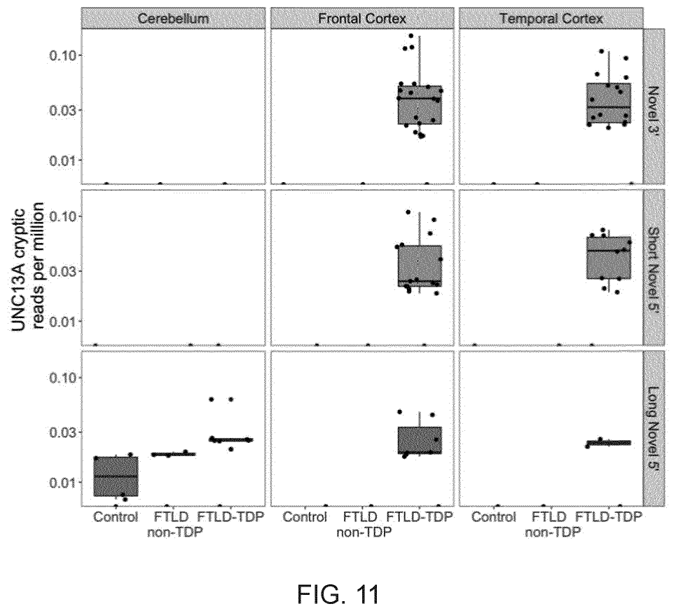

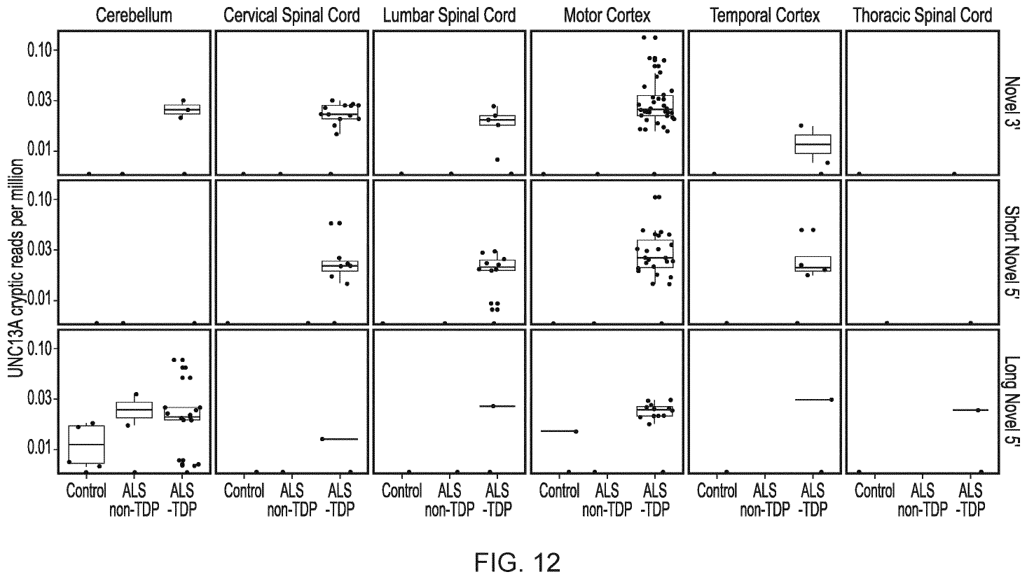

Strikingly, it was noted that UNC13A cryptic exon could be observed in tissue known to be affected by TDP-43 proteinopathy and that this effect was specific to these tissues, showing almost no presence in either controls patients or FTD non TDP subtypes (e.g., FUS and TAU), and no presence in the cerebellum of the TDP subtype patients, which is a tissue which is not affected by TDP-43 proteinopathy. (FIG. 11 and FIG. 12).

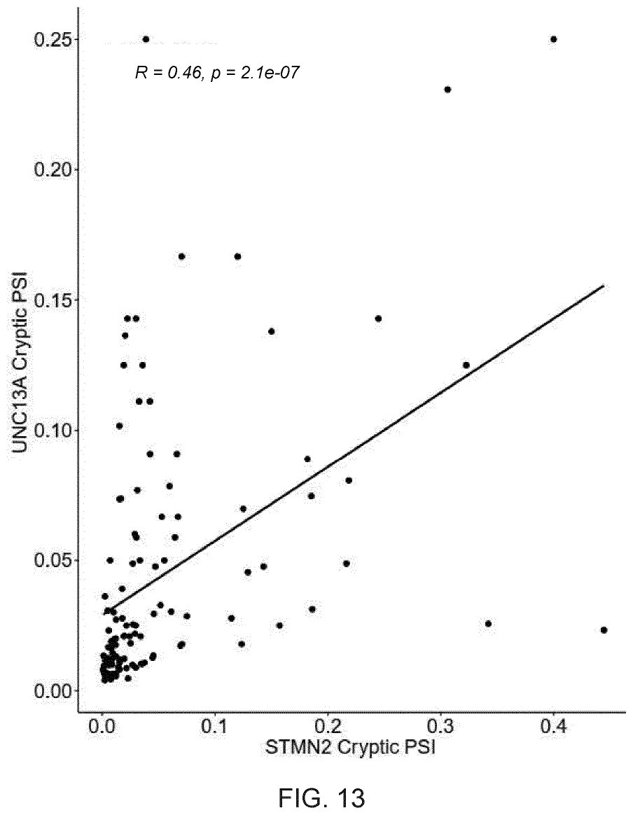

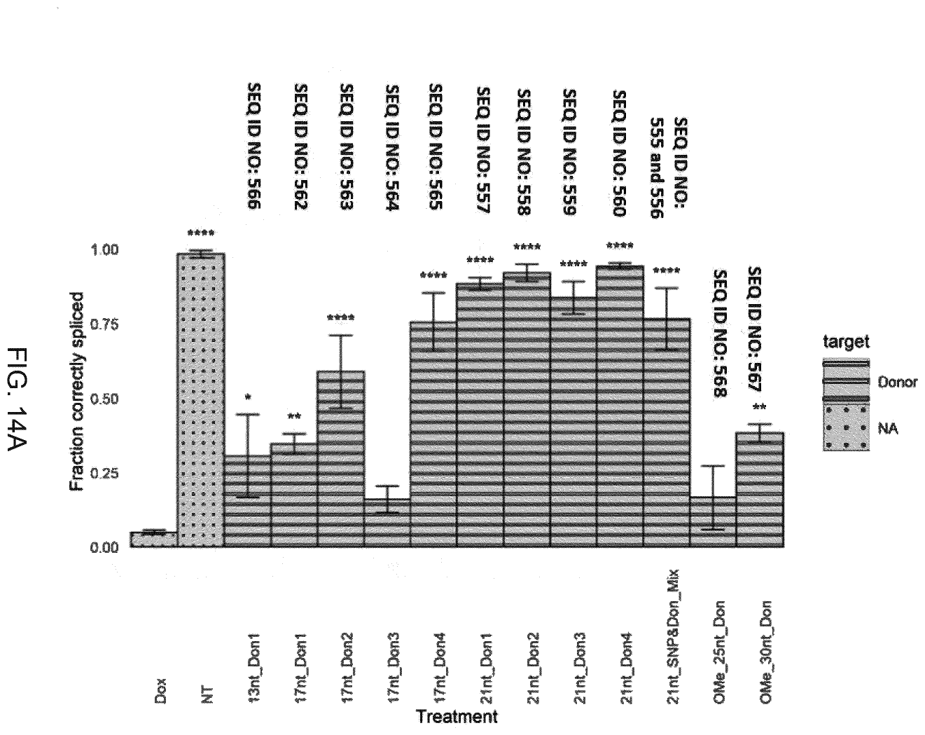

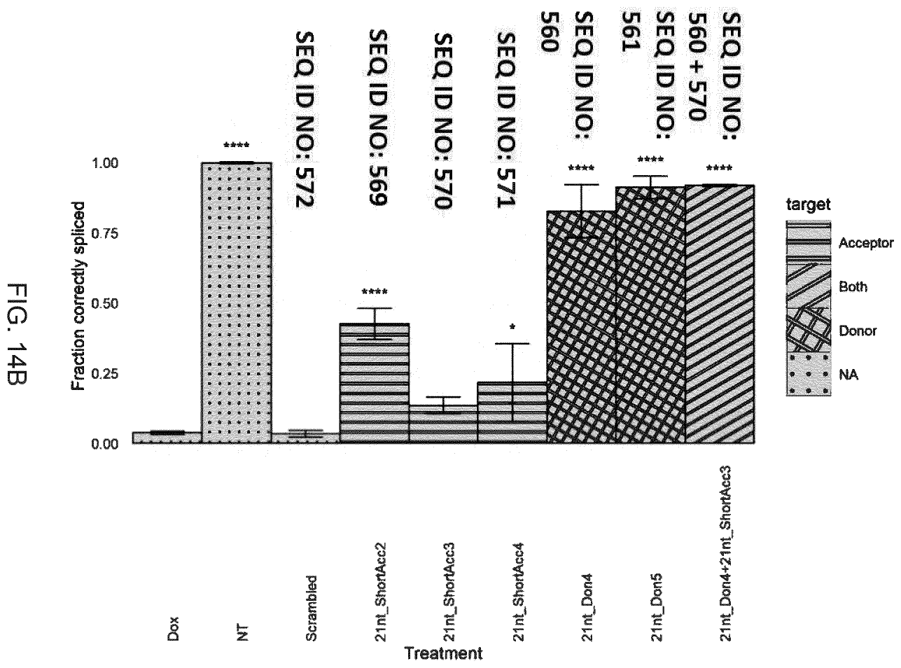

The search was expanded to include ALS cases to observe if UNC13A cryptic could be observed in tissues affected by TDP-43 proteinopathy. As direct staining information is not available for all samples in the database, the inclusion of truncated stathmin-2 (STMN2) was used as a marker for TDP-43 dysfunction. It has been previously demonstrated that the levels of truncated STMN2 correlate both with the burden of pTDP-43 and can serve as a proxy for the level of TDP-43 dysfunction (FIG. 13). UNC13A cryptic was detected in 92/277 of tissues affected by TDP-43 loss of function, as measured by truncated level of STMN2. The UNC13A cryptic was detected in 130/603 tissues in FTD/ALS TDP types, and 99/215 of patients with TDP-pathology in any of their tissue.

### Example 6. UNC13A Sequences

The UNC13A cryptic exon (CE) variants, their location on chromosome 19, the single nucleotide polymorphisms (SNPs) and branchpoints are given in SEQ ID NO. 1 below, where the long variant is underlined, the shorter variant is in italics, the SNPs are bolded (rs12973192 cryptic exon is within the UNC13A CE sequence (emboldened G), and rs12608932 (emboldened U) is within the intronic region) and the branch points are highlighted. Lower case bases denote the bases immediately flanking the splice sites.

SEQ ID NO 1 -Portion of UNC13A transcribed mRNA intronic sequence with cryptic
exon - cords, chr19:17,641,557 - 17642844. SEQ ID NO 1 has the sequence:
GUGAGGGUCAUUGCUCGGCCCCUCCCAUGCCACUUCCACUCACCAUUCCUG
CCUGCCCAGCUCUUCCUCUUUCUGGCCACACCAUCCACACUCUCCUGGCCC
UCUGAGACUGCCCGCCAUGCCAUUCCCUUUACCUGGAAAACUCCUCCCUAU
CCAUCAAAGUCCAGAUUCAGGGUCACCUCCUCUGGGAAGCCCACCUUGGCC

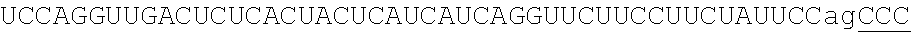

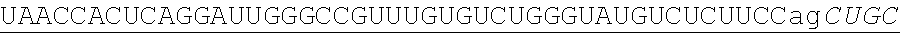

CUGGGUUUCCUGGAAAGAACUCUUAUCCCCAGGAACUAGUUUGUUGAAUAAA
UGCUGGUGAAUGAAUGAAUGAUUGAACAGAUGAAUGAGUGAUGAGUAGAUAAA
AGGAUGGAUGGAGAGAUGGguGAGUACAUGGAUGGAUAGAUGGAUGAGUUG
GUGGGUAGAUUCGUGGCUAGAUGGAUGAUGGAUGGAUGGACAGAUGGAUG
GAUAUAUGAUUGAACUAUUGAAAGUAUAGAUGUAUGGAUGGGUGAAUUU
GGGGGUAAUUGUUAGAUGAUGGAUGAGUAUAGAUGAAUGAUGGAUGGAU
AACUUGAUGAGUGGAUAGAUAGAUUGCUGGAUAGAUGAUUGACUGGGUGG
AUAGAUGAAAUGUUGGAUGAGCAGAUUAAGUUGUAUUGGAUGGGAUGGA
UGGAAGUGUGGUUGAGUUAUUAGAAGGAAGAUUGAGUAGAUAGGUGAAU
UUGUUGAUAGUCAGAUGGGUAGAUAGGUAGAUGGAUGGAUGGAUGGAUG
GAUGUAUAGGCAGAUGGACAAAUGGAUGAAUGGGUGGGUGGAUGAAUGGA
AGGAUGUGUGGUUGAACUAUUGCAAGUAUUGAUAAUUGGGUUCAUAAUUU
CUGAAUAUUUAGAUGGAUGGUUGUGAGUGGCUGGUGGACAGACGAAAAAU
GGAUGGUUGGAUAAAUUGAUGGGUGGAUGGAUGGUUGGUUGUAUGAAAG
AAUGAAUGAUUGGGUAGGUGGAUUAAGUUGCGGAUCAAUGUAUGGGAUGG
AUGAAUGGAUGGAUGGAUGGAUGUGUGGUUGAAUUACUGAAAGGUUGGA
AGAGUGGAUGGGUGAAAUUUGGGGUAGUUAGAUGGGUGGGUGUGUGGAU
GGAUAAAAGAGUAGAUGAAUGAAUUAAUGAAUAAACAGGCAGAUGGAUGA
UGUAAGCUGCCCCAGACCCUGGGACCUCUGACCCCCGGCGACCCCUUGCAC
UCUCCAUGACACUUUCUCUCCCAUGGUGGCAG

The splice sites are defined as follows: Long cryptic acceptor is the phosphodiester bond between chr19: 17,642,591-17,642,592; the Short cryptic acceptor is the phosphodiester bond between chr19:17,642,541-17,642,542 and the Cryptic donor is the phosphodiester bond between chr19:17,642,413-17,642,414.

SEQ ID NO: 1 may encompass the minor allele of the SNP (i.e., the risk variant) or the major allele at rs12973192 and/or rs12608932, therefore SEQ ID NO: 1 also encompasses the sequence wherein the emboldened G (at rs12973192) is replaced with a C, and the emboldened U (rs12608932) corresponding to the rs12608932 cryptic exon SNP may be replaced with a G.

SEQ ID NO 2 Shorter UNC13A cryptic exon sequence in transcribed UNC13A mRNA—cords chr19:17642414-17,642,541. SEQ ID NO 2 has the sequence: CUGCCUGGGUUUCCUGGAAAGAACUCUUAUCCCCAGGAACUAGUUUGUUGAAUA AAUGCUGGUGAAUGAAUGAAUGAUUGAACAGAUGAAUGAGUGAUGAGUAGAUAA AAGGAUGGAUGGAGAGAUGG). SEQ TD NO: 2 may encompass minor allele of the SNP (i.e., the risk variant), or the major allele at rs12973192, therefore SEQ TD NO: 2 also encompasses the sequence wherein the emboldened G (at rs12973192) is replaced with a C.

SEQ ID NO 3—Longer UNC13A cryptic exon sequence in transcribed UNC13A mRNA—cords chr19:17642414-17642591. SEQ ID NO 3 has the sequence (CCCUAACCACUCAGGAUUGGGCCGiUGUGUCUGGGUAUGUCUCUCCAGCUGC CUGGGUUUCCUGGAAAGAACUCUAUCCCCAGGAACUAGUUUGUGAAUAAAUG CUGGUGAAUGAAUGAAUGAnUGAACAGAUGAAUGAGUGAUGAGUAGAUAAAAGG AUGGAUGGAGAGAUGG). SEQ TD NO: 3 may encompass the risk variant of the SNP (i.e., minor allele), or the major allele at rs12973192, therefore SEQ ID NO: 3 also encompasses the sequence wherein the emboldened G (at rs2973192) is replaced with a C.

The following Tables provides a list of ASO sequences or portions of ASO sequences that are capable of binding to crucial elements involved in UNC13A cryptic exon splicing.

TABLE 1
Branchpoint

| | | Target/ | Portion |
|---|---|---|---|
| | | crucial | of ASO |
| SEQ ID NO | Name | element | sequence/ASO |
| SEQ ID NO: 4 | 285_maj | branchpoint | GGAGUUUUCCAGG |
| SEQ ID NO: 5 | 286_maj | branchpoint | AGGAGUUUUCCAG |
| SEQ ID NO: 6 | 287_maj | branchpoint | GAGGAGUUUUCCA |
| SEQ ID NO: 7 | 288_maj | branchpoint | GGAGGAGUUUUCC |
| SEQ ID NO: 8 | 289_maj | branchpoint | GGGAGGAGUUUUC |
| SEQ ID NO: 9 | 290_maj | branchpoint | AGGGAGGAGUUUU |
| SEQ ID NO: 10 | 291_maj | branchpoint | UAGGGAGGAGUUU |
| SEQ ID NO: 11 | 292_maj | branchpoint | AUAGGGAGGAGUU |
| SEQ ID NO: 12 | 293_maj | branchpoint | GAUAGGGAGGAGU |
| SEQ ID NO: 13 | 294_maj | branchpoint | GGAUAGGGAGGAG |
| SEQ ID NO: 14 | 295_maj | branchpoint | UGGAUAGGGAGGA |
| SEQ ID NO: 15 | 296_maj | branchpoint | AUGGAUAGGGAGG |
| SEQ ID NO: 16 | 297_maj | branchpoint | GAUGGAUAGGGAG |
| SEQ ID NO: 17 | 298_maj | branchpoint | UGAUGGAUAGGGA |
| SEQ ID NO: 18 | 299_maj | branchpoint | UUGAUGGAUAGGG |
| SEQ ID NO: 19 | 300_maj | branchpoint | UUUGAUGGAUAGG |
| SEQ ID NO: 20 | 301 maj | branchpoint | CUUUGAUGGAUAG |
| SEQ ID NO: 21 | 302_maj | branchpoint | ACUUUGAUGGAUA |
| SEQ ID NO: 22 | 303_maj | branchpoint | GACUUUGAUGGAU |
| SEQ ID NO: 23 | 304_maj | branchpoint | GGACUUUGAUGGA |
| SEQ ID NO: 24 | 305_maj | branchpoint | UGGACUUUGAUGG |
| SEQ ID NO: 25 | 306_maj | branchpoint | CUGGACUUUGAUG |
| SEQ ID NO: 26 | 307_maj | branchpoint | UCUGGACUUUGAU |
| SEQ ID NO: 27 | 308_maj | branchpoint | AUCUGGACUUUGA |
| SEQ ID NO: 28 | 309 maj | branchpoint | AAUCUGGACUUUG |
| SEQ ID NO: 29 | 310_maj | branchpoint | GAAUCUGGACUUU |
| SEQ ID NO: 30 | 311 maj | branchpoint | UGAAUCUGGACUU |
| SEQ ID NO: 31 | 312_maj | branchpoint | CUGAAUCUGGACU |
| SEQ ID NO: 32 | 313 maj | branchpoint | CCUGAAUCUGGAC |
| SEQ ID NO: 33 | 314_maj | branchpoint | CCCUGAAUCUGGA |
| SEQ ID NO: 34 | 315_maj | branchpoint | ACCCUGAAUCUGG |
| SEQ ID NO: 35 | 316_maj | branchpoint | GACCCUGAAUCUG |
| SEQ ID NO: 36 | 317_maj | branchpoint | UGACCCUGAAUCU |
| SEQ ID NO: 37 | 318_maj | branchpoint | GUGACCCUGAAUC |
| SEQ ID NO: 38 | 319_maj | branchpoint | GGUGACCCUGAAU |
| SEQ ID NO: 39 | 320_maj | branchpoint | AGGUGACCCUGAA |
| SEQ ID NO: 40 | 321_maj | branchpoint | GAGGUGACCCUGA |
| SEQ ID NO: 41 | 322_maj | branchpoint | GGAGGUGACCCUG |
| SEQ ID NO: 42 | 323_maj | branchpoint | AGGAGGUGACCCU |
| SEQ ID NO: 43 | 324_maj | branchpoint | GAGGAGGUGACCC |
| SEQ ID NO: 44 | 325_maj | branchpoint | AGAGGAGGUGACC |
| SEQ ID NO: 45 | 326_maj | branchpoint | CAGAGGAGGUGAC |
| SEQ ID NO: 46 | 327_maj | branchpoint | CCAGAGGAGGUGA |
| SEQ ID NO: 47 | 328_maj | branchpoint | CCCAGAGGAGGUG |
| SEQ ID NO: 48 | 329 maj | branchpoint | UCCCAGAGGAGGU |
| SEQ ID NO: 49 | 330_maj | branchpoint | UUCCCAGAGGAGG |
| SEQ ID NO: 50 | 331_maj | branchpoint | CUUCCCAGAGGAG |
| SEQ ID NO: 51 | 332_maj | branchpoint | GCUUCCCAGAGGA |
| SEQ ID NO: 52 | 333_maj | branchpoint | GGCUUCCCAGAGG |
| SEQ ID NO: 53 | 334_maj | branchpoint | GGGCUUCCCAGAG |
| SEQ ID NO: 54 | 335_maj | branchpoint | UGGGCUUCCCAGA |
| SEQ ID NO: 55 | 336_maj | branchpoint | GUGGGCUUCCCAG |
| SEQ ID NO: 56 | 337_maj | branchpoint | GGUGGGCUUCCCA |
| SEQ ID NO: 57 | 338_maj | branchpoint | AGGUGGGCUUCCC |
| SEQ ID NO: 58 | 339_maj | branchpoint | AAGGUGGGCUUCC |
| SEQ ID NO: 59 | 340_maj | branchpoint | CAAGGUGGGCUUC |
| SEQ ID NO: 60 | 341_maj | branchpoint | CCAAGGUGGGCUU |
| SEQ ID NO: 61 | 342_maj | branchpoint | GCCAAGGUGGGCU |
| SEQ ID NO: 62 | 343_maj | branchpoint | GGCCAAGGUGGGC |
| SEQ ID NO: 63 | 344_maj | branchpoint | AGGCCAAGGUGGG |
| SEQ ID NO: 64 | 345_maj | branchpoint | GAGGCCAAGGUGG |
| SEQ ID NO: 65 | 346_maj | branchpoint | GGAGGCCAAGGUG |
| SEQ ID NO: 66 | 347_maj | branchpoint | UGGAGGCCAAGGU |
| SEQ ID NO: 67 | 348_maj | branchpoint | CUGGAGGCCAAGG |
| SEQ ID NO: 68 | 349_maj | branchpoint | CCUGGAGGCCAAG |
| SEQ ID NO: 69 | 350_maj | branchpoint | ACCUGGAGGCCAA |
| SEQ ID NO: 70 | 351_maj | branchpoint | AACCUGGAGGCCA |
| SEQ ID NO: 71 | 352_maj | branchpoint | CAACCUGGAGGCC |
| SEQ ID NO: 72 | 353_maj | branchpoint | UCAACCUGGAGGC |
| SEQ ID NO: 73 | 354_maj | branchpoint | GUCAACCUGGAGG |
| SEQ ID NO: 74 | 355_maj | branchpoint | AGUCAACCUGGAG |
| SEQ ID NO: 75 | 356_maj | branchpoint | GAGUCAACCUGGA |
| SEQ ID NO: 76 | 357_maj | branchpoint | AGAGUCAACCUGG |
| SEQ ID NO: 77 | 358_maj | branchpoint | GAGAGUCAACCUG |
| SEQ ID NO: 78 | 359_maj | branchpoint | UGAGAGUCAACCU |
| SEQ ID NO: 79 | 360_maj | branchpoint | GUGAGAGUCAACC |
| SEQ ID NO: 80 | 361_maj | branchpoint | AGUGAGAGUCAAC |
| SEQ ID NO: 81 | 362_maj | branchpoint | UAGUGAGAGUCAA |
| SEQ ID NO: 82 | 363_maj | branchpoint | GUAGUGAGAGUCA |
| SEQ ID NO: 83 | 364 maj | branchpoint | AGUAGUGAGAGUC |
| SEQ ID NO: 84 | 365_maj | branchpoint | GAGUAGUGAGAGU |
| SEQ ID NO: 85 | 366_maj | branchpoint | UGAGUAGUGAGAG |
| SEQ ID NO: 86 | 367_maj | branchpoint | AUGAGUAGUGAGA |
| SEQ ID NO: 87 | 368_maj | branchpoint | GAUGAGUAGUGAG |
| SEQ ID NO: 88 | 369_maj | branchpoint | UGAUGAGUAGUGA |
| SEQ ID NO: 89 | 370_maj | branchpoint | AUGAUGAGUAGUG |
| SEQ ID NO: 90 | 371_maj | branchpoint | GAUGAUGAGUAGU |
| SEQ ID NO: 91 | 372_maj | branchpoint | UGAUGAUGAGUAG |
| SEQ ID NO: 92 | 373_maj | branchpoint | CUGAUGAUGAGUA |
| SEQ ID NO: 93 | 374_maj | branchpoint | CCUGAUGAUGAGU |
| SEQ ID NO: 94 | 375_maj | branchpoint | ACCUGAUGAUGAG |
| SEQ ID NO: 95 | 376_maj | branchpoint | AACCUGAUGAUGA |
| SEQ ID NO: 96 | 377_maj | branchpoint | GAACCUGAUGAUG |
| SEQ ID NO: 97 | 378_maj | branchpoint | AGAACCUGAUGAU |
| SEQ ID NO: 98 | 379_maj | branchpoint | AAGAACCUGAUGA |
| SEQ ID NO: 99 | 380_maj | branchpoint | GAAGAACCUGAUG |
| SEQ ID NO: 100 | 381_maj | branchpoint | GGAAGAACCUGAU |
| SEQ ID NO: 101 | 382_maj | branchpoint | AGGAAGAACCUGA |
| SEQ ID NO: 102 | 383_maj | branchpoint | AAGGAAGAACCUG |
| SEQ ID NO: 103 | 384_maj | branchpoint | GAAGGAAGAACCU |
| SEQ ID NO: 104 | 385_maj | branchpoint | AGAAGGAAGAACC |

TABLE 2
Splice sites

| | | Target/ | Portion of ASO |
|---|---|---|---|
| SEQ ID NO | Name | crucial element | sequence/ASO |
| SEQ ID NO: 105 | 386_maj | 3′-splice site | UAGAAGGAAGAAC |
| SEQ ID NO: 106 | 387_maj | 3′-splice site | AUAGAAGGAAGAA |
| SEQ ID NO: 107 | 388_maj | 3′-splice site | AAUAGAAGGAAGA |
| SEQ ID NO: 108 | 389_maj | 3′-splice site | GAAUAGAAGGAAG |
| SEQ ID NO: 109 | 390_maj | 3′-splice site | GGAAUAGAAGGAA |
| SEQ ID NO: 110 | 391_maj | 3′-splice site | UGGAAUAGAAGGA |
| SEQ ID NO: 111 | 392_maj | 3′-splice site | CUGGAAUAGAAGG |
| SEQ ID NO: 112 | 393_maj | 3′-splice site | GCUGGAAUAGAAG |
| SEQ ID NO: 113 | 394_maj | 3′-splice site | GGCUGGAAUAGAA |
| SEQ ID NO: 114 | 395_maj | 3′-splice site | GGGCUGGAAUAGA |
| SEQ ID NO: 115 | 396_maj | 3′-splice site | AGGGCUGGAAUAG |
| SEQ ID NO: 116 | 397_maj | 3′-splice site | UAGGGCUGGAAUA |
| SEQ ID NO: 117 | 398_maj | 3′-splice site | UUAGGGCUGGAAU |
| SEQ ID NO: 118 | 399_maj | 3′-splice site | GUUAGGGCUGGAA |
| SEQ ID NO: 119 | 400_maj | 3′-splice site | GGUUAGGGCUGGA |
| SEQ ID NO: 120 | 401_maj | 3′-splice site | UGGUUAGGGCUGG |
| SEQ ID NO: 121 | 402_maj | 3′-splice site | GUGGUUAGGGCUG |
| SEQ ID NO: 122 | 403_maj | 3′-splice site | AGUGGUUAGGGCU |
| SEQ ID NO: 123 | 404_maj | 3′-splice site | GAGUGGUUAGGGC |
| SEQ ID NO: 124 | 405_maj | 3′-splice site | UGAGUGGUUAGGG |
| SEQ ID NO: 125 | 406_maj | 3′-splice site | CUGAGUGGUUAGG |
| SEQ ID NO: 126 | 407_maj | 3′-splice site | CCUGAGUGGUUAG |
| SEQ ID NO: 127 | 408_maj | 3′-splice site | UCCUGAGUGGUUA |
| SEQ ID NO: 128 | 409_maj | 3′-splice site | AUCCUGAGUGGUU |
| SEQ ID NO: 129 | 410_maj | 3′-splice site | AAUCCUGAGUGGU |
| SEQ ID NO: 130 | 411_maj | 3′-splice site | CAAUCCUGAGUGG |
| SEQ ID NO: 131 | 412_maj | 3′-splice site | CCAAUCCUGAGUG |
| SEQ ID NO: 132 | 413_maj | 3′-splice site | CCCAAUCCUGAGU |
| SEQ ID NO: 133 | 414_maj | 3′-splice site | GCCCAAUCCUGAG |
| SEQ ID NO: 134 | 415_maj | 3′-splice site | GGCCCAAUCCUGA |
| SEQ ID NO: 135 | 416_maj | 3′-splice site | CGGCCCAAUCCUG |
| SEQ ID NO: 136 | 417_maj | 3′-splice site | ACGGCCCAAUCCU |
| SEQ ID NO: 137 | 418_maj | 3′-splice site | AACGGCCCAAUCC |
| SEQ ID NO: 138 | 419_maj | 3′-splice site | AAACGGCCCAAUC |
| SEQ ID NO: 139 | 420_maj | 3′-splice site | CAAACGGCCCAAU |
| SEQ ID NO: 140 | 421_maj | 3′-splice site | ACAAACGGCCCAA |
| SEQ ID NO: 141 | 422_maj | 3′-splice site | CACAAACGGCCCA |
| SEQ ID NO: 142 | 423_maj | 3′-splice site | ACACAAACGGCCC |
| SEQ ID NO: 143 | 424_maj | 3′-splice site | GACACAAACGGCC |
| SEQ ID NO: 144 | 425_maj | 3′-splice site | AGACACAAACGGC |
| SEQ ID NO: 145 | 426_maj | 3′-splice site | CAGACACAAACGG |
| SEQ ID NO: 146 | 427_maj | 3′-splice site | CCAGACACAAACG |
| SEQ ID NO: 147 | 428_maj | 3′-splice site | CCCAGACACAAAC |
| SEQ ID NO: 148 | 429_maj | 3′-splice site | ACCCAGACACAAA |
| SEQ ID NO: 149 | 430_maj | 3′-splice site | UACCCAGACACAA |
| SEQ ID NO: 150 | 431_maj | 3′-splice site | AUACCCAGACACA |
| SEQ ID NO: 151 | 432_maj | 3′-splice site | CAUACCCAGACAC |
| SEQ ID NO: 152 | 433_maj | 3′-splice site | ACAUACCCAGACA |
| SEQ ID NO: 153 | 434_maj | 3′-splice site | GACAUACCCAGAC |
| SEQ ID NO: 154 | 435_maj | 3′-splice site | AGACAUACCCAGA |
| SEQ ID NO: 155 | 436_maj | 3′-splice site | GAGACAUACCCAG |
| SEQ ID NO: 156 | 437_maj | 3′-splice site | AGAGACAUACCCA |
| SEQ ID NO: 157 | 438_maj | 3′-splice site | AAGAGACAUACCC |
| SEQ ID NO: 158 | 439_maj | 3′-splice site | GAAGAGACAUACC |
| SEQ ID NO: 159 | 440_maj | 3′-splice site | GGAAGAGACAUAC |
| SEQ ID NO: 160 | 441_maj | 3′-splice site | UGGAAGAGACAUA |
| SEQ ID NO: 161 | 442_maj | 3′-splice site | CUGGAAGAGACAU |
| SEQ ID NO: 162 | 443_maj | 3′-splice site | GCUGGAAGAGACA |
| SEQ ID NO: 163 | 444_maj | 3′-splice site | AGCUGGAAGAGAC |
| SEQ ID NO: 164 | 445_maj | 3′-splice site | CAGCUGGAAGAGA |
| SEQ ID NO: 165 | 446_maj | 3′-splice site | GCAGCUGGAAGAG |
| SEQ ID NO: 166 | 447_maj | 3′-splice site | GGCAGCUGGAAGA |
| SEQ ID NO: 167 | 448_maj | 3′-splice site | AGGCAGCUGGAAG |
| SEQ ID NO: 168 | 449_maj | 3′-splice site | CAGGCAGCUGGAA |
| SEQ ID NO: 169 | 450_maj | 3′-splice site | CCAGGCAGCUGGA |
| SEQ ID NO: 170 | 451_maj | 3′-splice site | CCCAGGCAGCUGG |
| SEQ ID NO: 171 | 452_maj | 3′-splice site | ACCCAGGCAGCUG |
| SEQ ID NO: 172 | 453_maj | 3′-splice site | AACCCAGGCAGCU |
| SEQ ID NO: 173 | 454_maj | 3′-splice site | AAACCCAGGCAGC |
| SEQ ID NO: 174 | 455_maj | 3′-splice site | GAAACCCAGGCAG |
| SEQ ID NO: 175 | 456_maj | 3′-splice site | GGAAACCCAGGCA |
| SEQ ID NO: 176 | 457_maj | 3′-splice site | AGGAAACCCAGGC |
| SEQ ID NO: 177 | 458_maj | 3′-splice site | CAGGAAACCCAGG |
| SEQ ID NO: 178 | 459_maj | 3′-splice site | CCAGGAAACCCAG |
| SEQ ID NO: 179 | 460_maj | 3′-splice site | UCCAGGAAACCCA |
| SEQ ID NO: 180 | 461_maj | 3′-splice site | UUCCAGGAAACCC |
| SEQ ID NO: 181 | 462_maj | 3′-splice site | UUUCCAGGAAACC |
| SEQ ID NO: 182 | 463_maj | 3′-splice site | CUUUCCAGGAAAC |
| SEQ ID NO: 183 | 464_maj | 3′-splice site | UCUUUCCAGGAAA |
| SEQ ID NO: 184 | 465_maj | 3′-splice site | UUCUUUCCAGGAA |
| SEQ ID NO: 185 | 466_maj | 3′-splice site | GUUCUUUCCAGGA |
| SEQ ID NO: 186 | 467_maj | 3′-splice site | AGUUCUUUCCAGG |
| SEQ ID NO: 187 | 468_maj | 3′-splice site | GAGUUCUUUCCAG |
| SEQ ID NO: 188 | 469_maj | 3′-splice site | AGAGUUCUUUCCA |
| SEQ ID NO: 189 | 470_maj | 3′-splice site | AAGAGUUCUUUCC |
| SEQ ID NO: 270 | 551_maj | 5′ splice-site | UUUAUCUACUCAU |
| SEQ ID NO: 271 | 552_maj | 5′ splice-site | UUUUAUCUACUCA |
| SEQ ID NO: 272 | 553_maj | 5′ splice-site | CUUUUAUCUACUC |
| SEQ ID NO: 273 | 554_maj | 5′ splice-site | CCUUUUAUCUACU |
| SEQ ID NO: 274 | 554_min | 5′ splice-site | GCUUUUAUCUACU |
| SEQ ID NO: 275 | 555_maj | 5′ splice-site | UCCUUUUAUCUAC |
| SEQ ID NO: 276 | 555_min | 5′ splice-site | UGCUUUUAUCUAC |
| SEQ ID NO: 277 | 556_maj | 5′ splice-site | AUCCUUUUAUCUA |
| SEQ ID NO: 278 | 556_min | 5′ splice-site | AUGCUUUUAUCUA |
| SEQ ID NO: 279 | 557_maj | 5′ splice-site | CAUCCUUUUAUCU |
| SEQ ID NO: 280 | 557_min | 5′ splice-site | CAUGCUUUUAUCU |
| SEQ ID NO: 281 | 558_maj | 5′ splice-site | CCAUCCUUUUAUC |
| SEQ ID NO: 282 | 558_min | 5′ splice-site | CCAUGCUUUUAUC |
| SEQ ID NO: 283 | 559_maj | 5′ splice-site | UCCAUCCUUUUAU |
| SEQ ID NO: 284 | 559_min | 5′ splice-site | UCCAUGCUUUUAU |
| SEQ ID NO: 285 | 560_maj | 5′ splice-site | AUCCAUCCUUUUA |
| SEQ ID NO: 286 | 560_min | 5′ splice-site | AUCCAUGCUUUUA |
| SEQ ID NO: 287 | 561_maj | 5′ splice-site | CAUCCAUCCUUUU |
| SEQ ID NO: 288 | 561_min | 5′ splice-site | CAUCCAUGCUUUU |
| SEQ ID NO: 289 | 562_maj | 5′ splice-site | CCAUCCAUCCUUU |
| SEQ ID NO: 290 | 562_min | 5′ splice-site | CCAUCCAUGCUUU |
| SEQ ID NO: 291 | 563_maj | 5′ splice-site | UCCAUCCAUCCUU |
| SEQ ID NO: 292 | 563_min | 5′ splice-site | UCCAUCCAUGCUU |
| SEQ ID NO: 293 | 564_maj | 5′ splice-site | CUCCAUCCAUCCU |
| SEQ ID NO: 294 | 564_min | 5′ splice-site | CUCCAUCCAUGCU |
| SEQ ID NO: 295 | 565_maj | 5′ splice-site | UCUCCAUCCAUCC |
| SEQ ID NO: 296 | 565_min | 5′ splice-site | UCUCCAUCCAUGC |
| SEQ ID NO: 297 | 566_maj | 5′ splice-site | CUCUCCAUCCAUC |
| SEQ ID NO: 298 | 566_min | 5′ splice-site | CUCUCCAUCCAUG |
| SEQ ID NO: 299 | 567_maj | 5′ splice-site | UCUCUCCAUCCAU |
| SEQ ID NO: 300 | 568_maj | 5′ splice-site | AUCUCUCCAUCCA |
| SEQ ID NO: 301 | 569_maj | 5′ splice-site | CAUCUCUCCAUCC |
| SEQ ID NO: 302 | 570_maj | 5′ splice-site | CCAUCUCUCCAUC |
| SEQ ID NO: 303 | 571_maj | 5′ splice-site | CCCAUCUCUCCAU |
| SEQ ID NO: 304 | 572_maj | 5′ splice-site | ACCCAUCUCUCCA |
| SEQ ID NO: 305 | 573_maj | 5′ splice-site | CACCCAUCUCUCC |
| SEQ ID NO: 306 | 574_maj | 5′ splice-site | UCACCCAUCUCUC |
| SEQ ID NO: 307 | 575_maj | 5′ splice-site | CUCACCCAUCUCU |
| SEQ ID NO: 308 | 576_maj | 5′ splice-site | ACUCACCCAUCUC |
| SEQ ID NO: 309 | 577_maj | 5′ splice-site | UACUCACCCAUCU |
| SEQ ID NO: 310 | 578_maj | 5′ splice-site | GUACUCACCCAUC |
| SEQ ID NO: 311 | 579_maj | 5′ splice-site | UGUACUCACCCAU |
| SEQ ID NO: 312 | 580_maj | 5′ splice-site | AUGUACUCACCCA |
| SEQ ID NO: 313 | 581_maj | 5′ splice-site | CAUGUACUCACCC |
| SEQ ID NO: 314 | 582_maj | 5′ splice-site | CCAUGUACUCACC |
| SEQ ID NO: 315 | 583_maj | 5′ splice-site | UCCAUGUACUCAC |
| SEQ ID NO: 316 | 584_maj | 5′ splice-site | AUCCAUGUACUCA |
| SEQ ID NO: 317 | 585_maj | 5′ splice-site | CAUCCAUGUACUC |
| SEQ ID NO: 318 | 586_maj | 5′ splice-site | CCAUCCAUGUACU |
| SEQ ID NO: 319 | 587_maj | 5′ splice-site | UCCAUCCAUGUAC |
| SEQ ID NO: 320 | 588_maj | 5′ splice-site | AUCCAUCCAUGUA |
| SEQ ID NO: 321 | 589_maj | 5′ splice-site | UAUCCAUCCAUGU |
| SEQ ID NO: 322 | 590_maj | 5′ splice-site | CUAUCCAUCCAUG |
| SEQ ID NO: 323 | 591_maj | 5′ splice-site | UCUAUCCAUCCAU |
| SEQ ID NO: 324 | 592_maj | 5′ splice-site | AUCUAUCCAUCCA |
| SEQ ID NO: 325 | 593_maj | 5′ splice-site | CAUCUAUCCAUCC |
| SEQ ID NO: 326 | 594_maj | 5′ splice-site | CCAUCUAUCCAUC |
| SEQ ID NO: 327 | 595_maj | 5′ splice-site | UCCAUCUAUCCAU |
| SEQ ID NO: 328 | 596_maj | 5′ splice-site | AUCCAUCUAUCCA |
| SEQ ID NO: 329 | 597_maj | 5′ splice-site | CAUCCAUCUAUCC |
| SEQ ID NO: 330 | 598_maj | 5′ splice-site | UCAUCCAUCUAUC |
| SEQ ID NO: 331 | 599_maj | 5′ splice-site | CUCAUCCAUCUAU |
| SEQ ID NO: 332 | 600_maj | 5′ splice-site | ACUCAUCCAUCUA |
| SEQ ID NO: 333 | 601_maj | 5′ splice-site | AACUCAUCCAUCU |
| SEQ ID NO: 334 | 602_maj | 5′ splice-site | CAACUCAUCCAUC |
| SEQ ID NO: 335 | 603_maj | 5′ splice-site | CCAACUCAUCCAU |
| SEQ ID NO: 336 | 604_maj | 5′ splice-site | ACCAACUCAUCCA |
| SEQ ID NO: 337 | 605_maj | 5′ splice-site | CACCAACUCAUCC |
| SEQ ID NO: 338 | 606_maj | 5′ splice-site | CCACCAACUCAUC |
| SEQ ID NO: 339 | 607_maj | 5′ splice-site | CCCACCAACUCAU |
| SEQ ID NO: 340 | 608_maj | 5′ splice-site | ACCCACCAACUCA |
| SEQ ID NO: 341 | 609_maj | 5′ splice-site | UACCCACCAACUC |
| SEQ ID NO: 342 | 610_maj | 5′ splice-site | CUACCCACCAACU |
| SEQ ID NO: 343 | 611_maj | 5′ splice-site | UCUACCCACCAAC |
| SEQ ID NO: 344 | 612_maj | 5′ splice-site | AUCUACCCACCAA |
| SEQ ID NO: 345 | 613_maj | 5′ splice-site | AAUCUACCCACCA |
| SEQ ID NO: 346 | 614_maj | 5′ splice-site | GAAUCUACCCACC |
| SEQ ID NO: 347 | 615_maj | 5′ splice-site | CGAAUCUACCCAC |
| SEQ ID NO: 348 | 616_maj | 5′ splice-site | ACGAAUCUACCCA |
| SEQ ID NO: 349 | 617_maj | 5′ splice-site | CACGAAUCUACCC |
| SEQ ID NO: 350 | 618_maj | 5′ splice-site | CCACGAAUCUACC |
| SEQ ID NO: 351 | 619_maj | 5′ splice-site | GCCACGAAUCUAC |
| SEQ ID NO: 352 | 620_maj | 5′ splice-site | AGCCACGAAUCUA |

TABLE 3
Splice Regulatory Elements (SREs)

| | | Target/ | Portion of ASO |
|---|---|---|---|
| SEQ ID NO | Name | crucial element | sequence/ASO |
| SEQ ID NO: 190 | 471_maj | cryptic | UAAGAGUUCUUUC |
| SEQ ID NO: 191 | 472_maj | cryptic | AUAAGAGUUCUUU |
| SEQ ID NO: 192 | 473_maj | cryptic | GAUAAGAGUUCUU |
| SEQ ID NO: 193 | 474_maj | cryptic | GGAUAAGAGUUCU |
| SEQ ID NO: 194 | 475_maj | cryptic | GGGAUAAGAGUUC |
| SEQ ID NO: 195 | 476_maj | cryptic | GGGGAUAAGAGUU |
| SEQ ID NO: 196 | 477_maj | cryptic | UGGGGAUAAGAGU |
| SEQ ID NO: 197 | 478_maj | cryptic | CUGGGGAUAAGAG |
| SEQ ID NO: 198 | 479_maj | cryptic | CCUGGGGAUAAGA |
| SEQ ID NO: 199 | 480_maj | cryptic | UCCUGGGGAUAAG |
| SEQ ID NO: 200 | 481_maj | cryptic | UUCCUGGGGAUAA |
| SEQ ID NO: 201 | 482_maj | cryptic | GUUCCUGGGGAUA |
| SEQ ID NO: 202 | 483_maj | cryptic | AGUUCCUGGGGAU |
| SEQ ID NO: 203 | 484_maj | cryptic | UAGUUCCUGGGGA |
| SEQ ID NO: 204 | 485_maj | cryptic | CUAGUUCCUGGGG |
| SEQ ID NO: 205 | 486_maj | cryptic | ACUAGUUCCUGGG |
| SEQ ID NO: 206 | 487_maj | cryptic | AACUAGUUCCUGG |
| SEQ ID NO: 207 | 488_maj | cryptic | AAACUAGUUCCUG |
| SEQ ID NO: 208 | 489_maj | cryptic | CAAACUAGUUCCU |
| SEQ ID NO: 209 | 490_maj | cryptic | ACAAACUAGUUCC |
| SEQ ID NO: 210 | 491_maj | cryptic | AACAAACUAGUUC |
| SEQ ID NO: 211 | 492_maj | cryptic | CAACAAACUAGUU |
| SEQ ID NO: 212 | 493_maj | cryptic | UCAACAAACUAGU |
| SEQ ID NO: 213 | 494_maj | cryptic | UUCAACAAACUAG |
| SEQ ID NO: 214 | 495_maj | cryptic | AUUCAACAAACUA |
| SEQ ID NO: 215 | 496_maj | cryptic | UAUUCAACAAACU |
| SEQ ID NO: 216 | 497_maj | cryptic | UUAUUCAACAAAC |
| SEQ ID NO: 217 | 498_maj | cryptic | UUUAUUCAACAAA |
| SEQ ID NO: 218 | 499_maj | cryptic | AUUUAUUCAACAA |
| SEQ ID NO: 219 | 500_maj | cryptic | CAUUUAUUCAACA |
| SEQ ID NO: 220 | 501_maj | cryptic | GCAUUUAUUCAAC |
| SEQ ID NO: 221 | 502_maj | cryptic | AGCAUUUAUUCAA |
| SEQ ID NO: 222 | 503_maj | cryptic | CAGCAUUUAUUCA |
| SEQ ID NO: 223 | 504_maj | cryptic | CCAGCAUUUAUUC |
| SEQ ID NO: 224 | 505_maj | cryptic | ACCAGCAUUUAUU |
| SEQ ID NO: 225 | 506_maj | cryptic | CACCAGCAUUUAU |
| SEQ ID NO: 226 | 507_maj | cryptic | UCACCAGCAUUUA |
| SEQ ID NO: 227 | 508_maj | cryptic | UUCACCAGCAUUU |
| SEQ ID NO: 228 | 509_maj | cryptic | AUUCACCAGCAUU |
| SEQ ID NO: 229 | 510_maj | cryptic | CAUUCACCAGCAU |
| SEQ ID NO: 230 | 511_maj | cryptic | UCAUUCACCAGCA |
| SEQ ID NO: 231 | 512_maj | cryptic | UUCAUUCACCAGC |
| SEQ ID NO: 232 | 513_maj | cryptic | AUUCAUUCACCAG |
| SEQ ID NO: 233 | 514_maj | cryptic | CAUUCAUUCACCA |
| SEQ ID NO: 234 | 515_maj | cryptic | UCAUUCAUUCACC |
| SEQ ID NO: 235 | 516_maj | cryptic | UUCAUUCAUUCAC |
| SEQ ID NO: 236 | 517_maj | cryptic | AUUCAUUCAUUCA |
| SEQ ID NO: 237 | 518_maj | cryptic | CAUUCAUUCAUUC |
| SEQ ID NO: 238 | 519_maj | cryptic | UCAUUCAUUCAUU |
| SEQ ID NO: 239 | 520_maj | cryptic | AUCAUUCAUUCAU |
| SEQ ID NO: 240 | 521_maj | cryptic | AAUCAUUCAUUCA |
| SEQ ID NO: 241 | 522_maj | cryptic | CAAUCAUUCAUUC |
| SEQ ID NO: 242 | 523_maj | cryptic | UCAAUCAUUCAUU |
| SEQ ID NO: 243 | 524_maj | cryptic | UUCAAUCAUUCAU |
| SEQ ID NO: 244 | 525_maj | cryptic | GUUCAAUCAUUCA |
| SEQ ID NO: 245 | 526_maj | cryptic | UGUUCAAUCAUUC |
| SEQ ID NO: 246 | 527_maj | cryptic | CUGUUCAAUCAUU |
| SEQ ID NO: 247 | 528_maj | cryptic | UCUGUUCAAUCAU |
| SEQ ID NO: 248 | 529_maj | cryptic | AUCUGUUCAAUCA |
| SEQ ID NO: 249 | 530_maj | cryptic | CAUCUGUUCAAUC |
| SEQ ID NO: 250 | 531_maj | cryptic | UCAUCUGUUCAAU |
| SEQ ID NO: 251 | 532_maj | cryptic | UUCAUCUGUUCAA |
| SEQ ID NO: 252 | 533_maj | cryptic | AUUCAUCUGUUCA |
| SEQ ID NO: 253 | 534_maj | cryptic | CAUUCAUCUGUUC |
| SEQ ID NO: 254 | 535_maj | cryptic | UCAUUCAUCUGUU |
| SEQ ID NO: 255 | 536_maj | cryptic | CUCAUUCAUCUGU |
| SEQ ID NO: 256 | 537_maj | cryptic | ACUCAUUCAUCUG |
| SEQ ID NO: 257 | 538_maj | cryptic | CACUCAUUCAUCU |
| SEQ ID NO: 258 | 539_maj | cryptic | UCACUCAUUCAUC |
| SEQ ID NO: 259 | 540_maj | cryptic | AUCACUCAUUCAU |
| SEQ ID NO: 260 | 541_maj | cryptic | CAUCACUCAUUCA |
| SEQ ID NO: 261 | 542_maj | cryptic | UCAUCACUCAUUC |
| SEQ ID NO: 262 | 543_maj | cryptic | CUCAUCACUCAUU |
| SEQ ID NO: 263 | 544_maj | cryptic | ACUCAUCACUCAU |
| SEQ ID NO: 264 | 545_maj | cryptic | UACUCAUCACUCA |
| SEQ ID NO: 265 | 546_maj | cryptic | CUACUCAUCACUC |
| SEQ ID NO: 266 | 547_maj | cryptic | UCUACUCAUCACU |
| SEQ ID NO: 267 | 548_maj | cryptic | AUCUACUCAUCAC |
| SEQ ID NO: 268 | 549_maj | cryptic | UAUCUACUCAUCA |
| SEQ ID NO: 269 | 550_maj | cryptic | UUAUCUACUCAUC |
| SEQ ID NO: 353 | 1080_maj | intron SNP | CAGCCACUCACAA |
| SEQ ID NO: 354 | 1081_maj | intron SNP | CCAGCCACUCACA |
| SEQ ID NO: 355 | 1082_maj | intron SNP | ACCAGCCACUCAC |
| SEQ ID NO: 356 | 1083_maj | intron SNP | CACCAGCCACUCA |
| SEQ ID NO: 357 | 1084_maj | intron SNP | CCACCAGCCACUC |
| SEQ ID NO: 358 | 1085_maj | intron SNP | UCCACCAGCCACU |
| SEQ ID NO: 359 | 1086_maj | intron SNP | GUCCACCAGCCAC |
| SEQ ID NO: 360 | 1087_maj | intron SNP | UGUCCACCAGCCA |
| SEQ ID NO: 361 | 1088_maj | intron SNP | CUGUCCACCAGCC |
| SEQ ID NO: 362 | 1089_maj | intron SNP | UCUGUCCACCAGC |
| SEQ ID NO: 363 | 1090_maj | intron SNP | GUCUGUCCACCAG |
| SEQ ID NO: 364 | 1091_maj | intron SNP | CGUCUGUCCACCA |
| SEQ ID NO: 365 | 1092_maj | intron SNP | UCGUCUGUCCACC |
| SEQ ID NO: 366 | 1093_maj | intron SNP | UUCGUCUGUCCAC |
| SEQ ID NO: 367 | 1094_maj | intron SNP | UUUCGUCUGUCCA |
| SEQ ID NO: 368 | 1095_maj | intron SNP | UUUUCGUCUGUCC |
| SEQ ID NO: 369 | 1096_maj | intron SNP | UUUUUCGUCUGUC |
| SEQ ID NO: 370 | 1097_maj | intron SNP | AUUUUUCGUCUGU |
| SEQ ID NO: 371 | 1098_maj | intron SNP | CAUUUUUCGUCUG |
| SEQ ID NO: 372 | 1099_maj | intron SNP | CCAUUUUUCGUCU |
| SEQ ID NO: 373 | 1100_maj | intron SNP | UCCAUUUUUCGUC |
| SEQ ID NO: 374 | 1101_maj | intron SNP | AUCCAUUUUUCGU |
| SEQ ID NO: 375 | 1102_maj | intron SNP | CAUCCAUUUUUCG |
| SEQ ID NO: 376 | 1103_maj | intron SNP | CCAUCCAUUUUUC |
| SEQ ID NO: 377 | 1104_maj | intron SNP | ACCAUCCAUUUUU |
| SEQ ID NO: 378 | 1104_min | intron SNP | CCCAUCCAUUUUU |
| SEQ ID NO: 379 | 1105_maj | intron SNP | AACCAUCCAUUUU |
| SEQ ID NO: 380 | 1105_min | intron SNP | ACCCAUCCAUUUU |
| SEQ ID NO: 381 | 1106_maj | intron SNP | CAACCAUCCAUUU |
| SEQ ID NO: 382 | 1106_min | intron SNP | CACCCAUCCAUUU |
| SEQ ID NO: 383 | 1107_maj | intron SNP | CCAACCAUCCAUU |
| SEQ ID NO: 384 | 1107_min | intron SNP | CCACCCAUCCAUU |
| SEQ ID NO: 385 | 1108_maj | intron SNP | UCCAACCAUCCAU |
| SEQ ID NO: 386 | 1108_min | intron SNP | UCCACCCAUCCAU |
| SEQ ID NO: 387 | 1109_maj | intron SNP | AUCCAACCAUCCA |
| SEQ ID NO: 388 | 1109_min | intron SNP | AUCCACCCAUCCA |
| SEQ ID NO: 389 | 1110_maj | intron SNP | UAUCCAACCAUCC |
| SEQ ID NO: 390 | 1110_min | intron SNP | UAUCCACCCAUCC |
| SEQ ID NO: 391 | 1111_maj | intron SNP | UUAUCCAACCAUC |
| SEQ ID NO: 392 | 1111_min | intron SNP | UUAUCCACCCAUC |
| SEQ ID NO: 393 | 1112_maj | intron SNP | UUUAUCCAACCAU |
| SEQ ID NO: 394 | 1112_min | intron SNP | UUUAUCCACCCAU |
| SEQ ID NO: 395 | 1113_maj | intron SNP | AUUUAUCCAACCA |
| SEQ ID NO: 396 | 1113_min | intron SNP | AUUUAUCCACCCA |
| SEQ ID NO: 397 | 1114_maj | intron SNP | AAUUUAUCCAACC |
| SEQ ID NO: 398 | 1114_min | intron SNP | AAUUUAUCCACCC |
| SEQ ID NO: 399 | 1115_maj | intron SNP | CAAUUUAUCCAAC |
| SEQ ID NO: 400 | 1115_min | intron SNP | CAAUUUAUCCACC |
| SEQ ID NO: 401 | 1116_maj | intron SNP | UCAAUUUAUCCAA |
| SEQ ID NO: 402 | 1116_min | intron SNP | UCAAUUUAUCCAC |
| SEQ ID NO: 403 | 1117_maj | intron SNP | AUCAAUUUAUCCA |
| SEQ ID NO: 404 | 1118_maj | intron SNP | CAUCAAUUUAUCC |
| SEQ ID NO: 405 | 1119_maj | intron SNP | CCAUCAAUUUAUC |
| SEQ ID NO: 406 | 1120_maj | intron SNP | CCCAUCAAUUUAU |
| SEQ ID NO: 407 | 1121_maj | intron SNP | ACCCAUCAAUUUA |
| SEQ ID NO: 408 | 1122_maj | intron SNP | CACCCAUCAAUUU |
| SEQ ID NO: 409 | 1123_maj | intron SNP | CCACCCAUCAAUU |
| SEQ ID NO: 410 | 1124_maj | intron SNP | UCCACCCAUCAAU |
| SEQ ID NO: 411 | 1125_maj | intron SNP | AUCCACCCAUCAA |
| SEQ ID NO: 412 | 1126_maj | intron SNP | CAUCCACCCAUCA |
| SEQ ID NO: 413 | 1127_maj | intron SNP | CCAUCCACCCAUC |
| SEQ ID NO: 414 | 1128_maj | intron SNP | UCCAUCCACCCAU |
| SEQ ID NO: 415 | 1129_maj | intron SNP | AUCCAUCCACCCA |
| SEQ ID NO: 416 | 1130_maj | intron SNP | CAUCCAUCCACCC |
| SEQ ID NO: 417 | 1131_maj | intron SNP | CCAUCCAUCCACC |
| SEQ ID NO: 418 | 1132_maj | intron SNP | ACCAUCCAUCCAC |
| SEQ ID NO: 419 | 1133_maj | intron SNP | AACCAUCCAUCCA |
| SEQ ID NO: 420 | 1134_maj | intron SNP | CAACCAUCCAUCC |
| SEQ ID NO: 421 | 1135_maj | intron SNP | CCAACCAUCCAUC |
| SEQ ID NO: 422 | 1136_maj | intron SNP | ACCAACCAUCCAU |
| SEQ ID NO: 423 | 1137_maj | intron SNP | AACCAACCAUCCA |
| SEQ ID NO: 424 | 1138_maj | intron SNP | CAACCAACCAUCC |
| SEQ ID NO: 425 | 1139_maj | intron SNP | ACAACCAACCAUC |
| SEQ ID NO: 426 | 1140_maj | intron SNP | UACAACCAACCAU |
| SEQ ID NO: 427 | 1180_maj | downstream TDP-43 | UCCGCAACUUAAU |
| | | binding site | |
| SEQ ID NO: 428 | 1181_maj | downstream TDP-43 | AUCCGCAACUUAA |
| | | binding site | |
| SEQ ID NO: 429 | 1182_maj | downstream TDP-43 | GAUCCGCAACUUA |
| | | binding site | |
| SEQ ID NO: 430 | 1183_maj | downstream TDP-43 | UGAUCCGCAACUU |
| | | binding site | |
| SEQ ID NO: 431 | 1184_maj | downstream TDP-43 | UUGAUCCGCAACU |
| | | binding site | |
| SEQ ID NO: 432 | 1185_maj | downstream TDP-43 | AUUGAUCCGCAAC |
| | | binding site | |
| SEQ ID NO: 433 | 1186_maj | downstream TDP-43 | CAUUGAUCCGCAA |
| | | binding site | |
| SEQ ID NO: 434 | 1187_maj | downstream TDP-43 | ACAUUGAUCCGCA |
| | | binding site | |
| SEQ ID NO: 435 | 1188_maj | downstream TDP-43 | UACAUUGAUCCGC |
| | | binding site | |
| SEQ ID NO: 436 | 1189_maj | downstream TDP-43 | AUACAUUGAUCCG |
| | | binding site | |
| SEQ ID NO: 437 | 1190_maj | downstream TDP-43 | CAUACAUUGAUCC |
| | | binding site | |
| SEQ ID NO: 438 | 1191_maj | downstream TDP-43 | CCAUACAUUGAUC |
| | | binding site | |
| SEQ ID NO: 439 | 1192_maj | downstream TDP-43 | CCCAUACAUUGAU |
| | | binding site | |
| SEQ ID NO: 440 | 1193_maj | downstream TDP-43 | UCCCAUACAUUGA |
| | | binding site | |
| SEQ ID NO: 441 | 1194_maj | downstream TDP-43 | AUCCCAUACAUUG |
| | | binding site | |
| SEQ ID NO: 442 | 1195_maj | downstream TDP-43 | CAUCCCAUACAUU |
| | | binding site | |
| SEQ ID NO: 443 | 1196_maj | downstream TDP-43 | CCAUCCCAUACAU |
| | | binding site | |
| SEQ ID NO: 444 | 1197_maj | downstream TDP-43 | UCCAUCCCAUACA |
| | | binding site | |
| SEQ ID NO: 445 | 1198_maj | downstream TDP-43 | AUCCAUCCCAUAC |
| | | binding site | |
| SEQ ID NO: 446 | 1199_maj | downstream TDP-43 | CAUCCAUCCCAUA |
| | | binding site | |
| SEQ ID NO: 447 | 1200_maj | downstream TDP-43 | UCAUCCAUCCCAU |
| | | binding site | |
| SEQ ID NO: 448 | 1201_maj | downstream TDP-43 | UUCAUCCAUCCCA |
| | | binding site | |
| SEQ ID NO: 449 | 1202_maj | downstream TDP-43 | AUUCAUCCAUCCC |
| | | binding site | |
| SEQ ID NO: 450 | 1203_maj | downstream TDP-43 | CAUUCAUCCAUCC |
| | | binding site | |
| SEQ ID NO: 451 | 1204_maj | downstream TDP-43 | CCAUUCAUCCAUC |
| | | binding site | |
| SEQ ID NO: 452 | 1205_maj | downstream TDP-43 | UCCAUUCAUCCAU |
| | | binding site | |
| SEQ ID NO: 453 | 1206_maj | downstream TDP-43 | AUCCAUUCAUCCA |
| | | binding site | |
| SEQ ID NO: 454 | 1207_maj | downstream TDP-43 | CAUCCAUUCAUCC |
| | | binding site | |
| SEQ ID NO: 455 | 1208_maj | downstream TDP-43 | CCAUCCAUUCAUC |
| | | binding site | |
| SEQ ID NO: 456 | 1209_maj | downstream TDP-43 | UCCAUCCAUUCAU |
| | | binding site | |
| SEQ ID NO: 457 | 1210_maj | downstream TDP-43 | AUCCAUCCAUUCA |
| | | binding site | |
| SEQ ID NO: 458 | 1211_maj | downstream TDP-43 | CAUCCAUCCAUUC |
| | | binding site | |
| SEQ ID NO: 459 | 1212_maj | downstream TDP-43 | CCAUCCAUCCAUU |
| | | binding site | |
| SEQ ID NO: 460 | 1213_maj | downstream TDP-43 | UCCAUCCAUCCAU |
| | | binding site | |
| SEQ ID NO: 461 | 1214_maj | downstream TDP-43 | AUCCAUCCAUCCA |
| | | binding site | |
| SEQ ID NO: 462 | 1215_maj | downstream TDP-43 | CAUCCAUCCAUCC |
| | | binding site | |
| SEQ ID NO: 463 | 1216_maj | downstream TDP-43 | CCAUCCAUCCAUC |
| | | binding site | |
| SEQ ID NO: 464 | 1220_maj | downstream TDP-43 | ACAUCCAUCCAUC |
| | | binding site | |
| SEQ ID NO: 465 | 1221_maj | downstream TDP-43 | CACAUCCAUCCAU |
| | | binding site | |
| SEQ ID NO: 466 | 1222_maj | downstream TDP-43 | ACACAUCCAUCCA |
| | | binding site | |
| SEQ ID NO: 467 | 1223_maj | downstream TDP-43 | CACACAUCCAUCC |
| | | binding site | |
| SEQ ID NO: 468 | 1224maj | downstream TDP-43 | CCACACAUCCAUC |
| | | binding site | |
| SEQ ID NO: 469 | 1225_maj | downstream TDP-43 | ACCACACAUCCAU |
| | | binding site | |
| SEQ ID NO: 470 | 1226_maj | downstream TDP-43 | AACCACACAUCCA |
| | | binding site | |
| SEQ ID NO: 471 | 1227_maj | downstream TDP-43 | CAACCACACAUCC |
| | | binding site | |
| SEQ ID NO: 472 | 1228_maj | downstream TDP-43 | UCAACCACACAUC |
| | | binding site | |
| SEQ ID NO: 473 | 1229_maj | downstream TDP-43 | UUCAACCACACAU |
| | | binding site | |
| SEQ ID NO: 474 | 1230_maj | downstream TDP-43 | AUUCAACCACACA |
| | | binding site | |
| SEQ ID NO: 475 | 785_maj | enhancer | UCUAUCCAGCAAU |
| SEQ ID NO: 476 | 786_maj | enhancer | AUCUAUCCAGCAA |
| SEQ ID NO: 477 | 787_maj | enhancer | CAUCUAUCCAGCA |
| SEQ ID NO: 478 | 788_maj | enhancer | UCAUCUAUCCAGC |
| SEQ ID NO: 479 | 789_maj | enhancer | AUCAUCUAUCCAG |
| SEQ ID NO: 480 | 790_maj | enhancer | AAUCAUCUAUCCA |
| SEQ ID NO: 481 | 791_maj | enhancer | CAAUCAUCUAUCC |
| SEQ ID NO: 482 | 792_maj | enhancer | UCAAUCAUCUAUC |
| SEQ ID NO: 483 | 793_maj | enhancer | GUCAAUCAUCUAU |
| SEQ ID NO: 484 | 794_maj | enhancer | AGUCAAUCAUCUA |
| SEQ ID NO: 485 | 795_maj | enhancer | CAGUCAAUCAUCU |
| SEQ ID NO: 486 | 796_maj | enhancer | CCAGUCAAUCAUC |
| SEQ ID NO: 487 | 797_maj | enhancer | CCCAGUCAAUCAU |
| SEQ ID NO: 488 | 798_maj | enhancer | ACCCAGUCAAUCA |
| SEQ ID NO: 489 | 799_maj | enhancer | CACCCAGUCAAUC |
| SEQ ID NO: 490 | 800_maj | enhancer | CCACCCAGUCAAU |
| SEQ ID NO: 491 | 801_maj | enhancer | UCCACCCAGUCAA |
| SEQ ID NO: 492 | 802_maj | enhancer | AUCCACCCAGUCA |
| SEQ ID NO: 493 | 803_maj | enhancer | UAUCCACCCAGUC |
| SEQ ID NO: 494 | 804_maj | enhancer | CUAUCCACCCAGU |
| SEQ ID NO: 495 | 805_maj | enhancer | UCUAUCCACCCAG |
| SEQ ID NO: 496 | 806_maj | enhancer | AUCUAUCCACCCA |
| SEQ ID NO: 497 | 807_maj | enhancer | CAUCUAUCCACCC |
| SEQ ID NO: 498 | 808_maj | enhancer | UCAUCUAUCCACC |
| SEQ ID NO: 499 | 809_maj | enhancer | UUCAUCUAUCCAC |
| SEQ ID NO: 500 | 810_maj | enhancer | UUUCAUCUAUCCA |
| SEQ ID NO: 501 | 811_maj | enhancer | AUUUCAUCUAUCC |
| SEQ ID NO: 502 | 812_maj | enhancer | CAUUUCAUCUAUC |
| SEQ ID NO: 503 | 813_maj | enhancer | ACAUUUCAUCUAU |
| SEQ ID NO: 504 | 814_maj | enhancer | AACAUUUCAUCUA |
| SEQ ID NO: 505 | 815_maj | enhancer | CAACAUUUCAUCU |
| SEQ ID NO: 506 | 1360_maj | enhancer | UCUGGGGCAGCUU |
| SEQ ID NO: 507 | 1361_maj | enhancer | GUCUGGGGCAGCU |
| SEQ ID NO: 508 | 1362_maj | enhancer | GGUCUGGGGCAGC |
| SEQ ID NO: 509 | 1363_maj | enhancer | GGGUCUGGGGCAG |
| SEQ ID NO: 510 | 1364_maj | enhancer | AGGGUCUGGGGCA |
| SEQ ID NO: 511 | 1365_maj | enhancer | CAGGGUCUGGGGC |
| SEQ ID NO: 512 | 1366_maj | enhancer | CCAGGGUCUGGGG |
| SEQ ID NO: 513 | 1367_maj | enhancer | CCCAGGGUCUGGG |
| SEQ ID NO: 514 | 1368_maj | enhancer | UCCCAGGGUCUGG |
| SEQ ID NO: 515 | 1369_maj | enhancer | GUCCCAGGGUCUG |
| SEQ ID NO: 516 | 1370_maj | enhancer | GGUCCCAGGGUCU |
| SEQ ID NO: 517 | 1371_maj | enhancer | AGGUCCCAGGGUC |
| SEQ ID NO: 518 | 1372_maj | enhancer | GAGGUCCCAGGGU |
| SEQ ID NO: 519 | 1373_maj | enhancer | AGAGGUCCCAGGG |
| SEQ ID NO: 520 | 1374_maj | enhancer | CAGAGGUCCCAGG |
| SEQ ID NO: 521 | 1375_maj | enhancer | UCAGAGGUCCCAG |
| SEQ ID NO: 522 | 1376_maj | enhancer | GUCAGAGGUCCCA |
| SEQ ID NO: 523 | 1377_maj | enhancer | GGUCAGAGGUCCC |
| SEQ ID NO: 524 | 1378_maj | enhancer | GGGUCAGAGGUCC |
| SEQ ID NO: 525 | 1379_maj | enhancer | GGGGUCAGAGGUC |
| SEQ ID NO: 526 | 1380_maj | enhancer | GGGGGUCAGAGGU |
| SEQ ID NO: 527 | 1381_maj | enhancer | CGGGGGUCAGAGG |
| SEQ ID NO: 528 | 1382_maj | enhancer | CCGGGGGUCAGAG |
| SEQ ID NO: 529 | 1383_maj | enhancer | GCCGGGGGUCAGA |
| SEQ ID NO: 530 | 1384_maj | enhancer | CGCCGGGGGUCAG |
| SEQ ID NO: 531 | 1385_maj | enhancer | UCGCCGGGGGUCA |
| SEQ ID NO: 532 | 1386_maj | enhancer | GUCGCCGGGGGUC |
| SEQ ID NO: 533 | 1387_maj | enhancer | GGUCGCCGGGGGU |
| SEQ ID NO: 534 | 1388_maj | enhancer | GGGUCGCCGGGGG |
| SEQ ID NO: 535 | 1389_maj | enhancer | GGGGUCGCCGGGG |
| SEQ ID NO: 536 | 1390_maj | enhancer | AGGGGUCGCCGGG |
| SEQ ID NO: 537 | 1391_maj | enhancer | AAGGGGUCGCCGG |
| SEQ ID NO: 538 | 1392_maj | enhancer | CAAGGGGUCGCCG |
| SEQ ID NO: 539 | 1393_maj | enhancer | GCAAGGGGUCGCC |
| SEQ ID NO: 540 | 1394_maj | enhancer | UGCAAGGGGUCGC |
| SEQ ID NO: 541 | 1395_maj | enhancer | GUGCAAGGGGUCG |
| SEQ ID NO: 542 | 1396_maj | enhancer | AGUGCAAGGGGUC |
| SEQ ID NO: 543 | 1397_maj | enhancer | GAGUGCAAGGGGU |
| SEQ ID NO: 544 | 1398_maj | enhancer | AGAGUGCAAGGGG |
| SEQ ID NO: 545 | 1399_maj | enhancer | GAGAGUGCAAGGG |
| SEQ ID NO: 546 | 1400_maj | enhancer | GGAGAGUGCAAGG |

TABLE 4
Target sites for UNC13A ASOs

| SEQ ID | Sequence | Target site |
|---|---|---|
| SEQ ID NO: 547 | AUGAGUAGAUAAAAGGAUGGAUGGA | donor splice site |
| | GAGAUGGGUGAGUACAUGGAUGGAU | |
| | AGAUGGAUGAGUUGGUGGGUAGAUU | |
| | CGUGC | |
| SEQ ID NO: 548 | AUGAGUAGAUAAAAGCAUGGAUGGA | donor splice site |
| | GAGUGGGUGAGUACAUGGAUGGAUA | |
| | GAUGGAUGAGUUGGUGGGUAGAUUC | |
| | GUGC | |
| SEQ ID NO: 549 | GGAUGGAUGGAGAGAUGGGUGAGUA | donor splice site |
| | CAUGGAUGGAUAGAU | |
| SEQ ID NO: 550 | GCAUGGAUGGAGAGAUGGGUGAGUA | donor splice site |
| | CAUGGAUGGAUAGAU | |
| SEQ ID NO: 551 | AUGGAGAGAUGGGUGAGUACAUGGA | donor splice site |
| | UGGA | |
| SEQ ID NO: 552 | GUUCUUCCUUCUAUUCCAGCCCUAAC | acceptor splice site |
| | CACUCAGGAUUGGGCCGUUUGUGUC | |
| | UGGGUAUGUCUCUUCCAGCUGCCUG | |
| | GGUUUCCUGGAAAGAACUCUU | |
| SEQ ID NO: 553 | GUGUCUGGGUAUGUCUCUUCCAGCU | acceptor splice site |
| | GCCUGGGUUUCCUGGAAA | |
| SEQ ID NO: 554 | UCAUCAGGUUCUUCCUUCUAUUCCA | acceptor splice site |
| | GCCCUAACCACUCAGGAUUGG | |

In the ASOs disclosed herein “U” and “T” nucleosides, i.e., uracil or thymine, may be used interchangeably. Therefore “U” in an RNA in any of SEQ ID NOS: 4-546 may be replaced by “T” in the DNA form.

It has been demonstrated that TDP-43 pathology and its nuclear loss induces a reduction of UNC13A. This has been identified to happen through the inclusion of a toxic CE within UNC13A. Furthermore, it was found that common genetic variation in UNC13A can facilitate the occurrence of this CE and make the disease more rapid and aggressive.

ASOs can be used as therapeutics to prevent the inclusion of this novel UNC13A cryptic exon. These therapeutics can modulate UNC13A splicing and prevent inclusion of the toxic cryptic exon within UNC13A, thereby preventing decreased levels of UNC13A.

The present inventors have identified two splice acceptors (chr19:17,642,541 or chr19:17642591) and one splice donor site (chr19:17642414) for the UNC13A cryptic event. Targeting the splice sites makes them less available for splicing. ASOs (i.e., or portions thereof) that target splice sites correspond to SEQ ID NO: 105-189 and SEQ ID NO: 270-352.

The present inventors have also identified a branchpoint (chr19:17642800). Targeting the branchpoint makes splicing less efficient. ASOs (or portions thereof) that target these sites correspond to SEQ ID NO 4-104.

The present inventors also identified splicing regulatory elements (SREs) within the CE itself and the intronic sequences flanking it up- and down-stream. These were determined by in silico methods. TDP-43 binding sites were determined using publicly available TDP-43 iCLIP datasets (as described above). Enhancer sites were determined using ESEfinder 3.0, http://krainer01.csh1.edu/cgibin/tools/ESE3/esefinder.cgi?process=home. Targeting SREs limits the binding of RNA binding proteins, for example, those that modulate or enhance the inclusion of the CE.

ASOs (or portions thereof) that target sequences within the UNC13A cryptic exon correspond to SEQ ID NO: 190-269.

ASOs (or portions thereof) that target splice enhancers, as identified by ESEfinder, correspond to SEQ ID NO: 475-576.

ASOs (or portions thereof) that target downstream TDP-43 binding sites correspond to SEQ TD NO: 427-474.

ASOs (or portions thereof) that target a SNP in the intronic flanking region of the UNC13A CE correspond to SEQ TD NO: 353-426.

### Example 7. Rescue in SK-N-DZ Cells

The following ASO sequences were tested for a rescue effect against the UNC13A cryptic exon where “*” is a phosphorothioate, “+” is an LNA and “in” is a 2′-O-methyl RNA.

TABLE 5
Sequences tested for rescue effect

| SEQ ID | | | | |
|---|---|---|---|---|
| NO: | Name | Sequence | Target | Chemistry |
| 555 | 21 nt _SNP & | +C*A*C*+C*C*A*+T*C*T*+C*T*C*+C*A*T*+C*C | Donor and | LNA |
| | DonMix1 | *A*+T*G*C | CE SNP | |
| 556 | 21 nt_SNP & | +C*A*C*+C*C*A*+T*C*T*+C*T*C*+C*A*T*+C*C | Donor and | LNA |
| | DonMix2 | *A*+T*C*C | CE SNP | |
| 557 | 21 nt_Don1 | +C*T*C*+A*C*C*+C*A*T*+C*T*C*+T*C*C*+A*T | Donor | LNA |
| | | *C*+C*A*+T | | |
| 558 | 21 nt_Don2 | +T*G*T*+A*C*T*+C*A*C*+C*C*A*+T*C*T*+C*T | Donor | LNA |
| | | *C*+C*A*+T | | |
| 559 | 21 nt_Don3 | +T*C*C*+A*T*G*+T*A*C*+T*C*A*+C*C*C*+A*T | Donor | LNA |
| | | *C*+T*C*+T | | |
| 560 | 21 nt_Don4 | +T*C*C*+A*T*C*+C*A*T*+G*T*A*+C*T*C*+A*C | Donor | LNA |
| | | *C*+C*A*+T | | |
| 561 | 21 nt_Don5 | +A*T*C*+T*A*T*+C*C*A*+T*C*C*+A*T*G*+T*A | Donor | LNA |
| | | *C*+T*C*+A | | |
| 562 | 17 nt_Don1 | +T*C*A*+C*C*C*+A*T*C*+T*C*T*+C*C*A*+T*+ | Donor | LNA |
| | | C | | |
| 563 | 17 nt_Don2 | +T*C*+A*C*+C*C*+A*T*+C*T*+C*T*+C*C*+A*T | Donor | LNA |
| | | *+C | | |
| 564 | 17 nt_Don3 | +G*T*A*+C*T*C*+A*C*C*+C*A*T*+C*T*C*+T*+ | Donor | LNA |
| | | C | | |
| 565 | 17 nt_Don4 | +G*T*+A*C*+T*C*+A*C*+C*C*+A*T*+C*T*+C*T | Donor | LNA |
| | | *+C | | |
| 566 | 13 nt_Don1 | C*+A*C*+C*C*+A*T*+C*T*+C*T*+C*C | Donor | LNA |
| 567 | OMe_25 nt | mC*mA*mU*mG*mU*mA*mC*mU*mC*mA*mC*m | Donor | 2-OMe RNA |
| | Don | C*mC*mA*mU*mC*mU*mC*mU*mC*mC*mA*mU | | |
| | | *mC*mC | | |
| 568 | OMe_30 nt | mA*mU*mC*mC*mA*mU*mG*mU*mA*mC*mU*m | Donor | 2-OMe RNA |
| | Don | C*mA*mC*mC*mC*mA*mU*mC*mU*mC*mU*mC | | |
| | | *mC*mA*mU*mC*mC*mA*mU | | |
| 569 | 21 nt_Short | +C*A*G*+G*C*A*+G*C*T*+G*G*A*+A*G*A*+G* | Acceptor | LNA |
| | Acc2 | A*C*+A*T*+A | | |
| 570 | 21 nt_Short | +A*A*C*+C*C*A*+G*G*C*+A*G*C*+T*G*G*+A* | Acceptor | LNA |
| | Acc3 | A*G*+A*G*+A | | |
| 571 | 21 nt_Short | +G*G*A*+A*A*C*+C*C*A*+G*G*C*+A*G*C*+T* | Acceptor | LNA |
| | Acc4 | G*G*+A*A*+G | | |
| 572 | Scrambled | A*+G*T*+T*T*+T*C*+A*A*+T*T*+C*C | Control | LNA |
| | Control | | | |
| 579 | 21 nt_LongA | +T*A*G*+G*G*C*+T*G*G*+A*A*T*+A*G*A*+A* | Acceptor | LNA |
| | cc1 | G*G*+A*A*+G | | |
| 580 | 21 nt_LongA | +T*G*A*+G*T*G*+G*T*T*+A*G*G*+G*C*T*+G* | Acceptor | LNA |
| | cc2 | G*A*+A*T*+A | | |

To assess the ability of different ASOs to induce the correct splicing event in disease-state cells, we transfected ASOs into SK-N-DZ cells with doxycycline inducible TDP-43 knockdown and assessed endogenous UNC13A splicing via reverse transcription PCR (RT-PCR). These human, neuron-like cells have previously been demonstrated to replicate numerous aberrant splicing events found in ALS/FTD patients and are thus a suitable model for this study.

It was found that ASOs targeting regions near the cryptic donor splice site and, to a lesser extent, the two cryptic acceptor splice sites, were able to significantly increase the ratio of correctly spliced to incorrectly spliced UNC13A. Most effective were 21 nucleotide ASOs featuring a full phosphorothioate (PS) backbone and 3350 locked nucleic acid (LNA) modified constructs, targeting regions near the donor splice site. These ASOs were able to increase the ratio to ˜90% correctly spliced, versus near-0% for doxycycline-treated cells without ASO transfection or cells treated with a scrambled control ASO (FIGS. 14A and 14B).

It was also found that ASOs targeting regions near the two cryptic acceptor splice sites (both long and short), ASOs of shorter length (minimum of 13 nucleotides), and ASOs of differing chemistries (100% 2′-O-methyl modified ASOs with full phosphorothioate backbones) were also able to significantly rescue this splicing event (see FIG. 14A and FIG. 14B). Thus, the design of ASOs that can rescue this splicing event is not limited to a single chemistry or binding site.

Of note, one ASO (21nt_Don_5; SEQ ID. NO. 561) was able to greatly increase the level of correct splicing despite not directly overlapping with a cryptic donor splice site, instead binding to an intronic flanking region of the donor splice site. This demonstrates that ASOs which interfere with the binding of splicing factors or regulators can inhibit the cryptic splicing event, without needing to directly mask the cryptic splice sites.

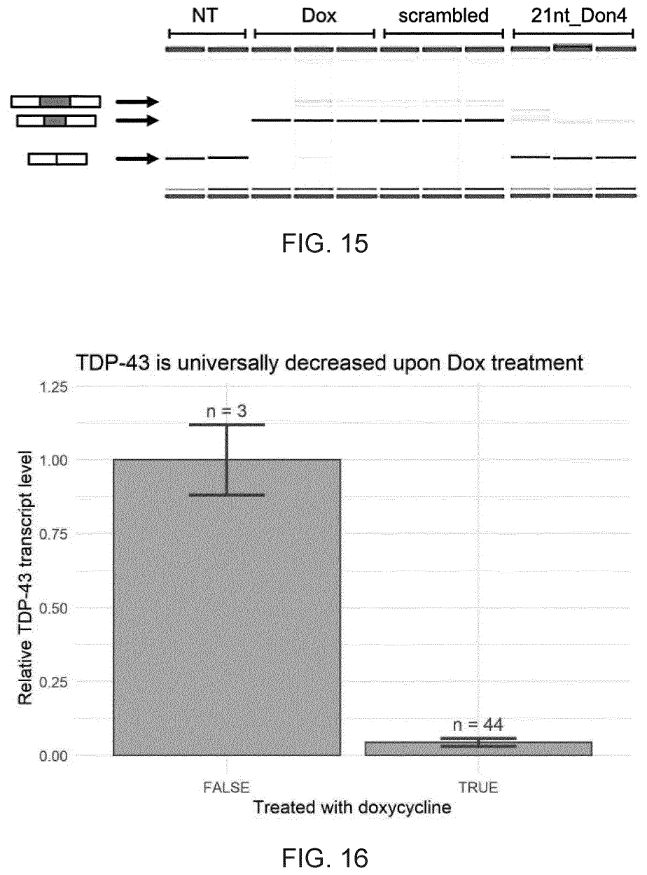

Additionally, we also found that mixtures of ASOs targeting different regions can result in efficient rescue of the correct splicing event (FIG. 14B). The fraction of correctly spliced mature RNA was determined and compared to the fraction in the Dox-treated control by RT-PCR. An example electrophoresis result is shown in FIG. 15.

To also ensure that the detected rescues were not simply due to inefficient TDP-43 knockdown, we assessed remaining TDP-43 transcript levels after doxycycline treatment via quantitative PCR for all samples (qPCR). We found that TDP-43 levels were universally decreased in all samples treated with doxycycline (including those transfected with ASOs), with very little variance between samples (FIG. 16). Therefore, changes in the correct:incorrect splicing ratio reflect genuine rescue of the correct splicing event, rather than inefficient TDP-43 knockdown.

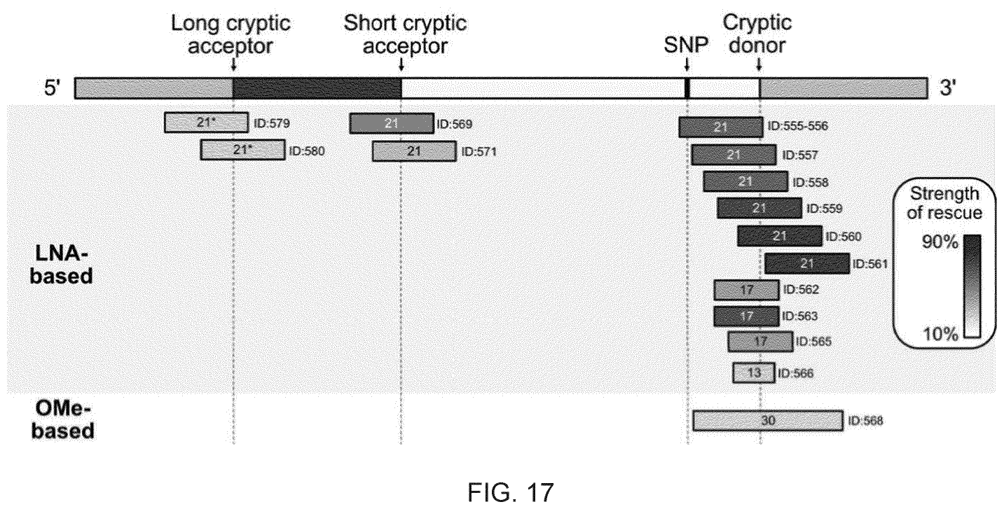

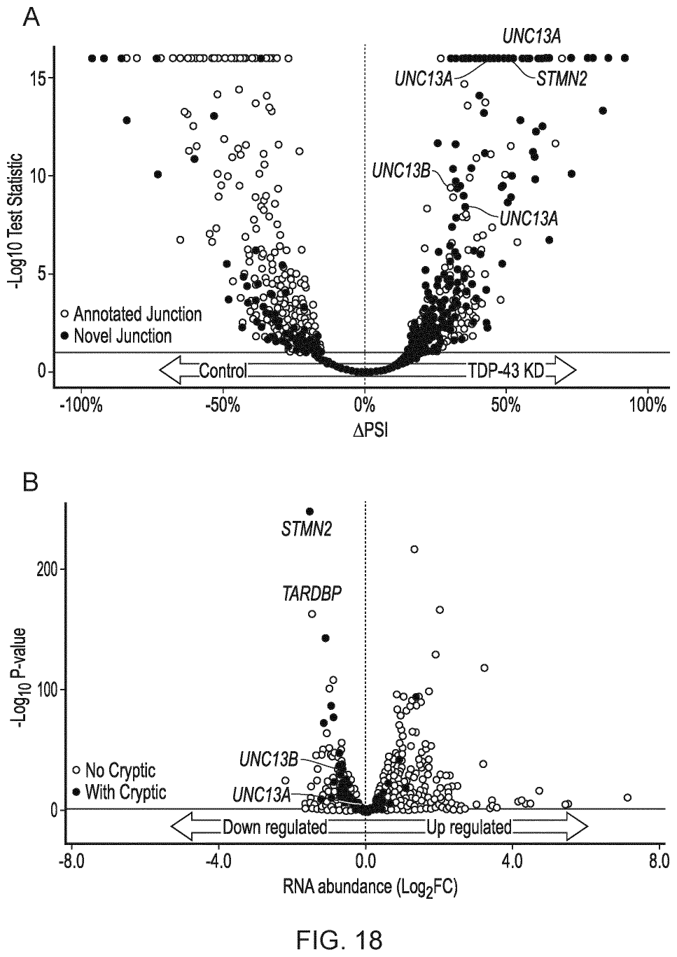

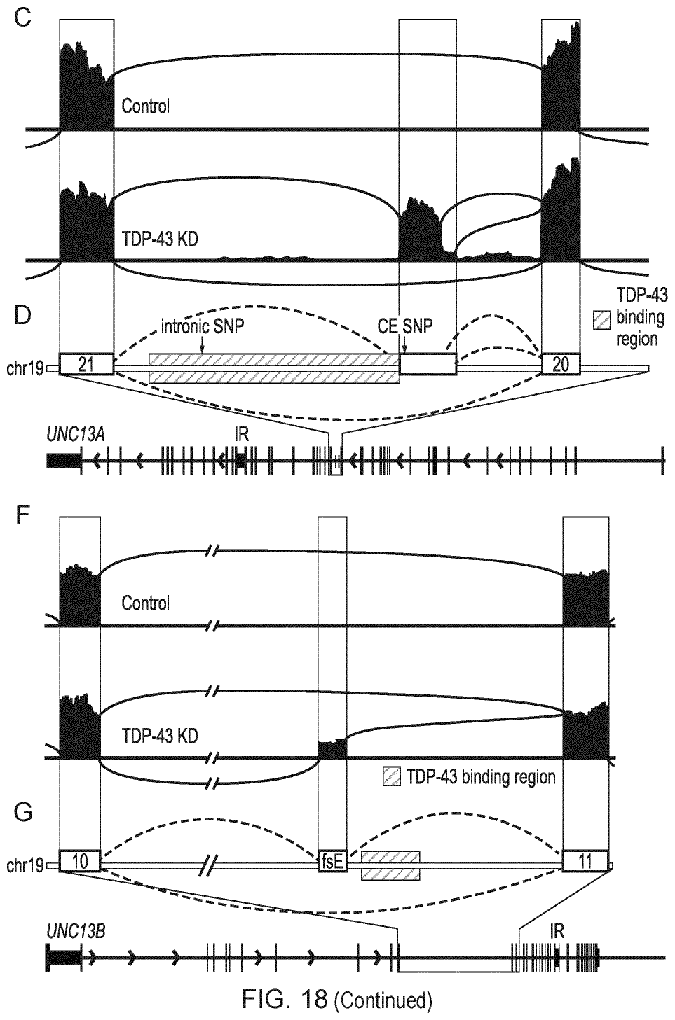

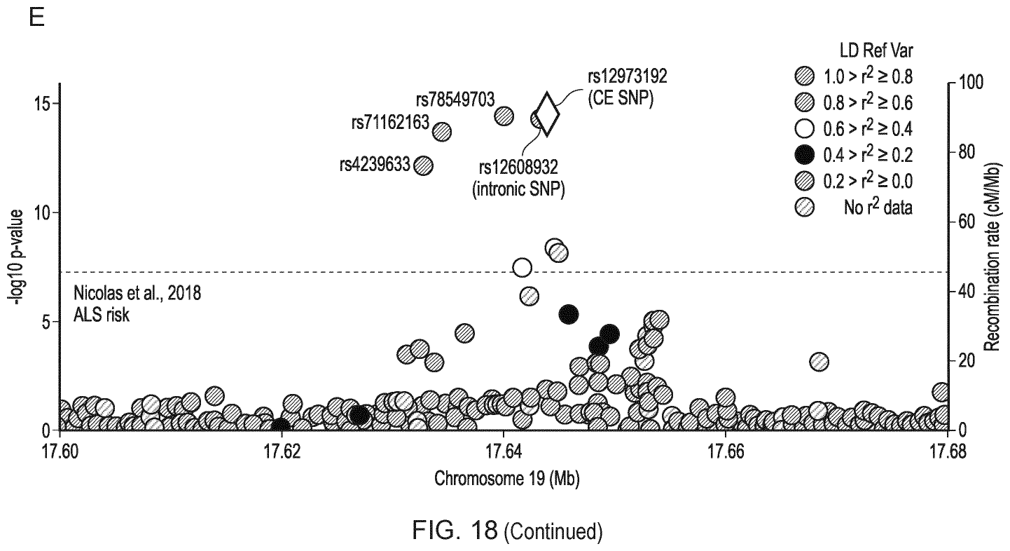

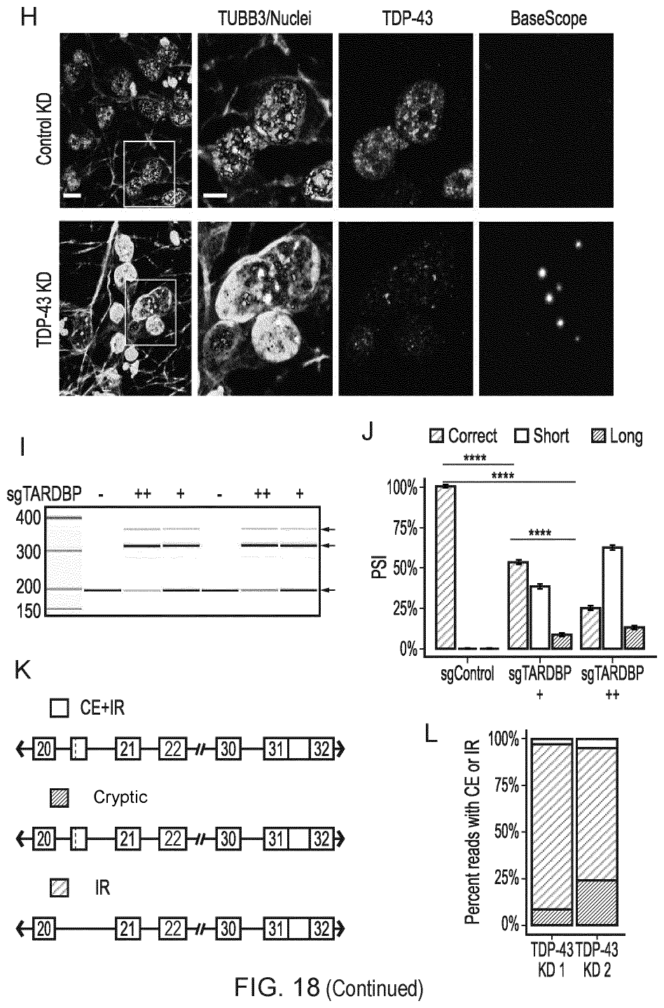

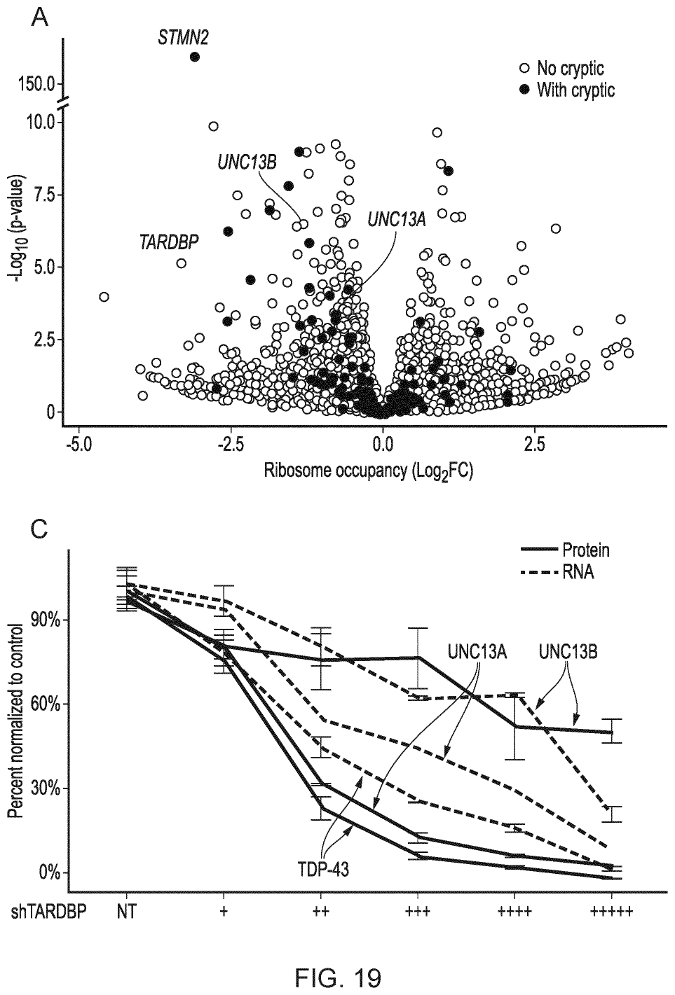

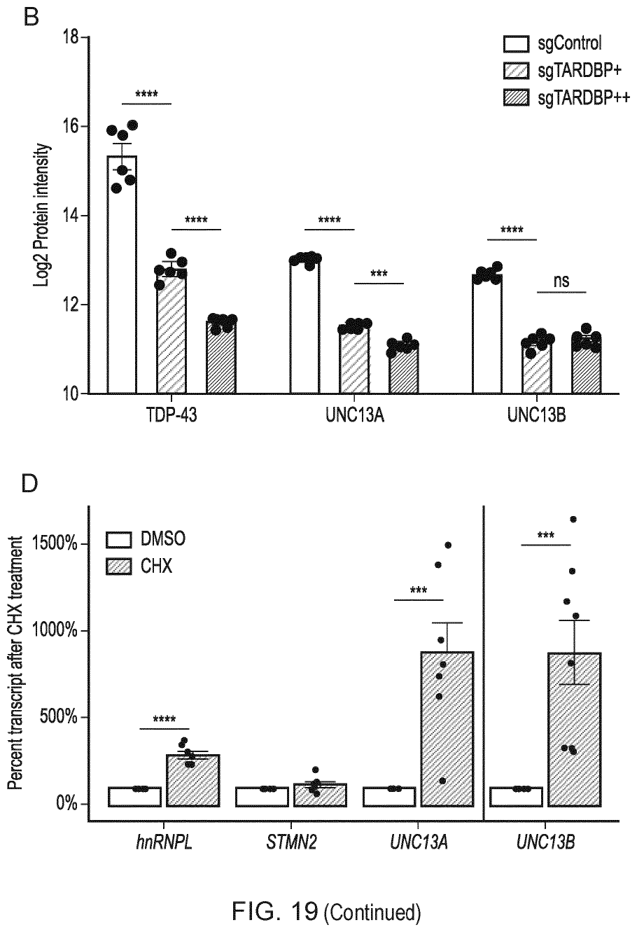

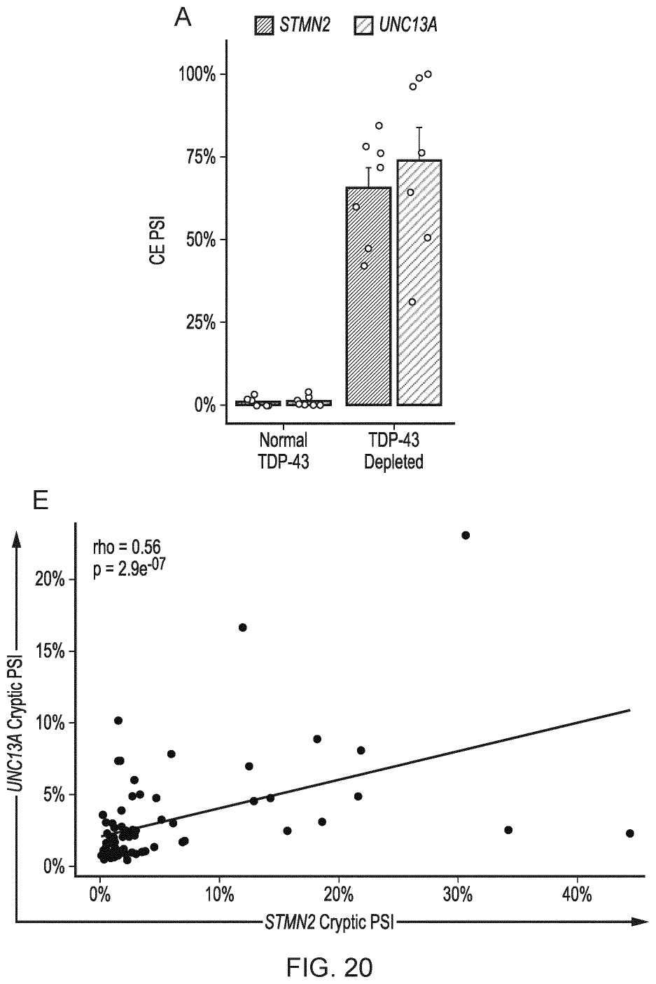

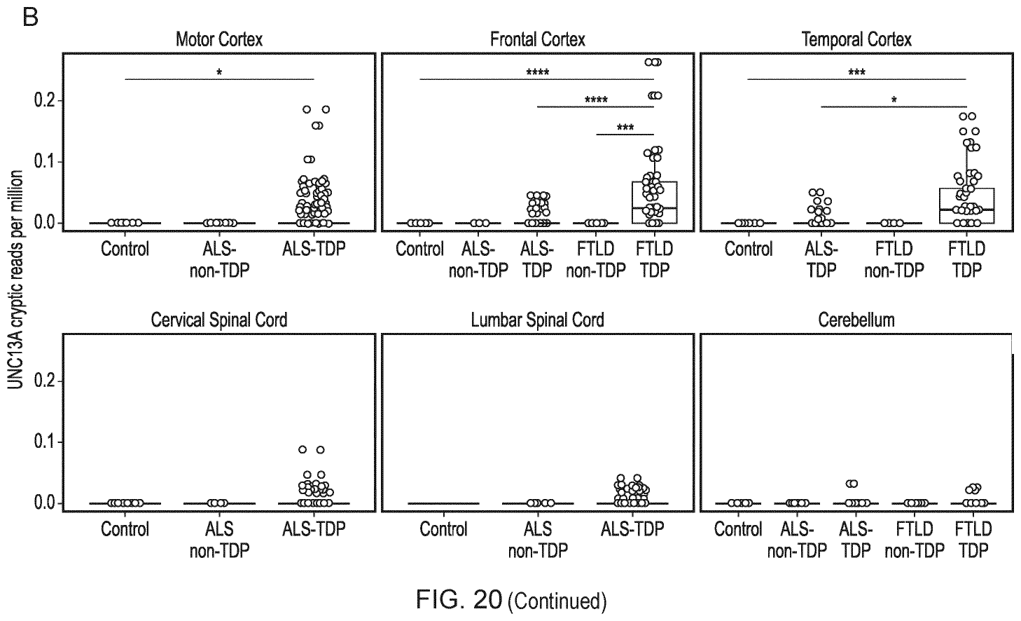

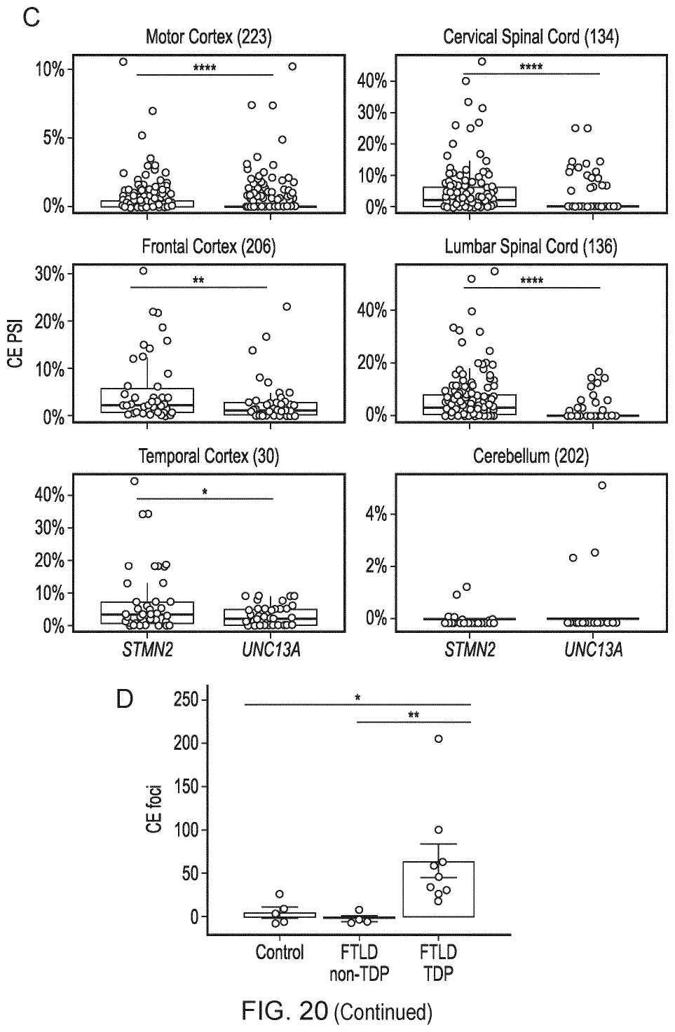

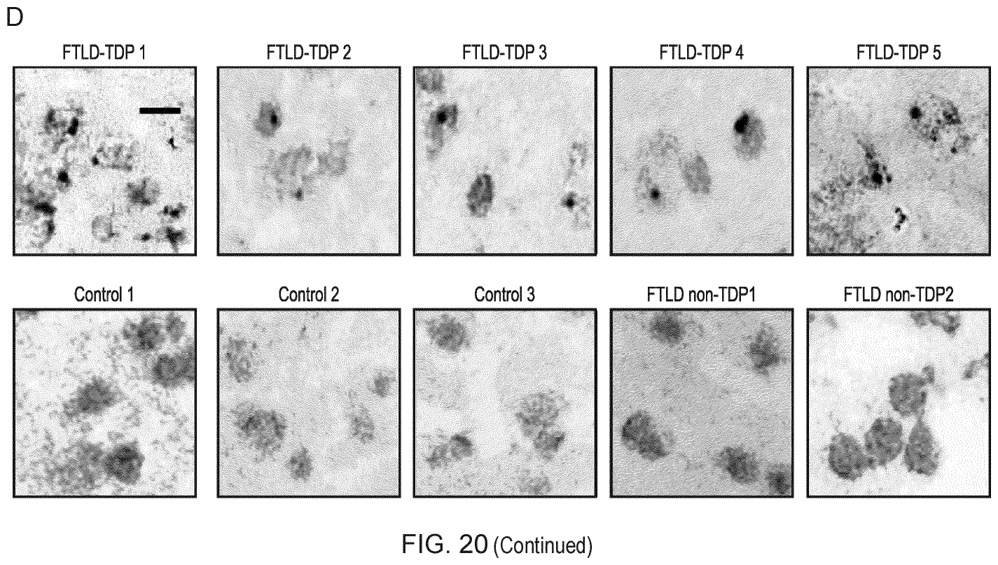

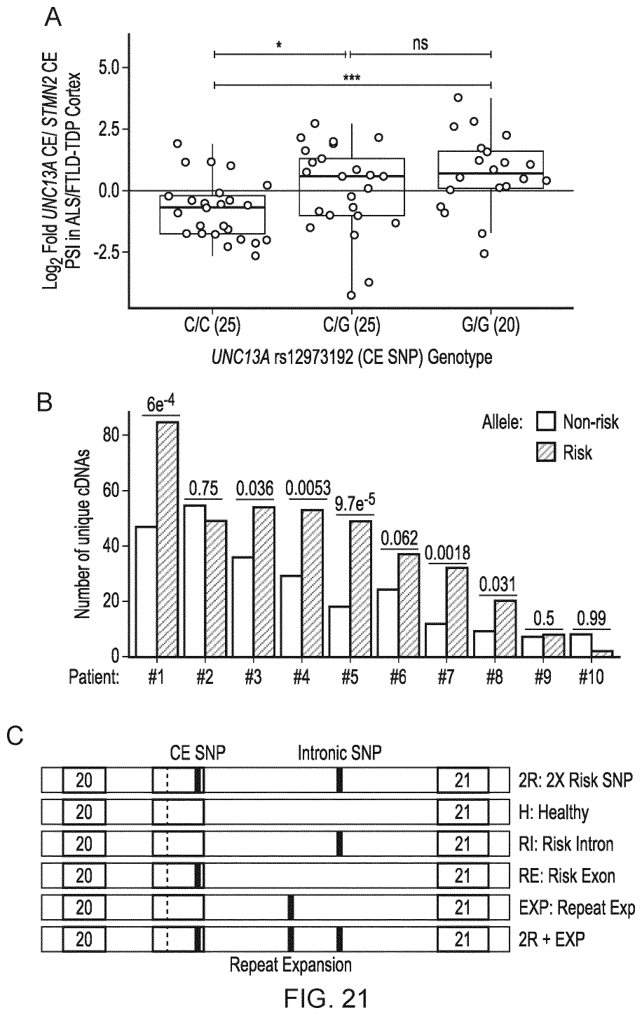

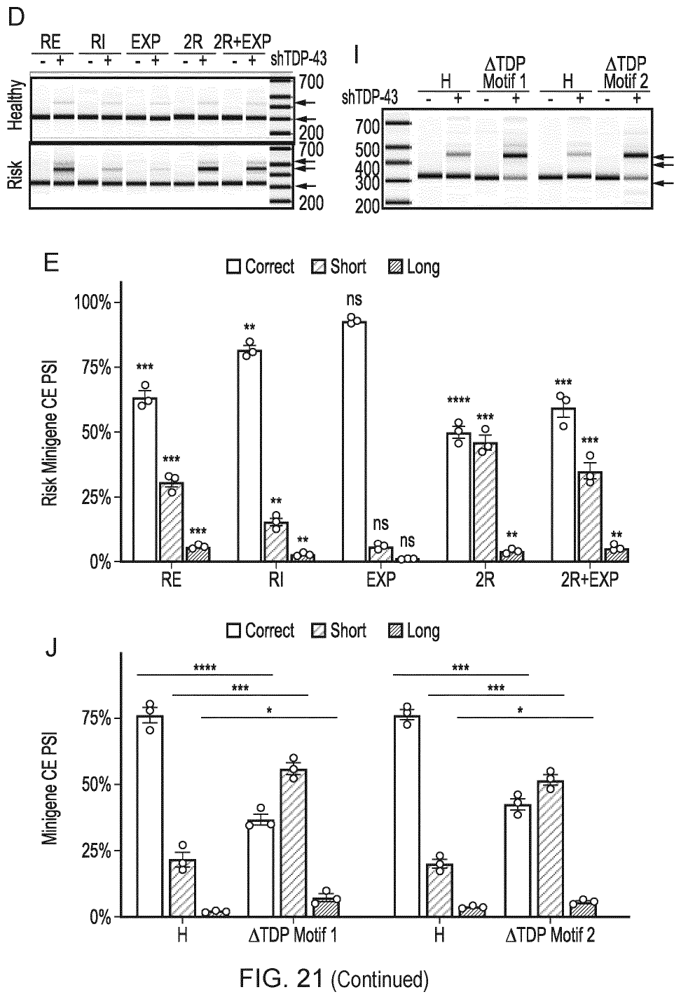

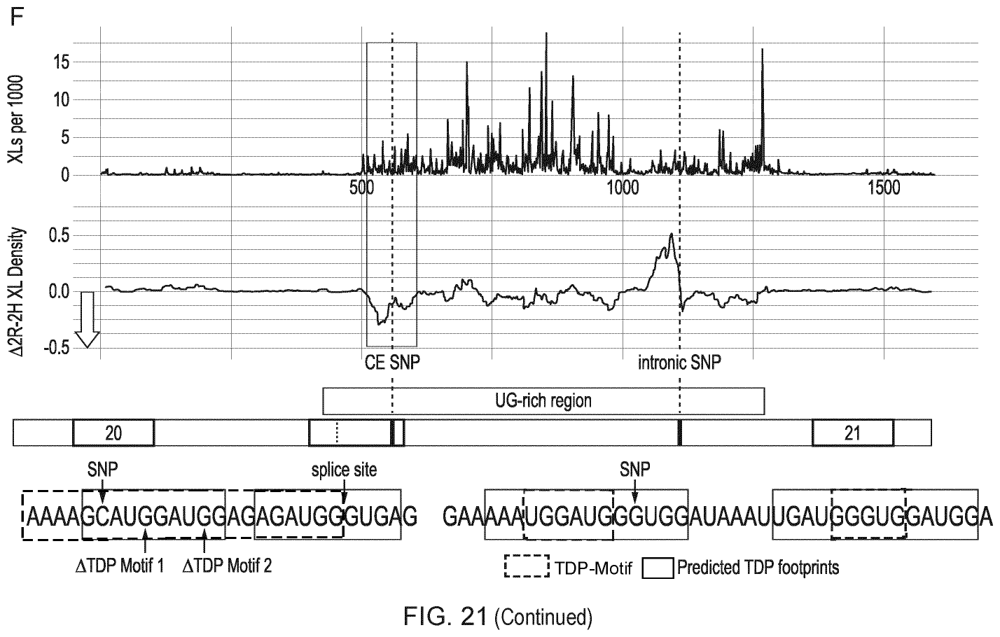

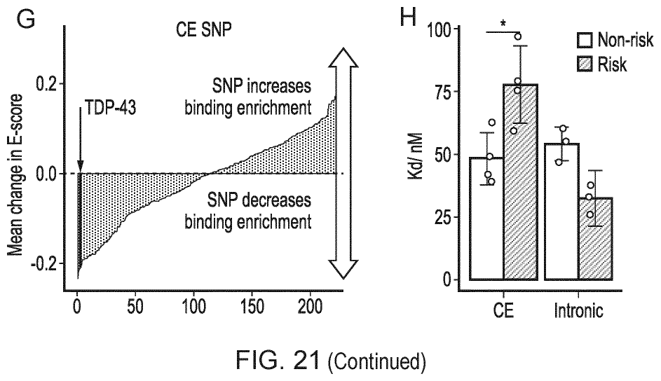

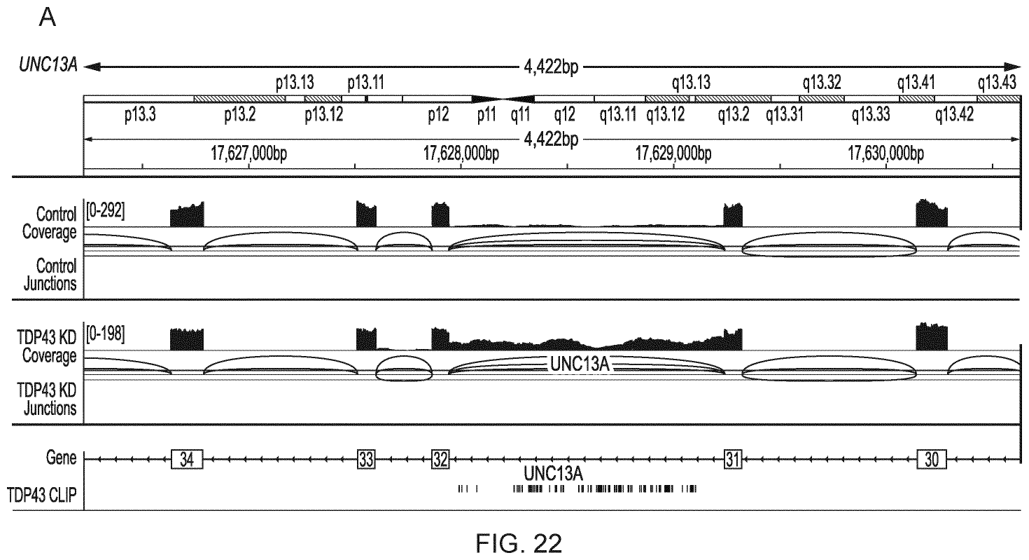

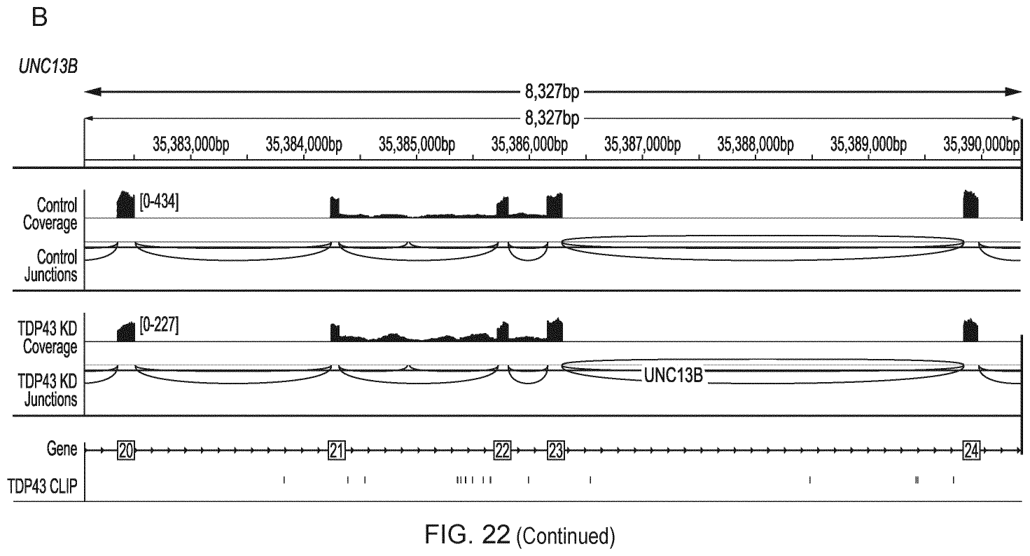

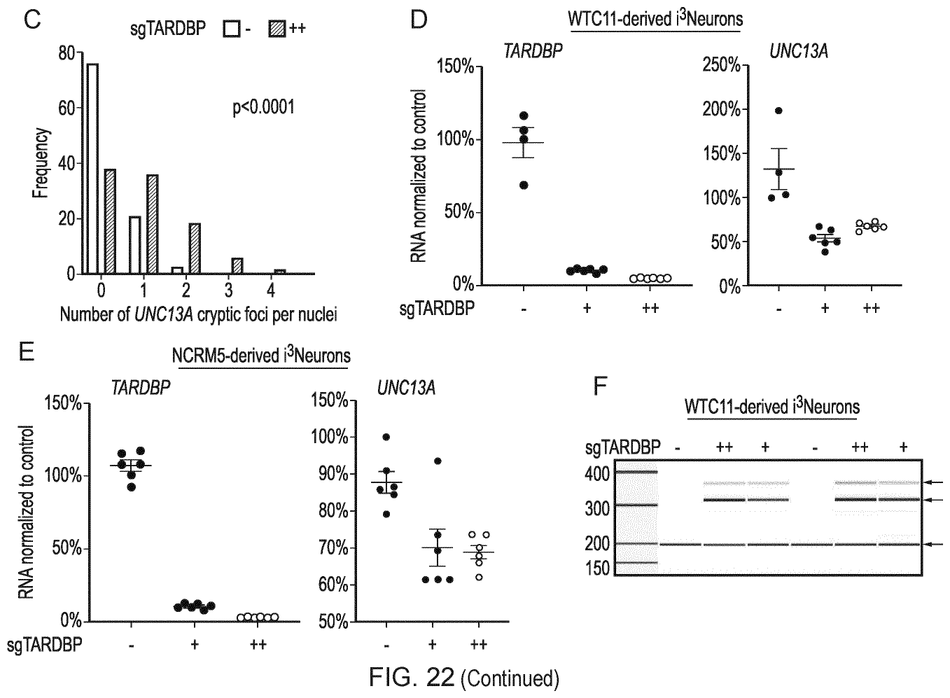

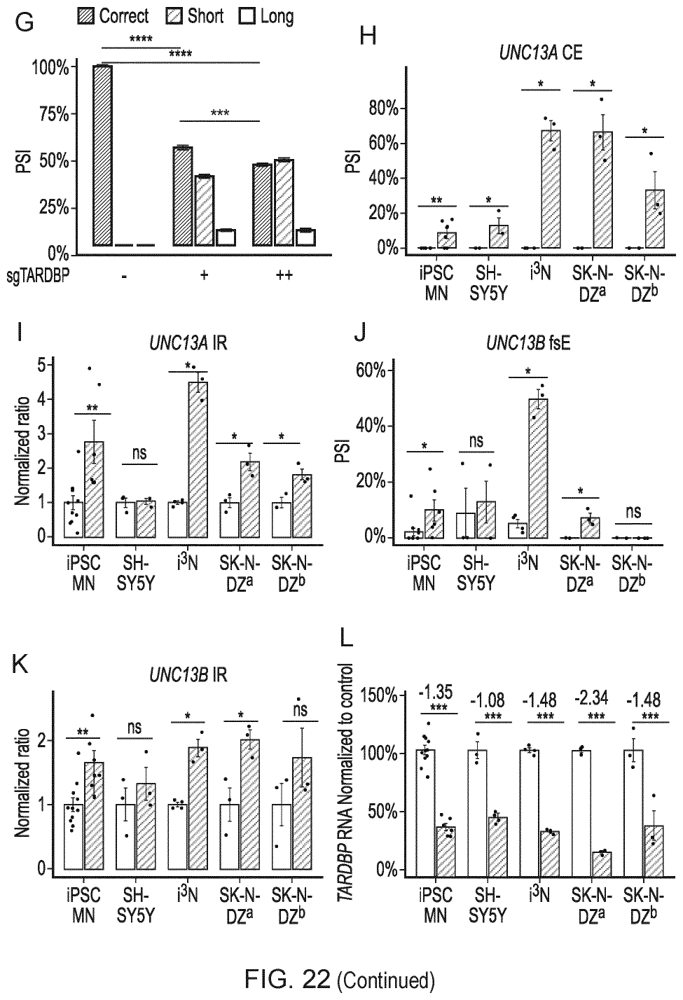

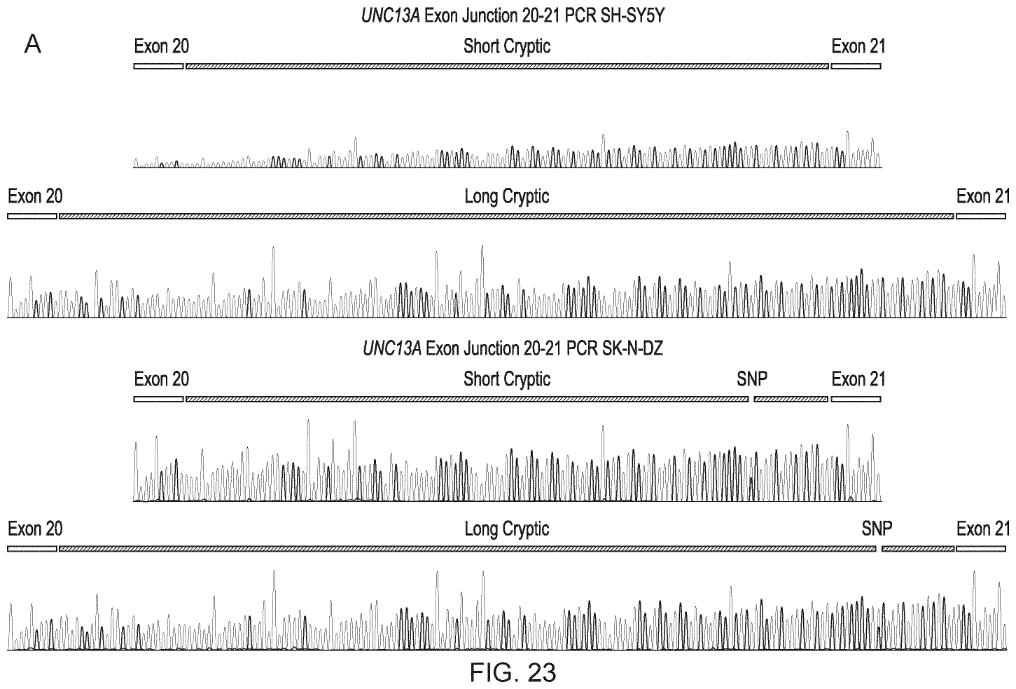

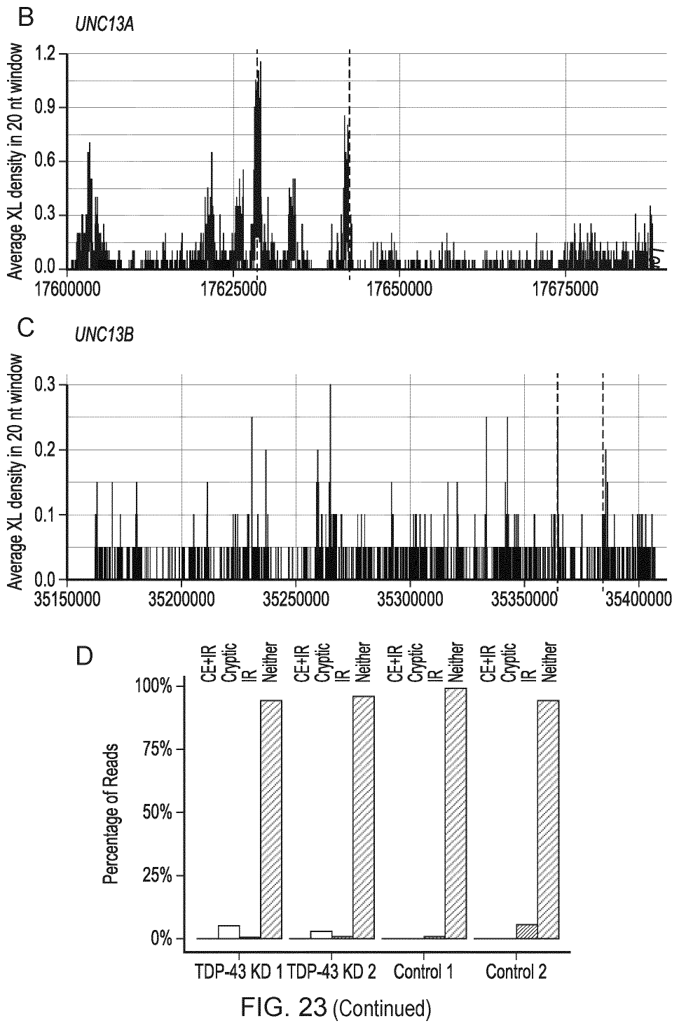

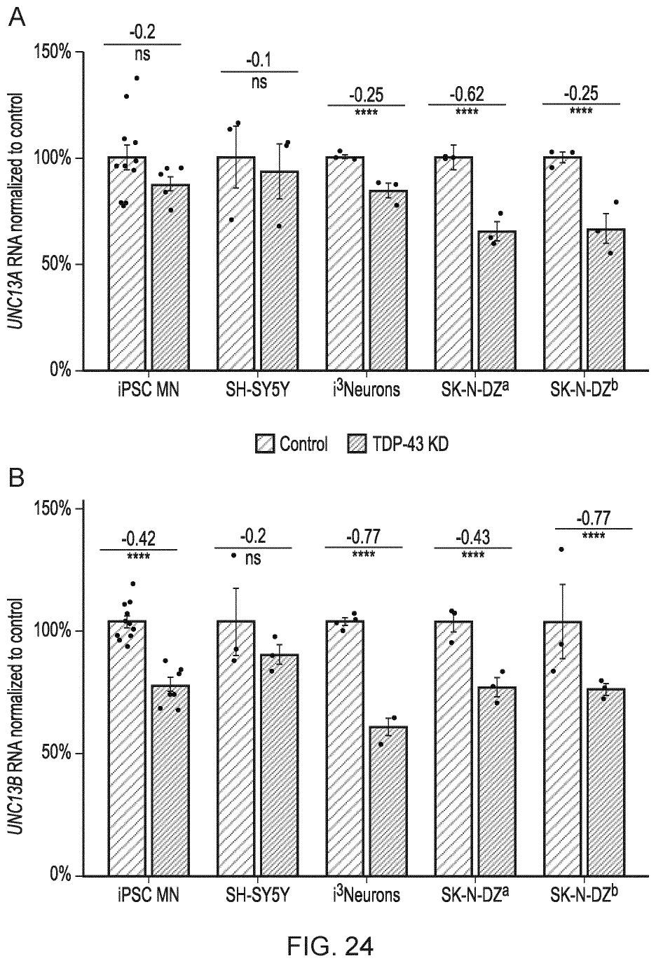

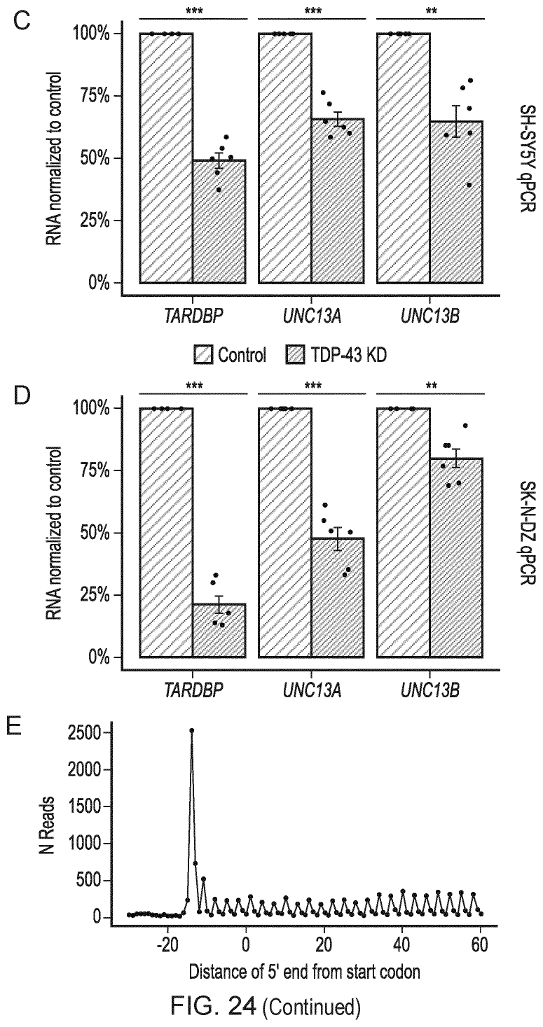

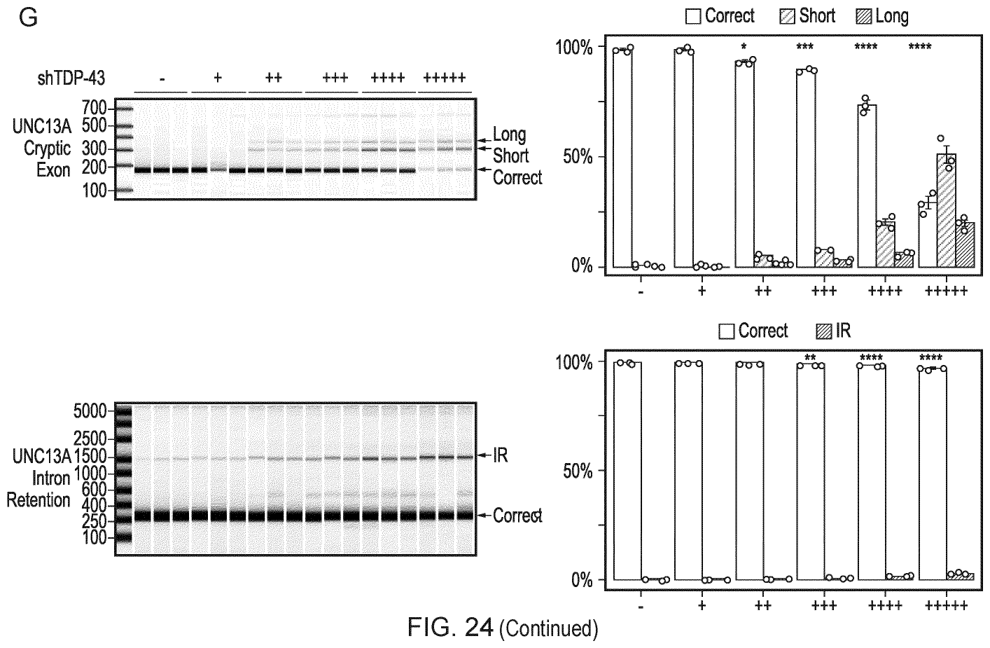

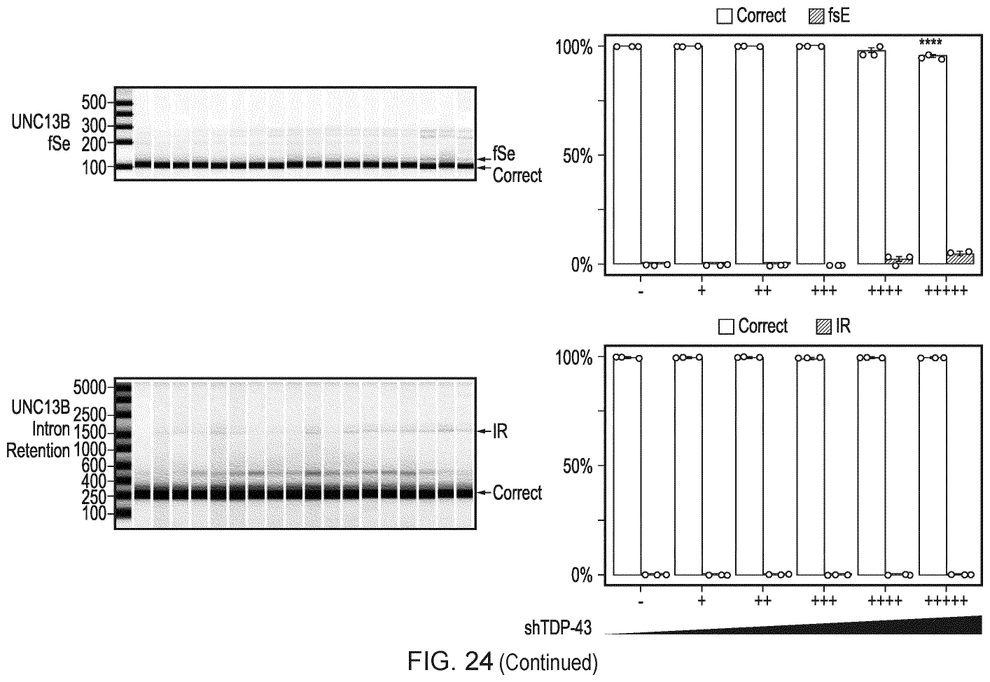

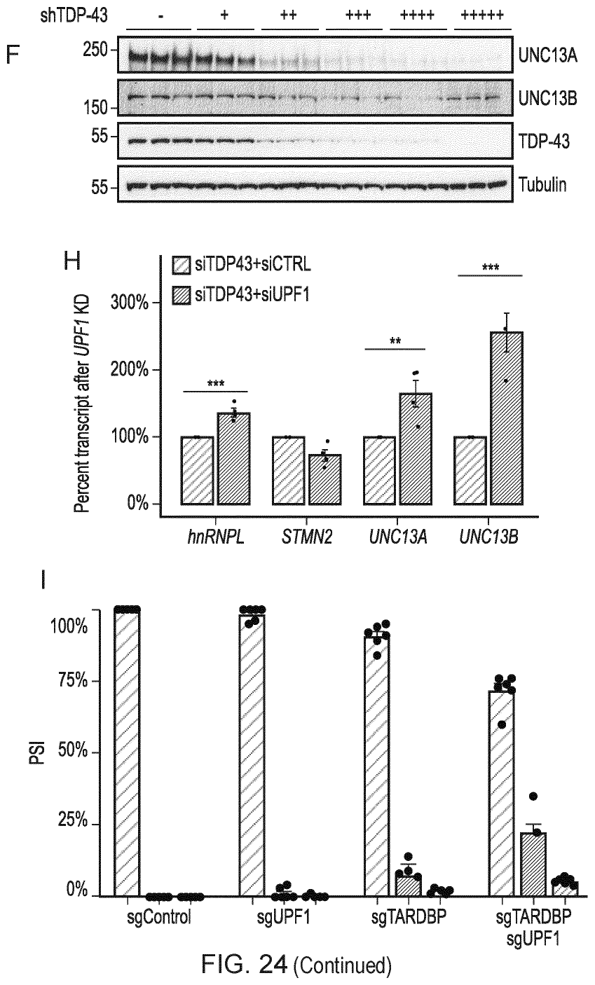

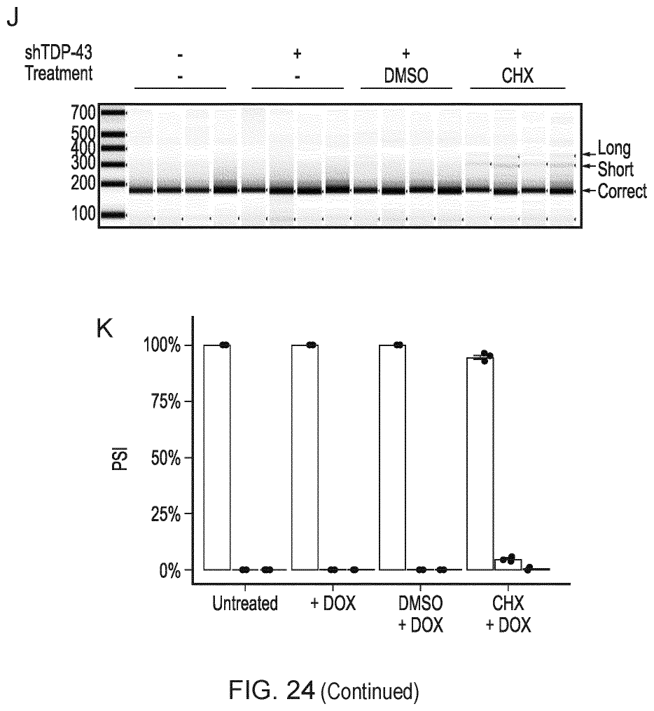

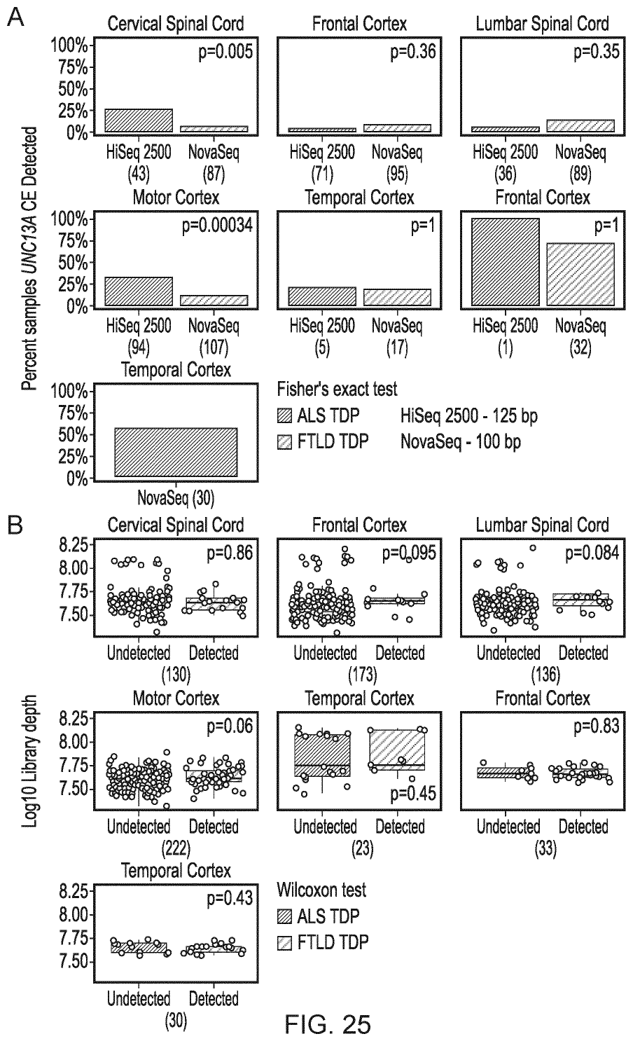

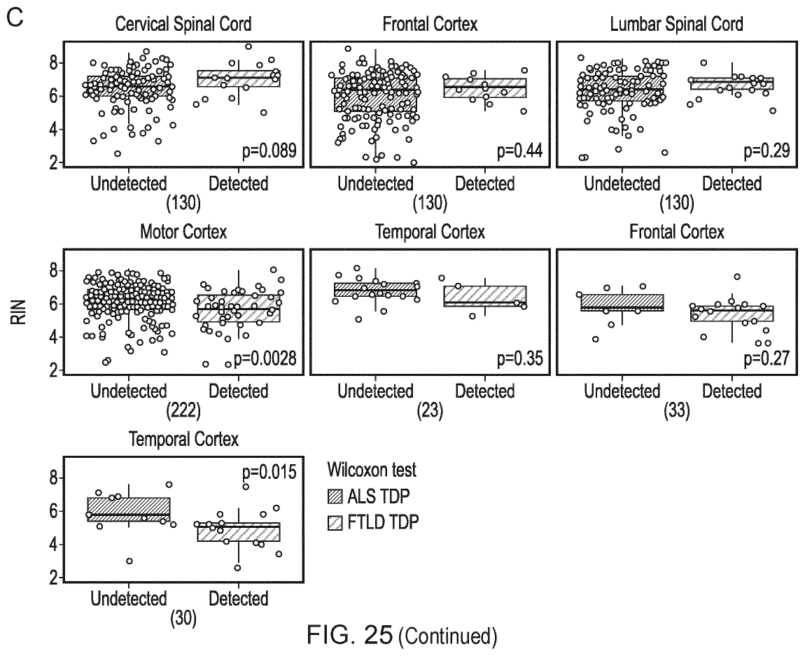

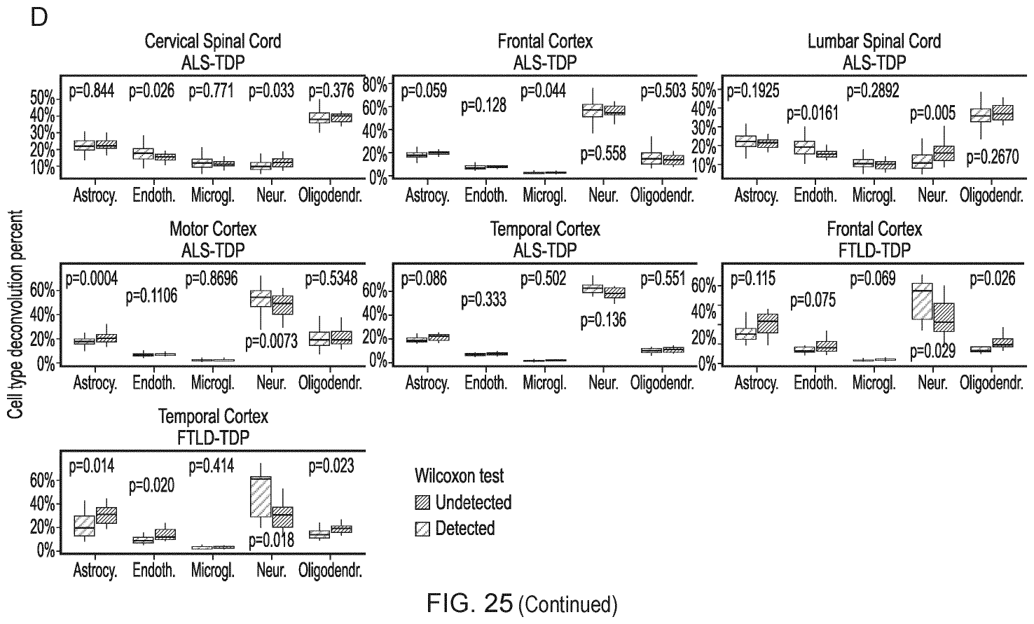

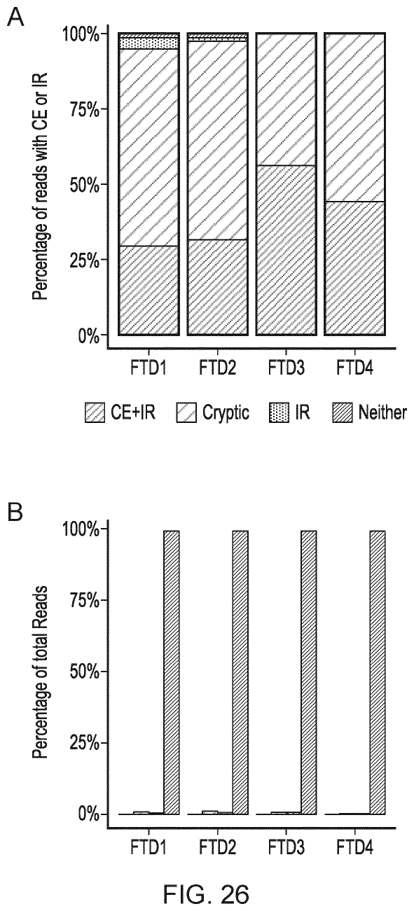

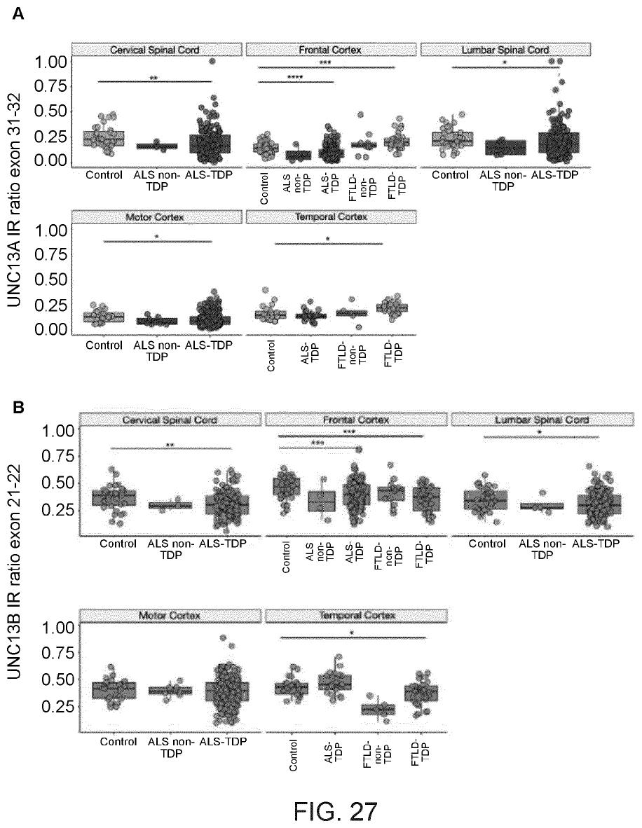

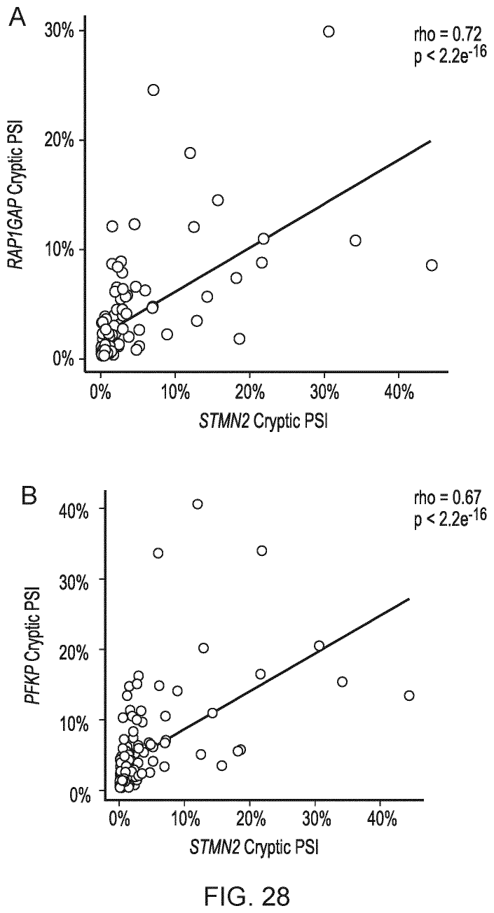

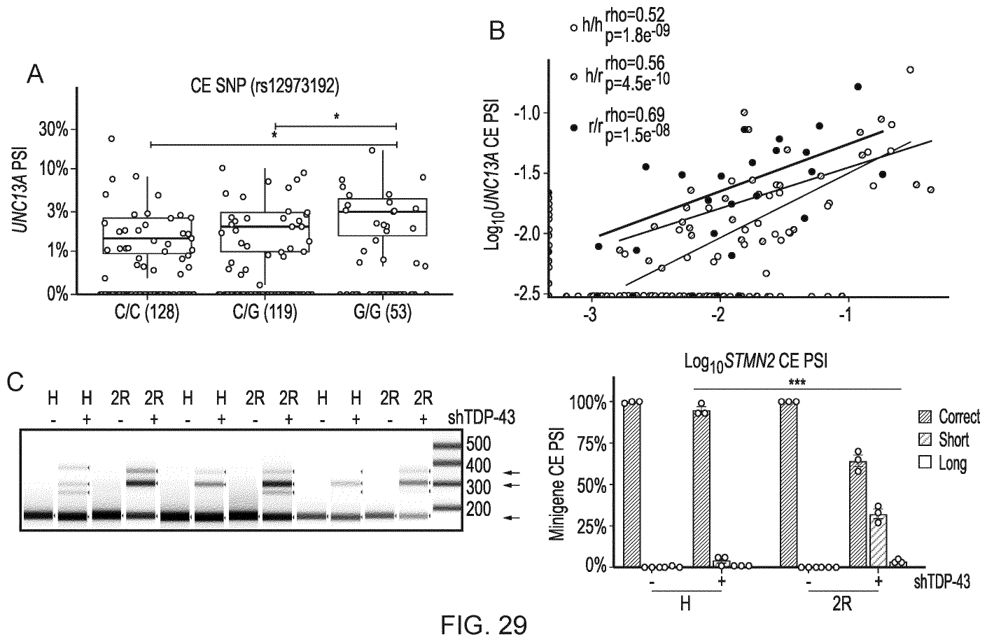

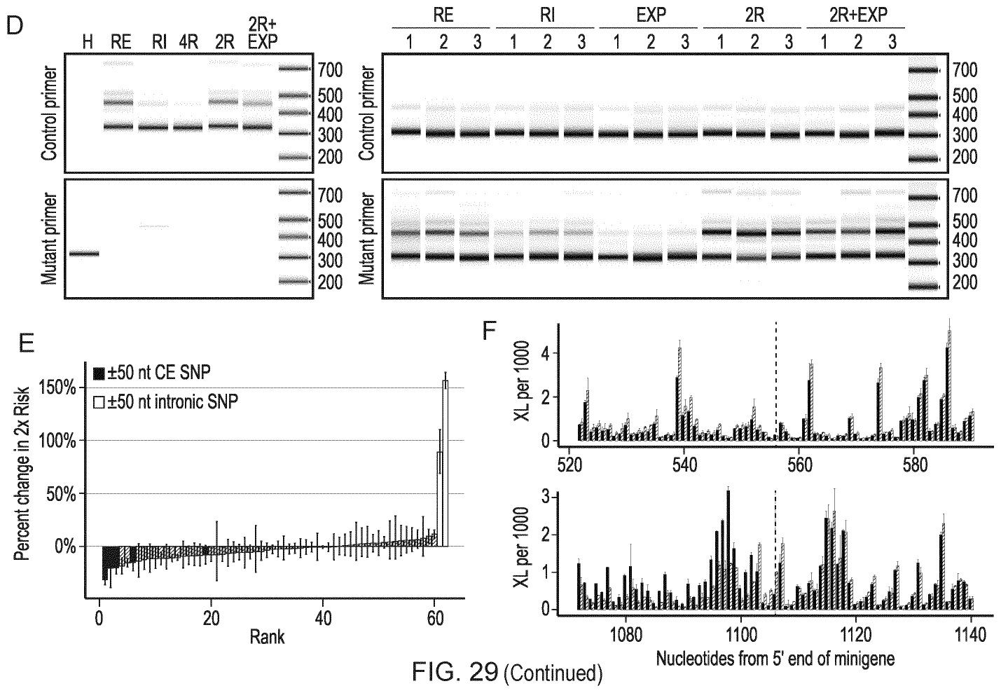

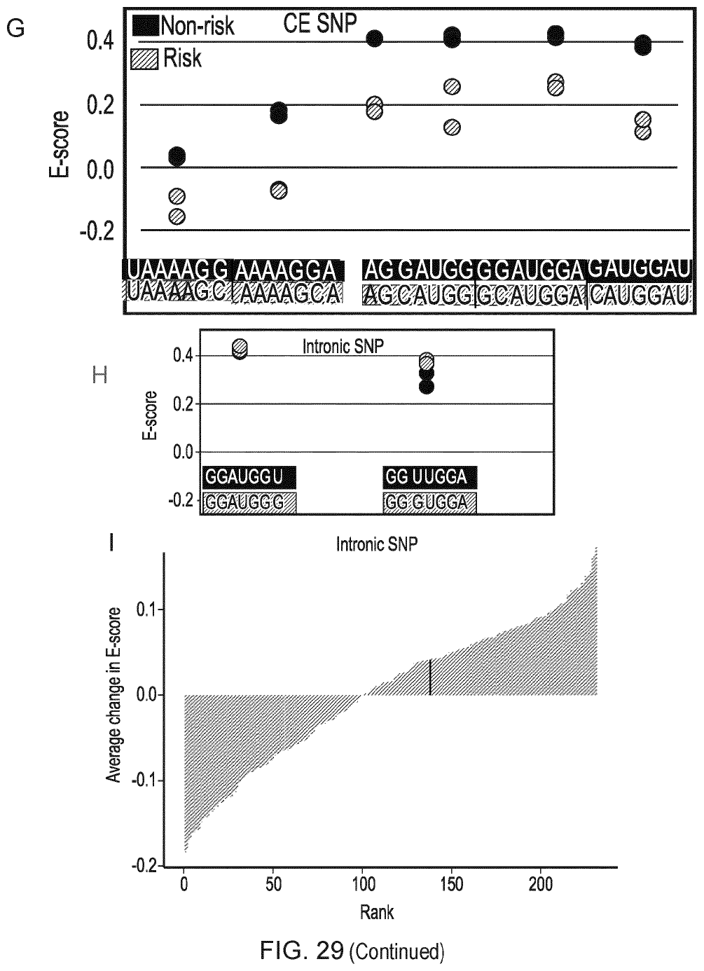

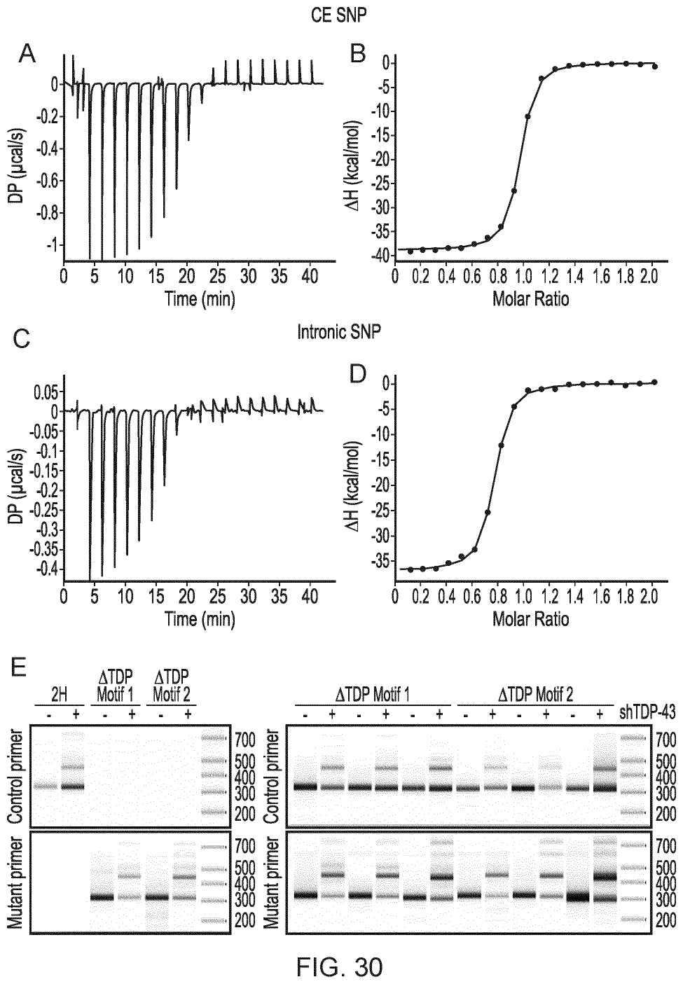

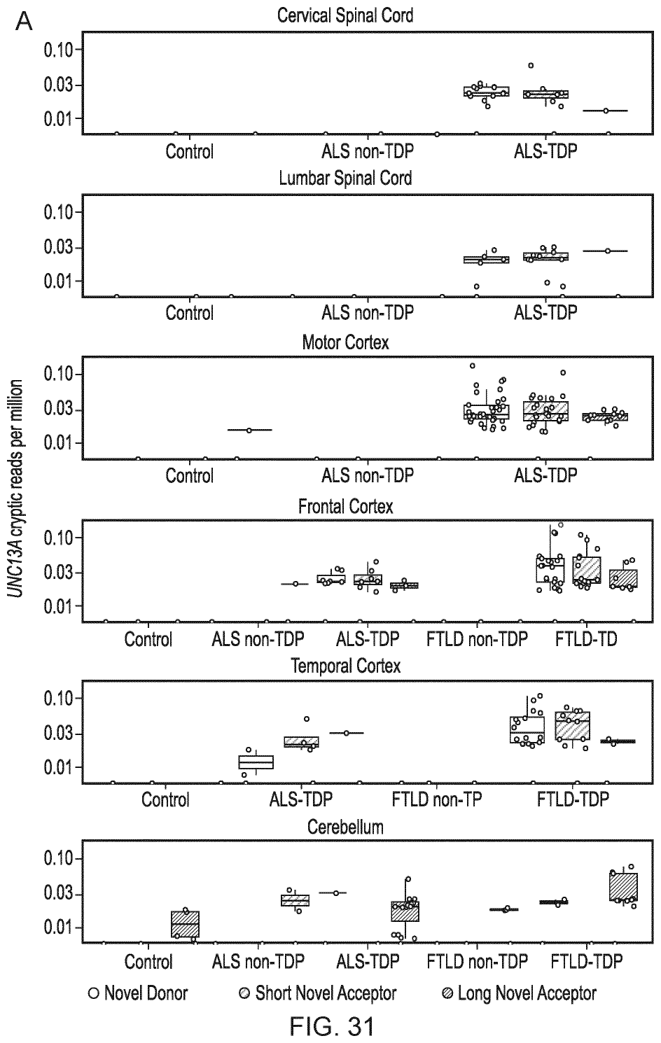

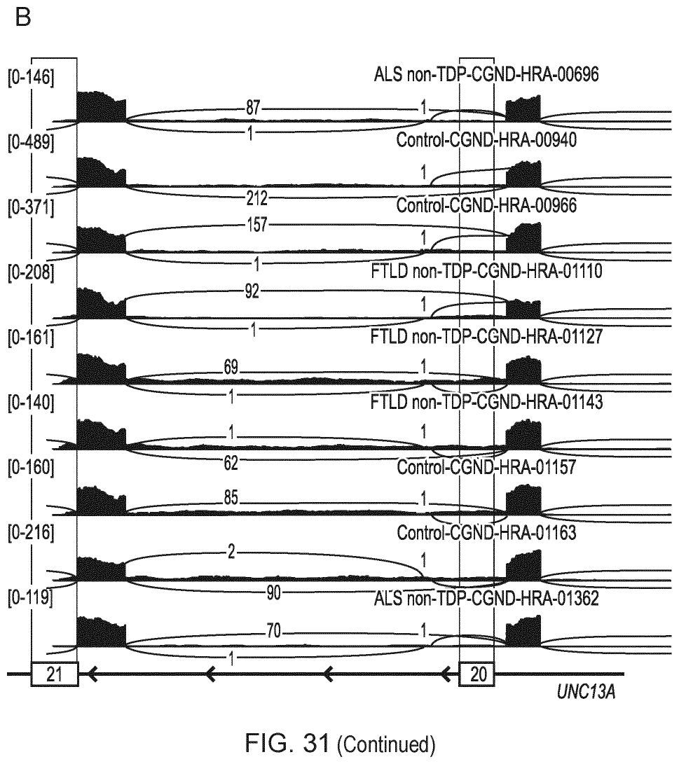

A schematic is provided in FIG. 17, which summarizes the strength of rescue of various ASOs which bind to the UNC13A cryptic exon and flanking intronic regions. The correct splicing event was rescued when ASOs with an asterisk (*) where used in combination with ASOs targeting the short acceptor or donor.

Furthermore, the rescue effect was effective both against target sequences featuring either variant of the rs12973192 SNP: the cell line used for these experiments expresses both alleles, and thus the large (50% to more than 90%) rescue observed would only be possible if ASOs block cryptic exon inclusion for transcripts with both variants

From these data it has been determined that through a cryptic exon, TDP-43 depletion in cells and patient brains induces a reduction of UNC13A transcripts and proteins, which are important players in synaptic function. Further, the UNC13A CE discovered is directly overlaps with one SNP and is in the same annotated intron as a second SNP, both of which have been identified in ALS and FTD GWAS: the risk variants of these SNPs increase the level of cryptic exon inclusion in vitro and in patients, thus implying that the CE directly contributes to ALS/FTD. These variants are associated with a more aggressive disease progression, supporting that increased inclusion of this CE and therefore a larger decrease in UNC13A protein, makes disease more aggressive. Novel therapeutics have been designed that target the UNC13A CE in order to inhibit inclusion of the UNC13A cryptic exon in the mature mRNA.

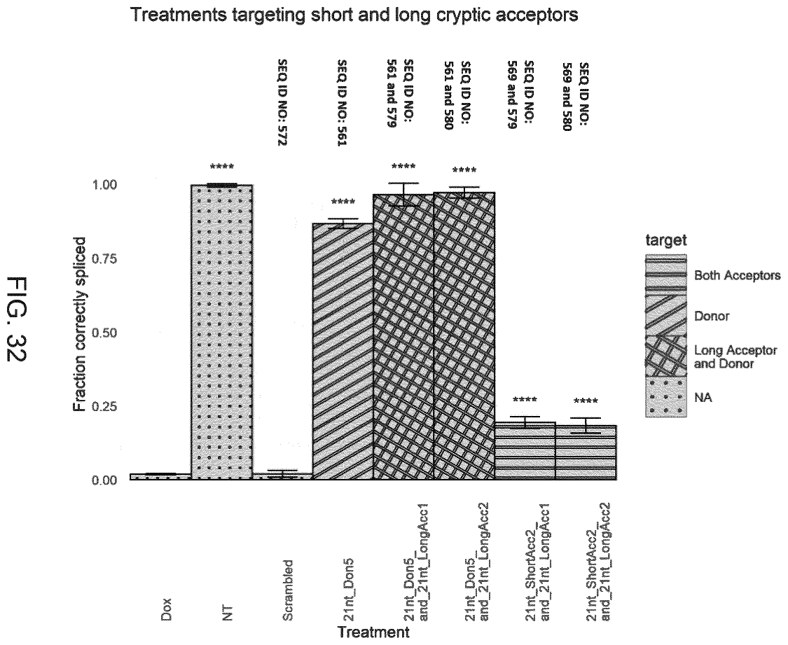

Combining ASOs targeting the acceptor splice site of the long CE with ASOs targeting either the acceptor splice site of the short CE, or the donor splice site of the CE, each at half of the normal concentration, also showed rescue (see FIG. 32).

These data suggest that targeting the acceptor splice site of the long CE together in combination with targeting the donor splice site or acceptor splice site of the short CE has a synergistic effect on positive rescue.

TABLE 6
Combinatorial targeting

| | | Seq IDs | Seq IDs | Seq IDs |
|---|---|---|---|---|
| | | of | of long | of short |
| | | donor | acceptor | acceptor |
| | Cell | targeting | targeting | targeting |
| Treatment | line | ASO | ASO | ASO |
| 21mer LNA donor + | SK-N- | SEQ ID | SEQ ID NO: | n/a |
| 21mer LNA long | DZ | NO: 561 | 579 or SEQ | |
| acceptor | | | ID NO: 580 | |
| 21mer LNA donor + | SK-N- | SEQ ID | n/a | SEQ ID |
| 21mer LNA short | DZ | NO: 560 | | NO: 570 |
| acceptor | | | | |
| 21mer LNA short | SK-N- | n/a | SEQ ID NO: | SEQ ID |
| acceptor + 21mer | DZ | | 579 or SEQ | NO: 569 |
| LNA long acceptor | | | ID NO: 580 | |

### Example 8: Rescue in SHSY5Y Cells by Donor and Acceptor Targeting ASOs

To assess the ability of different ASOs to induce the correct splicing event in disease-state cells, we transfected ASOs into SHSY5Y cells with doxycycline inducible TDP-43 knockdown and assessed endogenous UNC13A splicing via reverse transcription PCR (RT-PCR). These human neuroblastoma cells have served as a model for neurodegenerative disorders are thus a suitable model for this study.

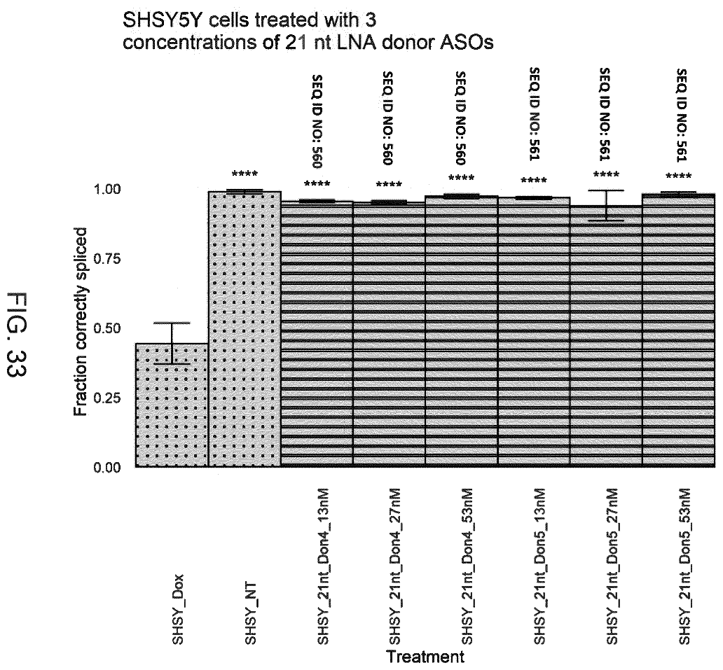

We found that ASOs targeting donor splice site can rescue the splicing in SHSY5Y cells (see FIG. 33).

### Example 9: Cell Culture and Differentiation of Human Induced Pluripotent Stem Cells into Neurons

Human induced pluripotent stem cell (hiPSC) line, WTC11 harboring an inducible neurogenin 2 transgene (Ngn2), and its TDP43 knockdown counterparts (n=4), were fully differentiated to glutamatergic “cortical-like” i3Neurons.

### Example 10: Quantification of TARDBP, UNC13A, and UNC13B Using Quantitative Proteomics

SP3 protein extraction was performed to extract intercellular proteins 1. Briefly, we harvested and lysed 2 million neurons per biological replicate in a very stringent buffer (50 mM HEPES, 50 mM NaCl, 5 mM EDTA 1% SDS, 1% Triton X-100, 1% NP-40, 1% Tween 20, 1% deoxycholate and 1% glycerol) supplemental with complete protease inhibitor cocktail at 1 tablet/10 ml ratio. The cell lysate was reduced by 10 mM dithiothreitol (30 min, 60° C.) and alkylated using 20 mM iodoacetamide (30 min, dark, room temperature). The denatured proteins were captured by hydrophilic magnetic beads, and tryptic on-beads digestion was conducted for 16 hours at 37° C. We injected 1 g resulting peptides to a nano liquid chromatography (LC) for separation, and subsequently those tryptic peptides were analyzed on an Orbitrap Eclipse mass spectrometer (MS) coupled with a FAIMS interface using data dependent acquisition (DDA) and data-independent acquisition (DIA). The peptides were separated on a 120-minute LC gradient with 2-35% solvent B (0.1% FA, 5% DSMO in acetonitrile), and FAIMS's compensation voltages were set to −50, −65 and −80. The DDA and DIA MS raw files were searched against Uniprot-Human-Proteome_UP000005640 database with 1% FDR using Proteome Discoverer (v2.4)2 and Spectronaut (v14.1)3, respectively. The raw intensity of quantified peptides was normalized by total peptides intensity identified in the same sample. The DDA quantified TARDBP- and UNC13A-derived unique and sharing peptides were parsed out and used for protein quantification. Specifically, we visualized and quantified the unique peptides of UNC13A using their MS/MS fragment ion intensity acquired by DIA.

### Example 11: RNA-Sequencing, Differential Gene Expression and Splicing Analysis

Public data for Klim was downloaded through Gene Expression Omnibus (GEO) series accession number: GSE121569, Appocher: GEO series accession number: GSE97262.

Samples were quality trimmed using Fastp with the parameter “qualified_quality_phred: 10” and aligned to the GRCh38 genome build using STAR (v2.7.0f) with gene models from GENCODE v31. Gene expression was quantified using FeatureCounts using gene models from GENCODE v31. Any gene which did not have an expression of at least 0.5 counts per million (CPM) in more than 2 samples was removed. For differential gene expression analysis, all samples were run in the same manner using the standard DESeq2 workflow without additional covariates, with the exception of the Klim hMN dataset, where we included the day of differentiation. Differential expression was defined at a Benjamini-Hochberg false discovery rate <0.1 Alignment pipeline available here: https://github.com/frattalab/ma_seq_snakemake.

Differential splicing was performed using MAJIQ (v2.1) using the GRCh38 reference genome. A threshold of 0.1 ΔPSI was used for calling the probability of significant change between groups. The results of the deltaPSI module were then parsed using custom R scripts to obtain a PSI and probability of change for each junction. Splicing pipeline available here: https://github.com/frattalab/splicing.

### Example 12: ALS GWAS Data

Harmonized summary statistics for the latest ALS GWAS (Nicolas, 2018, accession GCST005647) were downloaded from the NHGRI-EBI GWAS Catalog (Buniello, MacArthur et al., 2019). Locus plots were created using LocusZoom.

### Example 13: Analysis of New York Genome Center ALS Consortium Dataset

Patients with FTD were classified according to a pathologist's diagnosis of FTD with TDP-43 inclusions (FTLD-TDP), tau inclusions (FTLD-tau), or FUS inclusions (FTLD-FUS). ALS samples were divided into the following subcategories using the available Consortium metadata: ALS with or without reported SOD1 mutations (ALS-TDP and ALS-SOD1); ALS with frontotemporal dementia (ALS-FTLD); and ALS with AD (ALS-AD). All non-SOD1 ALS samples were grouped as “ALS-TDP” in this work for simplicity, although reporting of postmortem TDP-43 inclusions was not systematic and therefore not integrated into the metadata. Confirmed TDP-43 pathology postmortem was reported for all FTLD-TDP samples.

The NYGC ALS dataset contains 377 patients with 1349 neurological tissue samples, including non-neurological disease controls, FTLD, ALS, FTD with ALS (ALS-FTLD), or ALS with suspected Alzheimer's disease (ALS-AD). FTLD cases were further categorized based on neuropathological diagnosis, those with TDP-43 aggregates, FTLD-TDP-A, B, C or those with FUS or TAU aggregates. As the presence of TDP-43 proteinopathy has not been systematically assessed in the ALS cases, we separated patients into those with SOD1 or FUS mutations, which are presumed not to have TDP-43 proteinopathy, and all others.

Sample processing, library preparation, and RNA-seq quality control are known in the art have been extensively described in previous papers. In brief, RNA was extracted from flash-frozen postmortem tissue using TRIzol (Thermo Fisher Scientific) chloroform, and RNA-Seq libraries were prepared from 500 ng total RNA using the KAPA Stranded RNA-Seq Kit with RiboErase (KAPA Biosystems) for rRNA depletion. Pooled libraries (average insert size: 375 bp) passing the quality criteria were sequenced either on an Illumina HiSeq 2500 (125 bp paired end) or an Illumina NovaSeq (100 bp paired end). The samples had a median sequencing depth of 42 million read pairs, with a range between 16 and 167 million read pairs.

Samples were uniformly processed, including adapter trimming with Trimmomatic and alignment to the hg38 genome build using STAR (2.7.2a) with indexes from GENCODE v30. Extensive quality control was performed using SAMtools and Picard Tools to confirm sex and tissue of origin.

Uniquely mapped reads within the UNC13A locus were extracted from each sample using SAMtools. Any read marked as a PCR duplicate by Picard Tools was discarded. Splice junction reads were then extracted with RegTools using a minimum of 8 bp as an anchor on each side of the junction and a maximum intron size of 500 kb. Junctions from each sample were then clustered together using LeafCutter with relaxed junction filtering (minimum total reads per junction=30, minimum fraction of total cluster reads=0.0001). This produced a matrix of junction counts across all samples.

### Example 14: Generation of Stable TDP-43 Knockdown Cell Line

SK-N-DZ cells with doxycycline-inducible TDP-43 knockdown were generated by transducing SK-N-DZ cells with a SmartVector lentivirus (V3IHSHEG_6494503) containing a doxycycline-inducible shRNA cassette for TDP-43. Transduced cells were then selected with puromycin (1 μg/mL) for one week. Pooled TDP-43 knockdown SK-N-DZ cells were then plated as single cells and expanded to obtain a clonal population, to then select only the clone showing the strongest TDP-43 knockdown for subsequent stages.

### Example 15: Depletion of TDP-43 from Immortalized Human Cell Lines

SK-N-DZ cells were grown in DMEM/F12 containing Glutamax (Thermo) supplemented with 10% FBS (Thermo) and 1% PenStrep (Thermo). To induce shRNA against TDP-43, cells were treated with 5 μg/mL Doxycycline Hyclate (Sigma D9891). Antisense oligonucleotide treatment was performed after 3 days and, after a further 3 days, cells were harvested for RNA.

### Example 16: ASO Synthesis

ASO synthesis ASOs were ordered from Integrated DNA Technologies. Each featured a 100% phosphorothioate modified backbone, and either partial LNA substitutions (33-50%) or 100% 2′-O-methyl sugar substitutions.

### Example 17: ASO Treatment

In some embodiments as described, prior to transfection, 70-90% confluent cells were plated on 12-wells plates, with 3 replicates per condition. For each well, 6 μL of Lipofectamine RNAiMax (Thermo Fisher Scientific) and 4 μL of 10 μM ASO were diluted in 150 μL Optimem (Thermo Fisher Scientific) and, after 5 minutes incubation at room temperature, the mix was transferred into the well followed by 600 μL of growing medium (DMEM/F12+Glutamax) with 5 μg/mL doxycycline. For treatments in which a mixture of ASOs was used, 2 μL of each 10 μM ASO was used (4 μL total). For SHSY5Y cells, 4 μL of 2.5 μM, 5 μM or 10 μM ASO were diluted for a final concentration of 13, 27 or 53 nM.

### Example 18: RNA Extraction and Reverse Transcription

RNA extraction from SK-N-DZ cells was performed using the RNeasy kit (Qiagen) following the manufacturer's protocol including the on-column DNA digestion step. After measuring RNA concentrations by Nanodrop, 1000 ng of RNA was used for reverse transcription. First strand cDNA synthesis was performed with RevertAid (Thermo K1622) following the manufacturer's protocol with random hexamer primers.

### Example 19: PCR

The UNC13A transcript was amplified either via a nested approach or with a single primer set. For the nested approach, the cDNA was first amplified for 12 cycles using primers: SEQ ID NO: 573 Nest1_F: GACATCAAATCCCGCGTGAA; and SEQ ID NO: 574 Nest1_R: CATTGATGTTGGCGAGCAGG. This was followed by 24 cycles with primers: SEQ ID NO: 575 Nest2_F: CAGACGATCATTGAGGTGCG; and SEQ ID NO: 576 Nest2_R: ATACTTGGAGGAGAGGCAGG. For the single primer approach, cDNAs were amplified using primers: SEQ ID NO: 577 Single F: CAAGCGAACTGACAAATC; and SEQ ID NO: 578 Single_R: CTGGGATCTTCACGACC. In both cases, PCR was performed using Phusion HF 2× Master Mix (Thermo Fisher Scientific), using an annealing temperature of 64 degrees Celsius. PCR results were analyzed and quantified using a QIAxcel (Qiagen) with the DNA screening cassette, using a 30 s injection time.

1.-27. (canceled)

28. A method of treating a neurodegenerative disorder, comprising:
administering to a subject in need thereof a therapeutically effective amount of an antisense oligonucleotide (ASO) comprising a nucleotide sequence of 13-30 nucleotides; or
administering to a subject in need thereof a therapeutically effective amount of a pharmaceutical composition comprising one or more ASOs comprising a nucleotide sequence of 13-30 nucleotides;
wherein the ASO nucleotide sequence has sequence complementarity with SEQ ID NO 1.

29. The method of claim 28, wherein the neurodegenerative disorder is associated with TDP-43 pathology.

30. The method of claim 28, wherein the neurodegenerative disorder is amyotrophic lateral sclerosis (ALS), frontotemporal dementia (FTD), Alzheimer's disease, Parkinson's disease, facial onset sensory and motor neuronopathy (FOSMN), Perry Syndrome, or any combination thereof.

31. The method of claim 30, wherein the neurodegenerative disorder is ALS.

32. The method of claim 31, wherein the ALS is familial ALS or sporadic ALS.

33. The method of claim 28, wherein the ASO comprises 20-24 nucleotides.

34. The method of claim 28, wherein the ASO binds to an UNC13A cryptic exon or intronic flanking regions thereof.

35. The method of claim 28, wherein the ASO comprises a sequence corresponding to SEQ ID NO: 4-546.

36. The method of claim 28, wherein the ASO comprises a sequence corresponding to SEQ ID NO 270-352.

37. The method of claim 28, wherein the ASO comprises a sequence corresponding to SEQ ID NO 295-324.

38. The method of claim 28, wherein the ASO comprises a sequence corresponding to any one or more of SEQ ID NO: 105-189.

39. The method of claim 28, wherein the ASO has a backbone selected from RNA, locked nucleic acid (LNA), tri-cyclo DNA (tcDNA), hexitol nucleic acids (HNA), threose nucleic acid (TNA), morpholino oligomer (PMO), peptide nucleic acid (PNA), 2-OMe-RNA, 2′-O-methoxyethyl (MOE) nucleic acids, or 2-O-(2-methylcarbomoyl) (MCE) nucleotides, or any combination thereof.

40. The method of claim 28, wherein the ASO comprises one or more phosphorothioate linkages.

41. The method of claim 28, wherein the pharmaceutical composition comprising the one or more ASOs further comprises a pharmaceutical carrier, diluent or excipient.

42. An ASO comprising a nucleotide sequence of 13-30 nucleotides, wherein the nucleotide sequence has sequence complementarity with SEQ ID NO 1.

43. The ASO of claim 42, wherein the ASO comprises 20-24 nucleotides.

44. The ASO of claim 42, wherein the ASO binds to an UNC13A cryptic exon or intronic flanking regions thereof.

45. The ASO of claim 42, wherein the ASO comprises a sequence corresponding to SEQ ID NO: 4-546.

46. The ASO of claim 42, wherein the ASO comprises a sequence corresponding to SEQ ID NO 270-352.

47. The ASO of claim 46, wherein the ASO comprises a sequence corresponding to SEQ ID NO 295-324.

48. The ASO of claim 42, wherein the splice site is an acceptor splice site.

49. The ASO of claim 48, wherein the ASO comprises a sequence corresponding to any one or more of SEQ ID NO: 105-189.

50. The ASO of claim 42, wherein the ASO has a backbone selected from RNA, LNA, tcDNA, HNA, TNA, PMO, PNA, 2-OMe-RNA, 2′-O-MOE nucleic acids, or 2-O-(2-MCE) nucleotides, or any combination thereof.

51. The ASO of claim 42, wherein the ASO comprises one or more phosphorothioate linkages.

52. An ASO comprising a nucleotide sequence of 13-30 nucleotides, wherein the ASO modulates splicing by preventing inclusion of an UNC13A cryptic exon into an UNC13A mature mRNA.

53. A pharmaceutical composition comprising one or more ASOs according to claim 42.

54. A pharmaceutical composition according to claim 53, further comprising a pharmaceutical carrier, diluent or excipient.


SEQUENCE LISTING (580 sequences)

SEQ ID NO: 1 - RNA, 1288 nt, Homo sapiens
  misc_feature (414)..(414): G can be replaced with C at this position
  misc_feature (964)..(964): U can be replaced with G at this position
gugagggucauugcucggccccucccaugccacuuccacucaccauuccugccugcccagcucuuccucuuucuggccacaccauccacacucuccuggcccucugagacugcccgccaugccauucccuuuaccuggaaaacuccucccuauccaucaaaguccagauucagggucaccuccucugggaagcccaccuuggccuccagguugacucucacuacucaucaucagguucuuccuucuauuccagcccuaaccacucaggauugggccguuugugucuggguaugucucuuccagcugccuggguuuccuggaaagaacucuuauccccaggaacuaguuuguugaauaaaugcuggugaaugaaugaaugauugaacagaugaaugagugaugaguagauaaaaggauggauggagagaugggugaguacauggauggauagauggaugaguugguggguagauucguggcuagauggaugauggauggauggacagauggauggauauaugauugaacuauugaaaguauagauguauggaugggugaauuuggggguaauuguuagaugauggaugaguauagaugaaugauggauggauaacuugaugaguggauagauagauugcuggauagaugauugacuggguggauagaugaaauguuggaugagcagauuaaguuguauuggaugggauggauggaagugugguugaguuauuagaaggaagauugaguagauaggugaauuuguugauagucagauggguagauagguagauggauggauggauggauggauguauaggcagauggacaaauggaugaauggguggguggaugaauggaaggaugugugguugaacuauugcaaguauugauaauuggguucauaauuucugaauauuuagauggaugguugugaguggcugguggacagacgaaaaauggaugguuggauaaauugauggguggauggaugguugguuguaugaaagaaugaaugauuggguagguggauuaaguugcggaucaauguaugggauggaugaauggauggauggauggaugugugguugaauuacugaaagguuggaagaguggaugggugaaauuugggguaguuagauggguggguguguggauggauaaaagaguagaugaaugaauuaaugaauaaacaggcagauggaugauguaagcugccccagacccugggaccucugacccccggcgaccccuugcacucuccaugacacuuucucucccaugguggcag

SEQ ID NO: 2 - RNA, 128 nt, Homo sapiens
  misc_feature (112)..(112): G can be replaced with C at this position
cugccuggguuuccuggaaagaacucuuauccccaggaacuaguuuguugaauaaaugcuggugaaugaaugaaugauugaacagaugaaugagugaugaguagauaaaaggauggauggagagaugg

SEQ ID NO: 3 - RNA, 178 nt, Homo sapiens
  misc_feature (162)..(162): G at this position can be replaced with C
cccuaaccacucaggauugggccguuugugucuggguaugucucuuccagcugccuggguuuccuggaaagaacucuuauccccaggaacuaguuuguugaauaaaugcuggugaaugaaugaaugauugaacagaugaaugagugaugaguagauaaaaggauggauggagagaugg

SEQ ID NO: 4 - RNA, 13 nt, Artificial Sequence
  Synthetic sequence
  misc_feature (1)..(13): U can be replaced with T
  misc_feature (1)..(13): U can be replaced by T. Any base can be replaced with a modified base.
ggaguuuuccagg

SEQ ID NO: 5 - RNA, 13 nt, Artificial Sequence
  Synthetic sequence
  misc_feature: U can be replaced by T. Any base can be replaced with a modified base.
aggaguuuuccag

SEQ ID NO: 6 - RNA, 13 nt, Artificial Sequence
  Synthetic sequence
  misc_feature (1)..(13): U can be replaced by T. Any base can be replaced with a modified base.
gaggaguuuucca

SEQ ID NO: 7 - RNA, 13 nt, Artificial Sequence
  Synthetic sequence
  misc_feature (1)..(13): U can be replaced by T. Any base can be replaced with a modified base.
ggaggaguuuucc

SEQ ID NO: 8 - RNA, 13 nt, Artificial Sequence
  Synthetic sequence
  misc_feature (1)..(13): U can be replaced by T. Any base can be replaced with a modified base.
gggaggaguuuuc

SEQ ID NO: 9 - RNA, 13 nt, Artificial Sequence
  Synthetic sequence
  misc_feature (1)..(13): U can be replaced by T. Any base can be replaced with a modified base.
agggaggaguuuu

SEQ ID NO: 10 - RNA, 13 nt, Artificial Sequence
  Synthetic sequence
  misc_feature (1)..(13): U can be replaced by T. Any base can be replaced with a modified base.
uagggaggaguuu

SEQ ID NO: 11 - RNA, 13 nt, Artificial Sequence
  Synthetic sequence
  misc_feature (1)..(13): U can be replaced by T. Any base can be replaced with a modified base.
auagggaggaguu

SEQ ID NO: 12 - RNA, 13 nt, Artificial Sequence
  Synthetic sequence
  misc_feature (1)..(13): U can be replaced by T. Any base can be replaced with a modified base.
gauagggaggagu

SEQ ID NO: 13 - RNA, 13 nt, Artificial Sequence
  Synthetic sequence
  misc_feature (1)..(13): U can be replaced by T. Any base can be replaced with a modified base.
ggauagggaggag

SEQ ID NO: 14 - RNA, 13 nt, Artificial Sequence
  Synthetic sequence
  misc_feature (1)..(13): U can be replaced by T. Any base can be replaced with a modified base.
uggauagggagga

SEQ ID NO: 15 - RNA, 13 nt, Artificial Sequence
  Synthetic sequence
  misc_feature (1)..(13): U can be replaced by T. Any base can be replaced with a modified base.
auggauagggagg

SEQ ID NO: 16 - RNA, 13 nt, Artificial Sequence
  Synthetic sequence
  misc_feature (1)..(13): U can be replaced by T. Any base can be replaced with a modified base.
gauggauagggag

SEQ ID NO: 17 - RNA, 13 nt, Artificial Sequence
  Synthetic sequence
  misc_feature (1)..(13): U can be replaced by T. Any base can be replaced with a modified base.
ugauggauaggga

SEQ ID NO: 18 - RNA, 13 nt, Artificial Sequence
  Synthetic sequence
  misc_feature (1)..(13): U can be replaced by T. Any base can be replaced with a modified base.
uugauggauaggg

SEQ ID NO: 19 - RNA, 13 nt, Artificial Sequence
  Synthetic sequence
  misc_feature (1)..(13): U can be replaced by T. Any base can be replaced with a modified base.
uuugauggauagg

SEQ ID NO: 20 - RNA, 13 nt, Artificial Sequence
  Synthetic sequence
  misc_feature (1)..(13): U can be replaced by T. Any base can be replaced with a modified base.
cuuugauggauag

SEQ ID NO: 21 - RNA, 13 nt, Artificial Sequence
  Synthetic sequence
  misc_feature (1)..(13): U can be replaced by T. Any base can be replaced with a modified base.
acuuugauggaua

SEQ ID NO: 22 - RNA, 13 nt, Artificial Sequence
  Synthetic sequence
  misc_feature (1)..(13): U can be replaced by T. Any base can be replaced with a modified base.
gacuuugauggau

SEQ ID NO: 23 - RNA, 13 nt, Artificial Sequence
  Synthetic sequence
  misc_feature (1)..(13): U can be replaced by T. Any base can be replaced with a modified base.
ggacuuugaugga

SEQ ID NO: 24 - RNA, 13 nt, Artificial Sequence
  Synthetic sequence
  misc_feature (1)..(13): U can be replaced by T. Any base can be replaced with a modified base.
uggacuuugaugg

SEQ ID NO: 25 - RNA, 13 nt, Artificial Sequence
  Synthetic sequence
  misc_feature (1)..(13): U can be replaced by T. Any base can be replaced with a modified base.
cuggacuuugaug

SEQ ID NO: 26 - RNA, 13 nt, Artificial Sequence
  Synthetic sequence
  misc_feature (1)..(13): U can be replaced by T. Any base can be replaced with a modified base.
ucuggacuuugau

SEQ ID NO: 27 - RNA, 13 nt, Artificial Sequence
  Synthetic sequence
  misc_feature (1)..(13): U can be replaced by T. Any base can be replaced with a modified base.
aucuggacuuuga

SEQ ID NO: 28 - RNA, 13 nt, Artificial Sequence
  Synthetic sequence
  misc_feature (1)..(13): U can be replaced by T. Any base can be replaced with a modified base.
aaucuggacuuug

SEQ ID NO: 29 - RNA, 13 nt, Artificial Sequence
  Synthetic sequence
  misc_feature (1)..(13): U can be replaced by T. Any base can be replaced with a modified base.
gaaucuggacuuu

SEQ ID NO: 30 - RNA, 13 nt, Artificial Sequence
  Synthetic sequence
  misc_feature (1)..(13): U can be replaced by T. Any base can be replaced with a modified base.
ugaaucuggacuu

SEQ ID NO: 31 - RNA, 13 nt, Artificial Sequence
  Synthetic sequence
  misc_feature (1)..(13): U can be replaced by T. Any base can be replaced with a modified base.
cugaaucuggacu

SEQ ID NO: 32 - RNA, 13 nt, Artificial Sequence
  Synthetic sequence
  misc_feature (1)..(13): U can be replaced by T. Any base can be replaced with a modified base.
ccugaaucuggac

SEQ ID NO: 33 - RNA, 13 nt, Artificial Sequence
  Synthetic sequence
  misc_feature (1)..(13): U can be replaced by T. Any base can be replaced with a modified base.
cccugaaucugga

SEQ ID NO: 34 - RNA, 13 nt, Artificial Sequence
  Synthetic sequence
  misc_feature (1)..(13): U can be replaced by T. Any base can be replaced with a modified base.
acccugaaucugg

SEQ ID NO: 35 - RNA, 13 nt, Artificial Sequence
  Synthetic sequence
  misc_feature (1)..(13): U can be replaced by T. Any base can be replaced with a modified base.
gacccugaaucug

SEQ ID NO: 36 - RNA, 13 nt, Artificial Sequence
  Synthetic sequence
  misc_feature (1)..(13): U can be replaced by T. Any base can be replaced with a modified base.
ugacccugaaucu

SEQ ID NO: 37 - RNA, 13 nt, Artificial Sequence
  Synthetic sequence
  misc_feature (1)..(13): U can be replaced by T. Any base can be replaced with a modified base.
gugacccugaauc

SEQ ID NO: 38 - RNA, 13 nt, Artificial Sequence
  Synthetic sequence
  misc_feature (1)..(13): U can be replaced by T. Any base can be replaced with a modified base.
ggugacccugaau

SEQ ID NO: 39 - RNA, 13 nt, Artificial Sequence
  Synthetic sequence
  misc_feature (1)..(13): U can be replaced by T. Any base can be replaced with a modified base.
aggugacccugaa

SEQ ID NO: 40 - RNA, 13 nt, Artificial Sequence
  Synthetic sequence
  misc_feature (1)..(13): U can be replaced by T. Any base can be replaced with a modified base.
gaggugacccuga

SEQ ID NO: 41 - RNA, 13 nt, Artificial Sequence
  Synthetic sequence
  misc_feature (1)..(13): U can be replaced by T. Any base can be replaced with a modified base.
ggaggugacccug

SEQ ID NO: 42 - RNA, 13 nt, Artificial Sequence
  Synthetic sequence
  misc_feature (1)..(13): U can be replaced by T. Any base can be replaced with a modified base.
aggaggugacccu

SEQ ID NO: 43 - RNA, 13 nt, Artificial Sequence
  Synthetic sequence
  misc_feature (1)..(13): U can be replaced by T. Any base can be replaced with a modified base.
gaggaggugaccc

SEQ ID NO: 44 - RNA, 13 nt, Artificial Sequence
  Synthetic sequence
  misc_feature (1)..(13): U can be replaced by T. Any base can be replaced with a modified base.
agaggaggugacc

SEQ ID NO: 45 - RNA, 13 nt, Artificial Sequence
  Synthetic sequence
  misc_feature (1)..(13): U can be replaced by T. Any base can be replaced with a modified base.
cagaggaggugac

SEQ ID NO: 46 - RNA, 13 nt, Artificial Sequence
  Synthetic sequence
  misc_feature (1)..(13): U can be replaced by T. Any base can be replaced with a modified base.
ccagaggagguga

SEQ ID NO: 47 - RNA, 13 nt, Artificial Sequence
  Synthetic sequence
  misc_feature (1)..(13): U can be replaced by T. Any base can be replaced with a modified base.
cccagaggaggug

SEQ ID NO: 48 - RNA, 13 nt, Artificial Sequence
  Synthetic sequence
  misc_feature (1)..(13): U can be replaced by T. Any base can be replaced with a modified base.
ucccagaggaggu

SEQ ID NO: 49 - RNA, 13 nt, Artificial Sequence
  Synthetic sequence
  misc_feature (1)..(13): U can be replaced by T. Any base can be replaced with a modified base.
uucccagaggagg

SEQ ID NO: 50 - RNA, 13 nt, Artificial Sequence
  Synthetic sequence
  misc_feature (1)..(13): U can be replaced by T. Any base can be replaced with a modified base.
cuucccagaggag

SEQ ID NO: 51 - RNA, 13 nt, Artificial Sequence
  Synthetic sequence
  misc_feature (1)..(13): U can be replaced by T. Any base can be replaced with a modified base.
gcuucccagagga

SEQ ID NO: 52 - RNA, 13 nt, Artificial Sequence
  Synthetic sequence
  misc_feature (1)..(13): U can be replaced by T. Any base can be replaced with a modified base.
ggcuucccagagg

SEQ ID NO: 53 - RNA, 13 nt, Artificial Sequence
  Synthetic sequence
  misc_feature (1)..(13): U can be replaced by T. Any base can be replaced with a modified base.
gggcuucccagag

SEQ ID NO: 54 - RNA, 13 nt, Artificial Sequence
  Synthetic sequence
  misc_feature (1)..(13): U can be replaced by T. Any base can be replaced with a modified base.
ugggcuucccaga

SEQ ID NO: 55 - RNA, 13 nt, Artificial Sequence
  Synthetic sequence
  misc_feature (1)..(13): U can be replaced by T. Any base can be replaced with a modified base.
gugggcuucccag

SEQ ID NO: 56 - RNA, 13 nt, Artificial Sequence
  Synthetic sequence
  misc_feature (1)..(13): U can be replaced by T. Any base can be replaced with a modified base.
ggugggcuuccca

SEQ ID NO: 57 - RNA, 13 nt, Artificial Sequence
  Synthetic sequence
  misc_feature (1)..(13): U can be replaced by T. Any base can be replaced with a modified base.
aggugggcuuccc

SEQ ID NO: 58 - RNA, 13 nt, Artificial Sequence
  Synthetic sequence
  misc_feature (1)..(13): U can be replaced by T. Any base can be replaced with a modified base.
aaggugggcuucc

SEQ ID NO: 59 - RNA, 13 nt, Artificial Sequence
  Synthetic sequence
  misc_feature (1)..(13): U can be replaced by T. Any base can be replaced with a modified base.
caaggugggcuuc

SEQ ID NO: 60 - RNA, 13 nt, Artificial Sequence
  Synthetic sequence
  misc_feature (1)..(13): U can be replaced by T. Any base can be replaced with a modified base.
ccaaggugggcuu

SEQ ID NO: 61 - RNA, 13 nt, Artificial Sequence
  Synthetic sequence
  misc_feature (1)..(13): U can be replaced by T. Any base can be replaced with a modified base.
gccaaggugggcu

SEQ ID NO: 62 - RNA, 13 nt, Artificial Sequence
  Synthetic sequence
  misc_feature (1)..(13): U can be replaced by T. Any base can be replaced with a modified base.
ggccaaggugggc

SEQ ID NO: 63 - RNA, 13 nt, Artificial Sequence
  Synthetic sequence
  misc_feature (1)..(13): U can be replaced by T. Any base can be replaced with a modified base.
aggccaagguggg

SEQ ID NO: 64 - RNA, 13 nt, Artificial Sequence
  Synthetic sequence
  misc_feature (1)..(13): U can be replaced by T. Any base can be replaced with a modified base.
gaggccaaggugg

SEQ ID NO: 65 - RNA, 13 nt, Artificial Sequence
  Synthetic sequence
  misc_feature (1)..(13): U can be replaced by T. Any base can be replaced with a modified base.
ggaggccaaggug

SEQ ID NO: 66 - RNA, 13 nt, Artificial Sequence
  Synthetic sequence
  misc_feature (1)..(13): U can be replaced by T. Any base can be replaced with a modified base.
uggaggccaaggu

SEQ ID NO: 67 - RNA, 13 nt, Artificial Sequence
  Synthetic sequence
  misc_feature (1)..(13): U can be replaced by T. Any base can be replaced with a modified base.
cuggaggccaagg

SEQ ID NO: 68 - RNA, 13 nt, Artificial Sequence
  Synthetic sequence
  misc_feature (1)..(13): U can be replaced by T. Any base can be replaced with a modified base.
ccuggaggccaag

SEQ ID NO: 69 - RNA, 13 nt, Artificial Sequence
  Synthetic sequence
  misc_feature (1)..(13): U can be replaced by T. Any base can be replaced with a modified base.
accuggaggccaa

SEQ ID NO: 70 - RNA, 13 nt, Artificial Sequence
  Synthetic sequence
  misc_feature (1)..(13): U can be replaced by T. Any base can be replaced with a modified base.
aaccuggaggcca

SEQ ID NO: 71 - RNA, 13 nt, Artificial Sequence
  Synthetic sequence
  misc_feature (1)..(13): U can be replaced by T. Any base can be replaced with a modified base.
caaccuggaggcc

SEQ ID NO: 72 - RNA, 13 nt, Artificial Sequence
  Synthetic sequence
  misc_feature (1)..(13): U can be replaced by T. Any base can be replaced with a modified base.
ucaaccuggaggc

SEQ ID NO: 73 - RNA, 13 nt, Artificial Sequence
  Synthetic sequence
  misc_feature (1)..(13): U can be replaced by T. Any base can be replaced with a modified base.
gucaaccuggagg

SEQ ID NO: 74 - RNA, 13 nt, Artificial Sequence
  Synthetic sequence
  misc_feature (1)..(13): U can be replaced by T. Any base can be replaced with a modified base.
agucaaccuggag

SEQ ID NO: 75 - RNA, 13 nt, Artificial Sequence
  Synthetic sequence
  misc_feature (1)..(13): U can be replaced by T. Any base can be replaced with a modified base.
gagucaaccugga

SEQ ID NO: 76 - RNA, 13 nt, Artificial Sequence
  Synthetic sequence
  misc_feature (1)..(13): U can be replaced by T. Any base can be replaced with a modified base.
agagucaaccugg

SEQ ID NO: 77 - RNA, 13 nt, Artificial Sequence
  Synthetic sequence
  misc_feature (1)..(13): U can be replaced by T. Any base can be replaced with a modified base.
gagagucaaccug

SEQ ID NO: 78 - RNA, 13 nt, Artificial Sequence
  Synthetic sequence
  misc_feature (1)..(13): U can be replaced by T. Any base can be replaced with a modified base.
ugagagucaaccu

SEQ ID NO: 79 - RNA, 13 nt, Artificial Sequence
  Synthetic sequence
  misc_feature (1)..(13): U can be replaced by T. Any base can be replaced with a modified base.
gugagagucaacc

SEQ ID NO: 80 - RNA, 13 nt, Artificial Sequence
  Synthetic sequence
  misc_feature (1)..(13): U can be replaced by T. Any base can be replaced with a modified base.
agugagagucaac

SEQ ID NO: 81 - RNA, 13 nt, Artificial Sequence
  Synthetic sequence
  misc_feature (1)..(13): U can be replaced by T. Any base can be replaced with a modified base.
uagugagagucaa

SEQ ID NO: 82 - RNA, 13 nt, Artificial Sequence
  Synthetic sequence
  misc_feature (1)..(13): U can be replaced by T. Any base can be replaced with a modified base.
guagugagaguca

SEQ ID NO: 83 - RNA, 13 nt, Artificial Sequence
  Synthetic sequence
  misc_feature (1)..(13): U can be replaced by T. Any base can be replaced with a modified base.
aguagugagaguc

SEQ ID NO: 84 - RNA, 13 nt, Artificial Sequence
  Synthetic sequence
  misc_feature: U can be replaced by T. Any base can be replaced with a modified base.
gaguagugagagu

SEQ ID NO: 85 - RNA, 13 nt, Artificial Sequence
  Synthetic sequence
  misc_feature (1)..(13): U can be replaced by T. Any base can be replaced with a modified base.
ugaguagugagag

SEQ ID NO: 86 - RNA, 13 nt, Artificial Sequence
  Synthetic sequence
  misc_feature (1)..(13): U can be replaced by T. Any base can be replaced with a modified base.
augaguagugaga

SEQ ID NO: 87 - RNA, 13 nt, Artificial Sequence
  Synthetic sequence
  misc_feature (1)..(13): U can be replaced by T. Any base can be replaced with a modified base.
gaugaguagugag

SEQ ID NO: 88 - RNA, 13 nt, Artificial Sequence
  Synthetic sequence
  misc_feature (1)..(13): U can be replaced by T. Any base can be replaced with a modified base.
ugaugaguaguga

SEQ ID NO: 89 - RNA, 13 nt, Artificial Sequence
  Synthetic sequence
  misc_feature (1)..(13): U can be replaced by T. Any base can be replaced with a modified base.
augaugaguagug

SEQ ID NO: 90 - RNA, 13 nt, Artificial Sequence
  Synthetic sequence
  misc_feature (1)..(13): U can be replaced by T. Any base can be replaced with a modified base.
gaugaugaguagu

SEQ ID NO: 91 - RNA, 13 nt, Artificial Sequence
  Synthetic sequence
  misc_feature (1)..(13): U can be replaced by T. Any base can be replaced with a modified base.
ugaugaugaguag

SEQ ID NO: 92 - RNA, 13 nt, Artificial Sequence
  Synthetic sequence
  misc_feature (1)..(13): U can be replaced by T. Any base can be replaced with a modified base.
cugaugaugagua

SEQ ID NO: 93 - RNA, 13 nt, Artificial Sequence
  Synthetic sequence
  misc_feature (1)..(13): U can be replaced by T. Any base can be replaced with a modified base.
ccugaugaugagu

SEQ ID NO: 94 - RNA, 13 nt, Artificial Sequence
  Synthetic sequence
  misc_feature (1)..(13): U can be replaced by T. Any base can be replaced with a modified base.
accugaugaugag

SEQ ID NO: 95 - RNA, 13 nt, Artificial Sequence
  Synthetic sequence
  misc_feature (1)..(13): U can be replaced by T. Any base can be replaced with a modified base.
aaccugaugauga

SEQ ID NO: 96 - RNA, 13 nt, Artificial Sequence
  Synthetic sequence
  misc_feature (1)..(13): U can be replaced by T. Any base can be replaced with a modified base.
gaaccugaugaug

SEQ ID NO: 97 - RNA, 13 nt, Artificial Sequence
  Synthetic sequence
  misc_feature (1)..(13): U can be replaced by T. Any base can be replaced with a modified base.
agaaccugaugau

SEQ ID NO: 98 - RNA, 13 nt, Artificial Sequence
  Synthetic sequence
  misc_feature (1)..(13): U can be replaced by T. Any base can be replaced with a modified base.
aagaaccugauga

SEQ ID NO: 99 - RNA, 13 nt, Artificial Sequence
  Synthetic sequence
  misc_feature (1)..(13): U can be replaced by T. Any base can be replaced with a modified base.
gaagaaccugaug

SEQ ID NO: 100 - RNA, 13 nt, Artificial Sequence
  Synthetic sequence
  misc_feature (1)..(13): U can be replaced by T. Any base can be replaced with a modified base.
ggaagaaccugau

SEQ ID NO: 101 - RNA, 13 nt, Artificial Sequence
  Synthetic sequence
  misc_feature (1)..(13): U can be replaced by T. Any base can be replaced with a modified base.
aggaagaaccuga

SEQ ID NO: 102 - RNA, 13 nt, Artificial Sequence
  Synthetic sequence
  misc_feature (1)..(13): U can be replaced by T. Any base can be replaced with a modified base.
aaggaagaaccug

SEQ ID NO: 103 - RNA, 13 nt, Artificial Sequence
  Synthetic sequence
  misc_feature (1)..(13): U can be replaced by T. Any base can be replaced with a modified base.
gaaggaagaaccu

SEQ ID NO: 104 - RNA, 13 nt, Artificial Sequence
  Synthetic sequence
  misc_feature (1)..(13): U can be replaced by T. Any base can be replaced with a modified base.
agaaggaagaacc

SEQ ID NO: 105 - RNA, 13 nt, Artificial Sequence
  Synthetic sequence
  misc_feature (1)..(13): U can be replaced by T. Any base can be replaced with a modified base.
uagaaggaagaac

SEQ ID NO: 106 - RNA, 13 nt, Artificial Sequence
  Synthetic sequence
  misc_feature (1)..(13): U can be replaced by T. Any base can be replaced with a modified base.
auagaaggaagaa

SEQ ID NO: 107 - RNA, 13 nt, Artificial Sequence
  Synthetic sequence
  misc_feature (1)..(13): U can be replaced by T. Any base can be replaced with a modified base.
aauagaaggaaga

SEQ ID NO: 108 - RNA, 13 nt, Artificial Sequence
  Synthetic sequence
  misc_feature (1)..(13): U can be replaced by T. Any base can be replaced with a modified base.
gaauagaaggaag

SEQ ID NO: 109 - RNA, 13 nt, Artificial Sequence
  Synthetic sequence
  misc_feature (1)..(13): U can be replaced by T. Any base can be replaced with a modified base.
ggaauagaaggaa

SEQ ID NO: 110 - RNA, 13 nt, Artificial Sequence
  Synthetic sequence
  misc_feature (1)..(13): U can be replaced by T. Any base can be replaced with a modified base.
uggaauagaagga

SEQ ID NO: 111 - RNA, 13 nt, Artificial Sequence
  Synthetic sequence
  misc_feature (1)..(13): U can be replaced by T. Any base can be replaced with a modified base.
cuggaauagaagg

SEQ ID NO: 112 - RNA, 13 nt, Artificial Sequence
  Synthetic sequence
  misc_feature (1)..(13): U can be replaced by T. Any base can be replaced with a modified base.
gcuggaauagaag

SEQ ID NO: 113 - RNA, 13 nt, Artificial Sequence
  Synthetic sequence
  misc_feature (1)..(13): U can be replaced by T. Any base can be replaced with a modified base.
ggcuggaauagaa

SEQ ID NO: 114 - RNA, 13 nt, Artificial Sequence
  Synthetic sequence
  misc_feature (1)..(13): U can be replaced by T. Any base can be replaced with a modified base.
gggcuggaauaga

SEQ ID NO: 115 - RNA, 13 nt, Artificial Sequence
  Synthetic sequence
  misc_feature (1)..(13): U can be replaced by T. Any base can be replaced with a modified base.
agggcuggaauag

SEQ ID NO: 116 - RNA, 13 nt, Artificial Sequence
  Synthetic sequence
  misc_feature (1)..(13): U can be replaced by T. Any base can be replaced with a modified base.
uagggcuggaaua

SEQ ID NO: 117 - RNA, 13 nt, Artificial Sequence
  Synthetic sequence
  misc_feature (1)..(13): U can be replaced by T. Any base can be replaced with a modified base.
uuagggcuggaau

SEQ ID NO: 118 - RNA, 13 nt, Artificial Sequence
  Synthetic sequence
  misc_feature (1)..(13): U can be replaced by T. Any base can be replaced with a modified base.
guuagggcuggaa

SEQ ID NO: 119 - RNA, 13 nt, Artificial Sequence
  Synthetic sequence
  misc_feature (1)..(13): U can be replaced by T. Any base can be replaced with a modified base.
gguuagggcugga

SEQ ID NO: 120 - RNA, 13 nt, Artificial Sequence
  Synthetic sequence
  misc_feature (1)..(13): U can be replaced by T. Any base can be replaced with a modified base.
ugguuagggcugg

SEQ ID NO: 121 - RNA, 13 nt, Artificial Sequence
  Synthetic sequence
  misc_feature (1)..(13): U can be replaced by T. Any base can be replaced with a modified base.
gugguuagggcug

SEQ ID NO: 122 - RNA, 13 nt, Artificial Sequence
  Synthetic sequence
  misc_feature (1)..(13): U can be replaced by T. Any base can be replaced with a modified base.
agugguuagggcu

SEQ ID NO: 123 - RNA, 13 nt, Artificial Sequence
  Synthetic sequence
  misc_feature (1)..(13): U can be replaced by T. Any base can be replaced with a modified base.
gagugguuagggc

SEQ ID NO: 124 - RNA, 13 nt, Artificial Sequence
  Synthetic sequence
  misc_feature (1)..(13): U can be replaced by T. Any base can be replaced with a modified base.
ugagugguuaggg

SEQ ID NO: 125 - RNA, 13 nt, Artificial Sequence
  Synthetic sequence
  misc_feature (1)..(13): U can be replaced by T. Any base can be replaced with a modified base.
cugagugguuagg

SEQ ID NO: 126 - RNA, 13 nt, Artificial Sequence
  Synthetic sequence
  misc_feature (1)..(13): U can be replaced by T. Any base can be replaced with a modified base.
ccugagugguuag

SEQ ID NO: 127 - RNA, 13 nt, Artificial Sequence
  Synthetic sequence
  misc_feature (1)..(13): U can be replaced by T. Any base can be replaced with a modified base.
uccugagugguua

SEQ ID NO: 128 - RNA, 13 nt, Artificial Sequence
  Synthetic sequence
  misc_feature (1)..(13): U can be replaced by T. Any base can be replaced with a modified base.
auccugagugguu

SEQ ID NO: 129 - RNA, 13 nt, Artificial Sequence
  Synthetic sequence
  misc_feature (1)..(13): U can be replaced by T. Any base can be replaced with a modified base.
aauccugaguggu

SEQ ID NO: 130 - RNA, 13 nt, Artificial Sequence
  Synthetic sequence
  misc_feature (1)..(13): U can be replaced by T. Any base can be replaced with a modified base.
caauccugagugg

SEQ ID NO: 131 - RNA, 13 nt, Artificial Sequence
  Synthetic sequence
  misc_feature (1)..(13): U can be replaced by T. Any base can be replaced with a modified base.
ccaauccugagug

SEQ ID NO: 132 - RNA, 13 nt, Artificial Sequence
  Synthetic sequence
  misc_feature (1)..(13): U can be replaced by T. Any base can be replaced with a modified base.
cccaauccugagu

SEQ ID NO: 133 - RNA, 13 nt, Artificial Sequence
  Synthetic sequence
  misc_feature (1)..(13): U can be replaced by T. Any base can be replaced with a modified base.
gcccaauccugag

SEQ ID NO: 134 - RNA, 13 nt, Artificial Sequence
  Synthetic sequence
  misc_feature (1)..(13): U can be replaced by T. Any base can be replaced with a modified base.
ggcccaauccuga

SEQ ID NO: 135 - RNA, 13 nt, Artificial Sequence
  Synthetic sequence
  misc_feature (1)..(13): U can be replaced by T. Any base can be replaced with a modified base.
cggcccaauccug

SEQ ID NO: 136 - RNA, 13 nt, Artificial Sequence
  Synthetic sequence
  misc_feature (1)..(13): U can be replaced by T. Any base can be replaced with a modified base.
acggcccaauccu

SEQ ID NO: 137 - RNA, 13 nt, Artificial Sequence
  Synthetic sequence
  misc_feature (1)..(13): U can be replaced by T. Any base can be replaced with a modified base.
aacggcccaaucc

SEQ ID NO: 138 - RNA, 13 nt, Artificial Sequence
  Synthetic sequence
  misc_feature (1)..(13): U can be replaced by T. Any base can be replaced with a modified base.
aaacggcccaauc

SEQ ID NO: 139 - RNA, 13 nt, Artificial Sequence
  Synthetic sequence
  misc_feature (1)..(13): U can be replaced by T. Any base can be replaced with a modified base.
caaacggcccaau

SEQ ID NO: 140 - RNA, 13 nt, Artificial Sequence
  Synthetic sequence
  misc_feature (1)..(13): U can be replaced by T. Any base can be replaced with a modified base.
acaaacggcccaa

SEQ ID NO: 141 - RNA, 13 nt, Artificial Sequence
  Synthetic sequence
  misc_feature (1)..(13): U can be replaced by T. Any base can be replaced with a modified base.
cacaaacggccca

SEQ ID NO: 142 - RNA, 13 nt, Artificial Sequence
  Synthetic sequence
  misc_feature (1)..(13): U can be replaced by T. Any base can be replaced with a modified base.
acacaaacggccc

SEQ ID NO: 143 - RNA, 13 nt, Artificial Sequence
  Synthetic sequence
  misc_feature (1)..(13): U can be replaced by T. Any base can be replaced with a modified base.
gacacaaacggcc

SEQ ID NO: 144 - RNA, 13 nt, Artificial Sequence
  Synthetic sequence
  misc_feature (1)..(13): U can be replaced by T. Any base can be replaced with a modified base.
agacacaaacggc

SEQ ID NO: 145 - RNA, 13 nt, Artificial Sequence
  Synthetic sequence
  misc_feature (1)..(13): U can be replaced by T. Any base can be replaced with a modified base.
cagacacaaacgg

SEQ ID NO: 146 - RNA, 13 nt, Artificial Sequence
  Synthetic sequence
  misc_feature (1)..(13): U can be replaced by T. Any base can be replaced with a modified base.
ccagacacaaacg

SEQ ID NO: 147 - RNA, 13 nt, Artificial Sequence
  Synthetic sequence
  misc_feature (1)..(13): U can be replaced by T. Any base can be replaced with a modified base.
cccagacacaaac

SEQ ID NO: 148 - RNA, 13 nt, Artificial Sequence
  Synthetic sequence
  misc_feature (1)..(13): U can be replaced by T. Any base can be replaced with a modified base.
acccagacacaaa

SEQ ID NO: 149 - RNA, 13 nt, Artificial Sequence
  Synthetic sequence
  misc_feature (1)..(13): U can be replaced by T. Any base can be replaced with a modified base.
uacccagacacaa

SEQ ID NO: 150 - RNA, 13 nt, Artificial Sequence
  Synthetic sequence
  misc_feature (1)..(13): U can be replaced by T. Any base can be replaced with a modified base.
auacccagacaca

SEQ ID NO: 151 - RNA, 13 nt, Artificial Sequence
  Synthetic sequence
  misc_feature (1)..(13): U can be replaced by T. Any base can be replaced with a modified base.
cauacccagacac

SEQ ID NO: 152 - RNA, 13 nt, Artificial Sequence
  Synthetic sequence
  misc_feature (1)..(13): U can be replaced by T. Any base can be replaced with a modified base.
acauacccagaca

SEQ ID NO: 153 - RNA, 13 nt, Artificial Sequence
  Synthetic sequence
  misc_feature (1)..(13): U can be replaced by T. Any base can be replaced with a modified base.
gacauacccagac

SEQ ID NO: 154 - RNA, 13 nt, Artificial Sequence
  Synthetic sequence
  misc_feature (1)..(13): U can be replaced by T. Any base can be replaced with a modified base.
agacauacccaga

SEQ ID NO: 155 - RNA, 13 nt, Artificial Sequence
  Synthetic sequence
  misc_feature (1)..(13): U can be replaced by T. Any base can be replaced with a modified base.
gagacauacccag

SEQ ID NO: 156 - RNA, 13 nt, Artificial Sequence
  Synthetic sequence
  misc_feature (1)..(13): U can be replaced by T. Any base can be replaced with a modified base.
agagacauaccca

SEQ ID NO: 157 - RNA, 13 nt, Artificial Sequence
  Synthetic sequence
  misc_feature (1)..(13): U can be replaced by T. Any base can be replaced with a modified base.
aagagacauaccc

SEQ ID NO: 158 - RNA, 13 nt, Artificial Sequence
  Synthetic sequence
  misc_feature (1)..(13): U can be replaced by T. Any base can be replaced with a modified base.
gaagagacauacc

SEQ ID NO: 159 - RNA, 13 nt, Artificial Sequence
  Synthetic sequence
  misc_feature (1)..(13): U can be replaced by T. Any base can be replaced with a modified base.
ggaagagacauac

SEQ ID NO: 160 - RNA, 13 nt, Artificial Sequence
  Synthetic sequence
  misc_feature (1)..(13): U can be replaced by T. Any base can be replaced with a modified base.
uggaagagacaua

SEQ ID NO: 161 - RNA, 13 nt, Artificial Sequence
  Synthetic sequence
  misc_feature (1)..(13): U can be replaced by T. Any base can be replaced with a modified base.
cuggaagagacau

SEQ ID NO: 162 - RNA, 13 nt, Artificial Sequence
  Synthetic sequence
  misc_feature (1)..(13): U can be replaced by T. Any base can be replaced with a modified base.
gcuggaagagaca

SEQ ID NO: 163 - RNA, 13 nt, Artificial Sequence
  Synthetic sequence
  misc_feature (1)..(13): U can be replaced by T. Any base can be replaced with a modified base.
agcuggaagagac

SEQ ID NO: 164 - RNA, 13 nt, Artificial Sequence
  Synthetic sequence
cagcuggaagaga

SEQ ID NO: 165 - RNA, 13 nt, Artificial Sequence
  Synthetic sequence
  misc_feature (1)..(13): U can be replaced by T. Any base can be replaced with a modified base.
gcagcuggaagag

SEQ ID NO: 166 - RNA, 13 nt, Artificial Sequence
  Synthetic sequence
  misc_feature (1)..(13): U can be replaced by T. Any base can be replaced with a modified base.
ggcagcuggaaga

SEQ ID NO: 167 - RNA, 13 nt, Artificial Sequence
  Synthetic sequence
  misc_feature (1)..(13): U can be replaced by T. Any base can be replaced with a modified base.
aggcagcuggaag

SEQ ID NO: 168 - RNA, 13 nt, Artificial Sequence
  Synthetic sequence
  misc_feature (1)..(13): U can be replaced by T. Any base can be replaced with a modified base.
caggcagcuggaa

SEQ ID NO: 169 - RNA, 13 nt, Artificial Sequence
  Synthetic sequence
  misc_feature (1)..(13): U can be replaced by T. Any base can be replaced with a modified base.
ccaggcagcugga

SEQ ID NO: 170 - RNA, 13 nt, Artificial Sequence
  Synthetic sequence
  misc_feature (1)..(13): U can be replaced by T. Any base can be replaced with a modified base.
cccaggcagcugg

SEQ ID NO: 171 - RNA, 13 nt, Artificial Sequence
  Synthetic sequence
  misc_feature (1)..(13): U can be replaced by T. Any base can be replaced with a modified base.
acccaggcagcug

SEQ ID NO: 172 - RNA, 13 nt, Artificial Sequence
  Synthetic sequence
  misc_feature (1)..(13): U can be replaced by T. Any base can be replaced with a modified base.
aacccaggcagcu

SEQ ID NO: 173 - RNA, 13 nt, Artificial Sequence
  Synthetic sequence
  misc_feature (1)..(13): U can be replaced by T. Any base can be replaced with a modified base.
aaacccaggcagc

SEQ ID NO: 174 - RNA, 13 nt, Artificial Sequence
  Synthetic sequence
  misc_feature (1)..(13): U can be replaced by T. Any base can be replaced with a modified base.
gaaacccaggcag

SEQ ID NO: 175 - RNA, 13 nt, Artificial Sequence
  Synthetic sequence
  misc_feature (1)..(13): U can be replaced by T. Any base can be replaced with a modified base.
ggaaacccaggca

SEQ ID NO: 176 - RNA, 13 nt, Artificial Sequence
  Synthetic sequence
  misc_feature (1)..(13): U can be replaced by T. Any base can be replaced with a modified base.
aggaaacccaggc

SEQ ID NO: 177 - RNA, 13 nt, Artificial Sequence
  Synthetic sequence
  misc_feature (1)..(13): U can be replaced by T. Any base can be replaced with a modified base.
caggaaacccagg

SEQ ID NO: 178 - RNA, 13 nt, Artificial Sequence
  Synthetic sequence
  misc_feature (1)..(13): U can be replaced by T. Any base can be replaced with a modified base.
ccaggaaacccag

SEQ ID NO: 179 - RNA, 13 nt, Artificial Sequence
  Synthetic sequence
  misc_feature (1)..(13): U can be replaced by T. Any base can be replaced with a modified base.
uccaggaaaccca

SEQ ID NO: 180 - RNA, 13 nt, Artificial Sequence
  Synthetic sequence
  misc_feature (1)..(13): U can be replaced by T. Any base can be replaced with a modified base.
uuccaggaaaccc

SEQ ID NO: 181 - RNA, 13 nt, Artificial Sequence
  Synthetic sequence
  misc_feature (1)..(13): U can be replaced by T. Any base can be replaced with a modified base.
uuuccaggaaacc

SEQ ID NO: 182 - RNA, 13 nt, Artificial Sequence
  Synthetic sequence
  misc_feature (1)..(13): U can be replaced by T. Any base can be replaced with a modified base.
cuuuccaggaaac

SEQ ID NO: 183 - RNA, 13 nt, Artificial Sequence
  Synthetic sequence
  misc_feature (1)..(13): U can be replaced by T. Any base can be replaced with a modified base.
ucuuuccaggaaa

SEQ ID NO: 184 - RNA, 13 nt, Artificial Sequence
  Synthetic sequence
  misc_feature (1)..(13): U can be replaced by T. Any base can be replaced with a modified base.
uucuuuccaggaa

SEQ ID NO: 185 - RNA, 13 nt, Artificial Sequence
  Synthetic sequence
  misc_feature (1)..(13): U can be replaced by T. Any base can be replaced with a modified base.
guucuuuccagga

SEQ ID NO: 186 - RNA, 13 nt, Artificial Sequence
  Synthetic sequence
  misc_feature (1)..(13): U can be replaced by T. Any base can be replaced with a modified base.
aguucuuuccagg

SEQ ID NO: 187 - RNA, 13 nt, Artificial Sequence
  Synthetic sequence
  misc_feature (1)..(13): U can be replaced by T. Any base can be replaced with a modified base.
gaguucuuuccag

SEQ ID NO: 188 - RNA, 13 nt, Artificial Sequence
  Synthetic sequence
  misc_feature (1)..(13): U can be replaced by T. Any base can be replaced with a modified base.
agaguucuuucca

SEQ ID NO: 189 - RNA, 13 nt, Artificial Sequence
  Synthetic sequence
  misc_feature (1)..(13): U can be replaced by T. Any base can be replaced with a modified base.
aagaguucuuucc

SEQ ID NO: 190 - RNA, 13 nt, Artificial Sequence
  Synthetic sequence
  misc_feature (1)..(13): U can be replaced by T. Any base can be replaced with a modified base.
uaagaguucuuuc

SEQ ID NO: 191 - RNA, 13 nt, Artificial Sequence
  Synthetic sequence
  misc_feature (1)..(13): U can be replaced by T. Any base can be replaced with a modified base.
auaagaguucuuu

SEQ ID NO: 192 - RNA, 13 nt, Artificial Sequence
  Synthetic sequence
  misc_feature (1)..(13): U can be replaced by T. Any base can be replaced with a modified base.
gauaagaguucuu

SEQ ID NO: 193 - RNA, 13 nt, Artificial Sequence
  Synthetic sequence
  misc_feature (1)..(13): U can be replaced by T. Any base can be replaced with a modified base.
ggauaagaguucu

SEQ ID NO: 194 - RNA, 13 nt, Artificial Sequence
  Synthetic sequence
  misc_feature (1)..(13): U can be replaced by T. Any base can be replaced with a modified base.
gggauaagaguuc

SEQ ID NO: 195 - RNA, 13 nt, Artificial Sequence
  Synthetic sequence
  misc_feature (1)..(13): U can be replaced by T. Any base can be replaced with a modified base.
ggggauaagaguu

SEQ ID NO: 196 - RNA, 13 nt, Artificial Sequence
  Synthetic sequence
  misc_feature (1)..(13): U can be replaced by T. Any base can be replaced with a modified base.
uggggauaagagu

SEQ ID NO: 197 - RNA, 13 nt, Artificial Sequence
  Synthetic sequence
  misc_feature (1)..(13): U can be replaced by T. Any base can be replaced with a modified base.
cuggggauaagag

SEQ ID NO: 198 - RNA, 13 nt, Artificial Sequence
  Synthetic sequence
  misc_feature (1)..(13): U can be replaced by T. Any base can be replaced with a modified base.
ccuggggauaaga

SEQ ID NO: 199 - RNA, 13 nt, Artificial Sequence
  Synthetic sequence
  misc_feature (1)..(13): U can be replaced by T. Any base can be replaced with a modified base.
uccuggggauaag

SEQ ID NO: 200 - RNA, 13 nt, Artificial Sequence
  Synthetic sequence
  misc_feature (1)..(13): U can be replaced by T. Any base can be replaced with a modified base.
uuccuggggauaa

SEQ ID NO: 201 - RNA, 13 nt, Artificial Sequence
  Synthetic sequence
  misc_feature (1)..(13): U can be replaced by T. Any base can be replaced with a modified base.
guuccuggggaua

SEQ ID NO: 202 - RNA, 13 nt, Artificial Sequence
  Synthetic sequence
  misc_feature (1)..(13): U can be replaced by T. Any base can be replaced with a modified base.
aguuccuggggau

SEQ ID NO: 203 - RNA, 13 nt, Artificial Sequence
  Synthetic sequence
  misc_feature (1)..(13): U can be replaced by T. Any base can be replaced with a modified base.
uaguuccugggga

SEQ ID NO: 204 - RNA, 13 nt, Artificial Sequence
  Synthetic sequence
  misc_feature (1)..(13): U can be replaced by T. Any base can be replaced with a modified base.
cuaguuccugggg

SEQ ID NO: 205 - RNA, 13 nt, Artificial Sequence
  Synthetic sequence
  misc_feature (1)..(13): U can be replaced by T. Any base can be replaced with a modified base.
acuaguuccuggg

SEQ ID NO: 206 - RNA, 13 nt, Artificial Sequence
  Synthetic sequence
  misc_feature (1)..(13): U can be replaced by T. Any base can be replaced with a modified base.
aacuaguuccugg

SEQ ID NO: 207 - RNA, 13 nt, Artificial Sequence
  Synthetic sequence
  misc_feature (1)..(13): U can be replaced by T. Any base can be replaced with a modified base.
aaacuaguuccug

SEQ ID NO: 208 - RNA, 13 nt, Artificial Sequence
  Synthetic sequence
  misc_feature (1)..(13): U can be replaced by T. Any base can be replaced with a modified base.
caaacuaguuccu

SEQ ID NO: 209 - RNA, 13 nt, Artificial Sequence
  Synthetic sequence
  misc_feature (1)..(13): U can be replaced by T. Any base can be replaced with a modified base.
acaaacuaguucc

SEQ ID NO: 210 - RNA, 13 nt, Artificial Sequence
  Synthetic sequence
  misc_feature (1)..(13): U can be replaced by T. Any base can be replaced with a modified base.
aacaaacuaguuc

SEQ ID NO: 211 - RNA, 13 nt, Artificial Sequence
  Synthetic sequence
  misc_feature (1)..(13): U can be replaced by T. Any base can be replaced with a modified base.
caacaaacuaguu

SEQ ID NO: 212 - RNA, 13 nt, Artificial Sequence
  Synthetic sequence
  misc_feature (1)..(13): U can be replaced by T. Any base can be replaced with a modified base.
ucaacaaacuagu

SEQ ID NO: 213 - RNA, 13 nt, Artificial Sequence
  Synthetic sequence
  misc_feature (1)..(13): U can be replaced by T. Any base can be replaced with a modified base.
uucaacaaacuag

SEQ ID NO: 214 - RNA, 13 nt, Artificial Sequence
  Synthetic sequence
  misc_feature (1)..(13): U can be replaced by T. Any base can be replaced with a modified base.
auucaacaaacua

SEQ ID NO: 215 - RNA, 13 nt, Artificial Sequence
  Synthetic sequence
  misc_feature (1)..(13): U can be replaced by T. Any base can be replaced with a modified base.
uauucaacaaacu

SEQ ID NO: 216 - RNA, 13 nt, Artificial Sequence
  Synthetic sequence
  misc_feature (1)..(13): U can be replaced by T. Any base can be replaced with a modified base.
uuauucaacaaac

SEQ ID NO: 217 - RNA, 13 nt, Artificial Sequence
  Synthetic sequence
  misc_feature (1)..(13): U can be replaced by T. Any base can be replaced with a modified base.
uuuauucaacaaa

SEQ ID NO: 218 - RNA, 13 nt, Artificial Sequence
  Synthetic sequence
  misc_feature (1)..(13): U can be replaced by T. Any base can be replaced with a modified base.
auuuauucaacaa

SEQ ID NO: 219 - RNA, 13 nt, Artificial Sequence
  Synthetic sequence
  misc_feature (1)..(13): U can be replaced by T. Any base can be replaced with a modified base.
cauuuauucaaca

SEQ ID NO: 220 - RNA, 13 nt, Artificial Sequence
  Synthetic sequence
  misc_feature (1)..(13): U can be replaced by T. Any base can be replaced with a modified base.
gcauuuauucaac

SEQ ID NO: 221 - RNA, 13 nt, Artificial Sequence
  Synthetic sequence
  misc_feature (1)..(13): U can be replaced by T. Any base can be replaced with a modified base.
agcauuuauucaa

SEQ ID NO: 222 - RNA, 13 nt, Artificial Sequence
  Synthetic sequence
  misc_feature (1)..(13): U can be replaced by T. Any base can be replaced with a modified base.
cagcauuuauuca

SEQ ID NO: 223 - RNA, 13 nt, Artificial Sequence
  Synthetic sequence
  misc_feature (1)..(13): U can be replaced by T. Any base can be replaced with a modified base.
ccagcauuuauuc

SEQ ID NO: 224 - RNA, 13 nt, Artificial Sequence
  Synthetic sequence
  misc_feature (1)..(13): U can be replaced by T. Any base can be replaced with a modified base.
accagcauuuauu

SEQ ID NO: 225 - RNA, 13 nt, Artificial Sequence
  Synthetic sequence
  misc_feature (1)..(13): U can be replaced by T. Any base can be replaced with a modified base.
caccagcauuuau

SEQ ID NO: 226 - RNA, 13 nt, Artificial Sequence
  Synthetic sequence
  misc_feature (1)..(13): U can be replaced by T. Any base can be replaced with a modified base.
ucaccagcauuua

SEQ ID NO: 227 - RNA, 13 nt, Artificial Sequence
  Synthetic sequence
  misc_feature (1)..(13): U can be replaced by T. Any base can be replaced with a modified base.
uucaccagcauuu

SEQ ID NO: 228 - RNA, 13 nt, Artificial Sequence
  Synthetic sequence
  misc_feature (1)..(13): U can be replaced by T. Any base can be replaced with a modified base.
auucaccagcauu

SEQ ID NO: 229 - RNA, 13 nt, Artificial Sequence
  Synthetic sequence
  misc_feature (1)..(13): U can be replaced by T. Any base can be replaced with a modified base.
cauucaccagcau

SEQ ID NO: 230 - RNA, 13 nt, Artificial Sequence
  Synthetic sequence
  misc_feature (1)..(13): U can be replaced by T. Any base can be replaced with a modified base.
ucauucaccagca

SEQ ID NO: 231 - RNA, 13 nt, Artificial Sequence
  Synthetic sequence
  misc_feature (1)..(13): U can be replaced by T. Any base can be replaced with a modified base.
uucauucaccagc

SEQ ID NO: 232 - RNA, 13 nt, Artificial Sequence
  Synthetic sequence
  misc_feature (1)..(13): U can be replaced by T. Any base can be replaced with a modified base.
auucauucaccag

SEQ ID NO: 233 - RNA, 13 nt, Artificial Sequence
  Synthetic sequence
  misc_feature (1)..(13): U can be replaced by T. Any base can be replaced with a modified base.
cauucauucacca

SEQ ID NO: 234 - RNA, 13 nt, Artificial Sequence
  Synthetic sequence
  misc_feature (1)..(13): U can be replaced by T. Any base can be replaced with a modified base.
ucauucauucacc

SEQ ID NO: 235 - RNA, 13 nt, Artificial Sequence
  Synthetic sequence
  misc_feature (1)..(13): U can be replaced by T. Any base can be replaced with a modified base.
uucauucauucac

SEQ ID NO: 236 - RNA, 13 nt, Artificial Sequence
  Synthetic sequence
  misc_feature (1)..(13): U can be replaced by T. Any base can be replaced with a modified base.
auucauucauuca

SEQ ID NO: 237 - RNA, 13 nt, Artificial Sequence
  Synthetic sequence
  misc_feature (1)..(13): U can be replaced by T. Any base can be replaced with a modified base.
cauucauucauuc

SEQ ID NO: 238 - RNA, 13 nt, Artificial Sequence
  Synthetic sequence
  misc_feature (1)..(13): U can be replaced by T. Any base can be replaced with a modified base.
ucauucauucauu

SEQ ID NO: 239 - RNA, 13 nt, Artificial Sequence
  Synthetic sequence
  misc_feature (1)..(13): U can be replaced by T. Any base can be replaced with a modified base.
aucauucauucau

SEQ ID NO: 240 - RNA, 13 nt, Artificial Sequence
  Synthetic sequence
  misc_feature (1)..(13): U can be replaced by T. Any base can be replaced with a modified base.
aaucauucauuca

SEQ ID NO: 241 - RNA, 13 nt, Artificial Sequence
  Synthetic sequence
  misc_feature (1)..(13): U can be replaced by T. Any base can be replaced with a modified base.
caaucauucauuc

SEQ ID NO: 242 - RNA, 13 nt, Artificial Sequence
  Synthetic sequence
  misc_feature (1)..(13): U can be replaced by T. Any base can be replaced with a modified base.
ucaaucauucauu

SEQ ID NO: 243 - RNA, 13 nt, Artificial Sequence
  Synthetic sequence
  misc_feature (1)..(13): U can be replaced by T. Any base can be replaced with a modified base.
uucaaucauucau

SEQ ID NO: 244 - RNA, 13 nt, Artificial Sequence
  Synthetic sequence
  misc_feature (1)..(13): U can be replaced by T. Any base can be replaced with a modified base.
guucaaucauuca

SEQ ID NO: 245 - RNA, 13 nt, Artificial Sequence
  Synthetic sequence
  misc_feature (1)..(13): U can be replaced by T. Any base can be replaced with a modified base.
uguucaaucauuc

SEQ ID NO: 246 - RNA, 13 nt, Artificial Sequence
  Synthetic sequence
  misc_feature (1)..(13): U can be replaced by T. Any base can be replaced with a modified base.
cuguucaaucauu

SEQ ID NO: 247 - RNA, 13 nt, Artificial Sequence
  Synthetic sequence
  misc_feature (1)..(13): U can be replaced by T. Any base can be replaced with a modified base.
ucuguucaaucau

SEQ ID NO: 248 - RNA, 13 nt, Artificial Sequence
  Synthetic sequence
  misc_feature (1)..(13): U can be replaced by T. Any base can be replaced with a modified base.
aucuguucaauca

SEQ ID NO: 249 - RNA, 13 nt, Artificial Sequence
  Synthetic sequence
  misc_feature (1)..(13): U can be replaced by T. Any base can be replaced with a modified base.
caucuguucaauc

SEQ ID NO: 250 - RNA, 13 nt, Artificial Sequence
  Synthetic sequence
  misc_feature (1)..(13): U can be replaced by T. Any base can be replaced with a modified base.
ucaucuguucaau

SEQ ID NO: 251 - RNA, 13 nt, Artificial Sequence
  Synthetic sequence
  misc_feature (1)..(13): U can be replaced by T. Any base can be replaced with a modified base.
uucaucuguucaa

SEQ ID NO: 252 - RNA, 13 nt, Artificial Sequence
  Synthetic sequence
  misc_feature (1)..(13): U can be replaced by T. Any base can be replaced with a modified base.
auucaucuguuca

SEQ ID NO: 253 - RNA, 13 nt, Artificial Sequence
  Synthetic sequence
  misc_feature (1)..(13): U can be replaced by T. Any base can be replaced with a modified base.
cauucaucuguuc

SEQ ID NO: 254 - RNA, 13 nt, Artificial Sequence
  Synthetic sequence
  misc_feature (1)..(13): U can be replaced by T. Any base can be replaced with a modified base.
ucauucaucuguu

SEQ ID NO: 255 - RNA, 13 nt, Artificial Sequence
  Synthetic sequence
  misc_feature (1)..(13): U can be replaced by T. Any base can be replaced with a modified base.
cucauucaucugu

SEQ ID NO: 256 - RNA, 13 nt, Artificial Sequence
  Synthetic sequence
  misc_feature (1)..(13): U can be replaced by T. Any base can be replaced with a modified base.
acucauucaucug

SEQ ID NO: 257 - RNA, 13 nt, Artificial Sequence
  Synthetic sequence
  misc_feature (1)..(13): U can be replaced by T. Any base can be replaced with a modified base.
cacucauucaucu

SEQ ID NO: 258 - RNA, 13 nt, Artificial Sequence
  Synthetic sequence
  misc_feature (1)..(13): U can be replaced by T. Any base can be replaced with a modified base.
ucacucauucauc

SEQ ID NO: 259 - RNA, 13 nt, Artificial Sequence
  Synthetic sequence
  misc_feature (1)..(13): U can be replaced by T. Any base can be replaced with a modified base.
aucacucauucau

SEQ ID NO: 260 - RNA, 13 nt, Artificial Sequence
  Synthetic sequence
  misc_feature (1)..(13): U can be replaced by T. Any base can be replaced with a modified base.
caucacucauuca

SEQ ID NO: 261 - RNA, 13 nt, Artificial Sequence
  Synthetic sequence
  misc_feature (1)..(13): U can be replaced by T. Any base can be replaced with a modified base.
ucaucacucauuc

SEQ ID NO: 262 - RNA, 13 nt, Artificial Sequence
  Synthetic sequence
  misc_feature (1)..(13): U can be replaced by T. Any base can be replaced with a modified base.
cucaucacucauu

SEQ ID NO: 263 - RNA, 13 nt, Artificial Sequence
  Synthetic sequence
  misc_feature (1)..(13): U can be replaced by T. Any base can be replaced with a modified base.
acucaucacucau

SEQ ID NO: 264 - RNA, 13 nt, Artificial Sequence
  Synthetic sequence
  misc_feature (1)..(13): U can be replaced by T. Any base can be replaced with a modified base.
uacucaucacuca

SEQ ID NO: 265 - RNA, 13 nt, Artificial Sequence
  Synthetic sequence
  misc_feature (1)..(13): U can be replaced by T. Any base can be replaced with a modified base.
cuacucaucacuc

SEQ ID NO: 266 - RNA, 13 nt, Artificial Sequence
  Synthetic sequence
  misc_feature (1)..(13): U can be replaced by T. Any base can be replaced with a modified base.
ucuacucaucacu

SEQ ID NO: 267 - RNA, 13 nt, Artificial Sequence
  Synthetic sequence
  misc_feature (1)..(13): U can be replaced by T. Any base can be replaced with a modified base.
aucuacucaucac

SEQ ID NO: 268 - RNA, 13 nt, Artificial Sequence
  Synthetic sequence
  misc_feature (1)..(13): U can be replaced by T. Any base can be replaced with a modified base.
uaucuacucauca

SEQ ID NO: 269 - RNA, 13 nt, Artificial Sequence
  Synthetic sequence
  misc_feature (1)..(13): U can be replaced by T. Any base can be replaced with a modified base.
uuaucuacucauc

SEQ ID NO: 270 - RNA, 13 nt, Artificial Sequence
  Synthetic sequence
  misc_feature (1)..(13): U can be replaced by T. Any base can be replaced with a modified base.
uuuaucuacucau

SEQ ID NO: 271 - RNA, 13 nt, Artificial Sequence
  Synthetic sequence
  misc_feature (1)..(13): U can be replaced by T. Any base can be replaced with a modified base.
uuuuaucuacuca

SEQ ID NO: 272 - RNA, 13 nt, Artificial Sequence
  Synthetic sequence
  misc_feature (1)..(13): U can be replaced by T. Any base can be replaced with a modified base.
cuuuuaucuacuc

SEQ ID NO: 273 - RNA, 13 nt, Artificial Sequence
  Synthetic sequence
  misc_feature (1)..(13): U can be replaced by T. Any base can be replaced with a modified base.
ccuuuuaucuacu

SEQ ID NO: 274 - RNA, 13 nt, Artificial Sequence
  Synthetic sequence
  misc_feature (1)..(13): U can be replaced by T. Any base can be replaced with a modified base.
gcuuuuaucuacu

SEQ ID NO: 275 - RNA, 13 nt, Artificial Sequence
  Synthetic sequence
  misc_feature (1)..(13): U can be replaced by T. Any base can be replaced with a modified base.
uccuuuuaucuac

SEQ ID NO: 276 - RNA, 13 nt, Artificial Sequence
  Synthetic sequence
  misc_feature (1)..(13): U can be replaced by T. Any base can be replaced with a modified base.
ugcuuuuaucuac

SEQ ID NO: 277 - RNA, 13 nt, Artificial Sequence
  Synthetic sequence
  misc_feature (1)..(13): U can be replaced by T. Any base can be replaced with a modified base.
auccuuuuaucua

SEQ ID NO: 278 - RNA, 13 nt, Artificial Sequence
  Synthetic sequence
  misc_feature (1)..(13): U can be replaced by T. Any base can be replaced with a modified base.
augcuuuuaucua

SEQ ID NO: 279 - RNA, 13 nt, Artificial Sequence
  Synthetic sequence
  misc_feature (1)..(13): U can be replaced by T. Any base can be replaced with a modified base.
cauccuuuuaucu

SEQ ID NO: 280 - RNA, 13 nt, Artificial Sequence
  Synthetic sequence
  misc_feature (1)..(13): U can be replaced by T. Any base can be replaced with a modified base.
caugcuuuuaucu

SEQ ID NO: 281 - RNA, 13 nt, Artificial Sequence
  Synthetic sequence
ccauccuuuuauc

SEQ ID NO: 282 - RNA, 13 nt, Artificial Sequence
  Synthetic sequence
  misc_feature (1)..(13): U can be replaced by T. Any base can be replaced with a modified base.
ccaugcuuuuauc

SEQ ID NO: 283 - RNA, 13 nt, Artificial Sequence
  Synthetic sequence
  misc_feature (1)..(13): U can be replaced by T. Any base can be replaced with a modified base.
uccauccuuuuau

SEQ ID NO: 284 - RNA, 13 nt, Artificial Sequence
  Synthetic sequence
  misc_feature (1)..(13): U can be replaced by T. Any base can be replaced with a modified base.
uccaugcuuuuau

SEQ ID NO: 285 - RNA, 13 nt, Artificial Sequence
  Synthetic sequence
  misc_feature (1)..(13): U can be replaced by T. Any base can be replaced with a modified base.
auccauccuuuua

SEQ ID NO: 286 - RNA, 13 nt, Artificial Sequence
  Synthetic sequence
  misc_feature (1)..(13): U can be replaced by T. Any base can be replaced with a modified base.
auccaugcuuuua

SEQ ID NO: 287 - RNA, 13 nt, Artificial Sequence
  Synthetic sequence
  misc_feature (1)..(13): U can be replaced by T. Any base can be replaced with a modified base.
cauccauccuuuu

SEQ ID NO: 288 - RNA, 13 nt, Artificial Sequence
  Synthetic sequence
  misc_feature (1)..(13): U can be replaced by T. Any base can be replaced with a modified base.
cauccaugcuuuu

SEQ ID NO: 289 - RNA, 13 nt, Artificial Sequence
  Synthetic sequence
  misc_feature (1)..(13): U can be replaced by T. Any base can be replaced with a modified base.
ccauccauccuuu

SEQ ID NO: 290 - RNA, 13 nt, Artificial Sequence
  Synthetic sequence
  misc_feature (1)..(13): U can be replaced by T. Any base can be replaced with a modified base.
ccauccaugcuuu

SEQ ID NO: 291 - RNA, 13 nt, Artificial Sequence
  Synthetic sequence
  misc_feature (1)..(13): U can be replaced by T. Any base can be replaced with a modified base.
uccauccauccuu

SEQ ID NO: 292 - RNA, 13 nt, Artificial Sequence
  Synthetic sequence
  misc_feature (1)..(13): U can be replaced by T. Any base can be replaced with a modified base.
uccauccaugcuu

SEQ ID NO: 293 - RNA, 13 nt, Artificial Sequence
  Synthetic sequence
  misc_feature (1)..(13): U can be replaced by T. Any base can be replaced with a modified base.
cuccauccauccu

SEQ ID NO: 294 - RNA, 13 nt, Artificial Sequence
  Synthetic sequence
  misc_feature (1)..(13): U can be replaced by T. Any base can be replaced with a modified base.
cuccauccaugcu

SEQ ID NO: 295 - RNA, 13 nt, Artificial Sequence
  Synthetic sequence
  misc_feature (1)..(13): U can be replaced by T. Any base can be replaced with a modified base.
ucuccauccaucc

SEQ ID NO: 296 - RNA, 13 nt, Artificial Sequence
  Synthetic sequence
  misc_feature (1)..(13): U can be replaced by T. Any base can be replaced with a modified base.
ucuccauccaugc

SEQ ID NO: 297 - RNA, 13 nt, Artificial Sequence
  Synthetic sequence
  misc_feature (1)..(13): U can be replaced by T. Any base can be replaced with a modified base.
cucuccauccauc

SEQ ID NO: 298 - RNA, 13 nt, Artificial Sequence
  Synthetic sequence
  misc_feature (1)..(13): U can be replaced by T. Any base can be replaced with a modified base.
cucuccauccaug

SEQ ID NO: 299 - RNA, 13 nt, Artificial Sequence
  Synthetic sequence
  misc_feature (1)..(13): U can be replaced by T. Any base can be replaced with a modified base.
ucucuccauccau

SEQ ID NO: 300 - RNA, 13 nt, Artificial Sequence
  Synthetic sequence
  misc_feature (1)..(13): U can be replaced by T. Any base can be replaced with a modified base.
aucucuccaucca

SEQ ID NO: 301 - RNA, 13 nt, Artificial Sequence
  Synthetic sequence
  misc_feature (1)..(13): U can be replaced by T. Any base can be replaced with a modified base.
caucucuccaucc

SEQ ID NO: 302 - RNA, 13 nt, Artificial Sequence
  Synthetic sequence
  misc_feature (1)..(13): U can be replaced by T. Any base can be replaced with a modified base.
ccaucucuccauc

SEQ ID NO: 303 - RNA, 13 nt, Artificial Sequence
  Synthetic sequence
  misc_feature (1)..(13): U can be replaced by T. Any base can be replaced with a modified base.
cccaucucuccau

SEQ ID NO: 304 - RNA, 13 nt, Artificial Sequence
  Synthetic sequence
  misc_feature (1)..(13): U can be replaced by T. Any base can be replaced with a modified base.
acccaucucucca

SEQ ID NO: 305 - RNA, 13 nt, Artificial Sequence
  Synthetic sequence
  misc_feature (1)..(13): U can be replaced by T. Any base can be replaced with a modified base.
cacccaucucucc

SEQ ID NO: 306 - RNA, 13 nt, Artificial Sequence
  Synthetic sequence
  misc_feature (1)..(13): U can be replaced by T. Any base can be replaced with a modified base.
ucacccaucucuc

SEQ ID NO: 307 - RNA, 13 nt, Artificial Sequence
  Synthetic sequence
  misc_feature (1)..(13): U can be replaced by T. Any base can be replaced with a modified base.
cucacccaucucu

SEQ ID NO: 308 - RNA, 13 nt, Artificial Sequence
  Synthetic sequence
  misc_feature (1)..(13): U can be replaced by T. Any base can be replaced with a modified base.
acucacccaucuc

SEQ ID NO: 309 - RNA, 13 nt, Artificial Sequence
  Synthetic sequence
  misc_feature (1)..(13): U can be replaced by T. Any base can be replaced with a modified base.
uacucacccaucu

SEQ ID NO: 310 - RNA, 13 nt, Artificial Sequence
  Synthetic sequence
  misc_feature (1)..(13): U can be replaced by T. Any base can be replaced with a modified base.
guacucacccauc

SEQ ID NO: 311 - RNA, 13 nt, Artificial Sequence
  Synthetic sequence
  misc_feature (1)..(13): U can be replaced by T. Any base can be replaced with a modified base.
uguacucacccau

SEQ ID NO: 312 - RNA, 13 nt, Artificial Sequence
  Synthetic sequence
  misc_feature (1)..(13): U can be replaced by T. Any base can be replaced with a modified base.
auguacucaccca

SEQ ID NO: 313 - RNA, 13 nt, Artificial Sequence
  Synthetic sequence
  misc_feature (1)..(13): U can be replaced by T. Any base can be replaced with a modified base.
cauguacucaccc

SEQ ID NO: 314 - RNA, 13 nt, Artificial Sequence
  Synthetic sequence
  misc_feature (1)..(13): U can be replaced by T. Any base can be replaced with a modified base.
ccauguacucacc

SEQ ID NO: 315 - RNA, 13 nt, Artificial Sequence
  Synthetic sequence
  misc_feature (1)..(13): U can be replaced by T. Any base can be replaced with a modified base.
uccauguacucac

SEQ ID NO: 316 - RNA, 13 nt, Artificial Sequence
  Synthetic sequence
  misc_feature (1)..(13): U can be replaced by T. Any base can be replaced with a modified base.
auccauguacuca

SEQ ID NO: 317 - RNA, 13 nt, Artificial Sequence
  Synthetic sequence
  misc_feature (1)..(13): U can be replaced by T. Any base can be replaced with a modified base.
cauccauguacuc

SEQ ID NO: 318 - RNA, 13 nt, Artificial Sequence
  Synthetic sequence
  misc_feature (1)..(13): U can be replaced by T. Any base can be replaced with a modified base.
ccauccauguacu

SEQ ID NO: 319 - RNA, 13 nt, Artificial Sequence
  Synthetic sequence
  misc_feature (1)..(13): U can be replaced by T. Any base can be replaced with a modified base.
uccauccauguac

SEQ ID NO: 320 - RNA, 13 nt, Artificial Sequence
  Synthetic sequence
  misc_feature (1)..(13): U can be replaced by T. Any base can be replaced with a modified base.
auccauccaugua

SEQ ID NO: 321 - RNA, 13 nt, Artificial Sequence
  Synthetic sequence
  misc_feature (1)..(13): U can be replaced by T. Any base can be replaced with a modified base.
uauccauccaugu

SEQ ID NO: 322 - RNA, 13 nt, Artificial Sequence
  Synthetic sequence
  misc_feature (1)..(13): U can be replaced by T. Any base can be replaced with a modified base.
cuauccauccaug

SEQ ID NO: 323 - RNA, 13 nt, Artificial Sequence
  Synthetic sequence
  misc_feature (1)..(13): U can be replaced by T. Any base can be replaced with a modified base.
ucuauccauccau

SEQ ID NO: 324 - RNA, 13 nt, Artificial Sequence
  Synthetic sequence
  misc_feature (1)..(13): U can be replaced by T. Any base can be replaced with a modified base.
aucuauccaucca

SEQ ID NO: 325 - RNA, 13 nt, Artificial Sequence
  Synthetic sequence
  misc_feature (1)..(13): U can be replaced by T. Any base can be replaced with a modified base.
caucuauccaucc

SEQ ID NO: 326 - RNA, 13 nt, Artificial Sequence
  Synthetic sequence
  misc_feature (1)..(13): U can be replaced by T. Any base can be replaced with a modified base.
ccaucuauccauc

SEQ ID NO: 327 - RNA, 13 nt, Artificial Sequence
  Synthetic sequence
  misc_feature (1)..(13): U can be replaced by T. Any base can be replaced with a modified base.
uccaucuauccau

SEQ ID NO: 328 - RNA, 13 nt, Artificial Sequence
  Synthetic sequence
  misc_feature (1)..(13): U can be replaced by T. Any base can be replaced with a modified base.
auccaucuaucca

SEQ ID NO: 329 - RNA, 13 nt, Artificial Sequence
  Synthetic sequence
  misc_feature (1)..(13): U can be replaced by T. Any base can be replaced with a modified base.
cauccaucuaucc

SEQ ID NO: 330 - RNA, 13 nt, Artificial Sequence
  Synthetic sequence
  misc_feature (1)..(13): U can be replaced by T. Any base can be replaced with a modified base.
ucauccaucuauc

SEQ ID NO: 331 - RNA, 13 nt, Artificial Sequence
  Synthetic sequence
  misc_feature (1)..(13): U can be replaced by T. Any base can be replaced with a modified base.
cucauccaucuau

SEQ ID NO: 332 - RNA, 13 nt, Artificial Sequence
  Synthetic sequence
  misc_feature (1)..(13): U can be replaced by T. Any base can be replaced with a modified base.
acucauccaucua

SEQ ID NO: 333 - RNA, 13 nt, Artificial Sequence
  Synthetic sequence
  misc_feature (1)..(13): U can be replaced by T. Any base can be replaced with a modified base.
aacucauccaucu

SEQ ID NO: 334 - RNA, 13 nt, Artificial Sequence
  Synthetic sequence
  misc_feature (1)..(13): U can be replaced by T. Any base can be replaced with a modified base.
caacucauccauc

SEQ ID NO: 335 - RNA, 13 nt, Artificial Sequence
  Synthetic sequence
  misc_feature (1)..(13): U can be replaced by T. Any base can be replaced with a modified base.
ccaacucauccau

SEQ ID NO: 336 - RNA, 13 nt, Artificial Sequence
  Synthetic sequence
  misc_feature (1)..(13): U can be replaced by T. Any base can be replaced with a modified base.
accaacucaucca

SEQ ID NO: 337 - RNA, 13 nt, Artificial Sequence
  Synthetic sequence
  misc_feature (1)..(13): U can be replaced by T. Any base can be replaced with a modified base.
caccaacucaucc

SEQ ID NO: 338 - RNA, 13 nt, Artificial Sequence
  Synthetic sequence
  misc_feature (1)..(13): U can be replaced by T. Any base can be replaced with a modified base.
ccaccaacucauc

SEQ ID NO: 339 - RNA, 13 nt, Artificial Sequence
  Synthetic sequence
  misc_feature (1)..(13): U can be replaced by T. Any base can be replaced with a modified base.
cccaccaacucau

SEQ ID NO: 340 - RNA, 13 nt, Artificial Sequence
  Synthetic sequence
  misc_feature (1)..(13): U can be replaced by T. Any base can be replaced with a modified base.
acccaccaacuca

SEQ ID NO: 341 - RNA, 13 nt, Artificial Sequence
  Synthetic sequence
  misc_feature (1)..(13): U can be replaced by T. Any base can be replaced with a modified base.
uacccaccaacuc

SEQ ID NO: 342 - RNA, 13 nt, Artificial Sequence
  Synthetic sequence
  misc_feature (1)..(13): U can be replaced by T. Any base can be replaced with a modified base.
cuacccaccaacu

SEQ ID NO: 343 - RNA, 13 nt, Artificial Sequence
  Synthetic sequence
  misc_feature (1)..(13): U can be replaced by T. Any base can be replaced with a modified base.
ucuacccaccaac

SEQ ID NO: 344 - RNA, 13 nt, Artificial Sequence
  Synthetic sequence
  misc_feature (1)..(13): U can be replaced by T. Any base can be replaced with a modified base.
aucuacccaccaa

SEQ ID NO: 345 - RNA, 13 nt, Artificial Sequence
  Synthetic sequence
  misc_feature (1)..(13): U can be replaced by T. Any base can be replaced with a modified base.
aaucuacccacca

SEQ ID NO: 346 - RNA, 13 nt, Artificial Sequence
  Synthetic sequence
  misc_feature (1)..(13): U can be replaced by T. Any base can be replaced with a modified base.
gaaucuacccacc

SEQ ID NO: 347 - RNA, 13 nt, Artificial Sequence
  Synthetic sequence
cgaaucuacccac

SEQ ID NO: 348 - RNA, 13 nt, Artificial Sequence
  Synthetic sequence
acgaaucuaccca

SEQ ID NO: 349 - RNA, 13 nt, Artificial Sequence
  Synthetic sequence
  misc_feature (1)..(13): U can be replaced by T. Any base can be replaced with a modified base.
cacgaaucuaccc

SEQ ID NO: 350 - RNA, 13 nt, Artificial Sequence
  Synthetic sequence
  misc_feature (1)..(13): U can be replaced by T. Any base can be replaced with a modified base.
ccacgaaucuacc

SEQ ID NO: 351 - RNA, 13 nt, Artificial Sequence
  Synthetic sequence
  misc_feature (1)..(13): U can be replaced by T. Any base can be replaced with a modified base.
gccacgaaucuac

SEQ ID NO: 352 - RNA, 13 nt, Artificial Sequence
  Synthetic sequence
  misc_feature (1)..(13): U can be replaced by T. Any base can be replaced with a modified base.
agccacgaaucua

SEQ ID NO: 353 - RNA, 13 nt, Artificial Sequence
  Synthetic sequence
  misc_feature (1)..(13): U can be replaced by T. Any base can be replaced with a modified base.
cagccacucacaa

SEQ ID NO: 354 - RNA, 13 nt, Artificial Sequence
  Synthetic sequence
  misc_feature (1)..(13): U can be replaced by T. Any base can be replaced with a modified base.
ccagccacucaca

SEQ ID NO: 355 - RNA, 13 nt, Artificial Sequence
  Synthetic sequence
  misc_feature (1)..(13): U can be replaced by T. Any base can be replaced with a modified base.
accagccacucac

SEQ ID NO: 356 - RNA, 13 nt, Artificial Sequence
  Synthetic sequence
  misc_feature (1)..(13): U can be replaced by T. Any base can be replaced with a modified base.
caccagccacuca

SEQ ID NO: 357 - RNA, 13 nt, Artificial Sequence
  Synthetic sequence
  misc_feature (1)..(13): U can be replaced by T. Any base can be replaced with a modified base.
ccaccagccacuc

SEQ ID NO: 358 - RNA, 13 nt, Artificial Sequence
  Synthetic sequence
  misc_feature (1)..(13): U can be replaced by T. Any base can be replaced with a modified base.
uccaccagccacu

SEQ ID NO: 359 - RNA, 13 nt, Artificial Sequence
  Synthetic sequence
  misc_feature (1)..(13): U can be replaced by T. Any base can be replaced with a modified base.
guccaccagccac

SEQ ID NO: 360 - RNA, 13 nt, Artificial Sequence
  Synthetic sequence
  misc_feature (1)..(13): U can be replaced by T. Any base can be replaced with a modified base.
uguccaccagcca

SEQ ID NO: 361 - RNA, 13 nt, Artificial Sequence
  Synthetic sequence
  misc_feature (1)..(13): U can be replaced by T. Any base can be replaced with a modified base.
cuguccaccagcc

SEQ ID NO: 362 - RNA, 13 nt, Artificial Sequence
  Synthetic sequence
  misc_feature (1)..(13): U can be replaced by T. Any base can be replaced with a modified base.
ucuguccaccagc

SEQ ID NO: 363 - RNA, 13 nt, Artificial Sequence
  Synthetic sequence
  misc_feature (1)..(13): U can be replaced by T. Any base can be replaced with a modified base.
gucuguccaccag

SEQ ID NO: 364 - RNA, 13 nt, Artificial Sequence
  Synthetic sequence
  misc_feature (1)..(13): U can be replaced by T. Any base can be replaced with a modified base.
cgucuguccacca

SEQ ID NO: 365 - RNA, 13 nt, Artificial Sequence
  Synthetic sequence
  misc_feature (1)..(13): U can be replaced by T. Any base can be replaced with a modified base.
ucgucuguccacc

SEQ ID NO: 366 - RNA, 13 nt, Artificial Sequence
  Synthetic sequence
  misc_feature (1)..(13): U can be replaced by T. Any base can be replaced with a modified base.
uucgucuguccac

SEQ ID NO: 367 - RNA, 13 nt, Artificial Sequence
  Synthetic sequence
  misc_feature (1)..(13): U can be replaced by T. Any base can be replaced with a modified base.
uuucgucugucca

SEQ ID NO: 368 - RNA, 13 nt, Artificial Sequence
  Synthetic sequence
  misc_feature (1)..(13): U can be replaced by T. Any base can be replaced with a modified base.
uuuucgucugucc

SEQ ID NO: 369 - RNA, 13 nt, Artificial Sequence
  Synthetic sequence
  misc_feature (1)..(13): U can be replaced by T. Any base can be replaced with a modified base.
uuuuucgucuguc

SEQ ID NO: 370 - RNA, 13 nt, Artificial Sequence
  Synthetic sequence
  misc_feature (1)..(13): U can be replaced by T. Any base can be replaced with a modified base.
auuuuucgucugu

SEQ ID NO: 371 - RNA, 13 nt, Artificial Sequence
  Synthetic sequence
  misc_feature (1)..(13): U can be replaced by T. Any base can be replaced with a modified base.
cauuuuucgucug

SEQ ID NO: 372 - RNA, 13 nt, Artificial Sequence
  Synthetic sequence
  misc_feature (1)..(13): U can be replaced by T. Any base can be replaced with a modified base.
ccauuuuucgucu

SEQ ID NO: 373 - RNA, 13 nt, Artificial Sequence
  Synthetic sequence
  misc_feature (1)..(13): U can be replaced by T. Any base can be replaced with a modified base.
uccauuuuucguc

SEQ ID NO: 374 - RNA, 13 nt, Artificial Sequence
  Synthetic sequence
  misc_feature (1)..(13): U can be replaced by T. Any base can be replaced with a modified base.
auccauuuuucgu

SEQ ID NO: 375 - RNA, 13 nt, Artificial Sequence
  Synthetic sequence
  misc_feature (1)..(13): U can be replaced by T. Any base can be replaced with a modified base.
cauccauuuuucg

SEQ ID NO: 376 - RNA, 13 nt, Artificial Sequence
  Synthetic sequence
  misc_feature (1)..(13): U can be replaced by T. Any base can be replaced with a modified base.
ccauccauuuuuc

SEQ ID NO: 377 - RNA, 13 nt, Artificial Sequence
  Synthetic sequence
  misc_feature (1)..(13): U can be replaced by T. Any base can be replaced with a modified base.
accauccauuuuu

SEQ ID NO: 378 - RNA, 13 nt, Artificial Sequence
  Synthetic sequence
  misc_feature (1)..(13): U can be replaced by T. Any base can be replaced with a modified base.
cccauccauuuuu

SEQ ID NO: 379 - RNA, 13 nt, Artificial Sequence
  Synthetic sequence
  misc_feature (1)..(13): U can be replaced by T. Any base can be replaced with a modified base.
aaccauccauuuu

SEQ ID NO: 380 - RNA, 13 nt, Artificial Sequence
  Synthetic sequence
  misc_feature (1)..(13): U can be replaced by T. Any base can be replaced with a modified base.
acccauccauuuu

SEQ ID NO: 381 - RNA, 13 nt, Artificial Sequence
  Synthetic sequence
  misc_feature (1)..(13): U can be replaced by T. Any base can be replaced with a modified base.
caaccauccauuu

SEQ ID NO: 382 - RNA, 13 nt, Artificial Sequence
  Synthetic sequence
  misc_feature (1)..(13): U can be replaced by T. Any base can be replaced with a modified base.
cacccauccauuu

SEQ ID NO: 383 - RNA, 13 nt, Artificial Sequence
  Synthetic sequence
  misc_feature (1)..(13): U can be replaced by T. Any base can be replaced with a modified base.
ccaaccauccauu

SEQ ID NO: 384 - RNA, 13 nt, Artificial Sequence
  Synthetic sequence
  misc_feature (1)..(13): U can be replaced by T. Any base can be replaced with a modified base.
ccacccauccauu

SEQ ID NO: 385 - RNA, 13 nt, Artificial Sequence
  Synthetic sequence
  misc_feature (1)..(13): U can be replaced by T. Any base can be replaced with a modified base.
uccaaccauccau

SEQ ID NO: 386 - RNA, 13 nt, Artificial Sequence
  Synthetic sequence
  misc_feature (1)..(13): U can be replaced by T. Any base can be replaced with a modified base.
uccacccauccau

SEQ ID NO: 387 - RNA, 13 nt, Artificial Sequence
  Synthetic sequence
  misc_feature (1)..(13): U can be replaced by T. Any base can be replaced with a modified base.
auccaaccaucca

SEQ ID NO: 388 - RNA, 13 nt, Artificial Sequence
  Synthetic sequence
  misc_feature (1)..(13): U can be replaced by T. Any base can be replaced with a modified base.
auccacccaucca

SEQ ID NO: 389 - RNA, 13 nt, Artificial Sequence
  Synthetic sequence
  misc_feature (1)..(13): U can be replaced by T. Any base can be replaced with a modified base.
uauccaaccaucc

SEQ ID NO: 390 - RNA, 13 nt, Artificial Sequence
  Synthetic sequence
  misc_feature (1)..(13): U can be replaced by T. Any base can be replaced with a modified base.
uauccacccaucc

SEQ ID NO: 391 - RNA, 13 nt, Artificial Sequence
  Synthetic sequence
  misc_feature (1)..(13): U can be replaced by T. Any base can be replaced with a modified base.
uuauccaaccauc

SEQ ID NO: 392 - RNA, 13 nt, Artificial Sequence
  Synthetic sequence
  misc_feature (1)..(13): U can be replaced by T. Any base can be replaced with a modified base.
uuauccacccauc

SEQ ID NO: 393 - RNA, 13 nt, Artificial Sequence
  Synthetic sequence
  misc_feature (1)..(13): U can be replaced by T. Any base can be replaced with a modified base.
uuuauccaaccau

SEQ ID NO: 394 - RNA, 13 nt, Artificial Sequence
  Synthetic sequence
  misc_feature (1)..(13): U can be replaced by T. Any base can be replaced with a modified base.
uuuauccacccau

SEQ ID NO: 395 - RNA, 13 nt, Artificial Sequence
  Synthetic sequence
  misc_feature (1)..(13): U can be replaced by T. Any base can be replaced with a modified base.
auuuauccaacca

SEQ ID NO: 396 - RNA, 13 nt, Artificial Sequence
  Synthetic sequence
  misc_feature (1)..(13): U can be replaced by T. Any base can be replaced with a modified base.
auuuauccaccca

SEQ ID NO: 397 - RNA, 13 nt, Artificial Sequence
  Synthetic sequence
  misc_feature (1)..(13): U can be replaced by T. Any base can be replaced with a modified base.
aauuuauccaacc

SEQ ID NO: 398 - RNA, 13 nt, Artificial Sequence
  Synthetic sequence
  misc_feature (1)..(13): U can be replaced by T. Any base can be replaced with a modified base.
aauuuauccaccc

SEQ ID NO: 399 - RNA, 13 nt, Artificial Sequence
  Synthetic sequence
  misc_feature (1)..(13): U can be replaced by T. Any base can be replaced with a modified base.
caauuuauccaac

SEQ ID NO: 400 - RNA, 13 nt, Artificial Sequence
  Synthetic sequence
  misc_feature (1)..(13): U can be replaced by T. Any base can be replaced with a modified base.
caauuuauccacc

SEQ ID NO: 401 - RNA, 13 nt, Artificial Sequence
  Synthetic sequence
  misc_feature (1)..(13): U can be replaced by T. Any base can be replaced with a modified base.
ucaauuuauccaa

SEQ ID NO: 402 - RNA, 13 nt, Artificial Sequence
  Synthetic sequence
  misc_feature (1)..(13): U can be replaced by T. Any base can be replaced with a modified base.
ucaauuuauccac

SEQ ID NO: 403 - RNA, 13 nt, Artificial Sequence
  Synthetic sequence
  misc_feature (1)..(13): U can be replaced by T. Any base can be replaced with a modified base.
aucaauuuaucca

SEQ ID NO: 404 - RNA, 13 nt, Artificial Sequence
  Synthetic sequence
  misc_feature (1)..(13): U can be replaced by T. Any base can be replaced with a modified base.
caucaauuuaucc

SEQ ID NO: 405 - RNA, 13 nt, Artificial Sequence
  Synthetic sequence
  misc_feature (1)..(13): U can be replaced by T. Any base can be replaced with a modified base.
ccaucaauuuauc

SEQ ID NO: 406 - RNA, 13 nt, Artificial Sequence
  Synthetic sequence
  misc_feature (1)..(13): U can be replaced by T. Any base can be replaced with a modified base.
cccaucaauuuau

SEQ ID NO: 407 - RNA, 13 nt, Artificial Sequence
  Synthetic sequence
  misc_feature (1)..(13): U can be replaced by T. Any base can be replaced with a modified base.
acccaucaauuua

SEQ ID NO: 408 - RNA, 13 nt, Artificial Sequence
  Synthetic sequence
  misc_feature (1)..(13): U can be replaced by T. Any base can be replaced with a modified base.
cacccaucaauuu

SEQ ID NO: 409 - RNA, 13 nt, Artificial Sequence
  Synthetic sequence
  misc_feature (1)..(13): U can be replaced by T. Any base can be replaced with a modified base.
ccacccaucaauu

SEQ ID NO: 410 - RNA, 13 nt, Artificial Sequence
  Synthetic sequence
  misc_feature (1)..(13): U can be replaced by T. Any base can be replaced with a modified base.
uccacccaucaau

SEQ ID NO: 411 - RNA, 13 nt, Artificial Sequence
  Synthetic sequence
  misc_feature (1)..(13): U can be replaced by T. Any base can be replaced with a modified base.
auccacccaucaa

SEQ ID NO: 412 - RNA, 13 nt, Artificial Sequence
  Synthetic sequence
  misc_feature (1)..(13): U can be replaced by T. Any base can be replaced with a modified base.
cauccacccauca

SEQ ID NO: 413 - RNA, 13 nt, Artificial Sequence
  Synthetic sequence
  misc_feature (1)..(13): U can be replaced by T. Any base can be replaced with a modified base.
ccauccacccauc

SEQ ID NO: 414 - RNA, 13 nt, Artificial Sequence
  Synthetic sequence
  misc_feature (1)..(13): U can be replaced by T. Any base can be replaced with a modified base.
uccauccacccau

SEQ ID NO: 415 - RNA, 13 nt, Artificial Sequence
  Synthetic sequence
  misc_feature (1)..(13): U can be replaced by T. Any base can be replaced with a modified base.
auccauccaccca

SEQ ID NO: 416 - RNA, 13 nt, Artificial Sequence
  Synthetic sequence
  misc_feature (1)..(13): U can be replaced by T. Any base can be replaced with a modified base.
cauccauccaccc

SEQ ID NO: 417 - RNA, 13 nt, Artificial Sequence
  Synthetic sequence
  misc_feature (1)..(13): U can be replaced by T. Any base can be replaced with a modified base.
ccauccauccacc

SEQ ID NO: 418 - RNA, 13 nt, Artificial Sequence
  Synthetic sequence
  misc_feature (1)..(13): U can be replaced by T. Any base can be replaced with a modified base.
accauccauccac

SEQ ID NO: 419 - RNA, 13 nt, Artificial Sequence
  Synthetic sequence
  misc_feature (1)..(13): U can be replaced by T. Any base can be replaced with a modified base.
aaccauccaucca

SEQ ID NO: 420 - RNA, 13 nt, Artificial Sequence
  Synthetic sequence
  misc_feature (1)..(13): U can be replaced by T. Any base can be replaced with a modified base.
caaccauccaucc

SEQ ID NO: 421 - RNA, 13 nt, Artificial Sequence
  Synthetic sequence
  misc_feature (1)..(13): U can be replaced by T. Any base can be replaced with a modified base.
ccaaccauccauc

SEQ ID NO: 422 - RNA, 13 nt, Artificial Sequence
  Synthetic sequence
  misc_feature (1)..(13): U can be replaced by T. Any base can be replaced with a modified base.
accaaccauccau

SEQ ID NO: 423 - RNA, 13 nt, Artificial Sequence
  Synthetic sequence
  misc_feature (1)..(13): U can be replaced by T. Any base can be replaced with a modified base.
aaccaaccaucca

SEQ ID NO: 424 - RNA, 13 nt, Artificial Sequence
  Synthetic sequence
  misc_feature (1)..(13): U can be replaced by T. Any base can be replaced with a modified base.
caaccaaccaucc

SEQ ID NO: 425 - RNA, 13 nt, Artificial Sequence
  Synthetic sequence
  misc_feature (1)..(13): U can be replaced by T. Any base can be replaced with a modified base.
acaaccaaccauc

SEQ ID NO: 426 - RNA, 13 nt, Artificial Sequence
  Synthetic sequence
  misc_feature (1)..(13): U can be replaced by T. Any base can be replaced with a modified base.
uacaaccaaccau

SEQ ID NO: 427 - RNA, 13 nt, Artificial Sequence
  Synthetic sequence
  misc_feature (1)..(13): U can be replaced by T. Any base can be replaced with a modified base.
uccgcaacuuaau

SEQ ID NO: 428 - RNA, 13 nt, Artificial Sequence
  Synthetic sequence
  misc_feature (1)..(13): U can be replaced by T. Any base can be replaced with a modified base.
auccgcaacuuaa

SEQ ID NO: 429 - RNA, 13 nt, Artificial Sequence
  Synthetic sequence
  misc_feature (1)..(13): U can be replaced by T. Any base can be replaced with a modified base.
gauccgcaacuua

SEQ ID NO: 430 - RNA, 13 nt, Artificial Sequence
  Synthetic sequence
  misc_feature (1)..(13): U can be replaced by T. Any base can be replaced with a modified base.
ugauccgcaacuu

SEQ ID NO: 431 - RNA, 13 nt, Artificial Sequence
  Synthetic sequence
  misc_feature (1)..(13): U can be replaced by T. Any base can be replaced with a modified base.
uugauccgcaacu

SEQ ID NO: 432 - RNA, 13 nt, Artificial Sequence
  Synthetic sequence
  misc_feature (1)..(13): U can be replaced by T. Any base can be replaced with a modified base.
auugauccgcaac

SEQ ID NO: 433 - RNA, 13 nt, Artificial Sequence
  Synthetic sequence
  misc_feature (1)..(13): U can be replaced by T. Any base can be replaced with a modified base.
cauugauccgcaa

SEQ ID NO: 434 - RNA, 13 nt, Artificial Sequence
  Synthetic sequence
  misc_feature (1)..(13)
acauugauccgca

SEQ ID NO: 435 - RNA, 13 nt, Artificial Sequence
  Synthetic sequence
  misc_feature (1)..(13): U can be replaced by T. Any base can be replaced with a modified base.
uacauugauccgc

SEQ ID NO: 436 - RNA, 13 nt, Artificial Sequence
  Synthetic sequence
  misc_feature (1)..(13): U can be replaced by T. Any base can be replaced with a modified base.
auacauugauccg

SEQ ID NO: 437 - RNA, 13 nt, Artificial Sequence
  Synthetic sequence
  misc_feature (1)..(13): U can be replaced by T. Any base can be replaced with a modified base.
cauacauugaucc

SEQ ID NO: 438 - RNA, 13 nt, Artificial Sequence
  Synthetic sequence
  misc_feature (1)..(13): U can be replaced by T. Any base can be replaced with a modified base.
ccauacauugauc

SEQ ID NO: 439 - RNA, 13 nt, Artificial Sequence
  Synthetic sequence
  misc_feature (1)..(13): U can be replaced by T. Any base can be replaced with a modified base.
cccauacauugau

SEQ ID NO: 440 - RNA, 13 nt, Artificial Sequence
  Synthetic sequence
  misc_feature (1)..(13): U can be replaced by T. Any base can be replaced with a modified base.
ucccauacauuga

SEQ ID NO: 441 - RNA, 13 nt, Artificial Sequence
  Synthetic sequence
  misc_feature (1)..(13): U can be replaced by T. Any base can be replaced with a modified base.
aucccauacauug

SEQ ID NO: 442 - RNA, 13 nt, Artificial Sequence
  Synthetic sequence
  misc_feature (1)..(13): U can be replaced by T. Any base can be replaced with a modified base.
caucccauacauu

SEQ ID NO: 443 - RNA, 13 nt, Artificial Sequence
  Synthetic sequence
  misc_feature (1)..(13): U can be replaced by T. Any base can be replaced with a modified base.
ccaucccauacau

SEQ ID NO: 444 - RNA, 13 nt, Artificial Sequence
  Synthetic sequence
  misc_feature (1)..(13): U can be replaced by T. Any base can be replaced with a modified base.
uccaucccauaca

SEQ ID NO: 445 - RNA, 13 nt, Artificial Sequence
  Synthetic sequence
  misc_feature (1)..(13): U can be replaced by T. Any base can be replaced with a modified base.
auccaucccauac

SEQ ID NO: 446 - RNA, 13 nt, Artificial Sequence
  Synthetic sequence
  misc_feature (1)..(13): U can be replaced by T. Any base can be replaced with a modified base.
cauccaucccaua

SEQ ID NO: 447 - RNA, 13 nt, Artificial Sequence
  Synthetic sequence
  misc_feature (1)..(13): U can be replaced by T. Any base can be replaced with a modified base.
ucauccaucccau

SEQ ID NO: 448 - RNA, 13 nt, Artificial Sequence
  Synthetic sequence
  misc_feature (1)..(13): U can be replaced by T. Any base can be replaced with a modified base.
uucauccauccca

SEQ ID NO: 449 - RNA, 13 nt, Artificial Sequence
  Synthetic sequence
  misc_feature (1)..(13): U can be replaced by T. Any base can be replaced with a modified base.
auucauccauccc

SEQ ID NO: 450 - RNA, 13 nt, Artificial Sequence
  Synthetic sequence
  misc_feature (1)..(13): U can be replaced by T. Any base can be replaced with a modified base.
cauucauccaucc

SEQ ID NO: 451 - RNA, 13 nt, Artificial Sequence
  Synthetic sequence
  misc_feature (1)..(13): U can be replaced by T. Any base can be replaced with a modified base.
ccauucauccauc

SEQ ID NO: 452 - RNA, 13 nt, Artificial Sequence
  Synthetic sequence
  misc_feature (1)..(13): U can be replaced by T. Any base can be replaced with a modified base.
uccauucauccau

SEQ ID NO: 453 - RNA, 13 nt, Artificial Sequence
  Synthetic sequence
  misc_feature (1)..(13): U can be replaced by T. Any base can be replaced with a modified base.
auccauucaucca

SEQ ID NO: 454 - RNA, 12 nt, Artificial Sequence
  Synthetic sequence
  misc_feature (1)..(13): U can be replaced by T. Any base can be replaced with a modified base.
cauccauucauc

SEQ ID NO: 455 - RNA, 13 nt, Artificial Sequence
  Synthetic sequence
  misc_feature (1)..(13): U can be replaced by T. Any base can be replaced with a modified base.
ccauccauucauc

SEQ ID NO: 456 - RNA, 13 nt, Artificial Sequence
  Synthetic sequence
  misc_feature (1)..(13): U can be replaced by T. Any base can be replaced with a modified base.
uccauccauucau

SEQ ID NO: 457 - RNA, 13 nt, Artificial Sequence
  Synthetic sequence
  misc_feature (1)..(13): U can be replaced by T. Any base can be replaced with a modified base.
auccauccauuca

SEQ ID NO: 458 - RNA, 13 nt, Artificial Sequence
  Synthetic sequence
  misc_feature (1)..(13): U can be replaced by T. Any base can be replaced with a modified base.
cauccauccauuc

SEQ ID NO: 459 - RNA, 13 nt, Artificial Sequence
  Synthetic sequence
  misc_feature (1)..(13): U can be replaced by T. Any base can be replaced with a modified base.
ccauccauccauu

SEQ ID NO: 460 - RNA, 13 nt, Artificial Sequence
  Synthetic sequence
  misc_feature (1)..(13): U can be replaced by T. Any base can be replaced with a modified base.
uccauccauccau

SEQ ID NO: 461 - RNA, 13 nt, Artificial Sequence
  Synthetic sequence
  misc_feature (1)..(13): U can be replaced by T. Any base can be replaced with a modified base.
auccauccaucca

SEQ ID NO: 462 - RNA, 13 nt, Artificial Sequence
  Synthetic sequence
  misc_feature (1)..(13): U can be replaced by T. Any base can be replaced with a modified base.
cauccauccaucc

SEQ ID NO: 463 - RNA, 13 nt, Artificial Sequence
  Synthetic sequence
  misc_feature (1)..(13): U can be replaced by T. Any base can be replaced with a modified base.
ccauccauccauc

SEQ ID NO: 464 - RNA, 13 nt, Artificial Sequence
  Synthetic sequence
  misc_feature (1)..(13): U can be replaced by T. Any base can be replaced with a modified base.
acauccauccauc

SEQ ID NO: 465 - RNA, 13 nt, Artificial Sequence
  Synthetic sequence
  misc_feature (1)..(13): U can be replaced by T. Any base can be replaced with a modified base.
cacauccauccau

SEQ ID NO: 466 - RNA, 13 nt, Artificial Sequence
  Synthetic sequence
  misc_feature (1)..(13): U can be replaced by T. Any base can be replaced with a modified base.
acacauccaucca

SEQ ID NO: 467 - RNA, 13 nt, Artificial Sequence
  Synthetic sequence
  misc_feature (1)..(13): U can be replaced by T. Any base can be replaced with a modified base.
cacacauccaucc

SEQ ID NO: 468 - RNA, 13 nt, Artificial Sequence
  Synthetic sequence
  misc_feature (1)..(13): U can be replaced by T. Any base can be replaced with a modified base.
ccacacauccauc

SEQ ID NO: 469 - RNA, 13 nt, Artificial Sequence
  Synthetic sequence
  misc_feature (1)..(13): U can be replaced by T. Any base can be replaced with a modified base.
accacacauccau

SEQ ID NO: 470 - RNA, 13 nt, Artificial Sequence
  Synthetic sequence
  misc_feature (1)..(13): U can be replaced by T. Any base can be replaced with a modified base.
aaccacacaucca

SEQ ID NO: 471 - RNA, 13 nt, Artificial Sequence
  Synthetic sequence
  misc_feature (1)..(13): U can be replaced by T. Any base can be replaced with a modified base.
caaccacacaucc

SEQ ID NO: 472 - RNA, 13 nt, Artificial Sequence
  Synthetic sequence
  misc_feature (1)..(13): U can be replaced by T. Any base can be replaced with a modified base.
ucaaccacacauc

SEQ ID NO: 473 - RNA, 13 nt, Artificial Sequence
  Synthetic sequence
  misc_feature (1)..(13): U can be replaced by T. Any base can be replaced with a modified base.
uucaaccacacau

SEQ ID NO: 474 - RNA, 13 nt, Artificial Sequence
  Synthetic sequence
  misc_feature (1)..(13): U can be replaced by T. Any base can be replaced with a modified base.
auucaaccacaca

SEQ ID NO: 475 - RNA, 13 nt, Artificial Sequence
  Synthetic sequence
  misc_feature (1)..(13): U can be replaced by T. Any base can be replaced with a modified base.
ucuauccagcaau

SEQ ID NO: 476 - RNA, 13 nt, Artificial Sequence
  Synthetic sequence
  misc_feature (1)..(13): U can be replaced by T. Any base can be replaced with a modified base.
aucuauccagcaa

SEQ ID NO: 477 - RNA, 13 nt, Artificial Sequence
  Synthetic sequence
  misc_feature (1)..(13): U can be replaced by T. Any base can be replaced with a modified base.
caucuauccagca

SEQ ID NO: 478 - RNA, 13 nt, Artificial Sequence
  Synthetic sequence
  misc_feature (1)..(13): U can be replaced by T. Any base can be replaced with a modified base.
ucaucuauccagc

SEQ ID NO: 479 - RNA, 13 nt, Artificial Sequence
  Synthetic sequence
  misc_feature (1)..(13): U can be replaced by T. Any base can be replaced with a modified base.
aucaucuauccag

SEQ ID NO: 480 - RNA, 13 nt, Artificial Sequence
  Synthetic sequence
  misc_feature (1)..(13): U can be replaced by T. Any base can be replaced with a modified base.
aaucaucuaucca

SEQ ID NO: 481 - RNA, 13 nt, Artificial Sequence
  Synthetic sequence
  misc_feature (1)..(13): U can be replaced by T. Any base can be replaced with a modified base.
caaucaucuaucc

SEQ ID NO: 482 - RNA, 13 nt, Artificial Sequence
  Synthetic sequence
  misc_feature (1)..(13): U can be replaced by T. Any base can be replaced with a modified base.
ucaaucaucuauc

SEQ ID NO: 483 - RNA, 13 nt, Artificial Sequence
  Synthetic sequence
  misc_feature (1)..(13): U can be replaced by T. Any base can be replaced with a modified base.
gucaaucaucuau

SEQ ID NO: 484 - RNA, 13 nt, Artificial Sequence
  Synthetic sequence
  misc_feature (1)..(13): U can be replaced by T. Any base can be replaced with a modified base.
agucaaucaucua

SEQ ID NO: 485 - RNA, 13 nt, Artificial Sequence
  Synthetic sequence
  misc_feature (1)..(13): U can be replaced by T. Any base can be replaced with a modified base.
cagucaaucaucu

SEQ ID NO: 486 - RNA, 13 nt, Artificial Sequence
  Synthetic sequence
  misc_feature (1)..(13): U can be replaced by T. Any base can be replaced with a modified base.
ccagucaaucauc

SEQ ID NO: 487 - RNA, 13 nt, Artificial Sequence
  Synthetic sequence
  misc_feature (1)..(13): U can be replaced by T. Any base can be replaced with a modified base.
cccagucaaucau

SEQ ID NO: 488 - RNA, 13 nt, Artificial Sequence
  Synthetic sequence
  misc_feature (1)..(13): U can be replaced by T. Any base can be replaced with a modified base.
acccagucaauca

SEQ ID NO: 489 - RNA, 13 nt, Artificial Sequence
  Synthetic sequence
  misc_feature (1)..(13): U can be replaced by T. Any base can be replaced with a modified base.
cacccagucaauc

SEQ ID NO: 490 - RNA, 13 nt, Artificial Sequence
  Synthetic sequence
  misc_feature (1)..(13): U can be replaced by T. Any base can be replaced with a modified base.
ccacccagucaau

SEQ ID NO: 491 - RNA, 13 nt, Artificial Sequence
  Synthetic sequence
  misc_feature (1)..(13): U can be replaced by T. Any base can be replaced with a modified base.
uccacccagucaa

SEQ ID NO: 492 - RNA, 13 nt, Artificial Sequence
  Synthetic sequence
  misc_feature (1)..(13): U can be replaced by T. Any base can be replaced with a modified base.
auccacccaguca

SEQ ID NO: 493 - RNA, 13 nt, Artificial Sequence
  Synthetic sequence
  misc_feature (1)..(13): U can be replaced by T. Any base can be replaced with a modified base.
uauccacccaguc

SEQ ID NO: 494 - RNA, 13 nt, Artificial Sequence
  Synthetic sequence
  misc_feature (1)..(13): U can be replaced by T. Any base can be replaced with a modified base.
cuauccacccagu

SEQ ID NO: 495 - RNA, 13 nt, Artificial Sequence
  Synthetic sequence
  misc_feature (1)..(13): U can be replaced by T. Any base can be replaced with a modified base.
ucuauccacccag

SEQ ID NO: 496 - RNA, 13 nt, Artificial Sequence
  Synthetic sequence
  misc_feature (1)..(13): U can be replaced by T. Any base can be replaced with a modified base.
aucuauccaccca

SEQ ID NO: 497 - RNA, 13 nt, Artificial Sequence
  Synthetic sequence
  misc_feature (1)..(13): U can be replaced by T. Any base can be replaced with a modified base.
caucuauccaccc

SEQ ID NO: 498 - RNA, 13 nt, Artificial Sequence
  Synthetic sequence
  misc_feature (1)..(13): U can be replaced by T. Any base can be replaced with a modified base.
ucaucuauccacc

SEQ ID NO: 499 - RNA, 13 nt, Artificial Sequence
  Synthetic sequence
  misc_feature (1)..(13): U can be replaced by T. Any base can be replaced with a modified base.
uucaucuauccac

SEQ ID NO: 500 - RNA, 13 nt, Artificial Sequence
  Synthetic sequence
  misc_feature (1)..(13): U can be replaced by T. Any base can be replaced with a modified base.
uuucaucuaucca

SEQ ID NO: 501 - RNA, 13 nt, Artificial Sequence
  Synthetic sequence
auuucaucuaucc

SEQ ID NO: 502 - RNA, 13 nt, Artificial Sequence
  Synthetic sequence
  misc_feature (1)..(13): U can be replaced by T. Any base can be replaced with a modified base.
cauuucaucuauc

SEQ ID NO: 503 - RNA, 13 nt, Artificial Sequence
  Synthetic sequence
  misc_feature (1)..(13): U can be replaced by T. Any base can be replaced with a modified base.
acauuucaucuau

SEQ ID NO: 504 - RNA, 13 nt, Artificial Sequence
  Synthetic sequence
  misc_feature (1)..(13): U can be replaced by T. Any base can be replaced with a modified base.
aacauuucaucua

SEQ ID NO: 505 - RNA, 13 nt, Artificial Sequence
  Synthetic sequence
  misc_feature (1)..(13): U can be replaced by T. Any base can be replaced with a modified base.
caacauuucaucu

SEQ ID NO: 506 - RNA, 13 nt, Artificial Sequence
  Synthetic sequence
  misc_feature (1)..(13): U can be replaced by T. Any base can be replaced with a modified base.
ucuggggcagcuu

SEQ ID NO: 507 - RNA, 13 nt, Artificial Sequence
  Synthetic sequence
  misc_feature (1)..(13): U can be replaced by T. Any base can be replaced with a modified base.
gucuggggcagcu

SEQ ID NO: 508 - RNA, 13 nt, Artificial Sequence
  Synthetic sequence
  misc_feature (1)..(13): U can be replaced by T. Any base can be replaced with a modified base.
ggucuggggcagc

SEQ ID NO: 509 - RNA, 13 nt, Artificial Sequence
  Synthetic sequence
  misc_feature (1)..(13): U can be replaced by T. Any base can be replaced with a modified base.
gggucuggggcag

SEQ ID NO: 510 - RNA, 13 nt, Artificial Sequence
  Synthetic sequence
  misc_feature (1)..(13): U can be replaced by T. Any base can be replaced with a modified base.
agggucuggggca

SEQ ID NO: 511 - RNA, 13 nt, Artificial Sequence
  Synthetic sequence
  misc_feature (1)..(13): U can be replaced by T. Any base can be replaced with a modified base.
cagggucuggggc

SEQ ID NO: 512 - RNA, 13 nt, Artificial Sequence
  Synthetic sequence
  misc_feature (1)..(13): U can be replaced by T. Any base can be replaced with a modified base.
ccagggucugggg

SEQ ID NO: 513 - RNA, 13 nt, Artificial Sequence
  Synthetic sequence
  misc_feature (1)..(13): U can be replaced by T. Any base can be replaced with a modified base.
cccagggucuggg

SEQ ID NO: 514 - RNA, 13 nt, Artificial Sequence
  Synthetic sequence
  misc_feature (1)..(13): U can be replaced by T. Any base can be replaced with a modified base.
ucccagggucugg

SEQ ID NO: 515 - RNA, 13 nt, Artificial Sequence
  Synthetic sequence
  misc_feature (1)..(13): U can be replaced by T. Any base can be replaced with a modified base.
gucccagggucug

SEQ ID NO: 516 - RNA, 13 nt, Artificial Sequence
  Synthetic sequence
  misc_feature (1)..(13): U can be replaced by T. Any base can be replaced with a modified base.
ggucccagggucu

SEQ ID NO: 517 - RNA, 13 nt, Artificial Sequence
  Synthetic sequence
  misc_feature (1)..(13): U can be replaced by T. Any base can be replaced with a modified base.
aggucccaggguc

SEQ ID NO: 518 - RNA, 13 nt, Artificial Sequence
  Synthetic sequence
  misc_feature (1)..(13): U can be replaced by T. Any base can be replaced with a modified base.
gaggucccagggu

SEQ ID NO: 519 - RNA, 13 nt, Artificial Sequence
  Synthetic sequence
  misc_feature (1)..(13): U can be replaced by T. Any base can be replaced with a modified base.
agaggucccaggg

SEQ ID NO: 520 - RNA, 13 nt, Artificial Sequence
  Synthetic sequence
  misc_feature (1)..(13): U can be replaced by T. Any base can be replaced with a modified base.
cagaggucccagg

SEQ ID NO: 521 - RNA, 13 nt, Artificial Sequence
  Synthetic sequence
  misc_feature (1)..(13): U can be replaced by T. Any base can be replaced with a modified base.
ucagaggucccag

SEQ ID NO: 522 - RNA, 13 nt, Artificial Sequence
  Synthetic sequence
  misc_feature (1)..(13): U can be replaced by T. Any base can be replaced with a modified base.
gucagagguccca

SEQ ID NO: 523 - RNA, 13 nt, Artificial Sequence
  Synthetic sequence
  misc_feature (1)..(13): U can be replaced by T. Any base can be replaced with a modified base.
ggucagagguccc

SEQ ID NO: 524 - RNA, 13 nt, Artificial Sequence
  Synthetic sequence
  misc_feature (1)..(13): U can be replaced by T. Any base can be replaced with a modified base.
gggucagaggucc

SEQ ID NO: 525 - RNA, 13 nt, Artificial Sequence
  Synthetic sequence
  misc_feature (1)..(13): U can be replaced by T. Any base can be replaced with a modified base.
ggggucagagguc

SEQ ID NO: 526 - RNA, 13 nt, Artificial Sequence
  Synthetic sequence
  misc_feature (1)..(13): U can be replaced by T. Any base can be replaced with a modified base.
gggggucagaggu

SEQ ID NO: 527 - RNA, 13 nt, Artificial Sequence
  Synthetic sequence
  misc_feature (1)..(13): U can be replaced by T. Any base can be replaced with a modified base.
cgggggucagagg

SEQ ID NO: 528 - RNA, 13 nt, Artificial Sequence
  Synthetic sequence
  misc_feature (1)..(13): U can be replaced by T. Any base can be replaced with a modified base.
ccgggggucagag

SEQ ID NO: 529 - RNA, 13 nt, Artificial Sequence
  Synthetic sequence
  misc_feature (1)..(13): U can be replaced by T. Any base can be replaced with a modified base.
gccgggggucaga

SEQ ID NO: 530 - RNA, 13 nt, Artificial Sequence
  Synthetic sequence
  misc_feature (1)..(13): U can be replaced by T. Any base can be replaced with a modified base.
cgccgggggucag

SEQ ID NO: 531 - RNA, 13 nt, Artificial Sequence
  Synthetic sequence
  misc_feature (1)..(13): U can be replaced by T. Any base can be replaced with a modified base.
ucgccggggguca

SEQ ID NO: 532 - RNA, 13 nt, Artificial Sequence
  Synthetic sequence
  misc_feature (1)..(13): U can be replaced by T. Any base can be replaced with a modified base.
gucgccggggguc

SEQ ID NO: 533 - RNA, 13 nt, Artificial Sequence
  Synthetic sequence
  misc_feature (1)..(13): U can be replaced by T. Any base can be replaced with a modified base.
ggucgccgggggu

SEQ ID NO: 534 - RNA, 13 nt, Artificial Sequence
  Synthetic sequence
  misc_feature (1)..(13): U can be replaced by T. Any base can be replaced with a modified base.
gggucgccggggg

SEQ ID NO: 535 - RNA, 13 nt, Artificial Sequence
  Synthetic sequence
  misc_feature (1)..(13): U can be replaced by T. Any base can be replaced with a modified base.
ggggucgccgggg

SEQ ID NO: 536 - RNA, 13 nt, Artificial Sequence
  Synthetic sequence
  misc_feature (1)..(13): U can be replaced by T. Any base can be replaced with a modified base.
aggggucgccggg

SEQ ID NO: 537 - RNA, 13 nt, Artificial Sequence
  Synthetic sequence
  misc_feature (1)..(13): U can be replaced by T. Any base can be replaced with a modified base.
aaggggucgccgg

SEQ ID NO: 538 - RNA, 13 nt, Artificial Sequence
  Synthetic sequence
  misc_feature (1)..(13): U can be replaced by T. Any base can be replaced with a modified base.
caaggggucgccg

SEQ ID NO: 539 - RNA, 13 nt, Artificial Sequence
  Synthetic sequence
  misc_feature (1)..(13): U can be replaced by T. Any base can be replaced with a modified base.
gcaaggggucgcc

SEQ ID NO: 540 - RNA, 13 nt, Artificial Sequence
  Synthetic sequence
  misc_feature (1)..(13): U can be replaced by T. Any base can be replaced with a modified base.
ugcaaggggucgc

SEQ ID NO: 541 - RNA, 13 nt, Artificial Sequence
  Synthetic sequence
  misc_feature (1)..(13): U can be replaced by T. Any base can be replaced with a modified base.
gugcaaggggucg

SEQ ID NO: 542 - RNA, 13 nt, Artificial Sequence
  Synthetic sequence
  misc_feature (1)..(13): U can be replaced by T. Any base can be replaced with a modified base.
agugcaagggguc

SEQ ID NO: 543 - RNA, 13 nt, Artificial Sequence
  Synthetic sequence
  misc_feature (1)..(13): U can be replaced by T. Any base can be replaced with a modified base.
gagugcaaggggu

SEQ ID NO: 544 - RNA, 13 nt, Artificial Sequence
  Synthetic sequence
  misc_feature (1)..(13): U can be replaced by T. Any base can be replaced with a modified base.
agagugcaagggg

SEQ ID NO: 545 - RNA, 13 nt, Artificial Sequence
  Synthetic sequence
  misc_feature (1)..(13): U can be replaced by T. Any base can be replaced with a modified base.
gagagugcaaggg

SEQ ID NO: 546 - RNA, 13 nt, Artificial Sequence
  Synthetic sequence
  misc_feature (1)..(13): U can be replaced by T. Any base can be replaced with a modified base.
ggagagugcaagg

SEQ ID NO: 547 - RNA, 80 nt, Homo sapiens
augaguagauaaaaggauggauggagagaugggugaguacauggauggauagauggaugaguugguggguagauucgugc

SEQ ID NO: 548 - RNA, 80 nt, Homo sapiens
augaguagauaaaagcauggauggagagaugggugaguacauggauggauagauggaugaguugguggguagauucgugc

SEQ ID NO: 549 - RNA, 40 nt, Homo sapiens
ggauggauggagagaugggugaguacauggauggauagau

SEQ ID NO: 550 - RNA, 40 nt, Homo sapiens
gcauggauggagagaugggugaguacauggauggauagau

SEQ ID NO: 551 - RNA, 29 nt, Homo sapiens
auggagagaugggugaguacauggaugga

SEQ ID NO: 552 - RNA, 97 nt, Homo sapiens
guucuuccuucuauuccagcccuaaccacucaggauugggccguuugugucuggguaugucucuuccagcugccuggguuuccuggaaagaacucuu

SEQ ID NO: 553 - RNA, 43 nt, Homo sapiens
gugucuggguaugucucuuccagcugccuggguuuccuggaaa

SEQ ID NO: 554 - RNA, 46 nt, Homo sapiens
ucaucagguucuuccuucuauuccagcccuaaccacucaggauugg

SEQ ID NO: 555 - DNA, 21 bp, Artificial Sequence
  Synthetic sequence
  modified_base (1)..(21): * = a phosphorothioate, + = LNA
  modified_base (1)..(21): phosphorothioate linkage, LNA at positions 1, 4, 7, 10, 13, 16, 19
cacccatctctccatccatgc

SEQ ID NO: 556 - DNA, 21 bp, Artificial Sequence
  Synthetic sequence
  modified_base (1)..(21): phosphorthioate backbone, and LNA at positions 1, 4, 7, 10, 13, 16 and 19
  modified_base (1)..(21): phosphorthioate linkage, and LNA at positions 1, 4, 7, 10, 13, 16 and 19
cacccatctctccatccatcc

SEQ ID NO: 557 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
ctcacccatctctccatccat

SEQ ID NO: 558 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
tgtactcacccatctctccat

SEQ ID NO: 559 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
tccatgtactcacccatctct

SEQ ID NO: 560 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
tccatccatgtactcacccat

SEQ ID NO: 561 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
atctatccatccatgtactca

SEQ ID NO: 562 - DNA, 17 bp, Artificial Sequence
  Synthetic sequence
  modified_base (1)..(17): phosphorthioate backbone, LNA at postions 1, 4, 7, 10, 13, 16 and 17
  modified_base (1)..(17): phosphorthioate linkage, LNA at postions 1, 4, 7, 10, 13, 16 and 17
tcacccatctctccatc

SEQ ID NO: 563 - DNA, 17 bp, Artificial Sequence
  Synthetic sequence
  modified_base (1)..(17): phosphorothioate linkage, LNA at 1, 3, 5, 7, 9, 11, 13, 15 and 17
tcacccatctctccatc

SEQ ID NO: 564 - DNA, 17 bp, Artificial Sequence
  Synthetic sequence
  modified_base (1)..(17): phosphorthioate backbone, LNA bases at 1, 4, 7, 10, 13, 16 and 17
  modified_base (1)..(17): phosphorothioate linkage, LNA bases at 1, 4, 7, 10, 13, 16 and 17
gtactcacccatctctc

SEQ ID NO: 565 - DNA, 17 bp, Artificial Sequence
  Synthetic sequence
  modified_base (1)..(17): phosphorthioate backbone, LNA at positins 1, 3, 5, 7, 9, 11, 13, 15 and 17
gtactcacccatctctc

SEQ ID NO: 566 - DNA, 13 bp, Artificial Sequence
  Synthetic sequence
  modified_base (1)..(13): phosphorthioate backbone, LNA bases at 2, 4, 6, 8, 10 and 12
  modified_base (1)..(13): phosphorthioate linkage, LNA bases at 2, 4, 6, 8, 10 and 12
cacccatctctcc

SEQ ID NO: 567 - RNA, 25 nt, Artificial Sequence
  Synthetic sequence
  modified_base (1)..(25): phosphorthioate backbone and 2-OMe-RNA at all positions
  modified_base (1)..(25): phosphorthioate linkage and 2-OMe-RNA at all positions
cauguacucacccaucucuccaucc

SEQ ID NO: 568 - RNA, 30 nt, Artificial Sequence
  Synthetic sequence
  modified_base (1)..(30): phosphorthioate backbone, all bases substitued with 2--O-methyl-RNA
  modified_base (1)..(30): phosphorthioate linkage, all bases substitued with 2--O-methyl-RNA
auccauguacucacccaucucuccauccau

SEQ ID NO: 569 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
caggcagctggaagagacata

SEQ ID NO: 570 - DNA, 21 bp, Artificial Sequence
  Synthetic sequence
  modified_base (1)..(21): phosphorothioate linkage, LNA  at positions 2, 4, 7, 10, 13, 16, 19 and 21
aacccaggcagctggaagaga

SEQ ID NO: 571 - DNA, 21 bp, Artificial Sequence
  Synthetic sequence
  modified_base (1)..(21): phosphorthioate backbone, LNA at positions 1, 4, 7, 10, 13, 16, 19 and 21
  modified_base (1)..(21): phosphorthioate linkage, LNA at positions 1, 4, 7, 10, 13, 16, 19 and 21
ggaaacccaggcagctggaag

SEQ ID NO: 572 - DNA, 13 bp, Artificial Sequence
  Synthetic sequence
agttttcaattcc

SEQ ID NO: 573 - DNA, 20 bp, Artificial Sequence
  Synthetic sequence
gacatcaaatcccgcgtgaa

SEQ ID NO: 574 - DNA, 20 bp, Artificial Sequence
  Synthetic sequence
cattgatgttggcgagcagg

SEQ ID NO: 575 - DNA, 20 bp, Artificial Sequence
  Synthetic sequence
cagacgatcattgaggtgcg

SEQ ID NO: 576 - DNA, 20 bp, Artificial Sequence
  Synthetic sequence
atacttggaggagaggcagg

SEQ ID NO: 577 - DNA, 18 bp, Artificial Sequence
  Synthetic sequence
caagcgaactgacaaatc

SEQ ID NO: 578 - DNA, 17 bp, Artificial Sequence
  Synthetic sequence
ctgggatcttcacgacc

SEQ ID NO: 579 - DNA, 21 bp, Artificial Sequence
  Synthetic sequence
  modified_base (1)..(21): phosphorothioate linkage, LNA at bases 1, 4, 7, 10, 13, 16, 19, and 21
tagggctggaatagaaggaag

SEQ ID NO: 580 - DNA, 21 bp, Artificial Sequence
  Synthetic sequence
  modified_base (1)..(21): phosphorothioate linkage, LNA at bases 1, 4, 7, 10, 13, 16, 19, and 21
tgagtggttagggctggaata